(19) United States
(12) Patent Application Publication
(10) Pub. No.: US 2010/0221277 A1
(43) Pub. Date: Sep. 2, 2010

(54) CHLAMYDIA PNEUMONIAE ANTIGENS

(76) Inventors: Andreas Meinke, Pressbaum (AT); Eszter Nagy, Vienna (AT); Birgit Noiges (Winkler), Vienna (AT)

Correspondence Address: FULBRIGHT & JAWORSKI L.L.P., 600 CONGRESS AVE., SUITE 2400, AUSTIN, TX, 78701, US

(21) Appl. No.: 12/605,211

(22) Filed: Oct. 23, 2009

Related U.S. Application Data
(62) Division of application No. 10/561,506, filed on Dec. 19, 2005, now abandoned.

(30) Foreign Application Priority Data
Jun. 17, 2003 (EP) 03450148.6

(51) Int. Cl. A61K 39/118; C07K 14/295; A61P 31/04

(52) U.S. Cl. 424/190.1; 530/350

(57) ABSTRACT

The present invention discloses isolated nucleic acid molecules encoding hyperimmune serum-reactive antigens and fragments thereof, including hyperimmune serum-reactive antigens and fragments thereof derived from C. pneumoniae. Methods for isolating such antigens and specific uses thereof are also disclosed.

### CROSS-REFERENCE TO RELATED APPLICATIONS

This application is a divisional application of U.S. application Ser. No. 10/561,506 filed on 19 Dec. 2005, which is a national phase application under 35 U.S.C. 371 of International Application No. PCT/EP2004/006460 filed 16 Jun. 2004, which claims priority to European Application No. 03450148.6 filed 17 Jun. 2003. The entire text of each of the above-referenced disclosures is specifically incorporated herein by reference without disclaimer.

### BACKGROUND

The present invention relates to isolated nucleic acid molecules, which encode antigens for Chlamydia pneumoniae, which are suitable for use in preparation of pharmaceutical medicaments for the prevention and treatment of bacterial infections caused by Chlamydia pneumoniae.

Chlamydia pneumoniae is an obligate intracellular bacterium and recognized as a significant human pathogen. It is a common cause of pneumoniae and upper respiratory tract disease in both, hospital and outpatient settings, accounting for approximately 7 to 10% of cases of community-acquired pneumoniae among adults {Montigiani, S. et al., 2002}. Infection with Chlamydia pneumoniae has also been associated with other respiratory tract diseases such as bronchitis, sinusitis, asthmatic bronchitis, adult-onset asthma, and chronic obstructive pulmonary disease {Murdin, A. et al., 2000}. Importantly, Chlamydia pneumoniae infection has also been associated with atherosclerosis and cardiovascular disease, which was indicated for example by seroepidemiologic studies or detection of C. pneumoniae in atherosclerotic plaques {Montigiani, S. et al., 2002}.

It was recently suggested that the Gram-negative Chlamydiaceae, a family of uncertain origin and the only members of the order Chlamydiales, can been divided into two genera, Chlamydia and Chlamydophila, by 16S rRNA phylogeny {Everett, K. et al., 1999}. According to this suggestion, three species are described within the genus Chlamydia: Chlamydia trachomatis, Chlamydia muridarum and Chlamydia suis. The species Chlamydia psittaci, pecorum and pneumoniae were suggested to be renamed to Chlamydophila psittaci, pecorum and pneumoniae. Nevertheless, bacteria of both genera share biological and biochemical properties. For the present invention, the newly suggested nomenclature has not been used yet, but for reasons of completeness it should be mentioned that the species Chlamydia pneumoniae and Chlamydophila pneumoniae are identical.

Sequencing of seven Chlamydiaceae genomes from four different species, has demonstrated that profound differences in host range and disease can be caused by fairly subtle variations in gene content {Read, T. et al., 2003}. The Chlamydiaceae are classified among the eubacteria as a well-isolated group, with only a very weak link to the planctomyces. The Chlamydiaceae therefore exhibit some unique characteristics within the eubacteria, in particular their development cycle and the structure of their membranes. They have a unique two-phase cell cycle: the elementary body, a small extracellular form, which attaches to the host and is phagocytosed. Subsequently, it is converted in the phagosome to the replicative intracellular form, the reticulate body. As obligate intracellular bacteria, the Chlamydiaceae multiply in eukaryotic cells at the expense of their energy reserves and nucleotide pools; they are responsible for a wide variety of diseases in mammals and birds.

The species Chlamydia trachomatis is the best characterized. Besides a murine strain, it is divided into two groups which are distinguishable by the nature of the diseases for which they are responsible: trachoma, genital attack and venereal lymphogranulomatosis. There are fifteen human serotypes of Chlamydia trachomatis (A, K) and LGV (L1, L2, L3). Strains A to C are mainly found in eye infections, whereas strains D to K and LGV are essentially responsible for genital entry infections. It should be mentioned that the LGV strains are responsible for systemic diseases. Historically, the characterization of the Chlamydia trachomatis microorganism was only successfully carried out in 1957, after a series of isolations in cell cultures.

The species Chlamydia psittaci infects many animals, in particular birds, and is transmissible to humans. It is responsible for atypical pneumonia, for hepatic and renal dysfunction, for endocarditis and for conjunctivitis.

Chlamydia pecorum does not infect humans, but is rather a pathogen of ruminants.

It was in 1983 that Chlamydia pneumoniae was recognized as a human pathogen {Grayston, J. et al., 1986}. Thereafter, special attention has been paid to this bacterium and it is estimated {Gaydos, C. et al., 1994} that 10% of pneumonias, and 5% of bronchitides and sinusites are attributable to Chlamydia pneumoniae {Aldous, M. et al., 1992}. More recently, the association of this bacterium with the pathogenesis of asthmatic disease and of cardiovascular impairments is increasingly of interest.

Serological studies have shown that Chlamydia pneumoniae infection is common in children between 5 and 16 years of age. Before this age, it is rare to find antibodies and the best available data indicate that children begin to seroconvert at an age of about 5 years. The increase in the number of individuals carrying antibodies correlates then with age up to 20 years. Accordingly, 50% to 70% of adults are carriers of antibodies. Since the persistence of induced antibodies over time is limited to 3 or at most 5 years after a first infection, it is suggestive that frequent reinfection occurs during the entire lifespan. The annual seroconversion rate is about 6 to 8% between 8 and 16 years {Kuo, C. et al., 1995} and the seroprevalence of the disease before the age of 15 years is identical between both sexes. After this age, men are more frequently infected than women in all regions worldwide.

These Chlamydia infections are geographically highly widespread throughout the world {Tong, C. et al., 1993}, with the lowest infection rates observed in developed countries of the north such as Canada and the Scandinavian countries. In contrast, the highest prevalence rates are found in the less developed countries of tropical regions where the infection may occur before the age of 5 years. Humans are the only known reservoir for Chlamydia pneumoniae and it is probable that the infection is caused by person-to-person transmission by respiratory secretions {Aldous, M. et al., 1992}. The chain of transmission may also appear to be indirect {Kleemola, M. et al., 1988}, suggestive of an infection caused by an effective transmission and of the possibility that also asymptomatic carriers exist, which could be an explanation of the high prevalence of the disease. This is also in accordance with the finding that Chlamydia pneumoniae can survive for up to 30 hours in a hostile environment {Falsey, A. et al., 1993 }, although the infectivity of the microorganism in the open air decreases rapidly under conditions of high relative humidity. The period of incubation is with several weeks significantly longer than that of many other respiratory pathogenic bacteria.

The main clinical manifestations caused by Chlamydia pneumoniae are essentially respiratory diseases. Pneumonia and bronchitis are the most frequent, because they are clinically obvious and the infectious agent may be identified. Isolation of the etiologic agent is difficult though and paired acute- and convalescent-phase sera are required to confirm the diagnosis using antibody tests. The asymptomatic diseases caused by Chlamydia pneumoniae are probably numerous (e.g. {Grayston, J., 1992}). Other syndromes such as sinusitis, purulent otitis media {Ogawa, H. et al., 1992), or pharyngitis have been described, as well as infections with respiratory impairments similar to asthma {Hahn, D. et al., 1991}. Chlamydia pneumoniae has also been associated with sarcoidosis, with erythema nodosum {Sundelof, B. et al., 1993} and one case of Guillain-Barre syndrome has been described {Haidl, S. et al., 1992}. The involvement of Chlamydia pneumoniae in Reiter's syndrome has also been evaluated {Braun, J. et al., 1994}.

Cardiovascular diseases are the major cause of death in the countries of the Western world. The association of Chlamydia pneumoniae with the development of cardiovascular diseases such as coronary heart disease and myocardial infarction was first suspected due to the observation of high antibody levels in of patients with heart disease (e.g. {Shor, A. et al., 1992}). In addition, anatomicopathological and microbiological studies were able to detect in the vessels. Studies from several countries have also shown that Chlamydia pneumoniae infection correlates with atheromatous impairments in patients (e.g. {Grayston, J., 1996}). Thus it also appears that the bacterium is more frequently found in old atheromatous lesions, than in early lesions, but that it is not found in subjects free of atheromatous disease. It is therefore supported by these studies that the atheroma plaque is very strongly correlated with the presence of Chlamydia pneumoniae. Nevertheless, the role that the bacterium plays in vascular pathology is not yet defined.

For the treatment of Chlamydia pneumoniae infections, there are only limited data available from controlled clinical studies. Similar to Lymes disease and mycoplasma infection and due to the intracellular nature of C. pneumoniae, long term antimicrobial treatment is needed. This extensive antimicrobial treatment is required for eradication of C. pneumoniae from macrophages and endothelial cells of infected arteries. Unlike penicillin, ampicillin or the sulphamides, antibiotics such as erythromycin, tetracycline, doxycycline, ofloxacin, clinafloxacin, ciprofloxacin, azithromycin, clindamycin, and minocycline show an antibiotic activity in vitro against Chlamydia pneumoniae. However, any treatment at high doses should be continued for several weeks in order to avoid a recurrence of the infection. Accordingly, the use of two new macrolides, clarithromycin and azithromycin, whose diffusion, bioavailability and half-life allow shorter and better tolerated cures, is nowadays preferred. Unfortunately, many conventional treatments against Chlamydia still fail, resulting in a significant rate of recurrence and morbidity. In the absence of definitive proof based on the results of clinical studies, an effective, without recurrences, and well-tolerated treatment of Chlamydia pneumoniae infections therefore remains desirable.

A very important issue is the development of a specific and sensitive diagnosis, which can be carried out conveniently and rapidly, allowing early screening for the infection. Unfortunately, methods based on Chlamydia pneumoniae culture are slow and require a considerable know-how because of the difficulty involved in the collection, preservation and storage of the strain under appropriate conditions. On the other hand, methods based on antigen detection (EIA, DFA) or on nucleic acid amplification (PCR) provide tests, which are more suitable for laboratory practice. A reliable, sensitive and convenient test, which allows distinction between serogroups and a fortiori between Chlamydia pneumoniae species, is highly desirable. This is all the more important, because the symptoms of Chlamydia pneumoniae infection appear slowly, and because not all of the pathologies associated with these infections have yet been identified. In addition, an association is suspected between these infections and serious chronic infections, asthma or atherosclerosis. Although sensitive and specific tests based on antigen detection have been developed, there remains a need for standardized PCR based detection protocols and tests {Dowell, S. et al., 2001).

Chlamydial infections are often chronic and recurrent; suggesting that protective immunity against Chlamydia is weak and not necessarily bactericidal or sterilizing. There are currently no available vaccines against chlamydial infections. Although the number of studies and of animal models developed is high, the antigens used have not induced sufficient protective immunity to lead to the development of human vaccines.

A more detailed understanding of the biology of Chlamydia pneumoniae, the interactions of the bacteria with their hosts, their escape from immune defenses of the host in particular, but also their involvement in the development of the associated pathologies, will allow a better control; treatment or prevention of Chlamydia caused diseases. It is therefore essential, to use novel molecular tools, which allow to develop new preventive and therapeutic treatments, new diagnostic methods and new vaccine strategies which are specific, effective and tolerated.

The present inventors have developed a method for identification, isolation and production of hyperimmune serum reactive antigens from a specific pathogen, especially from Staphylococcus aureus and Staphylococcus epidermidis (WO 02/059148). However, given the differences in biological property, pathogenic function and genetic background, Chlamydia pneumoniae is very distinctive from Staphylococcus strains. Importantly, the selection of sera for the identification of antigens from C. pneumoniae is different from that applied to the S. aureus screens. Infections with Chlamydia pneumoniae are detected and diagnosed by serology, since the pathogen is not culturable with routine microbiological methods. We have selected patients' sera having high titer against C. pneumoniae detected by a standard Chlamydia ELISA kit routinely used in the clinic for diagnosis of acute, chronic and persistent infections caused by Chlamydia species. Our selection mainly relied on the presence of high affinity IgG antibodies, allowing the identification of patients in convalescent phase. The pre-selected sera having the highest titers were subsequently analyzed by immunoblotting to ensure antibody reactivities against multiple proteinaceous antigens present in C. pneumoniae. This approach for selection of human sera is basically very different from that used for S. aureus, where carriage or even disease cannot be always associated with high antibody levels.

The genomes of the two bacterial species C. pneumoniae and S. aureus by itself show a number of important differences. While the genome of S. aureus harbours 2.85 Mb, the genome of C. pneumoniae contains app. 1.23 Mb, less than half of the size of S. aureus and many other bacterial genomes. They have an average GC content of 33 and 40.6%, respectively and only 586 of the S. aureus genes have a match with a gene in C. pneumoniae with at least 40% identity on the amino acid level. This means that of the 1073 genes of C. pneumoniae less than 55% have a homologous sequence in S. aureus. In addition, the two bacterial species require not only different growth conditions and media for propagation, but C. pneumoniae is an obligate intracellular pathogen, while S. aureus mainly lives extracellularly. Furthermore, C. pneumoniae is a strictly human pathogen, but S. aureus can also be found infecting a range of warm-blooded animals. A list of the most important diseases, which can be inflicted by the two pathogens, is presented below. S. aureus causes mainly nosocomial, opportunistic infections: impetigo, folliculitis, abscesses, boils, infected lacerations, endocarditis, meningitis, septic arthritis, pneumonia, osteomyelitis, scalded skin syndrome (SSS), toxic shock syndrome. C. pneumoniae causes mainly pneumoniae and upper respiratory tract disease.

The complete genome sequence of a several isolates of C. pneumoniae, was determined by various institutions {Kalman, S. et al., 1999}; {Read, T. et al., 2000}; {Shirai, M. et al., 2000}; see also http://www.tignorg/tigr-scripts/CMR2/CMRHomePage.sp1). Although the two strains AR39 and CWL029 were isolated in the U.S.A. before 1987 and Japan in 1994, respectively, their sequence is to a high degree identical, indicating a divergence in recent human history. In addition to these three C. pneumoniae strains, the sequence of two C. trachomatis strains {Kalman, S. et al., 1999}; {Read, T. et al., 2000} and that of C. psittaci {Read, T. et al., 200356 have been determined.

The problem underlying the present invention was to provide means for the development of medicaments such as vaccines against C. pneumoniae infection. More particularly, the problem was to provide an efficient, relevant and comprehensive set of nucleic acid molecules or hyperimmune serum reactive antigens from C. pneumoniae that can be used for the manufacture of said medicaments.

Therefore, the present invention provides an isolated nucleic acid molecule encoding a hyperimmune serum reactive antigen or a fragment thereof comprising a nucleic acid sequence, which is selected from the group consisting of:
a) a nucleic acid molecule having at least 70% sequence identity to a nucleic acid molecule selected from Seq ID No 31-60.
b) a nucleic acid molecule which is complementary to the nucleic acid molecule of a),
c) a nucleic acid molecule comprising at least 15 sequential bases of the nucleic acid molecule of a) or b)
d) a nucleic acid molecule which anneals under stringent hybridization conditions to the nucleic acid molecule of a), b), or c)
e) a nucleic acid molecule which, but for the degeneracy of the genetic code, would hybridize to the nucleic acid molecule defined in a), b), c) or d).

According to a preferred embodiment of the present invention the sequence identity is at least 80%, preferably at least 95%, especially 100%.

Furthermore, the present invention provides an isolated nucleic acid molecule encoding a hyperimmune serum reactive antigen or a fragment thereof comprising a nucleic acid sequence selected from the group consisting of
a) a nucleic acid molecule having at least 96%, preferably at least 98%, especially 100% sequence identity to a nucleic acid molecule selected from Seq ID No 5, 7-8, 14-16, 18-22, 24-27, 29-30.
b) a nucleic acid molecule which is complementary to the nucleic acid molecule of a),
c) a nucleic acid molecule comprising at least 15 sequential bases of the nucleic acid molecule of a) or b)
d) a nucleic acid molecule which anneals under stringent hybridization conditions to the nucleic acid molecule of a), b) or c),
e) a nucleic acid molecule which, but for the degeneracy of the genetic code, would hybridize to the nucleic acid defined in a), b), c) or d).

Preferably, the nucleic acid molecule is DNA or RNA.

According to a preferred embodiment of the present invention, the nucleic acid molecule is isolated from a genomic DNA, especially from a C. pneumoniae genomic DNA.

According to the present invention a vector comprising a nucleic acid molecule according to any of the present invention is provided.

In a preferred embodiment the vector is adapted for recombinant expression of the hyperimmune serum reactive antigens or fragments thereof encoded by the nucleic acid molecule according to the present invention.

The present invention also provides a host cell comprising the vector according to the present invention.

According to another aspect the present invention further provides a hyperimmune serum-reactive antigen comprising an amino acid sequence being encoded by a nucleic acid molecule according to the present invention.

In a preferred embodiment the amino acid sequence (polypeptide) is selected from the group consisting of Seq ID No 91-120.

In another preferred embodiment the amino acid sequence (polypeptide) is selected from the group consisting of Seq ID No 65, 67-68, 74-76, 78-82, 84-87, 89-90.

According to a further aspect the present invention provides fragments of hyperimmune serum-reactive antigens selected from the group consisting of peptides comprising amino acid sequences of column “predicted immunogenic aa”, “Predicted class II restricted T-Cell epitopes/regions” “Predicted class I restricted T-Cell epitopes/regions”, and “location of identified immunogenic region” of Table 1; the serum reactive peptide epitopes of Table 2, especially peptides comprising amino acids 18-29, 60-78, 89-95, 100-105, 124-143, 166-180, 187-194, 196-208, 224-242, 285-294, 305-311, 313-320, 351-360, 368-373, 390-403, 411-429, 432-470, 483-489, 513-523, 535-543, 548-564, 579-587, 589-598, 604-612, 622-627, 632-648, 55-84, 190-207, 323-331, 370-390, 551-570, 606-614, 633-647, 39-129, 224-296 and 464-609 of Seq ID No 61; and fragments in 9 amino acid length starting from the position of: 60, 63, 67, 70, 126, 129, 133, 136, 169, 186, 200, 308, 371, 414, 421, 434, 444, 459, 503, 512, 532, 540, 547, 601, 625, 632, 634, 637, 99, 529, 25, 38, 59, 155, 278, 285, 412, 420, 441, 451, 457, 481, 506, 510, 524, 536, 539, 554, 578, 596, 638, 179 and 604 of Seq ID No 61; 4-29, 31-38, 46-64, 66-80, 109-115, 131-139, 152-160, 170-183, 198-234, 239-255, 267-290, 301-313, 318-324, 336-345, 350-365, 380-386, 65-82, 123-165, 268-290, 299-307, 320-329, 336-347, 76-103, 226-239 and 267-333 of Seq ID No 62; and fragments in 9 amino acid length starting from the position of: 4, 13, 69, 93, 149, 174, 273, 277, 298, 305, 312, 319, 375, 28, 303, 3, 58, 73, 100, 153, 191, 223, 227, 232, 251, 269, 286, 343, 374 and 238 of Seq ID No 62; 20-33, 35-43, 47-60, 77-92, 113-124, 137-145, 185-196, 66-75 and 92-214 of Seq ID No 63; and fragments in 9 amino acid length starting from the position of: 32, 48, 49, 113, 77, 118, 139, 185, 2, 24 and 120 of Seq ID No 63; 47-64, 137-155, 157-167, 182-198, 212-233, 247-259, 291-303, 315-337, 345-350, 355-368, 373-379, 58-72, 183-196, 249-261, 315-323, 334-342, 347-356, 358-366 and 6-188 of Seq ID No 64; and fragments in 9 amino acid length starting from the position of: 135, 160, 183, 184, 204, 249, 256, 293, 296, 318, 319, 356, 372, 94, 13, 60, 159, 163, 189, 204, 220, 233, 300, 333, 335, 356, 362, 198 and 289 of Seq ID No 64; 4-36, 43-49, 60-75, 96-107, 113-123, 132-172, 186-193, 217-229, 231-250, 260-282, 284-290, 298-312, 315-330, 5-38, 67-77, 113-127, 134-145, 147-156, 220-236, 271-283, 285-293, 296-304, 309-321 and 159-217 of Seq ID No 65; and fragments in 9 amino acid length starting from the position of: 3, 10, 14, 17, 24, 46, 59, 133, 155, 220, 270, 312, 233, 2, 22, 31, 36, 62, 65, 122, 140, 155, 162, 170, 189, 235, 248, 260, 286, 298, 156, 183 and 325 of Seq ID No 65; 5-26, 29-50, 52-61, 65-74, 89-96, 140-147, 153-162, 183-188, 191-197, 203-210, 213-225, 1-9, 30-38, 53-63, 70-78, 92-107, 141-149, 158-166, 174-191, 205-224 and 97-113 of Seq ID No 66; and fragments in 9 amino acid length starting from the position of: 31, 33, 39, 56, 63, 78, 119, 136, 196, 14, 35, 38, 55, 97, 98, 146, 156, 158, 215, 88 and 214 of Seq ID No 66; 31-36, 46-54, 65-80, 86-102, 168-175, 179-186, 188-194, 200-208, 210-216, 225-231, 243-257, 289-296, 362-387, 460-474, 476-486, 504-511, 518-525, 569-579, 581-600, 665-684, 688-694, 700-705, 717-735, 182-193, 202-211, 279-294, 311-319, 369-377, 468-476, 547-558, 579-587, 681-700, 731-740, 92-177 and 591-604 of Seq ID No 67; and fragments in 9 amino acid length starting from the position of: 28, 78, 285, 309, 321, 376, 379, 388, 468, 475, 479, 500, 571, 624, 668, 716, 360, 455, 669, 185, 190, 204, 264, 281, 292, 478, 502, 588, 675, 680, 716 and 730 of Seq ID No 67; 4-9, 17-24, 27-52, 66-77, 91-98, 104-124, 127-139, 178-199, 211-219, 221-228, 234-244, 246-255, 263-286, 303-312, 316-321, 337-346, 356-362, 367-372, 377-390, 402-416, 449-459, 465-479, 491-501, 503-508, 523-541, 551-558, 560-565, 31-69, 115-127, 132-143, 145-165, 176-187, 190-204, 212-220, 266-286, 304-316, 403-423, 440-456, 523-544 and 9-22 of Seq ID No 68; and fragments in 9 amino acid length starting from the position of: 17, 24, 31, 45, 53, 56, 63, 69, 107, 129, 150, 171, 178, 189, 191, 217, 255, 273, 277, 305, 312, 451, 458, 470, 478, 506, 522, 71, 379, 20, 29, 34, 44, 119, 133, 276, 284, 300, 328, 404, 465, 470, 529, 543, 182 and 551 of Seq ID No 68; 34-42, 52-63, 71-87, 112-120, 142-147, 154-159, 166-177, 180-197, 204-224, 237-256, 260-268, 280-286, 312-324, 338-343, 372-412, 456-463, 479-490, 494-504, 506-512, 518-524, 538-548, 562-573, 585-591, 597-606, 674-690, 703-712, 714-740, 749-766, 95-103, 114-123, 180-195, 205-220, 240-248, 370-400, 481-495, 588-596, 707-715, 750-765, 160-253 and 630-717 of Seq ID No 69; and fragments in 9 amino acid length starting from the position of: 179, 206, 209, 213, 216, 255, 286, 300, 304, 324, 365, 369, 373, 376, 377, 380, 381, 384, 562, 694, 720, 721, 729, 749, 752, 755, 197, 330, 559, 592, 600, 714, 751, 91, 111, 140, 167, 191, 315, 388, 393, 402, 458, 463, 587, 720, 762 and 748 of Seq ID No 69; 4-44, 50-55, 59-67, 73-83, 91-98, 101-109, 131-145, 230-236, 267-273, 293-300, 303-310, 349-354, 375-397, 404-416, 434-441, 445-452, 456-468, 479-485, 487-512, 544-568, 571-579, 593-599, 604-610, 614-621, 642-656, 665-678, 706-716, 729-736, 748-756, 780-795, 797-814, 827-844, 850-861, 864-882, 889-900, 906-933, 6-23, 28-36, 64-75, 134-150, 182-192, 227-236, 306-316, 340-350, 376-387, 421-435, 449-460, 527-535, 553-569, 587-595, 641-657, 668-676, 683-694, 743-755, 800-819, 843-865, 861-886, 894-915, 929-938 and 603-669 of Seq ID No 70; and fragments in 9 amino acid length starting from the position of: 7, 8, 15, 73, 80, 133, 134, 138, 182, 194, 271, 272, 298, 432, 438, 457, 458, 487, 490, 527, 548, 568, 616, 644, 647, 667, 741, 782, 801, 829, 866, 126, 259, 792, 15, 20, 133, 155, 160, 232, 299, 458, 464, 552, 558, 560, 605, 607, 654, 670, 672, 768, 810, 840, 852, 877, 900, 167, 380, 425, 593 and 907 of Seq ID No 70; 4-32, 73-82, 90-101, 116-132, 144-160, 171-182, 195-200, 227-234, 255-271, 293-300, 313-336, 344-350, 369-375, 381-398, 413-421, 436-465, 487-496, 503-508, 510-527, 538-546, 552-562, 608-614, 617-636, 663-674, 679-691, 705-730, 734-748, 769-807, 825-834, 848-861, 864-871, 891-902, 7-16, 90-107, 110-137, 170-187, 197-213, 233-251, 277-287, 291-314, 361-390, 412-425, 451-465, 489-498, 513-521, 570-580, 619-637, 662-679, 713-721, 725-733, 745-754, 766-781, 790-805, 817-834, 868-883, 888-903 and 529-542 of Seq ID No 71; and fragments in 9 amino acid length starting from the position of: 8, 23, 53, 57, 128, 169, 178, 239, 263, 290, 297, 310, 324, 331, 339, 365, 398, 436, 443, 450, 470, 485, 488, 513, 514, 520, 614, 669, 711, 723, 771, 824, 849, 895, 316, 861, 118, 135, 196, 225, 284, 290, 370, 454, 489, 492, 521, 557, 624, 632, 745, 778, 783, 850, 868, 910, 226 and 383 of Seq ID No 71; 10-18, 30-52, 63-70, 72-79, 96-133, 146-158, 168-175, 184-193, 203-210, 213-222, 227-234, 237-257, 263-273, 285-291, 297-312, 320-338, 359-378, 385-393, 395-410, 412-421, 490-510, 521-527, 540-548, 563-571, 573-585, 592-598, 615-620, 632-641, 652-661, 672-679, 704-711, 717-723, 729-736, 742-751, 766-778, 788-808, 817-824, 836-842, 34-56, 73-89, 103-130, 146-154, 184-205, 213-227, 245-257, 258-278, 292-316, 331-341, 358-369, 372-383, 388-397, 410-418, 503-514, 524-530, 548-556, 565-573, 584-595, 637-646, 656-663, 673-686, 734-742, 745-754, 757-768, 770-781, 816-828 and 14-101 of Seq ID No 72; and fragments in 9 amino acid length starting from the position of: 27, 32, 36, 65, 109, 112, 120, 127, 186, 249, 250, 262, 267, 297, 301, 353, 360, 367, 410, 418, 436, 465, 472, 505, 518, 522, 565, 576, 585, 638, 645, 650, 676, 687, 724, 745, 756, 763, 795, 164, 411, 510, 560, 569, 647, 766, 780, 14, 39, 48, 65, 74, 129, 175, 215, 217, 229, 230, 240, 253, 257, 262, 269, 308, 317, 322, 327, 352, 371, 372, 373, 374, 417, 443, 454, 472, 514, 525, 567, 629, 637, 657, 662, 683, 698, 731, 744, 752, 763, 769, 787, 790, 802, 815, 819, 26, 102, 381 and 704 of Seq ID No 72; 4-14, 20-33, 36-63, 71-93, 96-104, 106-117, 120-128, 131-147, 161-172, 174-186, 195-210, 212-247, 269-286, 288-301, 306-322, 324-332, 348-354, 356-363, 384-391, 35-66, 70-85, 107-118, 124-132, 165-179, 186-196, 197-205, 276-289, 292-300, 348-368, 369-381, 385-394 and 139-151 of Seq ID No 73; and fragments in 9 amino acid length starting from the position of: 34, 41, 50, 53, 109, 127, 134, 153, 165, 271, 286, 297, 340, 384, 80, 321, 334, 354, 33, 57, 110, 153, 178, 276, 284, 383, 79, 99 and 123 of Seq ID No 73; 12-20, 37-48, 51-58, 69-75, 86-98, 113-136, 141-161, 171-216, 222-254, 264-273, 291-301, 311-345, 351-361, 31-39, 40-55, 62-74, 121-137, 148-164, 170-178, 223-253, 309-329, 354-369 and 246-275 of Seq ID No 74; and fragments in 9 amino acid length starting from the position of: 46, 95, 103, 110, 143, 156, 178, 186, 190, 236, 242, 244, 291, 294, 315, 333, 353, 125, 183, 256, 326, 3, 68, 82, 102, 131, 177, 185, 190, 193, 223, 224, 244, 250, 295, 340, 349, 354, 88 and 89 of Seq ID No 74; 30-36, 50-56, 96-102, 110-116, 125-131, 162-174, 179-187, 189-201, 223-230, 232-239, 266-278, 320-328, 330-337, 339-350, 388-400, 408-413, 417-423, 435-447, 456-480, 499-524, 526-534, 53-62, 92-107, 192-203, 315-323, 436-452, 464-483, 502-524 and 61-138 of Seq ID No 75; and fragments in 9 amino acid length starting from the position of: 126, 174, 225, 267, 309, 316, 320, 337, 436, 466, 467, 473, 474, 14, 128, 143, 228, 347, 494, 2, 52, 112, 201, 209, 217, 230, 235, 236, 337, 381, 395, 413, 419, 454, 466, 510, 515 and 556 of Seq ID No 75; 7-32, 36-56, 77-82, 88-100, 117-144, 153-166, 173-180, 188-226, 256-297, 300-316, 323-337, 339-348, 361-384, 390-427, 438-455, 476-488, 516-523, 535-566, 580-586, 597-607, 615-621, 626-634, 639-649, 654-660, 668-673, 677-688, 707-714, 716-728, 730-742, 746-756, 763-772, 801-808, 820-829, 840-875, 882-888, 895-911, 914-920, 928-948, 953-961, 987-995, 999-1005, 1007-1026, 1053-1060, 1071-1079, 1082-1117, 1123-1129, 6-31, 37-48, 58-69, 90-105, 110-118, 134-142, 146-157, 210-220, 267-276, 291-300, 319-330, 362-372, 393-401, 405-421, 447-456, 463-471, 517-525, 574-582, 597-612, 618-626, 642-650, 656-668, 668-678, 683-695, 725-733, 778-791, 840-849, 894-917, 927-939, 954-963, 966-974, 978-998, 1010-1021, 1056-1067, 1070-1083, 1090-1104 and 325-389 of Seq ID No 76; and fragments in 9 amino acid length starting from the position of: 11, 18, 22, 41, 48, 86, 104, 156, 190, 197, 221, 286, 290, 334, 343, 345, 407, 442, 509, 538, 575, 596, 597, 598, 636, 678, 685, 723, 754, 757, 779, 818, 850, 857, 864, 893, 900, 901, 907, 918, 927, 934, 972, 988, 1018, 1025, 1034, 1048, 1065, 1072, 1089, 1094, 1101, 1108, 127, 336, 411, 806, 852, 28, 68, 90, 91, 93, 158, 293, 310, 350, 368, 380, 394, 425, 441, 461, 554, 569, 597, 628, 667, 684, 724, 737, 752, 761, 767, 804, 851, 897, 907, 933, 979, 1030, 1032, 1051, 1075, 1090, 1125, 133, 308, 502, 797, 939 and 960 of Seq ID No 76; 11-19, 34-53, 55-91, 113-119, 122-129, 131-140, 157-170, 173-179, 188-195, 200-206, 208-220, 222-232, 236-244, 250-265, 267-274, 282-290, 293-301, 317-323, 336-343, 355-361, 372-384, 33-54, 69-95, 210-221, 244-254, 257-269 and 324-351 of Seq ID No 77; and fragments in 9 amino acid length starting from the position of: 32, 37, 43, 47, 50, 53, 57, 64, 68, 71, 73, 74, 78, 80, 82, 113, 120, 155, 162, 194, 205, 209, 231, 235, 238, 252, 259, 266, 273, 280, 287, 294, 301, 308, 315, 333, 8, 16, 18, 66, 377, 36, 44, 81, 99, 124, 193, 261 and 319 of Seq ID No 77; 31-55, 58-64, 69-75, 81-90, 129-150, 154-167, 179-184, 189-208, 227-237, 248-271, 277-284, 313-340, 350-358, 361-368, 371-378, 384-390, 418-425, 438-444, 455-468, 487-506, 514-523, 525-550, 558-569, 572-578, 588-598, 607-618, 645-651, 653-665, 672-684, 708-715, 717-742, 754-771, 776-782, 786-802, 806-817, 1-9, 31-46, 52-61, 60-78, 132-148, 182-199, 214-229, 249-264, 280-293, 320-341, 347-355, 386-411, 486-502, 553-575, 624-634, 673-689, 690-700, 702-714, 721-735, 736-746, 757-777, 788-798, 810-818 and 90-100 of Seq ID No 78; and fragments in 9 amino acid length starting from the position of: 51, 82, 139, 186, 193, 197, 200, 239, 248, 249, 250, 257, 311, 325, 326, 520, 555, 556, 589, 606, 651, 716, 723, 730, 737, 758, 761, 772, 788, 39, 41, 569, 695, 709, 783, 51, 60, 89, 110, 141, 207, 216, 295, 301, 395, 404, 518, 527, 555, 568, 593, 596, 673, 691, 722, 757, 772, 790, 799, 130, 131, 179, 402, 414 and 701 of Seq ID No 78;13-19, 22-28, 61-67, 74-81, 86-103, 110-122, 141-155, 162-169, 171-177, 181-186, 192-199, 201-207, 225-238, 246-263, 273-279, 287-300, 307-313, 331-336, 351-367, 370-376, 380-392, 395-402, 415-422, 424-451, 454-465, 473-492, 496-509, 515-523, 541-547, 569-582, 589-601, 613-636, 638-647, 653-679, 702-714, 721-729, 739-748, 768-779, 799-813, 821-828, 832-840, 847-853, 857-873, 886-892, 894-905, 917-926, 958-971, 974-981, 983-989, 997-1004, 1006-1032, 1034-1049, 1054-1061, 1063-1069, 1073-1081, 1083-1095, 1097-1115, 1122-1132, 1143-1153, 1164-1171, 1178-1185, 1193-1213, 1216-1251, 1258-1272, 1277-1283, 1305-1317, 1324-1330, 1333-1355, 1383-1390, 25-43, 81-92, 111-141, 150-159, 213-220, 222-242, 243-254, 256-267, 276-288, 289-307, 381-397, 398-409, 422-438, 441-464, 485-500, 515-528, 542-553, 569-585, 591-601, 639-649, 656-664, 709-719, 725-734, 739-753, 841-850, 883-893, 902-911, 912-926, 935-948, 960-969, 976-984, 994-1008, 1037-1047, 1073-1085, 1100-1108, 1124-1134, 1167-1179, 1194-1203, 1220-1254, 1258-1277, 1308-1319, 1348-1366 and 273-290 of Seq ID No 79; and fragments in 9 amino acid length starting from the position of: 107, 110, 112, 133, 152, 200, 204, 223, 244, 251, 271, 289, 291, 305, 323, 360, 380, 407, 422, 428, 440, 491, 507, 512, 536, 616, 625, 628, 648, 650, 665, 668, 748, 768, 784, 797, 801, 826, 858, 859, 903, 910, 913, 925, 932, 959, 960, 968, 993, 1008, 1020, 1068, 1072, 1138, 1141, 1142, 1193, 1201, 1218, 1226, 1237, 1261, 1271, 1311, 1348, 1349, 1377, 126, 375, 433, 477, 608, 658, 852, 1106, 1121, 1303, 1362, 24, 102, 151, 164, 169, 211, 229, 245, 274, 279, 285, 333, 348, 361, 382, 391, 397, 428, 447, 453, 480, 496, 590, 591, 595, 615, 623, 629, 638, 664, 669, 672, 738, 744, 775, 789, 840, 910, 917, 939, 966, 977, 1057, 1084, 1096, 1119, 1127, 1128, 1145, 1163, 1167, 1202, 1214, 1238, 1244, 1260, 1279, 1335, 145, 355, 961, 1053, 1103 and 1245 of Seq ID No 79; 16-23, 25-47, 49-59, 64-72, 79-91, 95-105, 113-122, 133-145, 148-162, 169-176, 179-188, 190-200, 202-218, 232-239, 250-283, 299-333, 337-344, 349-355, 364-406, 430-437, 439-449, 452-460, 464-490, 492-503, 505-530, 533-562, 12-21, 28-39, 52-67, 115-124, 189-204, 224-232, 234-242, 263-284, 302-322, 363-385, 389-397, 446-463, 479-488, 513-522, 528-552 and 401-419 of Seq ID No 80; and fragments in 9 amino acid length starting from the position of: 23, 30, 58, 78, 84, 97, 98, 120, 123, 133, 162, 169, 189, 215, 218, 236, 309, 312, 316, 365, 372, 384, 388, 391, 426, 446, 453, 465, 466, 478, 508, 513, 515, 523, 530, 536, 543, 554, 333, 467, 13, 19, 115, 130, 181, 195, 225, 262, 270, 275, 311, 313, 325, 342, 390, 391, 398, 461, 530, 116, 188 and 229 of Seq ID No 80; 8-16, 36-54, 59-76, 85-92, 104-124, 137-180, 199-248, 255-298, 300-307, 324-339, 356-373, 381-393, 402-442, 448-455, 18-27, 36-56, 101-120, 145-158, 165-173, 179-189, 239-255, 255-270, 330-346, 355-375, 383-394, 403-421 and 83-232 of Seq ID No 81; and fragments in 9 amino acid length starting from the position of: 5, 102, 149, 156, 160, 164, 185, 186, 204, 208, 211, 221, 232, 264, 270, 273, 277, 280, 284, 287, 317, 329, 362, 387, 398, 402, 404, 422, 429, 431, 449, 37, 298, 359, 9, 17, 35, 40, 41, 105, 111, 146, 166, 234, 279, 343, 384, 412 and 365 of Seq ID No 81; 29-69, 71-88, 95-104, 106-130, 143-189, 205-232, 24-40, 46-64, 65-79, 83-105, 121-129, 144-199, 206-236 and 182-199 of Seq ID No 82; and fragments in 9 amino acid length starting from the position of: 30, 37, 66, 77, 81, 84, 112, 118, 141, 144, 145, 146, 149, 150, 153, 167, 169, 170, 178, 196, 213, 215, 220, 13, 21, 39, 44, 62, 75, 78, 97, 119, 124, 145, 148, 154, 177, 190, 207, 22 and 216 of Seq ID No 82; 4-46, 51-66, 77-88, 102-110, 115-126, 142-148, 171-181, 183-192, 202-212, 227-234, 251-261, 263-278, 283-316, 319-325, 336-352, 362-371, 386-393, 399-406, 410-425, 427-437, 441-450, 457-464, 471-476, 490-496, 514-521, 549-557, 571-578, 601-611, 618-623, 627-646, 657-670, 672-689, 696-704, 726-740, 742-756, 765-776, 778-784, 792-801, 822-836, 862-868, 875-881, 887-898, 914-919, 941-948, 963-969, 971-978, 996-1004, 1007-1016, 1036-1051, 1068-1080, 1082-1090, 1092-1098, 1104-1127, 1135-1144, 1156-1177, 1181-1195, 1197-1206, 1214-1231, 1243-1263, 1278-1284, 1295-1303, 1305-1323, 1337-1346, 1355-1374, 1376-1383, 1406-1423, 1455-1463, 1465-1489, 1506-1518, 1527-1552, 1555-1570, 1581-1589, 1-28, 109-124, 208-220, 261-280, 286-296, 310-324, 398-405, 425-433, 439-454, 504-517, 535-555, 570-591, 599-614, 620-630, 691-699, 711-719, 729-739, 751-760, 783-791, 843-855, 878-886, 890-900, 940-955, 984-1003, 1007-1026, 1065-1073, 1106-1122, 1136-1149, 1188-1198, 1203-1211, 1227-1235, 1249-1256, 1298-1308, 1374-1392, 1398-1409, 1414-1429, 1436-1444, 1456-1490, 1504-1521, 1530-1547, 1592-1609 and 911-935 of Seq ID No 83; and fragments in 9 amino acid length starting from the position of: 26, 33, 79, 170, 200, 265, 290, 297, 302, 304, 333, 334, 377, 412, 414, 415, 431, 436, 458, 465, 481, 494, 536, 546, 568, 605, 678, 690, 697, 703, 724, 729, 730, 735, 737, 767, 776, 797, 840, 861, 938, 968, 999, 1072, 1079, 1085, 1094, 1113, 1160, 1163, 1180, 1188, 1195, 1217, 1245, 1250, 1273, 1302, 1358, 1362, 1363, 1401, 1408, 1465, 1469, 1481, 1507, 178, 960, 1034, 6, 21, 38, 159, 204, 248, 260, 306, 337, 349, 384, 425, 438, 458, 481, 502, 521, 546, 605, 690, 730, 731, 819, 860, 915, 946, 967, 1007, 1018, 1065, 1113, 1187, 1188, 1205, 1223, 1409, 1414, 1495, 1526, 1531, 1537, 101, 255, 1421, 1457, 1538, 1580 and 1589, of Seq ID No 83;15-25, 41-102, 111-117, 127-134, 145-170, 194-201, 207-225, 10-30, 36-44, 46-59, 57-98, 122-138, 144-160, 162-173, 194-217 and 118-131 of Seq ID No 84; and fragments in 9 amino acid length starting from the position of: 12, 16, 37, 46, 61, 82, 121, 128, 149, 157, 162, 197, 204, 212, 39, 2, 23, 53, 68, 97, 107, 121, 127, 156, 169, 196, 9, 13 and 114 of Seq ID No 84; 7-54, 65-94, 97-103, 154-163, 170-180, 182-199, 216-222, 227-234, 243-256, 267-273, 286-298, 314-322, 324-353, 363-380, 393-401, 424-431, 434-441, 447-470, 475-495, 506-532, 540-548, 554-592, 594-607, 609-617, 619-626, 628-634, 656-662, 8-31, 43-59, 61-75, 93-104, 126-144, 179-201, 244-254, 289-302, 330-338, 364-382, 413-421, 428-466, 476-525, 582-599, 602-619 621-632 and 115-128 of Seq ID No 85; and fragments in 9 amino acid length starting from the position of: 9, 10, 13, 35, 46, 76, 77, 83, 151, 165, 179, 187, 195, 283, 326, 338, 342, 360, 365, 368, 375, 415, 450, 485, 508, 556, 565, 569, 576, 602, 5, 20, 130, 181, 251, 271, 288, 294, 333, 355, 356, 364, 446, 451, 467, 483, 486, 523, 544, 611, 214, 219, 323, 399, 424 and 458, of Seq ID No 85; 5-21, 32-56, 88-99, 117-124, 128-138, 143-150, 168-180, 183-189, 196-213, 220-240, 254-263, 266-289, 300-313, 321-330, 335-358, 361-371, 380-398, 50-65, 67-87, 96-104, 144-153, 156-164, 169-177, 199-220, 259-289, 324-333, 339-360, 372-385 and 74-93 of Seq ID No 86; and fragments in 9 amino acid length starting from the position of: 26, 33, 49, 88, 96, 129, 169, 170, 198, 257, 268, 281, 337, 342, 366, 391, 393, 39, 122, 248, 76, 106, 117, 185, 190, 198, 238, 257, 266, 280, 341, 344, 350, 367, 304 and 384 of Seq ID No 86; 12-23, 44-50, 54-60, 91-97, 103-109, 119-125, 131-137, 141-151, 172-183, 201-226, 230-238, 252-265, 315-321, 331-345, 360-370, 376-386, 392-406, 410-416, 422-431, 133-159, 208-222, 354-368 and 1-88 of Seq ID No 87; and fragments in 9 amino acid length starting from the position of: 47, 134, 140, 143, 203, 204, 210, 254, 355, 358, 359, 362, 369, 417, 119, 17, 128, 129, 141, 143, 153, 208, 232, 245, 278, 301, 313, 327, 328, 384 and 395 of Seq ID No 87; 4-16, 29-36, 39-64, 69-75, 79-87, 90-122, 126-134, 139-173, 184-190, 195-203, 206-213, 216-228, 234-246, 250-257, 260-266, 274-282, 291-312, 318-325, 340-345, 348-361, 364-388, 399-437, 439-448, 451-464, 467-473, 480-510, 514-520, 534-553, 561-574, 579-589, 593-599, 616-655, 658-671, 3-12, 23-38, 27-38, 43-56, 93-107, 123-137, 144-154, 175-199, 229-244, 288-303, 308-316, 323-337, 410-423, 455-473, 488-496, 531-551, 560-577, 577-591, 619-637, 646-660, 664-672 and 553-570 of Seq ID No 88; and fragments in 9 amino acid length starting from the position of: 36, 101, 123, 129, 136, 146, 156, 160, 194, 205, 219, 236, 245, 283, 289, 350, 402, 413, 437, 475, 505, 517, 542, 585, 605, 620, 627, 657, 34, 52, 88, 358, 540, 656, 3, 8, 13, 32, 82, 105, 111, 117, 137, 167, 173, 180, 182, 262, 300, 306, 350, 409, 412, 423, 499, 500, 563, 568, 581, 585, 627, 628, 554 and 638 of Seq ID No 88; 4-31, 50-80, 83-93, 97-103, 111-116, 123-132, 134-163, 170-199, 205-210, 215-220, 230-247, 249-278, 280-308, 311-329, 337-347, 349-358, 365-371, 376-401, 417-430, 434-446, 459-505, 511-518, 527-535, 537-545, 547-565, 573-581, 592-601, 1-17, 20-30, 66-80, 100-119, 139-150, 171-182, 186-198, 207-221, 228-242, 258-274, 286-308, 314-330, 337-352, 355-376, 383-391, 417-432, 437-446, 462-473, 479-488, 496-507, 514-522, 541-554, 557-565, 576-585, 589-605, 49-60 and 582-607 of Seq ID No 89; and fragments in 9 amino acid length starting from the position of: 4, 65, 66, 120, 121, 144, 170, 174, 208, 226, 233, 276, 278, 285, 286, 298, 336, 348, 355, 363, 382, 384, 395, 457, 458, 494, 501, 578, 133, 278, 294, 551, 53, 89, 110, 159, 186, 232, 290, 324, 406, 431, 458, 463, 480, 490, 513, 541, 549, 558, 585, 22, 137, 152, 189, 227, 255, 261, 291, 419 and 569 of Seq ID No 89; 9-60, 67-73, 79-93, 109-122, 134-142, 144-153, 165-192, 197-225, 235-244, 259-279, 289-299, 308-317, 321-332, 338-347, 350-361, 373-387, 402-409, 411-421, 439-445, 450-456, 462-468, 470-479, 490-501, 503-516, 16-27, 49-60, 99-122, 136-145, 148-162, 186-194, 213-221, 225-246, 261-275, 281-292, 353-361, 390-401, 451-470, 486-494, 497-516 and 478-490 of Seq ID No 90; and fragments in 9 amino acid length starting from the position of: 15, 22, 28, 29, 48, 49, 106, 107, 114, 147, 170, 177, 188, 208, 209, 212, 256, 280, 287, 316, 451, 468, 489, 33, 217, A03: 36, 98, 124, 136, 142, 153, 177, 188, 251, 262, 291, 320, 323, 383, 417, 464, 487, 491, 492, 505, 44, 86, 146, 411, 437 and 499 of Seq ID No 90; 4-10, 16-28, 3-14, 16-30 and 2-16 of Seq ID No 91; and fragments in 9 amino acid length starting from the position of: 1 and15 of Seq ID No 91; 8-18, 20-30 and 7-15 of Seq ID No 92; 4-16, 18-27, 2-13, 20-30 and 10-29 of Seq ID No 93; and fragments in 9 amino acid length starting from the position of: 22 and 1 of Seq ID No 93; 36-57, 62-92, 46-66 and 27-35 of Seq ID No 94; and fragments in 9 amino acid length starting from the position of: 84 of Seq ID No 94; 4-18, 1-16 and 5-12 of Seq ID No 95; and fragments in 9 amino acid length starting from the position of: 1, 9 and 2 of Seq ID No 95; 13-27, 38-52, 1-13, 11-25, 27-37 and 17-36 of Seq ID No 96; and fragments in 9 amino acid length starting from the position of: 16, 37 and 20 of Seq ID No 96; 4-17, 27-40, 55-62, 9-25, 34-46, 50-64 and 47-62 of Seq ID No 97; and fragments in 9 amino acid length starting from the position of: 7, 10, 11, 14 and 58 of Seq ID No 97; 4-9, 1-10 of Seq ID No 98; 3-14 and 7-20 of Seq ID No 99; and fragments in 9 amino acid length starting from the position of: 2 and 1 of Seq ID No 99; 7-12, 24-29, 22-30 and 7-21 of Seq ID No 100; and fragments in 9 amino acid length starting from the position of: 4 and 9 of Seq ID No 100; 14-30, 15-30 and 3-18 of Seq ID No 101; and fragments in 9 amino acid length starting from the position of: 1 and 20 of Seq ID No 101; 3-17 of Seq ID No 102; and fragments in 9 amino acid length starting from the position of: 1 of Seq ID No 102; 4-27, 31-59, 75-86, 93-103, 105-110, 15-44, 51-61, 79-95 and 41-50 of Seq ID No 103; and fragments in 9 amino acid length starting from the position of: 11, 15, 24, 28, 31, 35, 36, 42, 48, 49, 53, 78, 79, 97, 20, 28, 35, 37, 43, 49, 60, 65, 77, 85, 86, 21 and 103 of Seq ID No 103; 4-13 and 2-14 of Seq ID No 104; and fragments in 9 amino acid length starting from the position of: 7 and 10 of Seq ID No 104; 4-15, 17-23, 39-52, 4-13, 16-29, 40-50 and 33-41 of Seq ID No 105; and fragments in 9 amino acid length starting from the position of: 3, 38, 14 and 41 of Seq ID No 105; 4-25 of Seq ID No 106; 8-19, 40-47, 67-86, 88-125, 15-25, 48-59, 64-80, 108-118 and 60-70 of Seq ID No 107; and fragments in 9 amino acid length starting from the position of: 7, 110, 16, 34 and 109 of Seq ID No 107; 4-27, 41-46, and 30-47 of Seq ID No 108; and fragments in 9 amino acid length starting from the position of: 19, 1 and 23 of Seq ID No 108; 21-28, 34-43, 8-16 and 23-42 of Seq ID No 109; and fragments in 9 amino acid length starting from the position of: 34, 19, 28 and 39 of Seq ID No 109; 8-20, 24-37, 39-50, 61-67, 69-91, 4-16, 31-42, 84-93 and 42-59 of Seq ID No 110; and fragments in 9 amino acid length starting from the position of: 4, 24, 79, 83, 7, 25, 71, 79 and 91 of Seq ID No 110; 4-25, 31-39, 59-97, 100-118, 120-129, 26-40, 49-57, 66-95, 97-128, 131-139, 38-47 of Seq ID No 111; and fragments in 9 amino acid length starting from the position of: 8, 24, 61, 67, 72, 103, 112, 3, 39, 74, 110 and 119 of Seq ID No 111; 7-24, 32-43, 45-57, 32-48 and 27-43 of Seq ID No 112; and fragments in 9 amino acid length starting from the position of: 14, 18, 38, 47 and 14 of Seq ID No 112; 4-18, 20-26, 31-37, 3-17, 33-43 and 34-53 of Seq ID No 113; and fragments in 9 amino acid length starting from the position of: 3, 7, 10 and 9 of Seq ID No 113; 15-23, 25-39, 43-50, 62-70, 16-32, 61-73 and 67-84 of Seq ID No 114; and fragments in 9 amino acid length starting from the position of: 8 and 64 of Seq ID No 114; 4-13, 28-42, 3-14, 28-39 and 1-20 of Seq ID No115; and fragments in 9 amino acid length starting from the position of: 31, 7 and 5 of Seq ID No115; 4-10, 19-26, 21-29 and 5-13 of Seq ID No 116; 4-22, 40-46, 51-57, 64-76, 2-10, 45-53, 58-72, 73-82 and 33-45 of Seq ID No117; and fragments in 9 amino acid length starting from the position of: 35, 76, 3, 1 and 66 of Seq ID No117; 12-24, 27-42, 13-30, 34-44 and 1-9 of Seq ID No 118; and fragments in 9 amino acid length starting from the position of: 36, 15 and 18 of Seq ID No 118; 4-55, 5-15, 17-33 and 26-45 of Seq ID No 119; and fragments in 9 amino acid length starting from the position of: 14 and 53 of Seq ID No 119; 31-42, 45-52, 86-92, 8-16, 35-52, 83-91 and 27-93 of Seq ID No 120; and fragments in 9 amino acid length starting from the position of: 86, 56, 21 and 4 of Seq ID No 120; 237-256, 508-530 of Seq ID No 61; 227-239 of Seq ID No 62; 141-160, 168-187, 155-173 of Seq ID No 63; 101-124, 161-187, 59-85, 80-106 of Seq ID No 64; 97-112 of Seq ID No 66; 139-165 of Seq ID No 67; 10-21 of Seq ID No 68; 667-688, 677-696, 161-187, 183-209, 205-231, 226-252 of Seq ID No 69; 603-629, 622-648, 643-669 of Seq ID No 70; 529-541 of Seq ID No 71; 12-34, 29-51, 46-67, 62-83 of Seq ID No 72; 139-151 of Seq ID No 73; 246-262, 251-275 of Seq ID No 74; 61-84, 79-102, 97-120, 115-138 of Seq ID No 75; 325-350, 345-370, 365-389 of Seq ID No 76; 324-349, 336-351 of Seq ID No 77; 90-100 of Seq ID No 78; 274-290 of Seq ID No 79; 401-419 of Seq ID No 80; 84-107, 101-123, 117-139 of Seq ID No 81; 182-199 of Seq ID No 82; 911-935 of Seq ID No 83; 118-131 of Seq ID No 84; 115-128 of Seq ID No 85; 74-93 of Seq ID No 86; 21-43, 54-76 of Seq ID No 87; 554-570 of Seq ID No 88; 478-490 of Seq ID No 90; 2-14 of Seq ID No 91; 7-15 of Seq ID No 92; 10-28 of Seq ID No 93; 27-34 of Seq ID No 94; 17-35 of Seq ID No 96; 47-61 of Seq ID No 97; 1-10 of Seq ID No 98; 7-20 of Seq ID No 99; 7-20 of Seq ID No 100; 3-17 of Seq ID No 101; 3-17 of Seq ID No 102; 41-50 of Seq ID No 103; 2-14 of Seq ID No 104; 33-41 of Seq ID No 105; 4-25 of Seq ID No 106; 60-69 of Seq ID No 107; 23-41 of Seq ID No 109; 42-59 of Seq ID No 110; 38-46 of Seq ID No 111; 27-43 of Seq ID No 112; 34-53 of Seq ID No 113; 67-84 of Seq ID No 114; 1-20 of Seq ID No 115; 33-45 of Seq ID No 117; 26-45 of Seq ID No 119; 27-53 of Seq ID No 120.

The present invention also provides a process for producing a C. pneumoniae hyperimmune serum reactive antigen or a fragment thereof according to the present invention comprising expressing one or more of the nucleic acid molecules according to the present invention in a suitable expression system.

Moreover, the present invention provides a process for producing a cell, which expresses a C. pneumoniae hyperimmune serum reactive antigen or a fragment thereof according to the present invention comprising transforming or transfecting a suitable host cell with the vector according to the present invention.

According to the present invention a pharmaceutical composition, especially a vaccine, comprising a hyperimmune serum-reactive antigen or a fragment thereof as defined in the present invention or a nucleic acid molecule as defined in the present invention is provided.

In a preferred embodiment the pharmaceutical composition further comprises an immunostimulatory substance, preferably selected from the group comprising polycationic polymers, especially polycationic peptides, immunostimulatory deoxynucleotides (ODNs), peptides containing at least two LysLeuLys motifs, especially KLKLLLLLKLK (SEQ ID NO:121), neuroactive compounds, especially human growth hormone, alumn, Freund's complete or incomplete adjuvants or combinations thereof.

In a more preferred embodiment the immunostimulatory substance is a combination of either a polycationic polymer and immunostimulatory deoxynucleotides or of a peptide containing at least two LysLeuLys motifs and immunostimulatory deoxynucleotides.

In a still more preferred embodiment the polycationic polymer is a polycationic peptide, especially polyarginine.

According to the present invention the use of a nucleic acid molecule according to the present invention or a hyperimmune serum-reactive antigen or fragment thereof according to the present invention for the manufacture of a pharmaceutical preparation, especially for the manufacture of a vaccine against C. pneumoniae infection, is provided.

Also an antibody, or at least an effective part thereof, which binds at least to a selective part of the hyperimmune serum-reactive antigen or a fragment thereof according to the present invention, is provided herewith.

In a preferred embodiment the antibody is a monoclonal antibody.

In another preferred embodiment the effective part of the antibody comprises Fab fragments.

In a further preferred embodiment the antibody is a chimeric antibody.

In a still preferred embodiment the antibody is a humanized antibody.

The present invention also provides a hybridoma cell line, which produces an antibody according to the present invention.

Moreover, the present invention provides a method for producing an antibody according to the present invention, characterized by the following steps:
initiating an immune response in a non-human animal by administrating an hyperimmune serum-reactive antigen or a fragment thereof, as defined in the invention, to said animal,
removing an antibody containing body fluid from said animal, and
producing the antibody by subjecting said antibody containing body fluid to further purification steps.

Accordingly, the present invention also provides a method for producing an antibody according to the present invention, characterized by the following steps:
initiating an immune response in a non-human animal by administrating an hyperimmune serum-reactive antigen or a fragment thereof, as defined in the present invention, to said animal,
removing the spleen or spleen cells from said animal,
producing hybridoma cells of said spleen or spleen cells,
selecting and cloning hybridoma cells specific for said hyperimmune serum-reactive antigens or a fragment thereof,
producing the antibody by cultivation of said cloned hybridoma cells and optionally further purification steps.

The antibodies provided or produced according to the above methods may be used for the preparation of a medicament for treating or preventing C. pneumoniae infections.

According to another aspect the present invention provides an antagonist, which binds to a hyperimmune serum-reactive antigen or a fragment thereof according to the present invention.

Such an antagonist capable of binding to a hyperimmune serum-reactive antigen or fragment thereof according to the present invention may be identified by a method comprising the following steps:
a) contacting an isolated or immobilized hyperimmune serum-reactive antigen or a fragment thereof according to the present invention with a candidate antagonist under conditions to permit binding of said candidate antagonist to said hyperimmune serum-reactive antigen or fragment, in the presence of a component capable of providing a detectable signal in response to the binding of the candidate antagonist to said hyperimmune serum reactive antigen or fragment thereof; and
b) detecting the presence or absence of a signal generated in response to the binding of the antagonist to the hyperimmune serum reactive antigen or the fragment thereof.

An antagonist capable of reducing or inhibiting the interaction activity of a hyperimmune serum-reactive antigen or a fragment thereof according to the present invention to its interaction partner may be identified by a method comprising the following steps:
a) providing a hyperimmune serum reactive antigen or a hyperimmune fragment thereof according to the present invention,
b) providing an interaction partner to said hyperimmune serum reactive antigen or a fragment thereof, especially an antibody according to the present invention,
c) allowing interaction of said hyperimmune serum reactive antigen or fragment thereof to said interaction partner to form an interaction complex,
d) providing a candidate antagonist,
e) allowing a competition reaction to occur between the candidate antagonist and the interaction complex,
f) determining whether the candidate antagonist inhibits or reduces the interaction activities of the hyperimmune serum reactive antigen or the fragment thereof with the interaction partner.

The hyperimmune serum reactive antigens or fragments thereof according to the present invention may be used for the isolation and/or purification and/or identification of an interaction partner of said hyperimmune serum reactive antigen or fragment thereof.

The present invention also provides a process for in vitro diagnosing a disease related to expression of a hyperimmune serum-reactive antigen or a fragment thereof according to the present invention comprising determining the presence of a nucleic acid sequence encoding said hyperimmune serum reactive antigen or fragment thereof according to the present invention or the presence of the hyperimmune serum reactive antigen or fragment thereof according to the present invention.

The present invention also provides a process for in vitro diagnosis of a bacterial infection, especially a C. pneumoniae infection, comprising analyzing for the presence of a nucleic acid sequence encoding said hyperimmune serum reactive antigen or fragment thereof according to the present invention or the presence of the hyperimmune serum reactive antigen or fragment thereof according to the present invention.

Moreover, the present invention provides the use of a hyperimmune serum reactive antigen or fragment thereof according to the present invention for the generation of a peptide binding to said hyperimmune serum reactive antigen or fragment thereof, wherein the peptide is an anticaline.

The present invention also provides the use of a hyperimmune serum-reactive antigen or fragment thereof according to the present invention for the manufacture of a functional nucleic acid, wherein the functional nucleic acid is selected from the group comprising aptamers and spiegelmers.

The nucleic acid molecule according to the present invention may also be used for the manufacture of a functional ribonucleic acid, wherein the functional ribonucleic acid is selected from the group comprising ribozymes, antisense nucleic acids and siRNA.

The present invention advantageously provides an efficient, relevant and comprehensive set of isolated nucleic acid molecules and their encoded hyperimmune serum reactive antigens or fragments thereof identified from C. pneumoniae using an antibody preparation from multiple human plasma pools and surface expression libraries derived from the genome of C. pneumoniae. Thus, the present invention fulfils a widely felt demand for C. pneumoniae antigens, vaccines, diagnostics and products useful in procedures for preparing antibodies and for identifying compounds effective against C. pneumoniae infection.

An effective vaccine should be composed of proteins or polypeptides, which are expressed by all strains and are able to induce high affinity, abundant antibodies against cell surface components of C. pneumoniae or a sustained T-cell response capable of eradicating infected cells of the host. The antibodies should be IgG1 and/or IgG3 for opsonization, and any IgG subtype and IgA for neutralization of adherence and toxin action. A chemically defined vaccine must be definitely superior compared to a whole cell vaccine (attenuated or killed), since components of C. pneumoniae, which cross-react with human tissues or inhibit opsonization can be eliminated, and the individual proteins inducing protective antibodies and/or a protective immune response can be selected.

The approach, which has been employed for the present invention, is based on the interaction of Chlamydial proteins or peptides with the antibodies present in human sera. The antibodies produced against C. pneumoniae by the human immune system and present in human sera are indicative of the in vivo expression of the antigenic proteins and their immunogenicity. In addition, the antigenic proteins as identified by the bacterial surface display expression libraries using pools of pre-selected sera are processed in a second and third round of screening by individual selected or generated sera. Thus the present invention supplies an efficient, relevant, comprehensive set of chlamydial antigens as a pharmaceutical composition, especially a vaccine preventing infection by C. pneumoniae.

In the antigen identification program for identifying a comprehensive set of antigens according to the present invention, at least two different bacterial surface expression libraries are screened with several serum pools or plasma fractions or other pooled antibody containing body fluids (antibody pools). The antibody pools are derived from a serum collection, which has been tested against antigenic compounds of C. pneumoniae—highly enriched outer membrane preparation for ELISA and elementary body (EB) isolated from C. pneumoniae infected eukaryotic cells. Preferably, two distinct serum collections are used: 1. For antigen identification: sera from patients with clinical symptoms characterized with high anti-C. pneumoniae antibody levels and 2. For antigen validation: sera from healthy people and patients characterized with low, medium and high anti-C. pneumoniae antibody levels. Sera have to react with multiple Chlamydia-specific antigens in order to be considered hyperimmune and therefore relevant in the screening method applied for the present invention. Sera with low specific antibodies serve as negative controls.

The expression libraries as used in the present invention should allow expression of all potential antigens, e.g. derived from all secreted and surface proteins of C. pneumoniae. Bacterial surface display libraries will be represented by a recombinant library of a bacterial host displaying a (total) set of expressed peptide sequences of C. pneumoniae on two selected outer membrane proteins (LamB and FhuA) at the bacterial host membrane {Georgiou, G., 1997}; {Etz, H. et al., 2001}. One of the advantages of using recombinant expression libraries is that the identified hyperimmune serum-reactive antigens may be instantly produced by expression of the coding sequences of the screened and selected clones expressing the hyperimmune serum-reactive antigens without further recombinant DNA technology or cloning steps necessary.

The comprehensive set of antigens identified by the described program according to the present invention is analyzed further by one or more additional rounds of screening. Therefore individual antibody preparations or antibodies generated against selected peptides, which were identified as immunogenic are used. According to a preferred embodiment the individual antibody preparations for the second round of screening are derived from patients who have suffered from infection with C. pneumoniae, especially from patients who show an IgG antibody titer above a certain minimum level, for example an antibody titer being higher than 80 percentile, preferably higher than 90 percentile, especially higher than 95 percentile of the human (patient or healthy individual) sera tested. These thresholds are above of a titer of 400, meaning that individual serum samples can be diluted more than 400 times to give positive serological (ELISA) results.

Using such high titer individual antibody preparations in the second screening round allows a very selective identification of the hyperimmune serum-reactive antigens and fragments thereof from C. pneumoniae.

Following the comprehensive screening procedure, the selected antigenic proteins, produced as synthetic peptides corresponding to identified immunogenic epitopes are tested in a second screening by a series of ELISA assays for the assessment of their immunogenicity with a large human serum collection.

It is important that the individual antibody preparations (which may also be the selected serum) allow a selective identification of the most promising candidates of all the hyperimmune serum-reactive antigens from all the promising candidates from the first round. Therefore, preferably at least 10 individual antibody preparations (i.e. antibody preparations (e.g. sera) from at least 10 different individuals having suffered from an infection to the chosen pathogen) should be used in identifying these antigens in the second screening round. Of course, it is possible to use also less than 10 individual preparations, however, selectivity of the step may not be optimal with a low number of individual antibody preparations. On the other hand, if a given hyperimmune serum-reactive antigen (or an antigenic fragment thereof) is recognized by at least 10 individual antibody preparations, preferably at least 30, especially at least 50 individual antibody preparations, identification of the hyperimmune serum-reactive antigen is also selective enough for a proper identification. Hyperimmune serum-reactivity may of course be tested with as many individual preparations as possible (e.g. with more than 100 or even with more than 1,000).

Therefore, the relevant portion of the hyperimmune serum-reactive antibody preparations according to the method of the present invention should preferably be at least 10, more preferred at least 30, especially at least 50 individual antibody preparations. Alternatively (or in combination) hyperimmune serum-reactive antigens may preferably be also identified with at least 20%, preferably at least 30%, especially at least 40% of all individual antibody preparations used in the second screening round.

According to a preferred embodiment of the present invention, the sera from which the individual antibody preparations for the second round of screening are prepared (or which are used as antibody preparations), are selected by their titer against C. pneumoniae (e.g. against a preparation of this pathogen, such as a lysate, cell wall components and recombinant proteins).

Preferably, some are selected with a total IgG titer above 200, especially above 400 measured by a commercially available IgG ELISA kit.

The antibodies produced against Chlamydia by the human immune system and present in human sera are indicative of the in vivo expression of the antigenic proteins and their immunogenicity. The recognition of linear epitopes recognized by serum antibodies can be based on sequences as short as 4-5 amino acids. Of course it does not necessarily mean that these short peptides are capable of inducing the given antibody in vivo. For that reason the defined epitopes, polypeptides and proteins are further to be tested in animals (mainly in mice) for their capacity to induce T cells and antibodies against the selected proteins in vivo.

C. pneumoniae as an obligate intracellular parasite, has a unique biphasic life cycle with a smaller extracellular form, the infectious, non-replicating, relatively metabolically inert elementary body (EB), and a larger intracellular form, the infectious, replicating and metabolically active reticulate body. The EBs attach to susceptible host cells and are taken up by phagocytosis. Within the cell they revert to reticulate bodies and replicate before they revert to EBs prior to host cell lysis. Although the immune correlates of protection against C. pneumoniae are not well defined, studies using mouse models faithfully mimicking important aspects of human infection indicate that particularly CD8⁺ T cells and IFN-□ are critical for protection {Wizel, B. et al., 2002}. Since C. pneumoniae resides in the membrane bound vacuole, the preferred antigens have to reach the cytosol of infected cells and need to be subsequently recognized as MHC class I-peptide complex by CD8⁺ T cells. Most of the previously reported antigens—which seem to be therefore capable of reaching the cytosol—are located on the cell surface (e.g. outer membrane proteins) or are secreted (e.g. {Murdin, A. et al., 2000}; {Wizel, B. et al., 2002}). It has been shown that C. pneumoniae peptide specific CD8⁺ CTL and their soluble factors can inhibit chlamydial growth in vitro {Wizel, B. et al., 2002}. In addition, to the T cell-mediated immune response, antibodies against cell wall proteins induced by B cell epitopes may aid the T cell-mediated immune response and serve multiple purposes: they may inhibit adhesion, interfere with nutrient acquisition, inhibit immune evasion and promote phagocytosis {Hornef, M. et al., 2002}. Antibodies against secreted proteins are potentially beneficial in neutralization of their function as toxin or virulence component. It is also known that bacteria communicate with each other through secreted proteins. Neutralizing antibodies against these proteins will interrupt growth-promoting cross-talk between or within chlamydial species. The described experimental approach is based on the use of antibodies specifically induced by C. pneumoniae purified from human serum. The antigens identified by the genomic screens are thereby shown to be expressed in vivo in the host and to be capable of inducing an antibody response. Since it has been shown for many proteins that B cell and T cell epitopes reside in the same protein, the most promising candidates identified by the genomic screens can be further evaluated for the induction of a potent T cell response in vivo. As a first step, bioinformatic analyses have been used to identify potential T cell epitopes in silico, which can then be tested in the appropriate murine model of infection. Thus the present invention combines the experimental identification of immunogenic proteins with the bioinformatic prediction of T cell epitopes in order to provide candidates for an efficient vaccine to treat or prevent Chlamydial infections.

The method according to the present invention provides thus an optimal tool for the identification of chlamydial antigenic proteins as vaccine candidates. The selection of antigens as provided by the present invention is also well suited to identify those proteins that harbour B and T cell epitopes necessary to induce a protective immune response against infection by C. pneumoniae in animal models or in humans.

According to the antigen identification method used herein, the present invention can surprisingly provide a set of comprehensive novel nucleic acids and novel hyperimmune serum reactive antigens and fragments thereof of C. pneumoniae, among other things, as described below. According to one aspect, the invention particularly relates to the nucleotide sequences encoding hyperimmune serum reactive antigens which sequences are set forth in the Sequence listing Seq ID No: 1-60 and the corresponding encoded amino acid sequences representing hyperimmune serum reactive antigens are set forth in the Sequence Listing Seq ID No 61-120.

In a preferred embodiment of the present invention, a nucleic acid molecule is provided which exhibits 70% identity over their entire length to a nucleotide sequence set forth with Seq ID No 31-60. Most highly preferred are nucleic acids that comprise a region that is at least 80% or at least 85% identical over their entire length to a nucleic acid molecule set forth with Seq ID No 31-60. In this regard, nucleic acid molecules at least 90%, 91%, 92%, 93%, 94%, 95%, or 96% identical over their entire length to the same are particularly preferred. Furthermore, those with at least 97% are highly preferred, those with at least 98% and at least 99% are particularly highly preferred, with at least 99% or 99.5% being the more preferred, with 100% identity being especially preferred. Moreover, preferred embodiments in this respect are nucleic acids, which encode hyperimmune serum reactive antigens or fragments thereof (polypeptides) which retain substantially the same biological function or activity as the mature polypeptide encoded by said nucleic acids set forth in the Seq ID No 31-60.

Identity, as known in the art and used herein, is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity can be readily calculated. While there exist a number of methods to measure identity between two polynucleotide or two polypeptide sequences, the term is well known to skilled artisans (e.g. Sequence Analysis in Molecular Biology, von Heinje, G., Academic Press, 1987). Preferred methods to determine identity are designed to give the largest match between the sequences tested. Methods to determine identity are codified in computer programs. Preferred computer program methods to determine identity between two sequences include, but are not limited to, GCG program package {Devereux, J. et al., 1984}, BLASTP, BLASTN, and FASTA {Altschul, S. et al., 1990}.

According to another aspect of the invention, nucleic acid molecules are provided which exhibit at least 96%, preferably at least 98%, especially 100% identity to the nucleic acid sequence set forth with Seq ID No 5, 7-8, 14-16, 18-22, 24-27, 29-30.

The nucleic acid molecule according to the present invention can, as a second alternative, also be a nucleic acid molecule, which is at least essentially complementary to the nucleic acid described as the first alternative above. As used herein complementary means that a nucleic acid strand is base pairing via Watson-Crick base pairing with a second nucleic acid strand. Essentially complementary as used herein means that the base pairing is not occurring for all of the bases of the respective strands but leaves a certain number or percentage of the bases unpaired or wrongly paired. The percentage of correctly pairing bases is preferably at least 70%, more preferably 80% %, even more preferably 90% and most preferably any percentage higher than 90%. It is to be noted that a percentage of 70% matching bases is considered as homology and the hybridization having this extent of matching base pairs is considered as stringent. Hybridization conditions for this kind of stringent hybridization may be taken from Current Protocols in Molecular Biology (John Wiley & Sons, 1987). More particularly, the hybridization conditions can be as follows:
Hybridization performed e.g. in 5×SSPE, 5×Denhardt's reagent, 0.1% SDS, 100 g/mL sheared DNA at 68° C.
Moderate stringency wash in 0.2×SSC, 0.1% SDS at 42° C.
High stringency wash in 0.1×SSC, 0.1% SDS at 68° C.

Genomic DNA with a GC content of 50% has an approximate T_{M} of 96° C. For 1% mismatch, the T_{M} is reduced by approximately 1° C.

In addition, any of the further hybridization conditions described herein are in principle applicable as well.

Of course, all nucleic acid sequence molecules which encode the same polypeptide molecule as those identified by the present invention are encompassed by any disclosure of a given coding sequence, since the degeneracy of the genetic code is directly applicable to unambiguously determine all possible nucleic acid molecules which encode a given polypeptide molecule, even if the number of such degenerated nucleic acid molecules may be high. This is also applicable for fragments of a given polypeptide, as long as the fragments encode a polypeptide being suitable to be used in a vaccination connection, e.g. as an active or passive vaccine.

The nucleic acid molecule according to the present invention can as a third alternative also be a nucleic acid which comprises a stretch of at least 15 bases of the nucleic acid molecule according to the first and second alternative of the nucleic acid molecules according to the present invention as outlined above. Preferably, the bases form a contiguous stretch of bases. However, it is also within the scope of the present invention that the stretch consists of two or more moieties, which are separated by a number of bases.

The present nucleic acids may preferably consist of at least 20, even more preferred at least 30, especially at least 50 contiguous bases from the sequences disclosed herein. The suitable length may easily be optimized due to the planned area of use (e.g. as (PCR) primers, probes, capture molecules (e.g. on a (DNA) chip), etc.). Preferred nucleic acid molecules contain at least a contiguous 15 base portion of one or more of the predicted immunogenic amino acid sequences listed in tables 1 and 2, especially the sequences of table 2 with scores of more than 10, preferably more than 20, especially with a score of more than 25. Specifically preferred are nucleic acids containing a contiguous portion of a DNA sequence of any sequence in the sequence protocol of the present application which shows 1 or more, preferably more than 2, especially more than 5, non-identical nucleic acid residues compared to the published Chlamydia pneumoniae strain AR39 genome ({Read, T. et al., 2000}; GenBank accession AE002161) and/or any other published C. pneumoniae genome sequence or parts thereof, especially of the strains CWL029 ({Kalman, S. et al., 1999}; GenBank accession AE001363) and J138 ({Shirai, M. et al., 2000}; GenBank accession AB036071-AB036089). Specifically preferred non-identical nucleic acid residues are residues, which lead to a non-identical amino acid residue. Preferably, the nucleic acid sequences encode for polypeptides having at least 1, preferably at least 2, preferably at least 3 different amino acid residues compared to the published C. pneumoniae counterparts mentioned above. Also such isolated polypeptides, being fragments of the proteins (or the whole protein) mentioned herein e.g. in the sequence listing, having at least 6, 7, or 8 amino acid residues and being encoded by these nucleic acids are preferred.

The nucleic acid molecule according to the present invention can as a fourth alternative also be a nucleic acid molecule which anneals under stringent hybridization conditions to any of the nucleic acids of the present invention according to the above outlined first, second, and third alternative. Stringent hybridization conditions are typically those described herein.

Finally, the nucleic acid molecule according to the present invention can as a fifth alternative also be a nucleic acid molecule which, but for the degeneracy of the genetic code, would hybridize to any of the nucleic acid molecules according to any nucleic acid molecule of the present invention according to the first, second, third, and fourth alternative as outlined above. This kind of nucleic acid molecule refers to the fact that preferably the nucleic acids according to the present invention code for the hyperimmune serum reactive antigens or fragments thereof according to the present invention. This kind of nucleic acid molecule is particularly useful in the detection of a nucleic acid molecule according to the present invention and thus the diagnosis of the respective microorganisms such as C. pneumoniae and any disease or diseased condition where this kind of microorganisms is involved. Preferably, the hybridization would occur or be preformed under stringent conditions as described in connection with the fourth alternative described above.

Nucleic acid molecule as used herein generally refers to any ribonucleic acid molecule or deoxyribonucleic acid molecule, which may be unmodified RNA or DNA or modified RNA or DNA. Thus, for instance, nucleic acid molecule as used herein refers to, among other, single-and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or triple-stranded, or a mixture of single- and double-stranded regions. In addition, nucleic acid molecule as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term nucleic acid molecule includes DNAs or RNAs as described above that contain one or more modified bases. Thus, DNAs or RNAs with backbones modified for stability or for other reasons are “nucleic acid molecule” as that term is intended herein. Moreover, DNAs or RNAs comprising unusual bases, such as inosine, or modified bases, such as tritylated bases, to name just two examples, are nucleic acid molecule as the term is used herein. It will be appreciated that a great variety of modifications have been made to DNA and RNA that serve many useful purposes known to those of skill in the art. The term nucleic acid molecule as it is employed herein embraces such chemically, enzymatically or metabolically modified forms of nucleic acid molecule, as well as the chemical forms of DNA and RNA characteristic of viruses and cells, including simple and complex cells, inter alia. The term nucleic acid molecule also embraces short nucleic acid molecules often referred to as oligonucleotide(s). “Polynucleotide” and “nucleic acid” or “nucleic acid molecule” are often used interchangeably herein.

Nucleic acid molecules provided in the present invention also encompass numerous unique fragments, both longer and shorter than the nucleic acid molecule sequences set forth in the sequencing listing of the C. pneumoniae coding regions, which can be generated by standard cloning methods. To be unique, a fragment must be of sufficient size to distinguish it from other known nucleic acid sequences, most readily determined by comparing any selected C. pneumoniae fragment to the nucleotide sequences in computer databases such as GenBank.

Additionally, modifications can be made to the nucleic acid molecules and polypeptides that are encompassed by the present invention. For example, nucleotide substitutions can be made which do not affect the polypeptide encoded by the nucleic acid, and thus any nucleic acid molecule which encodes a hyperimmune serum reactive antigen or fragments thereof is encompassed by the present invention.

Furthermore, any of the nucleic acid molecules encoding hyperimmune serum reactive antigens or fragments thereof provided by the present invention can be functionally linked, using standard techniques such as standard cloning techniques, to any desired regulatory sequences, whether a C. pneumoniae regulatory sequence or a heterologous regulatory sequence, heterologous leader sequence, heterologous marker sequence or a heterologous coding sequence to create a fusion protein.

Nucleic acid molecules of the present invention may be in the form of RNA, such as mRNA or cRNA, or in the form of DNA, including, for instance, cDNA and genomic DNA obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand.

The present invention further relates to variants of the herein and above described nucleic acid molecules which encode fragments, analogs and derivatives of the hyperimmune serum reactive antigens and fragments thereof having a deducted C. pneumoniae amino acid sequence set forth in the Sequence Listing. A variant of the nucleic acid molecule may be a naturally occurring variant such as a naturally occurring allelic variant, or it may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or additions. The substitutions, deletions or additions may involve one or more nucleotides. The variants may be altered in coding or non-coding regions or both. Alterations in the coding regions may produce conservative or non-conservative amino acid substitutions, deletions or additions. Preferred are nucleic acid molecules encoding a variant, analog, derivative or fragment, or a variant, analogue or derivative of a fragment, which have a C. pneumoniae sequence as set forth in the Sequence Listing, in which several, a few, 5 to 10, 1 to 5, 1 to 3, 2, 1 or no amino acid(s) is substituted, deleted or added, in any combination. Especially preferred among these are silent substitutions, additions and deletions, which do not alter the properties and activities of the C. pneumoniae polypeptides set forth in the Sequence Listing. Also especially preferred in this regard are conservative substitutions.

The peptides and fragments according to the present invention also include modified epitopes wherein preferably one or two of the amino acids of a given epitope are modified or replaced according to the rules disclosed in e.g. {Tourdot, S. et al., 2000}, as well as the nucleic acid sequences encoding such modified epitopes.

It is clear that also epitopes derived from the present epitopes by amino acid exchanges improving, conserving or at least not significantly impeding the T cell activating capability of the epitopes are covered by the epitopes according to the present invention. Therefore the present epitopes also cover epitopes, which do not contain the original sequence as derived from C. pneumoniae, but trigger the same or preferably an improved T cell response. These epitope are referred to as “heteroclitic”; they need to have a similar or preferably greater affinity to MHC/HLA molecules, and the need the ability to stimulate the T cell receptors (TCR) directed to the original epitope in a similar or preferably stronger manner.

Heteroclitic epitopes can be obtained by rational design i.e. taking into account the contribution of individual residues to binding to MHC/HLA as for instance described by {Rammensee, H. et al., 1999}, combined with a systematic exchange of residues potentially interacting with the TCR and testing the resulting sequences with T cells directed against the original epitope. Such a design is possible for a skilled man in the art without much experimentation.

Another possibility includes the screening of peptide libraries with T cells directed against the original epitope. A preferred way is the positional scanning of synthetic peptide libraries. Such approaches have been described in detail for instance by {Hemmer, B. et al., 1999} and the references given therein.

As an alternative to epitopes represented by the present derived amino acid sequences or heteroclitic epitopes, also substances mimicking these epitopes e.g. “peptidemimetica” or “retro-inverso-peptides” can be applied.

Another aspect of the design of improved epitopes is their formulation or modification with substances increasing their capacity to stimulate T cells. These include T helper cell epitopes, lipids or liposomes or preferred modifications as described in WO 01/78767.

Another way to increase the T cell stimulating capacity of epitopes is their formulation with immune stimulating substances for instance cytokines or chemokines like interleukin-2, -7, -12, -18, class I and II interferons (IFN), especially IFN-gamma, GM-CSF, TNF-alpha, flt3-ligand and others.

As discussed additionally herein regarding nucleic acid molecule assays of the invention, for instance, nucleic acid molecules of the invention as discussed above, may be used as a hybridization probe for RNA, cDNA and genomic DNA to isolate full-length cDNAs and genomic clones encoding polypeptides of the present invention and to isolate cDNA and genomic clones of other genes that have a high sequence similarity to the nucleic acid molecules of the present invention. Such probes generally will comprise at least 15 bases. Preferably, such probes will have at least 20, at least 25 or at least 30 bases, and may have at least 50 bases. Particularly preferred probes will have at least 30 bases, and will have 50 bases or less, such as 30, 35, 40, 45, or 50 bases.

For example, the coding region of a nucleic acid molecule of the present invention may be isolated by screening a relevant library using the known DNA sequence to synthesize an oligonucleotide probe. A labelled oligonucleotide having a sequence complementary to that of a gene of the present invention is then used to screen a library of cDNA, genomic DNA or mRNA to determine to which members of the library the probe hybridizes.

The nucleic acid molecules and polypeptides of the present invention may be employed as reagents and materials for development of treatments of and diagnostics for disease, particularly human disease, as further discussed herein relating to nucleic acid molecule assays, inter alia.

The nucleic acid molecules of the present invention that are oligonucleotides can be used in the processes herein as described, but preferably for PCR, to determine whether or not the C. pneumoniae genes identified herein in whole or in part are present and/or transcribed in infected tissue such as blood. It is recognized that such sequences will also have utility in diagnosis of the stage of infection and type of infection the pathogen has attained. For this and other purposes the arrays comprising at least one of the nucleic acids according to the present invention as described herein, may be used.

The nucleic acid molecules according to the present invention may be used for the detection of nucleic acid molecules and organisms or samples containing these nucleic acids. Preferably such detection is for diagnosis, more preferable for the diagnosis of a disease related or linked to the present or abundance of C. pneumoniae.

Eukaryotes (herein also “individual(s)”), particularly mammals, and especially humans, infected with C. pneumoniae may be identifiable by detecting any of the nucleic acid molecules according to the present invention detected at the DNA level by a variety of techniques. Preferred nucleic acid molecules candidates for distinguishing a C. pneumoniae from other organisms can be obtained.

The invention provides a process for diagnosing disease, arising from infection with C. pneumoniae, comprising determining from a sample isolated or derived from an individual an increased level of expression of a nucleic acid molecule having the sequence of a nucleic acid molecule set forth in the Sequence Listing. Expression of nucleic acid molecules can be measured using any one of the methods well known in the art for the quantitation of nucleic acid molecules, such as, for example, PCR, RT-PCR, RNase protection, Northern blotting, other hybridization methods and the arrays described herein.

Isolated as used herein means separated “by the hand of man” from its natural state; i.e., that, if it occurs in nature, it has been changed or removed from its original environment, or both. For example, a naturally occurring nucleic acid molecule or a polypeptide naturally present in a living organism in its natural state is not “isolated,” but the same nucleic acid molecule or polypeptide separated from the coexisting materials of its natural state is “isolated”, as the term is employed herein. As part of or following isolation, such nucleic acid molecules can be joined to other nucleic acid molecules, such as DNAs, for mutagenesis, to form fusion proteins, and for propagation or expression in a host, for instance. The isolated nucleic acid molecules, alone or joined to other nucleic acid molecules such as vectors, can be introduced into host cells, in culture or in whole organisms. Introduced into host cells in culture or in whole organisms, such DNAs still would be isolated, as the term is used herein, because they would not be in their naturally occurring form or environment. Similarly, the nucleic acid molecules and polypeptides may occur in a composition, such as a media formulations, solutions for introduction of nucleic acid molecules or polypeptides, for example, into cells, compositions or solutions for chemical or enzymatic reactions, for instance, which are not naturally occurring compositions, and, therein remain isolated nucleic acid molecules or polypeptides within the meaning of that term as it is employed herein.

The nucleic acids according to the present invention may be chemically synthesized. Alternatively, the nucleic acids can be isolated from C. pneumoniae by methods known to the one skilled in the art.

According to another aspect of the present invention, a comprehensive set of novel hyperimmune serum reactive antigens and fragments thereof are provided by using the herein described antigen identification approach. In a preferred embodiment of the invention, a hyperimmune serum-reactive antigen comprising an amino acid sequence being encoded by any one of the nucleic acids molecules herein described and fragments thereof are provided. In another preferred embodiment of the invention a novel set of hyperimmune serum-reactive antigens which comprises amino acid sequences selected from a group consisting of the polypeptide sequences as represented in Seq ID No 91-120 and fragments thereof are provided. In a further preferred embodiment of the invention hyperimmune serum-reactive antigens, which comprise amino acid sequences selected from a group consisting of the polypeptide sequences as represented in Seq ID No 65, 67-68, 74-76, 78-82, 84-87, 89-90 and fragments thereof are provided.

The hyperimmune serum reactive antigens and fragments thereof as provided in the invention include any polypeptide set forth in the Sequence Listing as well as polypeptides which have at least 70% identity to a polypeptide set forth in the Sequence Listing, preferably at least 80% or 85% identity to a polypeptide set forth in the Sequence Listing, and more preferably at least 90% similarity (more preferably at least 90% identity) to a polypeptide set forth in the Sequence Listing and still more preferably at least 95%, 96%, 97%, 98%, 99% or 99.5% similarity (still more preferably at least 95%, 96%, 97%, 98%, 99%, or 99.5% identity) to a polypeptide set forth in the Sequence Listing and also include portions of such polypeptides with such portion of the polypeptide generally containing at least 4 amino acids and more preferably at least 8, still more preferably at least 30, still more preferably at least 50 amino acids, such as 4, 8, 10, 20, 30, 35, 40, 45 or 50 amino acids.

The invention also relates to fragments, analogs, and derivatives of these hyperimmune serum reactive antigens and fragments thereof The terms “fragment”, “derivative” and “analog” when referring to an antigen whose amino acid sequence is set forth in the Sequence Listing, means a polypeptide which retains essentially the same or a similar biological function or activity as such hyperimmune serum reactive antigen and fragment thereof.

The fragment, derivative or analog of a hyperimmune serum reactive antigen and fragment thereof may be 1) one in which one or more of the amino acid residues are substituted with a conserved or non-conserved amino acid residue (preferably a conserved amino acid residue) and such substituted amino acid residue may or may not be one encoded by the genetic code, or 2) one in which one or more of the amino acid residues includes a substituent group, or 3) one in which the mature hyperimmune serum reactive antigen or fragment thereof is fused with another compound, such as a compound to increase the half-life of the hyperimmune serum reactive antigen and fragment thereof (for example, polyethylene glycol), or 4) one in which the additional amino acids are fused to the mature hyperimmune serum reactive antigen or fragment thereof, such as a leader or secretory sequence or a sequence which is employed for purification of the mature hyperimmune serum reactive antigen or fragment thereof or a proprotein sequence. Such fragments, derivatives and analogs are deemed to be within the scope of those skilled in the art from the teachings herein.

The present invention also relates to antigens of different C. pneumoniae isolates. Such homologues may easily be isolated based on the nucleic acid and amino acid sequences disclosed herein. The genomes of different C. pneumoniae isolates are highly conserved as typified by the high degree of identity between the two published genomes of C. pneumoniae CWL029 and J138 {Shirai, M. et al., 2000}, which were isolated from a patient with pneumonia in the United States before 1987 and from the pharyngeal mucosa of a 5-year-old boy with acute bronchitis in 1994 in Japan, respectively. There are only 8 regions showing variation between these two strains isolated in different geographic regions and with a large gap in time. The remainder of the sequence is to more than 99.9% identical, indicating the high degree of conservation. The third C. pneumoniae strain that was sequenced, AR39, which is isolated from a human case of respiratory tract infection that is epidemiologically distinct from CWL029, confirmed the high degree of conservation between the C. pneumoniae strains {Read, T. et al., 2000}. It is therefore assumed that the majority of antigens will be conserved among all C. pneumoniae strains. Nevertheless, the presence of any antigen can be determined for every strain by appropriate means such as PCR or Southern blot analysis. In addition, it is possible to determine the variability of a particular antigen in the various strains by sequencing, as described for example for the S. pyogenes sic gene {Hoe, N. et al., 2001}. It is an important aspect that the most valuable protective antigens are expected to be conserved among most, if not all, various clinical strains.

Among the particularly preferred embodiments of the invention in this regard are the hyperimmune serum reactive antigens set forth in the Sequence Listing, variants, analogs, derivatives and fragments thereof, and variants, analogs and derivatives of fragments. Additionally, fusion polypeptides comprising such hyperimmune serum reactive antigens, variants, analogs, derivatives and fragments thereof, and variants, analogs and derivatives of the fragments are also encompassed by the present invention. Such fusion polypeptides and proteins, as well as nucleic acid molecules encoding them, can readily be made using standard techniques, including standard recombinant techniques for producing and expression of a recombinant polynucleic acid encoding a fusion protein.

Among preferred variants are those that vary from a reference by conservative amino acid substitutions. Such substitutions are those that substitute a given amino acid in a polypeptide by another amino acid of like characteristics. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr.

Further particularly preferred in this regard are variants, analogs, derivatives and fragments, and variants, analogs and derivatives of the fragments, having the amino acid sequence of any polypeptide set forth in the Sequence Listing, in which several, a few, 5 to 10, 1 to 5, 1 to 3, 2, 1 or no amino acid residues are substituted, deleted or added, in any combination. Especially preferred among these are silent substitutions, additions and deletions, which do not alter the properties and activities of the polypeptide of the present invention. Also especially preferred in this regard are conservative substitutions. Most highly preferred are polypeptides having an amino acid sequence set forth in the Sequence Listing without substitutions.

The hyperimmune serum reactive antigens and fragments thereof of the present invention are preferably provided in an isolated form, and preferably are purified to homogeneity.

Also among preferred embodiments of the present invention are polypeptides comprising fragments of the polypeptides having the amino acid sequence set forth in the Sequence Listing, and fragments of variants and derivatives of the polypeptides set forth in the Sequence Listing.

In this regard a fragment is a polypeptide having an amino acid sequence that entirely is the same as part but not all of the amino acid sequence of the afore mentioned hyperimmune serum reactive antigen and fragment thereof, and variants or derivative, analogs, fragments thereof. Such fragments may be “free-standing”, i.e., not part of or fused to other amino acids or polypeptides, or they may be comprised within a larger polypeptide of which they form a part or region. Also preferred in this aspect of the invention are fragments characterized by structural or functional attributes of the polypeptide of the present invention, i.e. fragments that comprise alpha-helix and alpha-helix forming regions, beta-sheet and beta-sheet forming regions, turn and turn-forming regions, coil and coil-forming regions, hydrophilic regions, hydrophobic regions, alpha amphipathic regions, beta-amphipathic regions, flexible regions, surface-forming regions, substrate binding regions, and high antigenic index regions of the polypeptide of the present invention, and combinations of such fragments. Preferred regions are those that mediate activities of the hyperimmune serum reactive antigens and fragments thereof of the present invention. Most highly preferred in this regard are fragments that have a chemical, biological or other activity of the hyperimmune serum reactive antigen and fragments thereof of the present invention, including those with a similar activity or an improved activity, or with a decreased undesirable activity. Particularly preferred are fragments comprising receptors or domains of enzymes that confer a function essential for viability of C. pneumoniae or the ability to cause disease in humans. Further preferred polypeptide fragments are those that comprise or contain antigenic or immunogenic determinants in an animal, especially in a human.

An antigenic fragment is defined as a fragment of the identified antigen, which is for itself antigenic or may be made antigenic when provided as a hapten. Therefore, also antigens or antigenic fragments showing one or (for longer fragments) only a few amino acid exchanges are enabled with the present invention, provided that the antigenic capacities of such fragments with amino acid exchanges are not severely deteriorated on the exchange(s), i.e., suited for eliciting an appropriate immune response in an individual vaccinated with this antigen and identified by individual antibody preparations from individual sera.

Preferred examples of such fragments of a hyperimmune serum-reactive antigen are selected from the group consisting of peptides comprising amino acid sequences of column “predicted immunogenic aa”, “Predicted class II restricted T-Cell epitopes/regions” “Predicted class I restricted T-Cell epitopes/regions”, and “Location of identified immunogenic region” of Table 1; the serum reactive peptide epitopes of Table 2, especially peptides comprising amino acid 18-29, 60-78, 89-95, 100-105, 124-143, 166-180, 187-194, 196-208, 224-242, 285-294, 305-311, 313-320, 351-360, 368-373, 390-403, 411-429, 432-470, 483-489, 513-523, 535-543, 548-564, 579-587, 589-598, 604-612, 622-627, 632-648, 55-84, 190-207, 323-331, 370-390, 551-570, 606-614, 633-647, 39-129, 224-296 and 464-609 of Seq ID No 61; and fragments in 9 amino acid length starting from the position of: 60, 63, 67, 70, 126, 129, 133, 136, 169, 186, 200, 308, 371, 414, 421, 434, 444, 459, 503, 512, 532, 540, 547, 601, 625, 632, 634, 637, 99, 529, 25, 38, 59, 155, 278, 285, 412, 420, 441, 451, 457, 481, 506, 510, 524, 536, 539, 554, 578, 596, 638, 179 and 604 of Seq ID No 61; 4-29, 31-38, 46-64, 66-80, 109-115, 131-139, 152-160, 170-183, 198-234, 239-255, 267-290, 301-313, 318-324, 336-345, 350-365, 380-386, 65-82, 123-165, 268-290, 299-307, 320-329, 336-347, 76-103, 226-239 and 267-333 of Seq ID No 62; and fragments in 9 amino acid length starting from the position of: 4, 13, 69, 93, 149, 174, 273, 277, 298, 305, 312, 319, 375, 28, 303, 3, 58, 73, 100, 153, 191, 223, 227, 232, 251, 269, 286, 343, 374 and 238 of Seq ID No 62; 20-33, 35-43, 47-60, 77-92, 113-124, 137-145, 185-196, 66-75 and 92-214 of Seq ID No 63; and fragments in 9 amino acid length starting from the position of: 32, 48, 49, 113, 77, 118, 139, 185, 2, 24 and 120 of Seq ID No 63; 47-64, 137-155, 157-167, 182-198, 212-233, 247-259, 291-303, 315-337, 345-350, 355-368, 373-379, 58-72, 183-196, 249-261, 315-323, 334-342, 347-356, 358-366 and 6-188 of Seq ID No 64; and fragments in 9 amino acid length starting from the position of: 135, 160, 183, 184, 204, 249, 256, 293, 296, 318, 319, 356, 372, 94, 13, 60, 159, 163, 189, 204, 220, 233, 300, 333, 335, 356, 362, 198 and 289 of Seq ID No 64; 4-36, 43-49, 60-75, 96-107, 113-123, 132-172, 186-193, 217-229, 231-250, 260-282, 284-290, 298-312, 315-330, 5-38, 67-77, 113-127, 134-145, 147-156, 220-236, 271-283, 285-293, 296-304, 309-321 and 159-217 of Seq ID No 65; and fragments in 9 amino acid length starting from the position of: 3, 10, 14, 17, 24, 46, 59, 133, 155, 220, 270, 312, 233, 2, 22, 31, 36, 62, 65, 122, 140, 155, 162, 170, 189, 235, 248, 260, 286, 298, 156, 183 and 325 of Seq ID No 65; 5-26, 29-50, 52-61, 65-74, 89-96, 140-147, 153-162, 183-188, 191-197, 203-210, 213-225, 1-9, 30-38, 53-63, 70-78, 92-107, 141-149, 158-166, 174-191, 205-224 and 97-113 of Seq ID No 66; and fragments in 9 amino acid length starting from the position of: 31, 33, 39, 56, 63, 78, 119, 136, 196, 14, 35, 38, 55, 97, 98, 146, 156, 158, 215, 88 and 214 of Seq ID No 66; 31-36, 46-54, 65-80, 86-102, 168-175, 179-186, 188-194, 200-208, 210-216, 225-231, 243-257, 289-296, 362-387, 460-474, 476-486, 504-511, 518-525, 569-579, 581-600, 665-684, 688-694, 700-705, 717-735, 182-193, 202-211, 279-294, 311-319, 369-377, 468-476, 547-558, 579-587, 681-700, 731-740, 92-177 and 591-604 of Seq ID No 67; and fragments in 9 amino acid length starting from the position of: 28, 78, 285, 309, 321, 376, 379, 388, 468, 475, 479, 500, 571, 624, 668, 716, 360, 455, 669, 185, 190, 204, 264, 281, 292, 478, 502, 588, 675, 680, 716 and 730 of Seq ID No 67; 4-9, 17-24, 27-52, 66-77, 91-98, 104-124, 127-139, 178-199, 211-219, 221-228, 234-244, 246-255, 263-286, 303-312, 316-321, 337-346, 356-362, 367-372, 377-390, 402-416, 449-459, 465-479, 491-501, 503-508, 523-541, 551-558, 560-565, 31-69, 115-127, 132-143, 145-165, 176-187, 190-204, 212-220, 266-286, 304-316, 403-423, 440-456, 523-544 and 9-22 of Seq ID No 68; and fragments in 9 amino acid length starting from the position of: 17, 24, 31, 45, 53, 56, 63, 69, 107, 129, 150, 171, 178, 189, 191, 217, 255, 273, 277, 305, 312, 451, 458, 470, 478, 506, 522, 71, 379, 20, 29, 34, 44, 119, 133, 276, 284, 300, 328, 404, 465, 470, 529, 543, 182 and 551 of Seq ID No 68; 34-42, 52-63, 71-87, 112-120, 142-147, 154-159, 166-177, 180-197, 204-224, 237-256, 260-268, 280-286, 312-324, 338-343, 372-412, 456-463, 479-490, 494-504, 506-512, 518-524, 538-548, 562-573, 585-591, 597-606, 674-690, 703-712, 714-740, 749-766, 95-103, 114-123, 180-195, 205-220, 240-248, 370-400, 481-495, 588-596, 707-715, 750-765, 160-253 and 630-717 of Seq ID No 69; and fragments in 9 amino acid length starting from the position of: 179, 206, 209, 213, 216, 255, 286, 300, 304, 324, 365, 369, 373, 376, 377, 380, 381, 384, 562, 694, 720, 721, 729, 749, 752, 755, 197, 330, 559, 592, 600, 714, 751, 91, 111, 140, 167, 191, 315, 388, 393, 402, 458, 463, 587, 720, 762 and 748 of Seq ID No 69; 4-44, 50-55, 59-67, 73-83, 91-98, 101-109, 131-145, 230-236, 267-273, 293-300, 303-310, 349-354, 375-397, 404-416, 434-441, 445-452, 456-468, 479-485, 487-512, 544-568, 571-579, 593-599, 604-610, 614-621, 642-656, 665-678, 706-716, 729-736, 748-756, 780-795, 797-814, 827-844, 850-861, 864-882, 889-900, 906-933, 6-23, 28-36, 64-75, 134-150, 182-192, 227-236, 306-316, 340-350, 376-387, 421-435, 449-460, 527-535, 553-569, 587-595, 641-657, 668-676, 683-694, 743-755, 800-819, 843-865, 861-886, 894-915, 929-938 and 603-669 of Seq ID No 70; and fragments in 9 amino acid length starting from the position of: 7, 8, 15, 73, 80, 133, 134, 138, 182, 194, 271, 272, 298, 432, 438, 457, 458, 487, 490, 527, 548, 568, 616, 644, 647, 667, 741, 782, 801, 829, 866, 126, 259, 792, 15, 20, 133, 155, 160, 232, 299, 458, 464, 552, 558, 560, 605, 607, 654, 670, 672, 768, 810, 840, 852, 877, 900, 167, 380, 425, 593 and 907 of Seq ID No 70; 4-32, 73-82, 90-101, 116-132, 144-160, 171-182, 195-200, 227-234, 255-271, 293-300, 313-336, 344-350, 369-375, 381-398, 413-421, 436-465, 487-496, 503-508, 510-527, 538-546, 552-562, 608-614, 617-636, 663-674, 679-691, 705-730, 734-748, 769-807, 825-834, 848-861, 864-871, 891-902, 7-16, 90-107, 110-137, 170-187, 197-213, 233-251, 277-287, 291-314, 361-390, 412-425, 451-465, 489-498, 513-521, 570-580, 619-637, 662-679, 713-721, 725-733, 745-754, 766-781, 790-805, 817-834, 868-883, 888-903 and 529-542 of Seq ID No 71; and fragments in 9 amino acid length starting from the position of: 8, 23, 53, 57, 128, 169, 178, 239, 263, 290, 297, 310, 324, 331, 339, 365, 398, 436, 443, 450, 470, 485, 488, 513, 514, 520, 614, 669, 711, 723, 771, 824, 849, 895, 316, 861, 118, 135, 196, 225, 284, 290, 370, 454, 489, 492, 521, 557, 624, 632, 745, 778, 783, 850, 868, 910, 226 and 383 of Seq ID No 71; 10-18, 30-52, 63-70, 72-79, 96-133, 146-158, 168-175, 184-193, 203-210, 213-222, 227-234, 237-257, 263-273, 285-291, 297-312, 320-338, 359-378, 385-393, 395-410, 412-421, 490-510, 521-527, 540-548, 563-571, 573-585, 592-598, 615-620, 632-641, 652-661, 672-679, 704-711, 717-723, 729-736, 742-751, 766-778, 788-808, 817-824, 836-842, 34-56, 73-89, 103-130, 146-154, 184-205, 213-227, 245-257, 258-278, 292-316, 331-341, 358-369, 372-383, 388-397, 410-418, 503-514, 524-530, 548-556, 565-573,. 584-595, 637-646, 656-663, 673-686, 734-742, 745-754, 757-768, 770-781, 816-828 and 14-101 of Seq ID No 72; and fragments in 9 amino acid length starting from the position of: 27, 32, 36, 65, 109, 112, 120, 127, 186, 249, 250, 262, 267, 297, 301, 353, 360, 367, 410, 418, 436, 465, 472, 505, 518, 522, 565, 576, 585, 638, 645, 650, 676, 687, 724, 745, 756, 763, 795, 164, 411, 510, 560, 569, 647, 766, 780, 14, 39, 48, 65, 74, 129, 175, 215, 217, 229, 230, 240, 253, 257, 262, 269, 308, 317, 322, 327, 352, 371, 372, 373, 374, 417, 443, 454, 472, 514, 525, 567, 629, 637, 657, 662, 683, 698, 731, 744, 752, 763, 769, 787, 790, 802, 815, 819, 26, 102, 381 and 704 of Seq ID No 72; 4-14, 20-33, 36-63, 71-93, 96-104, 106-117, 120-128, 131-147, 161-172, 174-186, 195-210, 212-247, 269-286, 288-301, 306-322, 324-332, 348-354, 356-363, 384-391, 35-66, 70-85, 107-118, 124-132, 165-179, 186-196, 197-205, 276-289, 292-300, 348-368, 369-381, 385-394 and 139-151 of Seq ID No 73; and fragments in 9 amino acid length starting from the position of: 34, 41, 50, 53, 109, 127, 134, 153, 165, 271, 286, 297, 340, 384, 80, 321, 334, 354, 33, 57, 110, 153, 178, 276, 284, 383, 79, 99 and 123 of Seq ID No 73; 12-20, 37-48, 51-58, 69-75, 86-98, 113-136, 141-161, 171-216, 222-254, 264-273, 291-301, 311-345, 351-361, 31-39, 40-55, 62-74, 121-137, 148-164, 170-178, 223-253, 309-329, 354-369 and 246-275 of Seq ID No 74; and fragments in 9 amino acid length starting from the position of: 46, 95, 103, 110, 143, 156, 178, 186, 190, 236, 242, 244, 291, 294, 315, 333, 353, 125, 183, 256, 326, 3, 68, 82, 102, 131, 177, 185, 190, 193, 223, 224, 244, 250, 295, 340, 349, 354, 88 and 89 of Seq ID No 74; 30-36, 50-56, 96-102, 110-116, 125-131, 162-174, 179-187, 189-201, 223-230, 232-239, 266-278, 320-328, 330-337, 339-350, 388-400, 408-413, 417-423, 435-447, 456-480, 499-524, 526-534, 53-62, 92-107, 192-203, 315-323, 436-452, 464-483, 502-524 and 61-138 of Seq ID No 75; and fragments in 9 amino acid length starting from the position of: 126, 174, 225, 267, 309, 316, 320, 337, 436, 466, 467, 473, 474, 14, 128, 143, 228, 347, 494, 2, 52, 112, 201, 209, 217, 230, 235, 236, 337, 381, 395, 413, 419, 454, 466, 510, 515 and 556 of Seq ID No 75; 7-32, 36-56, 77-82, 88-100, 117-144, 153-166, 173-180, 188-226, 256-297, 300-316, 323-337, 339-348, 361-384, 390-427, 438-455, 476-488, 516-523, 535-566, 580-586, 597-607, 615-621, 626-634, 639-649, 654-660, 668-673, 677-688, 707-714, 716-728, 730-742, 746-756, 763-772, 801-808, 820-829, 840-875, 882-888, 895-911, 914-920, 928-948, 953-961, 987-995, 999-1005, 1007-1026, 1053-1060, 1071-1079, 1082-1117, 1123-1129, 6-31, 37-48, 58-69, 90-105, 110-118, 134-142, 146-157, 210-220, 267-276, 291-300, 319-330, 362-372, 393-401, 405-421, 447-456, 463-471, 517-525, 574-582, 597-612, 618-626, 642-650, 656-668, 668-678, 683-695, 725-733, 778-791, 840-849, 894-917, 927-939, 954-963, 966-974, 978-998, 1010-1021, 1056-1067, 1070-1083, 1090-1104 and 325-389 of Seq ID No 76; and fragments in 9 amino acid length starting from the position of: 11, 18, 22, 41, 48, 86, 104, 156, 190, 197, 221, 286, 290, 334, 343, 345, 407, 442, 509, 538, 575, 596, 597, 598, 636, 678, 685, 723, 754, 757, 779, 818, 850, 857, 864, 893, 900, 901, 907, 918, 927, 934, 972, 988, 1018, 1025, 1034, 1048, 1065, 1072, 1089, 1094, 1101, 1108, 127, 336, 411, 806, 852, 28, 68, 90, 91, 93, 158, 293, 310, 350, 368, 380, 394, 425, 441, 461, 554, 569, 597, 628, 667, 684, 724, 737, 752, 761, 767, 804, 851, 897, 907, 933, 979, 1030, 1032, 1051, 1075, 1090, 1125, 133, 308, 502, 797, 939 and 960 of Seq ID No 76; 11-19, 34-53, 55-91, 113-119, 122-129, 131-140, 157-170, 173-179, 188-195, 200-206, 208-220, 222-232, 236-244, 250-265, 267-274, 282-290, 293-301, 317-323, 336-343, 355-361, 372-384, 33-54, 69-95, 210-221, 244-254, 257-269 and 324-351 of Seq ID No 77; and fragments in 9 amino acid length starting from the position of: 32, 37, 43, 47, 50, 53, 57, 64, 68, 71, 73, 74, 78, 80, 82, 113, 120, 155, 162, 194, 205, 209, 231, 235, 238, 252, 259, 266, 273, 280, 287, 294, 301, 308, 315, 333, 8, 16, 18, 66, 377, 36, 44, 81, 99, 124, 193, 261 and 319 of Seq ID No 77; 31-55, 58-64, 69-75, 81-90, 129-150, 154-167, 179-184, 189-208, 227-237, 248-271, 277-284, 313-340, 350-358, 361-368, 371-378, 384-390, 418-425, 438-444, 455-468, 487-506, 514-523, 525-550, 558-569, 572-578, 588-598, 607-618, 645-651, 653-665, 672-684, 708-715, 717-742, 754-771, 776-782, 786-802, 806-817, 1-9, 31-46, 52-61, 60-78, 132-148, 182-199, 214-229, 249-264, 280-293, 320-341, 347-355, 386-411, 486-502, 553-575, 624-634, 673-689, 690-700, 702-714, 721-735, 736-746, 757-777, 788-798, 810-818 and 90-100 of Seq ID No 78; and fragments in 9 amino acid length starting from the position of: 51, 82, 139, 186, 193, 197, 200, 239, 248, 249, 250, 257, 311, 325, 326, 520, 555, 556, 589, 606, 651, 716, 723, 730, 737, 758, 761, 772, 788, 39, 41, 569, 695, 709, 783, 51, 60, 89, 110, 141, 207, 216, 295, 301, 395, 404, 518, 527, 555, 568, 593, 596, 673, 691, 722, 757, 772, 790, 799, 130, 131, 179, 402, 414 and 701 of Seq ID No 78;13-19, 22-28, 61-67, 74-81, 86-103, 110-122, 141-155, 162-169, 171-177, 181-186, 192-199, 201-207, 225-238, 246-263, 273-279, 287-300, 307-313, 331-336, 351-367, 370-376, 380-392, 395-402, 415-422, 424-451, 454-465, 473-492, 496-509, 515-523, 541-547, 569-582, 589-601, 613-636, 638-647, 653-679, 702-714, 721-729, 739-748, 768-779, 799-813, 821-828, 832-840, 847-853, 857-873, 886-892, 894-905, 917-926, 958-971, 974-981, 983-989, 997-1004, 1006-1032, 1034-1049, 1054-1061, 1063-1069, 1073-1081, 1083-1095, 1097-1115, 1122-1132, 1143-1153, 1164-1171, 1178-1185, 1193-1213, 1216-1251, 1258-1272, 1277-1283, 1305-1317, 1324-1330, 1333-1355, 1383-1390, 25-43, 81-92, 111-141, 150-159, 213-220, 222-242, 243-254, 256-267, 276-288, 289-307, 381-397, 398-409, 422-438, 441-464, 485-500, 515-528, 542-553, 569-585, 591-601, 639-649, 656-664, 709-719, 725-734, 739-753, 841-850, 883-893, 902-911, 912-926, 935-948, 960-969, 976-984, 994-1008, 1037-1047, 1073-1085, 1100-1108, 1124-1134, 1167-1179, 1194-1203, 1220-1254, 1258-1277, 1308-1319, 1348-1366 and 273-290 of Seq ID No 79; and fragments in 9 amino acid length starting from the position of: 107, 110, 112, 133, 152, 200, 204, 223, 244, 251, 271, 289, 291, 305, 323, 360, 380, 407, 422, 428, 440, 491, 507, 512, 536, 616, 625, 628, 648, 650, 665, 668, 748, 768, 784, 797, 801, 826, 858, 859, 903, 910, 913, 925, 932, 959, 960, 968, 993, 1008, 1020, 1068, 1072, 1138, 1141, 1142, 1193, 1201, 1218, 1226, 1237, 1261, 1271, 1311, 1348, 1349, 1377, 126, 375, 433, 477, 608, 658, 852, 1106, 1121, 1303, 1362, 24, 102, 151, 164, 169, 211, 229, 245, 274, 279, 285, 333, 348, 361, 382, 391, 397, 428, 447, 453, 480, 496, 590, 591, 595, 615, 623, 629, 638, 664, 669, 672, 738, 744, 775, 789, 840, 910, 917, 939, 966, 977, 1057, 1084, 1096, 1119, 1127, 1128, 1145, 1163, 1167, 1202, 1214, 1238, 1244, 1260, 1279, 1335, 145, 355, 961, 1053, 1103 and 1245 of Seq ID No 79; 16-23, 25-47, 49-59, 64-72, 79-91, 95-105, 113-122, 133-145, 148-162, 169-176, 179-188, 190-200, 202-218, 232-239, 250-283, 299-333, 337-344, 349-355, 364-406, 430-437, 439-449, 452-460, 464-490, 492-503, 505-530, 533-562, 12-21, 28-39, 52-67, 115-124, 189-204, 224-232, 234-242, 263-284, 302-322, 363-385, 389-397, 446-463, 479-488, 513-522, 528-552 and 401-419 of Seq ID No 80; and fragments in 9 amino acid length starting from the position of: 23, 30, 58, 78, 84, 97, 98, 120, 123, 133, 162, 169, 189, 215, 218, 236, 309, 312, 316, 365, 372, 384, 388, 391, 426, 446, 453, 465, 466, 478, 508, 513, 515, 523, 530, 536, 543, 554, 333, 467, 13, 19, 115, 130, 181, 195, 225, 262, 270, 275, 311, 313, 325, 342, 390, 391, 398, 461, 530, 116, 188 and 229 of Seq ID No 80;8-16, 36-54, 59-76, 85-92, 104-124, 137-180, 199-248, 255-298, 300-307, 324-339, 356-373, 381-393, 402-442, 448-455, 18-27, 36-56, 101-120, 145-158, 165-173, 179-189, 239-255, 255-270, 330-346, 355-375, 383-394, 403-421 and 83-232 of Seq ID No 81; and fragments in 9 amino acid length starting from the position of: 5, 102, 149, 156, 160, 164, 185, 186, 204, 208, 211, 221, 232, 264, 270, 273, 277, 280, 284, 287, 317, 329, 362, 387, 398, 402, 404, 422, 429, 431, 449, 37, 298, 359, 9, 17, 35, 40, 41, 105, 111, 146, 166, 234, 279, 343, 384, 412 and 365 of Seq ID No 81; 29-69, 71-88, 95-104, 106-130, 143-189, 205-232, 24-40, 46-64, 65-79, 83-105, 121-129, 144-199, 206-236 and 182-199 of Seq ID No 82; and fragments in 9 amino acid length starting from the position of: 30, 37, 66, 77, 81, 84, 112, 118, 141, 144, 145, 146, 149, 150, 153, 167, 169, 170, 178, 196, 213, 215, 220, 13, 21, 39, 44, 62, 75, 78, 97, 119, 124, 145, 148, 154, 177, 190, 207, 22 and 216 of Seq ID No 82; 4-46, 51-66, 77-88, 102-110, 115-126, 142-148, 171-181, 183-192, 202-212, 227-234, 251-261, 263-278, 283-316, 319-325, 336-352, 362-371, 386-393, 399-406, 410-425, 427-437, 441-450, 457-464, 471-476, 490-496, 514-521, 549-557, 571-578, 601-611, 618-623, 627-646, 657-670, 672-689, 696-704, 726-740, 742-756, 765-776, 778-784, 792-801, 822-836, 862-868, 875-881, 887-898, 914-919, 941-948, 963-969, 971-978, 996-1004, 1007-1016, 1036-1051, 1068-1080, 1082-1090, 1092-1098, 1104-1127, 1135-1144, 1156-1177, 1181-1195, 1197-1206, 1214-1231, 1243-1263, 1278-1284, 1295-1303, 1305-1323, 1337-1346, 1355-1374, 1376-1383, 1406-1423, 1455-1463, 1465-1489, 1506-1518, 1527-1552, 1555-1570, 1581-1589, 1-28, 109-124, 208-220, 261-280, 286-296, 310-324, 398-405, 425-433, 439-454, 504-517, 535-555, 570-591, 599-614, 620-630, 691-699, 711-719, 729-739, 751-760, 783-791, 843-855, 878-886, 890-900, 940-955, 984-1003, 1007-1026, 1065-1073, 1106-1122, 1136-1149, 1188-1198, 1203-1211, 1227-1235, 1249-1256, 1298-1308, 1374-1392, 1398-1409, 1414-1429, 1436-1444, 1456-1490, 1504-1521, 1530-1547, 1592-1609 and 911-935 of Seq ID No 83; and fragments in 9 amino acid length starting from the position of: 26, 33, 79, 170, 200, 265, 290, 297, 302, 304, 333, 334, 377, 412, 414, 415, 431, 436, 458, 465, 481, 494, 536, 546, 568, 605, 678, 690, 697, 703, 724, 729, 730, 735, 737, 767, 776, 797, 840, 861, 938, 968, 999, 1072, 1079, 1085, 1094, 1113, 1160, 1163, 1180, 1188, 1195, 1217, 1245, 1250, 1273, 1302, 1358, 1362, 1363, 1401, 1408, 1465, 1469, 1481, 1507, 178, 960, 1034, 6, 21, 38, 159, 204, 248, 260, 306, 337, 349, 384, 425, 438, 458, 481, 502, 521, 546, 605, 690, 730, 731, 819, 860, 915, 946, 967, 1007, 1018, 1065, 1113, 1187, 1188, 1205, 1223, 1409, 1414, 1495, 1526, 1531, 1537, 101, 255, 1421, 1457, 1538, 1580 and 1589, of Seq ID No 83;15-25, 41-102, 111-117, 127-134, 145-170, 194-201, 207-225, 10-30, 36-44, 46-59, 57-98, 122-138, 144-160, 162-173, 194-217 and 118-131 of Seq ID No 84; and fragments in 9 amino acid length starting from the position of: 12, 16, 37, 46, 61, 82, 121, 128, 149, 157, 162, 197, 204, 212, 39, 2, 23, 53, 68, 97, 107, 121, 127, 156, 169, 196, 9, 13 and 114 of Seq ID No 84; 7-54, 65-94, 97-103, 154-163, 170-180, 182-199, 216-222, 227-234, 243-256, 267-273, 286-298, 314-322, 324-353, 363-380, 393-401, 424-431, 434-441, 447-470, 475-495, 506-532, 540-548, 554-592, 594-607, 609-617, 619-626, 628-634, 656-662, 8-31, 43-59, 61-75, 93-104, 126-144, 179-201, 244-254, 289-302, 330-338, 364-382, 413-421, 428-466, 476-525, 582-599, 602-619 621-632 and 115-128 of Seq ID No 85; and fragments in 9 amino acid length starting from the position of: 9, 10, 13, 35, 46, 76, 77, 83, 151, 165, 179, 187, 195, 283, 326, 338, 342, 360, 365, 368, 375, 415, 450, 485, 508, 556, 565, 569, 576, 602, 5, 20, 130, 181, 251, 271, 288, 294, 333, 355, 356, 364, 446, 451, 467, 483, 486, 523, 544, 611, 214, 219, 323, 399, 424 and 458, of Seq ID No 85; 5-21, 32-56, 88-99, 117-124, 128-138, 143-150, 168-180, 183-189, 196-213, 220-240, 254-263, 266-289, 300-313, 321-330, 335-358, 361-371, 380-398, 50-65, 67-87, 96-104, 144-153, 156-164, 169-177, 199-220, 259-289, 324-333, 339-360, 372-385 and 74-93 of Seq ID No 86; and fragments in 9 amino acid length starting from the position of: 26, 33, 49, 88, 96, 129, 169, 170, 198, 257, 268, 281, 337, 342, 366, 391, 393, 39, 122, 248, 76, 106, 117, 185, 190, 198, 238, 257, 266, 280, 341, 344, 350, 367, 304 and 384 of Seq ID No 86; 12-23, 44-50, 54-60, 91-97, 103-109, 119-125, 131-137, 141-151, 172-183, 201-226, 230-238, 252-265, 315-321, 331-345, 360-370, 376-386, 392-406, 410-416, 422-431, 133-159, 208-222, 354-368 and 1-88 of Seq ID No 87; and fragments in 9 amino acid length starting from the position of: 47, 134, 140, 143, 203, 204, 210, 254, 355, 358, 359, 362, 369, 417, 119, 17, 128, 129, 141, 143, 153, 208, 232, 245, 278, 301, 313, 327, 328, 384 and 395 of Seq ID No 87; 4-16, 29-36, 39-64, 69-75, 79-87, 90-122, 126-134, 139-173, 184-190, 195-203, 206-213, 216-228, 234-246, 250-257, 260-266, 274-282, 291-312, 318-325, 340-345, 348-361, 364-388, 399-437, 439-448, 451-464, 467-473, 480-510, 514-520, 534-553, 561-574, 579-589, 593-599, 616-655, 658-671, 3-12, 23-38, 27-38, 43-56, 93-107, 123-137, 144-154, 175-199, 229-244, 288-303, 308-316, 323-337, 410-423, 455-473, 488-496, 531-551, 560-577, 577-591, 619-637, 646-660, 664-672 and 553-570 of Seq ID No 88; and fragments in 9 amino acid length starting from the position of: 36, 101, 123, 129, 136, 146, 156, 160, 194, 205, 219, 236, 245, 283, 289, 350, 402, 413, 437, 475, 505, 517, 542, 585, 605, 620, 627, 657, 34, 52, 88, 358, 540, 656, 3, 8, 13, 32, 82, 105, 111, 117, 137, 167, 173, 180, 182, 262, 300, 306, 350, 409, 412, 423, 499, 500, 563, 568, 581, 585, 627, 628, 554 and 638 of Seq ID No 88; 4-31, 50-80, 83-93, 97-103, 111-116, 123-132, 134-163, 170-199, 205-210, 215-220, 230-247, 249-278, 280-308, 311-329, 337-347, 349-358, 365-371, 376-401, 417-430, 434-446, 459-505, 511-518, 527-535, 537-545, 547-565, 573-581, 592-601, 1-17, 20-30, 66-80, 100-119, 139-150, 171-182, 186-198, 207-221, 228-242, 258-274, 286-308, 314-330, 337-352, 355-376, 383-391, 417-432, 437-446, 462-473, 479-488, 496-507, 514-522, 541-554, 557-565, 576-585, 589-605, 49-60 and 582-607 of Seq ID No 89; and fragments in 9 amino acid length starting from the position of: 4, 65, 66, 120, 121, 144, 170, 174, 208, 226, 233, 276, 278, 285, 286, 298, 336, 348, 355, 363, 382, 384, 395, 457, 458, 494, 501, 578, 133, 278, 294, 551, 53, 89, 110, 159, 186, 232, 290, 324, 406, 431, 458, 463, 480, 490, 513, 541, 549, 558, 585, 22, 137, 152, 189, 227, 255, 261, 291, 419 and 569 of Seq ID No 89; 9-60, 67-73, 79-93, 109-122, 134-142, 144-153, 165-192, 197-225, 235-244, 259-279, 289-299, 308-317, 321-332, 338-347, 350-361, 373-387, 402-409, 411-421, 439-445, 450-456, 462-468, 470-479, 490-501, 503-516, 16-27, 49-60, 99-122, 136-145, 148-162, 186-194, 213-221, 225-246, 261-275, 281-292, 353-361, 390-401, 451-470, 486-494, 497-516 and 478-490 of Seq ID No 90; and fragments in 9 amino acid length starting from the position of: 15, 22, 28, 29, 48, 49, 106, 107, 114, 147, 170, 177, 188, 208, 209, 212, 256, 280, 287, 316, 451, 468, 489, 33, 217, A03: 36, 98, 124, 136, 142, 153, 177, 188, 251, 262, 291, 320, 323, 383, 417, 464, 487, 491, 492, 505, 44, 86, 146, 411, 437 and 499 of Seq ID No 90; 4-10, 16-28, 3-14, 16-30 and 2-16 of Seq ID No 91; and fragments in 9 amino acid length starting from the position of: 1 and15 of Seq ID No 91; 8-18, 20-30 and 7-15 of Seq ID No 92; 4-16, 18-27, 2-13, 20-30 and 10-29 of Seq ID No 93; and fragments in 9 amino acid length starting from the position of: 22 and 1 of Seq ID No 93; 36-57, 62-92, 46-66 and 27-35 of Seq ID No 94; and fragments in 9 amino acid length starting from the position of: 84 of Seq ID No 94; 4-18, 1-16 and 5-12 of Seq ID No 95; and fragments in 9 amino acid length starting from the position of: 1, 9 and 2 of Seq ID No 95; 13-27, 38-52, 1-13, 11-25, 27-37 and 17-36 of Seq ID No 96; and fragments in 9 amino acid length starting from the position of: 16, 37 and 20 of Seq ID No 96; 4-17, 27-40, 55-62, 9-25, 34-46, 50-64 and 47-62 of Seq ID No 97; and fragments in 9 amino acid length starting from the position of: 7, 10, 11, 14 and 58 of Seq ID No 97; 4-9, 1-10 of Seq ID No 98; 3-14 and 7-20 of Seq ID No 99; and fragments in 9 amino acid length starting from the position of: 2 and 1 of Seq ID No 99; 7-12, 24-29, 22-30 and 7-21 of Seq ID No 100; and fragments in 9 amino acid length starting from the position of: 4 and 9 of Seq ID No 100; 14-30, 15-30 and 3-18 of Seq ID No 101; and fragments in 9 amino acid length starting from the position of: 1 and 20 of Seq ID No 101; 3-17 of Seq ID No 102; and fragments in 9 amino acid length starting from the position of: 1 of Seq ID No 102; 4-27, 31-59, 75-86, 93-103, 105-110, 15-44, 51-61, 79-95 and 41-50 of Seq ID No 103; and fragments in 9 amino acid length starting from the position of: 11, 15, 24, 28, 31, 35, 36, 42, 48, 49, 53, 78, 79, 97, 20, 28, 35, 37, 43, 49, 60, 65, 77, 85, 86, 21 and 103 of Seq ID No 103; 4-13 and 2-14 of Seq ID No 104; and fragments in 9 amino acid length starting from the position of: 7 and 10 of Seq ID No 104; 4-15, 17-23, 39-52, 4-13, 16-29, 40-50 and 33-41 of Seq ID No 105; and fragments in 9 amino acid length starting from the position of: 3, 38, 14 and 41 of Seq ID No 105; 4-25 of Seq ID No 106; 8-19, 40-47, 67-86, 88-125, 15-25, 48-59, 64-80, 108-118 and 60-70 of Seq ID No 107; and fragments in 9 amino acid length starting from the position of: 7, 110, 16, 34 and 109 of Seq ID No 107; 4-27, 41-46, and 30-47 of Seq ID No 108; and fragments in 9 amino acid length starting from the position of: 19, 1 and 23 of Seq ID No 108; 21-28, 34-43, 8-16 and 23-42 of Seq ID No 109; and fragments in 9 amino acid length starting from the position of: 34, 19, 28 and 39 of Seq ID No 109; 8-20, 24-37, 39-50, 61-67, 69-91, 4-16, 31-42, 84-93 and 42-59 of Seq ID No 110; and fragments in 9 acid length starting from the position of: 4, 24, 79, 83, 7, 25, 71, 79 and 91 of Seq ID No 110; 4-25, 31-39, 59-97, 100-118, 120-129, 26-40, 49-57, 66-95, 97-128, 131-139, 38-47 of Seq ID No 111; and fragments in 9 amino acid length starting from the position of: 8, 24, 61, 67, 72, 103, 112, 3, 39, 74, 110 and 119 of Seq ID No 111; 7-24, 32-43, 45-57, 32-48 and 27-43 of Seq ID No 112; and fragments in 9 amino acid length starting from the position of: 14, 18, 38, 47 and 14 of Seq ID No 112; 4-18, 20-26, 31-37, 3-17, 33-43 and 34-53 of Seq ID No 113; and fragments in 9 amino acid length starting from the position of: 3, 7, 10 and 9 of Seq ID No 113; 15-23, 25-39, 43-50, 62-70, 16-32, 61-73 and 67-84 of Seq ID No 114; and fragments in 9 amino acid length starting from the position of: 8 and 64 of Seq ID No 114; 4-13, 28-42, 3-14, 28-39 and 1-20 of Seq ID No115; and fragments in 9 amino acid length starting from the position of: 31, 7 and 5 of Seq ID No115; 4-10, 19-26, 21-29 and 5-13 of Seq ID No 116; 4-22, 40-46, 51-57, 64-76, 2-10, 45-53, 58-72, 73-82 and 33-45 of Seq ID No117; and fragments in 9 amino acid length starting from the position of: 35, 76, 3, 1 and 66 of Seq ID No117; 12-24, 27-42, 13-30, 34-44 and 1-9 of Seq ID No 118; and fragments in 9 amino acid length starting from the position of: 36, 15 and 18 of Seq ID No 118; 4-55, 5-15, 17-33 and 26-45 of Seq ID No 119; and fragments in 9 amino acid length starting from the position of: 14 and 53 of Seq ID No 119; 31-42, 45-52, 86-92, 8-16, 35-52, 83-91 and 27-93 of Seq ID No 120; and fragments in 9 amino acid length starting from the position of: 86, 56, 21 and 4 of Seq ID No 120; 237-256, 508-530 of Seq ID No 61; 227-239 of Seq ID No 62; 141-160, 168-187, 155-173 of Seq ID No 63; 101-124, 161-187, 59-85, 80-106 of Seq ID No 64; 97-112 of Seq ID No 66; 139-165 of Seq ID No 67; 10-21 of Seq ID No 68; 667-688, 677-696, 161-187, 183-209, 205-231, 226-252 of Seq ID No 69; 603-629, 622-648, 643-669 of Seq ID No 70; 529-541 of Seq ID No 71; 12-34, 29-51, 46-67, 62-83 of Seq ID No 72; 139-151 of Seq ID No 73; 246-262, 251-275 of Seq ID No 74; 61-84, 79-102, 97-120, 115-138 of Seq ID No 75; 325-350, 345-370, 365-389 of Seq ID No 76; 324-349, 336-351 of Seq ID No 77; 90-100 of Seq ID No 78; 274-290 of Seq ID No 79; 401-419 of Seq ID No 80; 84-107, 101-123, 117-139 of Seq ID No 81; 182-199 of Seq ID No 82; 911-935 of Seq ID No 83; 118-131 of Seq ID No 84; 115-128 of Seq ID No 85; 74-93 of Seq ID No 86; 21-43, 54-76 of Seq ID No 87; 554-570 of Seq ID No 88; 478-490 of Seq ID No 90; 2-14 of Seq ID No 91; 7-15 of Seq ID No 92; 10-28 of Seq ID No 93; 27-34 of Seq ID No 94; 17-35 of Seq ID No 96; 47-61 of Seq ID No 97; 1-10 of Seq ID No 98; 7-20 of Seq ID No 99; 7-20 of Seq ID No 100; 3-17 of Seq ID No 101; 3-17 of Seq ID No 102; 41-50 of Seq ID No 103; 2-14 of Seq ID No 104; 33-41 of Seq ID No 105; 4-25 of Seq ID No 106; 60-69 of Seq ID No 107; 23-41 of Seq ID No 109; 42-59 of Seq ID No 110; 38-46 of Seq ID No 111; 27-43 of Seq ID No 112; 34-53 of Seq ID No 113; 67-84 of Seq ID No 114; 1-20 of Seq ID No 115; 33-45 of Seq ID No 117; 26-45 of Seq ID No 119; 27-53 of Seq ID No 120, and fragments comprising at least 6, preferably more than 8, especially more than 10 aa and preferably not more than 70, 50, 40, 20, 15, 11 aa of said sequences. All these fragments individually and each independently form a preferred selected aspect of the present invention.

All linear hyperimmune serum reactive fragments of a particular antigen may be identified by analyzing the entire sequence of the protein antigen by a set of peptides overlapping by 1 amino acid with a length of at least 10 amino acids. Subsequently, non-linear epitopes can be identified by analysis of the protein antigen with hyperimmune sera using the expressed full-length protein or domain polypeptides thereof. Assuming that a distinct domain of a protein is sufficient to form the 3D structure independent from the native protein, the analysis of the respective recombinant or synthetically produced domain polypeptide with hyperimmune serum would allow the identification of conformational epitopes within the individual domains of multi-domain proteins. For those antigens where a domain possesses linear as well as conformational epitopes, competition experiments with peptides corresponding to the linear epitopes may be used to confirm the presence of conformational epitopes.

It will be appreciated that the invention also relates to, among others, nucleic acid molecules encoding the aforementioned fragments, nucleic acid molecules that hybridize to nucleic acid molecules encoding the fragments, particularly those that hybridize under stringent conditions, and nucleic acid molecules, such as PCR primers, for amplifying nucleic acid molecules that encode the fragments. In these regards, preferred nucleic acid molecules are those that correspond to the preferred fragments, as discussed above.

The present invention also relates to vectors, which comprise a nucleic acid molecule or nucleic acid molecules of the present invention, host cells which are genetically engineered with vectors of the invention and the production of hyperimmune serum reactive antigens and fragments thereof by recombinant techniques.

A great variety of expression vectors can be used to express a hyperimmune serum reactive antigen or fragment thereof according to the present invention. Generally, any vector suitable to maintain, propagate or express nucleic acids to express a polypeptide in a host may be used for expression in this regard. In accordance with this aspect of the invention the vector may be, for example, a plasmid vector, a single or double-stranded phage vector, a single or double-stranded RNA or DNA viral vector. Starting plasmids disclosed herein are either commercially available, publicly available, or can be constructed from available plasmids by routine application of well-known, published procedures. Preferred among vectors, in certain respects, are those for expression of nucleic acid molecules and hyperimmune serum reactive antigens or fragments thereof of the present invention. Nucleic acid constructs in host cells can be used in a conventional manner to produce the gene product encoded by the recombinant sequence. Alternatively, the hyperimmune serum reactive antigens and fragments thereof of the invention can be synthetically produced by conventional peptide synthesizers. Mature proteins can be expressed in mammalian cells, yeast, bacteria, or other cells under the control of appropriate promoters. Cell-free translation systems can also be employed to produce such proteins using RNAs derived from the DNA construct of the present invention.

Host cells can be genetically engineered to incorporate nucleic acid molecules and express nucleic acid molecules of the present invention. Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, Hela, C127, 3T3, BHK, 293 and Bowes melanoma cells; and plant cells.

The invention also provides a process for producing a C. pneumoniae hyperimmune serum reactive antigen and a fragment thereof comprising expressing from the host cell a hyperimmune serum reactive antigen or fragment thereof encoded by the nucleic acid molecules provided by the present invention. The invention further provides a process for producing a cell, which expresses a C. pneumoniae hyperimmune serum reactive antigen or a fragment thereof comprising transforming or transfecting a suitable host cell with the vector according to the present invention such that the transformed or transfected cell expresses the polypeptide encoded by the nucleic acid contained in the vector.

The polypeptide may be expressed in a modified form, such as a fusion protein, and may include not only secretion signals but also additional heterologous functional regions. Thus, for instance, a region of additional amino acids, particularly charged amino acids, may be added to the N- or C-terminus of the polypeptide to improve stability and persistence in the host cell, during purification or during subsequent handling and storage. Also, regions may be added to the polypeptide to facilitate purification. Such regions may be removed prior to final preparation of the polypeptide. The addition of peptide moieties to polypeptides to engender secretion or excretion, to improve stability or to facilitate purification, among others, are familiar and routine techniques in the art. A preferred fusion protein comprises a heterologous region from immunoglobulin that is useful to solubilize or purify polypeptides. For example, EP-A-0 464 533 (Canadian counterpart 2045869) discloses fusion proteins comprising various portions of constant region of immunoglobin molecules together with another protein or part thereof. In drug discovery, for example, proteins have been fused with antibody Fc portions for the purpose of high-throughout screening assays to identify antagonists. See for example, {Bennett, D. et al., 1995} and {Johanson, K. et al., 1995}.

The C. pneumoniae hyperimmune serum reactive antigen or a fragment thereof can be recovered and purified from recombinant cell cultures by well-known methods including ammonium sulfate or ethanol precipitation, acid extraction, anion or cation exchange chromatography, phosphocellulose chromatography, hydrophobic interaction chromatography, hydroxylapatite chromatography and lectin chromatography.

The hyperimmune serum reactive antigens and fragments thereof according to the present invention can be produced by chemical synthesis as well as by biotechnological means. The latter comprise the transfection or transformation of a host cell with a vector containing a nucleic acid according to the present invention and the cultivation of the transfected or transformed host cell under conditions, which are known to the ones skilled in the art. The production method may also comprise a purification step in order to purify or isolate the polypeptide to be manufactured. In a preferred embodiment the vector is a vector according to the present invention.

The hyperimmune serum reactive antigens and fragments thereof according to the present invention may be used for the detection of the organism or organisms in a sample containing these organisms or polypeptides derived thereof. Preferably such detection is for diagnosis, more preferable for the diagnosis of a disease, most preferably for the diagnosis of a disease related or linked to the presence or abundance of the family of Gram-negative Chlamydiaceae bacteria. More preferably, the microorganisms are selected from the group comprising Chlamydia trachomatis, Chlamydia psittaci and Chlamydia muridarum, especially the microorganism is Chlamydia pneumoniae.

The present invention also relates to diagnostic assays such as quantitative and diagnostic assays for detecting levels of the hyperimmune serum reactive antigens and fragments thereof of the present invention in cells and tissues, including determination of normal and abnormal levels. Thus, for instance, a diagnostic assay in accordance with the invention for detecting over-expression of the polypeptide compared to normal control tissue samples may be used to detect the presence of an infection, for example, and to identify the infecting organism. Assay techniques that can be used to determine levels of a polypeptide, in a sample derived from a host are well known to those of skill in the art. Such assay methods include radioimmunoassays, competitive-binding assays, Western Blot analysis and ELISA assays. Among these, ELISAs frequently are preferred. An ELISA assay initially comprises preparing an antibody specific to the polypeptide, preferably a monoclonal antibody. In addition, a reporter antibody generally is prepared which binds to the monoclonal antibody. The reporter antibody is attached to a detectable reagent such as radioactive, fluorescent or enzymatic reagent, such as horseradish peroxidase enzyme.

The hyperimmune serum reactive antigens and fragments thereof according to the present invention may also be used for the purpose of or in connection with an array. More particularly, at least one of the hyperimmune serum reactive antigens and fragments thereof according to the present invention may be immobilized on a support. Said support typically comprises a variety of hyperimmune serum reactive antigens and fragments thereof whereby the variety may be created by using one or several of the hyperimmune serum reactive antigens and fragments thereof according to the present invention and/or hyperimmune serum reactive antigens and fragments thereof being different. The characterizing feature of such array as well as of any array in general is the fact that at a distinct or predefined region or position on said support or a surface thereof, a distinct polypeptide is immobilized. Because of this any activity at a distinct position or region of an array can be correlated with a specific polypeptide. The number of different hyperimmune serum reactive antigens and fragments thereof immobilized on a support may range from as little as 10 to several 1000 different hyperimmune serum reactive antigens and fragments thereof. The density of hyperimmune serum reactive antigens and fragments thereof per cm² is in a preferred embodiment as little as 10 peptides/polypeptides per cm² to at least 400 different peptides/polypeptides per cm² and more particularly at least 1000 different hyperimmune serum reactive antigens and fragments thereof per cm².

The manufacture of such arrays is known to the one skilled in the art and, for example, described in U.S. Pat. No. 5,744,309. The array preferably comprises a planar, porous or non-porous solid support having at least a first surface. The hyperimmune serum reactive antigens and fragments thereof as disclosed herein, are immobilized on said surface. Preferred support materials are, among others, glass or cellulose. It is also within the present invention that the array is used for any of the diagnostic applications described herein. Apart from the hyperimmune serum reactive antigens and fragments thereof according to the present invention also the nucleic acid molecules according to the present invention may be used for the generation of an array as described above. This applies as well to an array made of antibodies, preferably monoclonal antibodies as, among others, described herein.

In a further aspect the present invention relates to an antibody directed to any of the hyperimmune serum reactive antigens and fragments thereof, derivatives or fragments thereof according to the present invention. The present invention includes, for example, monoclonal and polyclonal antibodies, chimeric, single chain, and humanized antibodies, as well as Fab fragments, or the product of a Fab expression library. It is within the present invention that the antibody may be chimeric, i. e. that different parts thereof stem from different species or at least the respective sequences are taken from different species.

Antibodies generated against the hyperimmune serum reactive antigens and fragments thereof corresponding to a sequence of the present invention can be obtained by direct injection of the hyperimmune serum reactive antigens and fragments thereof into an animal or by administering the hyperimmune serum reactive antigens and fragments thereof to an animal, preferably a non-human. The antibody so obtained will then bind the hyperimmune serum reactive antigens and fragments thereof itself In this manner, even a sequence encoding only a fragment of a hyperimmune serum reactive antigen and fragments thereof can be used to generate antibodies binding the whole native hyperimmune serum reactive antigen and fragments thereof Such antibodies can then be used to isolate the hyperimmune serum reactive antigens and fragments thereof from tissue expressing those hyperimmune serum reactive antigens and fragments thereof.

For preparation of monoclonal antibodies, any technique known in the art, which provides antibodies produced by continuous cell line cultures can be used (as described originally in {Kohler, G. et al., 1975}.

Techniques described for the production of single chain antibodies (U.S. Pat. No. 4,946,778) can be adapted to produce single chain antibodies to immunogenic hyperimmune serum reactive antigens and fragments thereof according to this invention. Also, transgenic mice, or other organisms such as other mammals, may be used to express humanized antibodies to immunogenic hyperimmune serum reactive antigens and fragments thereof according to this invention.

Alternatively, phage display technology or ribosomal display could be utilized to select antibody genes with binding activities towards the hyperimmune serum reactive antigens and fragments thereof either from repertoires of PCR amplified v-genes of lymphocytes from humans screened for possessing respective target antigens or from nave libraries {McCafferty, J. et al., 1990}; {Marks, J. et al., 1992}. The affinity of these antibodies can also be improved by chain shuffling {Clackson, T. et al., 1991}.

If two antigen binding domains are present, each domain may be directed against a different epitope—termed ‘bispecific’ antibodies.

The above-described antibodies may be employed to isolate or to identify clones expressing the hyperimmune serum reactive antigens and fragments thereof or purify the hyperimmune serum reactive antigens and fragments thereof of the present invention by attachment of the antibody to a solid support for isolation and/or purification by affinity chromatography.

Thus, among others, antibodies against the hyperimmune serum reactive antigens and fragments thereof of the present invention may be employed to inhibit and/or treat infections, particularly bacterial infections and especially infections arising from C. pneumoniae.

Hyperimmune serum reactive antigens and fragments thereof include antigenically, epitopically or immunologically equivalent derivatives, which form a particular aspect of this invention. The term “antigenically equivalent derivative” as used herein encompasses a hyperimmune serum reactive antigen and fragments thereof or its equivalent which will be specifically recognized by certain antibodies which, when raised to the protein or hyperimmune serum reactive antigen and fragments thereof according to the present invention, interfere with the interaction between pathogen and mammalian host. The term “immunologically equivalent derivative” as used herein encompasses a peptide or its equivalent which when used in a suitable formulation to raise antibodies in a vertebrate, the antibodies act to interfere with the interaction between pathogen and mammalian host.

The hyperimmune serum reactive antigens and fragments thereof, such as an antigenically or immunologically equivalent derivative or a fusion protein thereof can be used as an antigen to immunize a mouse or other animal such as a rat or chicken. The fusion protein may provide stability to the hyperimmune serum reactive antigens and fragments thereof. The antigen may be associated, for example by conjugation, with an immunogenic carrier protein, for example bovine serum albumin (BSA) or keyhole limpet haemocyanin (KLH). Alternatively, an antigenic peptide comprising multiple copies of the protein or hyperimmune serum reactive antigen and fragments thereof, or an antigenically or immunologically equivalent hyperimmune serum reactive antigen and fragments thereof, may be sufficiently antigenic to improve immunogenicity so as to obviate the use of a carrier.

Preferably the antibody or derivative thereof is modified to make it less immunogenic in the individual. For example, if the individual is human the antibody may most preferably be “humanized”, wherein the complimentarity determining region(s) of the hybridoma-derived antibody has been transplanted into a human monoclonal antibody, for example as described in {Jones, P. et al., 1986} or {Tempest, P. et al., 1991}.

The use of a polynucleotide of the invention in genetic immunization will preferably employ a suitable delivery method such as direct injection of plasmid DNA into muscle, delivery of DNA complexed with specific protein carriers, coprecipitation of DNA with calcium phosphate, encapsulation of DNA in various forms of liposomes, particle bombardment {Tang, D. et al., 1992}; {Eisenbraun, M. et al., 1993} and in vivo infection using cloned retroviral vectors {Seeger, C. et al., 1984}.

In a further aspect the present invention relates to a peptide binding to any of the hyperimmune serum reactive antigens and fragments thereof according to the present invention, and a method for the manufacture of such peptides whereby the method is characterized by the use of the hyperimmune serum reactive antigens and fragments thereof according to the present invention and the basic steps are known to the one skilled in the art.

Such peptides may be generated by using methods according to the state of the art such as phage display or ribosome display. In case of phage display, basically a library of peptides is generated, in form of phages, and this kind of library is contacted with the target molecule, in the present case a hyperimmune serum reactive antigen and fragments thereof according to the present invention. Those peptides binding to the target molecule are subsequently removed, preferably as a complex with the target molecule, from the respective reaction. It is known to the one skilled in the art that the binding characteristics, at least to a certain extent, depend on the particularly realized experimental set-up such as the salt concentration and the like. After separating those peptides binding to the target molecule with a higher affinity or a bigger force, from the non-binding members of the library, and optionally also after removal of the target molecule from the complex of target molecule and peptide, the respective peptide(s) may subsequently be characterized. Prior to the characterization optionally an amplification step is realized such as, e.g. by propagating the peptide encoding phages. The characterization preferably comprises the sequencing of the target binding peptides. Basically, the peptides are not limited in their lengths, however, preferably peptides having a lengths from about 8 to 20 amino acids are preferably obtained in the respective methods. The size of the libraries may be about 10² to 10¹⁸, preferably 10⁸ to 10¹⁵ different peptides, however, is not limited thereto.

A particular form of target binding hyperimmune serum reactive antigens and fragments thereof are the so-called “anticalines” which are, among others, described in German patent application DE 197 42 706.

In a further aspect the present invention relates to functional nucleic acids interacting with any of the hyperimmune serum reactive antigens and fragments thereof according to the present invention, and a method for the manufacture of such functional nucleic acids whereby the method is characterized by the use of the hyperimmune serum reactive antigens and fragments thereof according to the present invention and the basic steps are known to the one skilled in the art. The functional nucleic acids are preferably aptamers and spiegelmers.

Aptamers are D-nucleic acids, which are either single stranded or double stranded and which specifically interact with a target molecule. The manufacture or selection of aptamers is, e.g. described in European patent EP 0 533 838. Basically the following steps are realized. First, a mixture of nucleic acids, i. e. potential aptamers, is provided whereby each nucleic acid typically comprises a segment of several, preferably at least eight subsequent randomized nucleotides. This mixture is subsequently contacted with the target molecule whereby the nucleic acid(s) bind to the target molecule, such as based on an increased affinity towards the target or with a bigger force thereto, compared to the candidate mixture. The binding nucleic acid(s) are/is subsequently separated from the remainder of the mixture. Optionally, the thus obtained nucleic acid(s) is amplified using, e.g. polymerase chain reaction. These steps may be repeated several times giving at the end a mixture having an increased ratio of nucleic acids specifically binding to the target from which the final binding nucleic acid is then optionally selected. These specifically binding nucleic acid(s) are referred to as aptamers. It is obvious that at any stage of the method for the generation or identification of the aptamers samples of the mixture of individual nucleic acids may be taken to determine the sequence thereof using standard techniques. It is within the present invention that the aptamers may be stabilized such as, e. g., by introducing defined chemical groups which are known to the one skilled in the art of generating aptamers. Such modification may for example reside in the introduction of an amino group at the 2′-position of the sugar moiety of the nucleotides. Aptamers are currently used as therapeutical agents. However, it is also within the present invention that the thus selected or generated aptamers may be used for target validation and/or as lead substance for the development of medicaments, preferably of medicaments based on small molecules. This is actually done by a competition assay whereby the specific interaction between the target molecule and the aptamer is inhibited by a candidate drug whereby upon replacement of the aptamer from the complex of target and aptamer it may be assumed that the respective drug candidate allows a specific inhibition of the interaction between target and aptamer, and if the interaction is specific, said candidate drug will, at least in principle, be suitable to block the target and thus decrease its biological availability or activity in a respective system comprising such target. The thus obtained small molecule may then be subject to further derivatization and modification to optimize its physical, chemical, biological and/or medical characteristics such as toxicity, specificity, biodegradability and bioavailability.

Spiegelmers and their generation or manufacture is based on a similar principle. The manufacture of spiegelmers is described in international patent application WO 98/08856. Spiegelmers are L-nucleic acids, which means that they are composed of L-nucleotides rather than D-nucleotides as aptamers are. Spiegelmers are characterized by the fact that they have a very high stability in biological systems and, comparable to aptamers, specifically interact with the target molecule against which they are directed. In the process of generating spiegelmers, a heterogeneous population of D-nucleic acids is created and this population is contacted with the optical antipode of the target molecule, in the present case for example with the D-enantiomer of the naturally occurring L-enantiomer of the hyperimmune serum reactive antigens and fragments thereof according to the present invention. Subsequently, those D-nucleic acids are separated which do not interact with the optical antipode of the target molecule. But those D-nucleic acids interacting with the optical antipode of the target molecule are separated, optionally identified and/or sequenced and subsequently the corresponding L-nucleic acids are synthesized based on the nucleic acid sequence information obtained from the D-nucleic acids. These L-nucleic acids, which are identical in terms of sequence with the aforementioned D-nucleic acids interacting with the optical antipode of the target molecule, will specifically interact with the naturally occurring target molecule rather than with the optical antipode thereof. Similar to the method for the generation of aptamers it is also possible to repeat the various steps several times and thus to enrich those nucleic acids specifically interacting with the optical antipode of the target molecule.

In a further aspect the present invention relates to functional nucleic acids interacting with any of the nucleic acid molecules according to the present invention, and a method for the manufacture of such functional nucleic acids whereby the method is characterized by the use of the nucleic acid molecules and their respective sequences according to the present invention and the basic steps are known to the one skilled in the art. The functional nucleic acids are preferably ribozymes, antisense oligonucleotides and siRNA.

Ribozymes are catalytically active nucleic acids, which preferably consist of RNA, which basically comprises two moieties. The first moiety shows a catalytic activity whereas the second moiety is responsible for the specific interaction with the target nucleic acid, in the present case the nucleic acid coding for the hyperimmune serum reactive antigens and fragments thereof according to the present invention. Upon interaction between the target nucleic acid and the second moiety of the ribozyme, typically by hybridisation and Watson-Crick base pairing of essentially complementary stretches of bases on the two hybridising strands, the catalytically active moiety may become active which means that it catalyses, either intramolecularly or intermolecularly, the target nucleic acid in case the catalytic activity of the ribozyme is a phosphodiesterase activity. Subsequently, there may be a further degradation of the target nucleic acid, which in the end results in the degradation of the target nucleic acid as well as the protein derived from the said target nucleic acid. Ribozymes, their use and design principles are known to the one skilled in the art, and, for example described in {Doherty, E. et al., 2001} and {Lewin, A. et al., 2001}.

The activity and design of antisense oligonucleotides for the manufacture of a medicament and as a diagnostic agent, respectively, is based on a similar mode of action. Basically, antisense oligonucleotides hybridise based on base complementarity, with a target RNA, preferably with a mRNA, thereby activating RNase H. RNase H is activated by both phosphodiester and phosphorothioate-coupled DNA. Phosphodiester-coupled DNA, however, is rapidly degraded by cellular nucleases with the exception of phosphorothioate-coupled DNA. These resistant, non-naturally occurring DNA derivatives do not inhibit RNase H upon hybridisation with RNA. In other words, antisense polynucleotides are only effective as DNA RNA hybride complexes. Examples for this kind of antisense oligonucleotides are described, among others, in U.S. Pat. No. 5,849,902 and U.S. Pat. No. 5,989,912. In other words, based on the nucleic acid sequence of the target molecule which in the present case are the nucleic acid molecules for the hyperimmune serum reactive antigens and fragments thereof according to the present invention, either from the target protein from which a respective nucleic acid sequence may in principle be deduced, or by knowing the nucleic acid sequence as such, particularly the mRNA, suitable antisense oligonucleotides may be designed base on the principle of base complementarity.

Particularly preferred are antisense-oligonucleotides, which have a short stretch of phosphorothioate DNA (3 to 9 bases). A minimum of 3 DNA bases is required for activation of bacterial RNase H and a minimum of 5 bases is required for mammalian RNase H activation. In these chimeric oligonucleotides there is a central region that forms a substrate for RNase H that is flanked by hybridising “arms” comprised of modified nucleotides that do not form substrates for RNase H. The hybridising arms of the chimeric oligonucleotides may be modified such as by 2′-O-methyl or 2′-fluoro. Alternative approaches used methylphosphonate or phosphoramidate linkages in said arms. Further embodiments of the antisense oligonucleotide useful in the practice of the present invention are P-methoxyoligonucleotides, partial P-methoxyoligodeoxyribonucleotides or P-methoxyoligonucleotides.

Of particular relevance and usefulness for the present invention are those antisense oligonucleotides as more particularly described in the above two mentioned US patents. These oligonucleotides contain no naturally occurring 5′→3′-linked nucleotides. Rather the oligonucleotides have two types of nucleotides: 2′-deoxyphosphorothioate, which activate RNase H, and 2′-modified nucleotides, which do not. The linkages between the 2′-modified nucleotides can be phosphodiesters, phosphorothioate or P-ethoxyphosphodiester. Activation of RNase H is accomplished by a contiguous RNase H-activating region, which contains between 3 and 5 2′-deoxyphosphorothioate nucleotides to activate bacterial RNase H and between 5 and 10 2′-deoxyphosphorothioate nucleotides to activate eucaryotic and, particularly, mammalian RNase H. Protection from degradation is accomplished by making the 5′ and 3′ terminal bases highly nuclease resistant and, optionally, by placing a 3′ terminal blocking group.

More particularly, the antisense oligonucleotide comprises a 5′ terminus and a 3′ terminus; and from position 11 to 59 5′→3′-linked nucleotides independently selected from the group consisting of 2′-modified phosphodiester nucleotides and 2′-modified P-alkyloxyphosphotriester nucleotides; and wherein the 5′-terminal nucleoside is attached to an RNase H-activating region of between three and ten contiguous phosphorothioate-linked deoxyribonucleotides, and wherein the 3′-terminus of said oligonucleotide is selected from the group consisting of an inverted deoxyribonucleotide, a contiguous stretch of one to three phosphorothioate 2′-modified ribonucleotides, a biotin group and a P-alkyloxyphosphotriester nucleotide.

Also an antisense oligonucleotide may be used wherein not the 5′ terminal nucleoside is attached to an RNase H-activating region but the 3′ terminal nucleoside as specified above. Also, the 5′ terminus is selected from the particular group rather than the 3′ terminus of said oligonucleotide.

The nucleic acids as well as the hyperimmune serum reactive antigens and fragments thereof according to the present invention may be used as or for the manufacture of pharmaceutical compositions, especially vaccines. Preferably such pharmaceutical composition, preferably vaccine is for the prevention or treatment of diseases caused by, related to or associated with C. pneumoniae. In so far another aspect of the invention relates to a method for inducing an immunological response in an individual, particularly a mammal, which comprises inoculating the individual with the hyperimmune serum reactive antigens and fragments thereof of the invention, or a fragment or variant thereof, adequate to produce antibodies to protect said individual from infection, particularly chlamydial infection and most particularly C. pneumoniae infections.

Yet another aspect of the invention relates to a method of inducing an immunological response in an individual which comprises, through gene therapy or otherwise, delivering a nucleic acid functionally encoding hyperimmune serum reactive antigens and fragments thereof, or a fragment or a variant thereof, for expressing the hyperimmune serum reactive antigens and fragments thereof, or a fragment or a variant thereof in vivo in order to induce an immunological response to produce antibodies or a cell mediated T cell response, either cytokine-producing T cells or cytotoxic T cells, to protect said individual from disease, whether that disease is already established within the individual or not. One way of administering the gene is by accelerating it into the desired cells as a coating on particles or otherwise.

A further aspect of the invention relates to an immunological composition which, when introduced into a host capable of having induced within it an immunological response, induces an immunological response in such host, wherein the composition comprises recombinant DNA which codes for and expresses an antigen of the hyperimmune serum reactive antigens and fragments thereof of the present invention. The immunological response may be used therapeutically or prophylactically and may take the form of antibody immunity or cellular immunity such as that arising from CTL or CD4+ T cells.

The hyperimmune serum reactive antigens and fragments thereof of the invention or a fragment thereof may be fused with a co-protein which may not by itself produce antibodies, but is capable of stabilizing the first protein and producing a fused protein which will have immunogenic and protective properties. This fused recombinant protein preferably further comprises an antigenic co-protein, such as Glutathione-S-transferase (GST) or beta-galactosidase, relatively large co-proteins which solubilise the protein and facilitate production and purification thereof. Moreover, the co-protein may act as an adjuvant in the sense of providing a generalized stimulation of the immune system. The co-protein may be attached to either the amino or carboxy terminus of the first protein.

Also, provided by this invention are methods using the described nucleic acid molecule or particular fragments thereof in such genetic immunization experiments in animal models of infection with Chlamydia pneumoniae. Such fragments will be particularly useful for identifying protein epitopes able to provoke a prophylactic or therapeutic immune response. This approach can allow for the subsequent preparation of monoclonal antibodies of particular value from the requisite organ of the animal successfully resisting or clearing infection for the development of prophylactic agents or therapeutic treatments of C. pneumoniae infection in mammals, particularly humans.

The hyperimmune serum reactive antigens and fragments thereof may be used as an antigen for vaccination of a host to produce specific antibodies which protect against invasion of bacteria, for example by blocking adherence of bacteria to damaged tissue. Examples of tissue damage include wounds in skin or connective tissue and mucosal tissues caused e.g. by viral infection (esp. respiratory, such as the flu) mechanical, chemical or thermal damage or by implantation of indwelling devices, or wounds in the mucous membranes, such as the mouth, mammary glands, urethra or vagina.

The present invention also includes a vaccine formulation, which comprises the immunogenic recombinant protein together with a suitable carrier. Since the protein may be broken down in the stomach, it is preferably administered parenterally, including, for example, administration that is subcutaneous, intramuscular, intravenous, intradermal intranasal or transdermal. Formulations suitable for parenteral administration include aqueous and non-aqueous sterile injection solutions which may contain anti-oxidants, buffers, bacteriostats and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampoules and vials, and may be stored in a freeze-dried condition requiring only the addition of the sterile liquid carrier immediately prior to use. The vaccine formulation may also include adjuvant systems for enhancing the immunogenicity of the formulation, such as oil-in-water systems and other systems known in the art. The dosage will depend on the specific activity of the vaccine and can be readily determined by routine experimentation.

According to another aspect, the present invention relates to a pharmaceutical composition comprising such a hyperimmune serum-reactive antigen or a fragment thereof as provided in the present invention for C. pneumoniae. Such a pharmaceutical composition may comprise one, preferably at least two, or more hyperimmune serum reactive antigens or fragments thereof against C. pneumoniae. Optionally, such C. pneumoniae hyperimmune serum reactive antigens or fragments thereof may also be combined with antigens against other pathogens in a combination pharmaceutical composition. Preferably, said pharmaceutical composition is a vaccine for preventing or treating an infection caused by C. pneumoniae and/or other pathogens against which the antigens have been included in the vaccine.

According to a further aspect, the present invention relates to a pharmaceutical composition comprising a nucleic acid molecule encoding a hyperimmune serum-reactive antigen or a fragment thereof as identified above for C. pneumoniae. Such a pharmaceutical composition may comprise one or more nucleic acid molecules encoding hyperimmune serum reactive antigens or fragments thereof against C. pneumoniae. Optionally, such C. pneumoniae nucleic acid molecules encoding hyperimmune serum reactive antigens or fragments thereof may also be combined with nucleic acid molecules encoding antigens against other pathogens in a combination pharmaceutical composition. Preferably, said pharmaceutical composition is a vaccine for preventing or treating an infection caused by C. pneumoniae and/or other pathogens against which the antigens have been included in the vaccine.

The pharmaceutical composition may contain any suitable auxiliary substances, such as buffer substances, stabilisers or further active ingredients, especially ingredients known in connection of pharmaceutical composition and/or vaccine production.

A preferable carrier/or excipient for the hyperimmune serum-reactive antigens, fragments thereof or a coding nucleic acid molecule thereof according to the present invention is an immunostimulatory compound for further stimulating the immune response to the given hyperimmune serum-reactive antigen, fragment thereof or a coding nucleic acid molecule thereof. Preferably the immunostimulatory compound in the pharmaceutical preparation according to the present invention is selected from the group of polycationic substances, especially polycationic peptides, immunostimulatory nucleic acids molecules, preferably immunostimulatory deoxynucleotides, alum, Freund's complete adjuvants, Freund's incomplete adjuvants, neuroactive compounds, especially human growth hormone, or combinations thereof

It is also within the scope of the present invention that the pharmaceutical composition, especially vaccine, comprises apart from the hyperimmune serum reactive antigens, fragments thereof and/or coding nucleic acid molecules thereof according to the present invention other compounds which are biologically or pharmaceutically active. Preferably, the vaccine composition comprises at least one polycationic peptide. The polycationic compound(s) to be used according to the present invention may be any polycationic compound, which shows the characteristic effects according to the WO 97/30721. Preferred polycationic compounds are selected from basic polypeptides, organic polycations, basic polyamino acids or mixtures thereof These polyamino acids should have a chain length of at least 4 amino acid residues (WO 97/30721). Especially preferred are substances like polylysine, polyarginine and polypeptides containing more than 20%, especially more than 50% of basic amino acids in a range of more than 8, especially more than 20, amino acid residues or mixtures thereof Other preferred polycations and their pharmaceutical compositions are described in WO 97/30721 (e.g. polyethyleneimine) and WO 99/38528. Preferably these polypeptides contain between 20 and 500 amino acid residues, especially between 30 and 200 residues.

These polycationic compounds may be produced chemically or recombinantly or may be derived from natural sources.

Cationic (poly)peptides may also be anti-microbial with properties as reviewed in {Ganz, T., 1999}. These (poly)peptides may be of prokaryotic or animal or plant origin or may be produced chemically or recombinantly (WO 02/13857). Peptides may also belong to the class of defensins (WO 02/13857). Sequences of such peptides can be, for example, found in the Antimicrobial Sequences Database under the following internet address:
http://www.bbcm.univ.trieste.it/˜tossi/pag2.html

Such host defence peptides or defensives are also a preferred form of the polycationic polymer according to the present invention. Generally, a compound allowing as an end product activation (or down-regulation) of the adaptive immune system, preferably mediated by APCs (including dendritic cells) is used as polycationic polymer.

Especially preferred for use as polycationic substances in the present invention are cathelicidin derived antimicrobial peptides or derivatives thereof (International patent application WO 02/13857, incorporated herein by reference), especially antimicrobial peptides derived from mammalian cathelicidin, preferably from human, bovine or mouse.

Polycationic compounds derived from natural sources include HIV-REV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelin. For example, mouse cathelin is a peptide, which has the amino acid sequence NH2-RLAGLLRKGGEKIGEKLKKIGOKIKNFFQKLVPQPE-COOH NH2-RLAGLLRKGGEKIGEKLKKIGQKIKNFFQKLVPQPE-COOH (SEQ ID NO:122). Related or derived cathelin substances contain the whole or parts of the cathelin sequence with at least 15-20 amino acid residues. Derivations may include the substitution or modification of the natural amino acids by amino acids, which are not among the 20 standard amino acids. Moreover, further cationic residues may be introduced into such cathelin molecules. These cathelin molecules are preferred to be combined with the antigen. These cathelin molecules surprisingly have turned out to be also effective as an adjuvant for an antigen without the addition of further adjuvants. It is therefore possible to use such cathelin molecules as efficient adjuvants in vaccine formulations with or without further immunactivating immunoactivating substances.

Another preferred polycationic substance to be used according to the present invention is a synthetic peptide containing at least 2 KLK-motifs separated by a linker of 3 to 7 hydrophobic amino acids (International patent application WO 02/32451, incorporated herein by reference).

The pharmaceutical composition of the present invention may further comprise immunostimulatory nucleic acid(s). Immunostimulatory nucleic acids are e. g. neutral or artificial CpG containing nucleic acids, short stretches of nucleic acids derived from non-vertebrates or in form of short oligonucleotides (ODNs) containing non-methylated cytosine-guanine di-nucleotides (CpG) in a certain base context (e.g. described in WO 96/02555). Alternatively, also nucleic acids based on inosine and cytidine as e.g. described in the WO 01/93903, or deoxynucleic acids containing deoxy-inosine and/or deoxyuridine residues (described in WO 01/93905 and PCT/EP 02/05448, incorporated herein by reference) may preferably be used as immunostimulatory nucleic acids for the present invention. Preferably, the mixtures of different immunostimulatory nucleic acids may be used according to the present invention.

It is also within the present invention that any of the aforementioned polycationic compounds is combined with any of the immunostimulatory nucleic acids as aforementioned. Preferably, such combinations are according to the ones as described in WO 01/93905, WO 02/32451, WO 01/54720, WO 01/93903, WO 02/13857 and PCT/EP 02/05448 and the Austrian patent application A 1924/2001, incorporated herein by reference.

In addition or alternatively such vaccine composition may comprise apart from the hyperimmune serum reactive antigens and fragments thereof, and the coding nucleic acid molecules thereof according to the present invention a neuroactive compound. Preferably, the neuroactive compound is human growth factor as, e.g. described in WO 01/24822. Also preferably, the neuroactive compound is combined with any of the polycationic compounds and/or immunostimulatory nucleic acids as afore-mentioned.

In a further aspect the present invention is related to a pharmaceutical composition. Such pharmaceutical composition is, for example, the vaccine described herein. Also a pharmaceutical composition is a pharmaceutical composition which comprises any of the following compounds or combinations thereof: the nucleic acid molecules according to the present invention, the hyperimmune serum reactive antigens and fragments thereof according to the present invention, the vector according to the present invention, the cells according to the present invention, the antibody according to the present invention, the functional nucleic acids according to the present invention and the binding peptides such as the anticalines according to the present invention, any agonists and antagonists screened as described herein. In connection therewith any of these compounds may be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of a hyperimmune serum reactive antigen and fragments thereof of the invention and a pharmaceutically acceptable carrier or excipient. Such carriers may include, but are not limited to, saline, buffered saline, dextrose, water, glycerol, ethanol and combinations thereof. The formulation should suit the mode of administration.

The pharmaceutical compositions may be administered in any effective, convenient manner including, for instance, administration by topical, oral, anal, vaginal, intravenous, intraperitoneal, intramuscular, subcutaneous, intranasal, intratracheal or intradermal routes among others.

In therapy or as a prophylactic, the active agent may be administered to an individual as an injectable composition, for example as a sterile aqueous dispersion, preferably isotonic.

Alternatively the composition may be formulated for topical application, for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

In addition to the therapy described above, the compositions of this invention may be used generally as a wound treatment agent to prevent adhesion of bacteria to matrix proteins exposed in wound tissue and for prophylactic use in dental treatment as an alternative to, or in conjunction with, antibiotic prophylaxis.

A vaccine composition is conveniently in injectable form. Conventional adjuvants may be employed to enhance the immune response. A suitable unit dose for vaccination is 0.05-5 μg antigen/per kg of body weight, and such dose is preferably administered 1-3 times and with an interval of 1-3 weeks.

With the indicated dose range, no adverse toxicological effects should be observed with the compounds of the invention, which would preclude their administration to suitable individuals.

In a further embodiment the present invention relates to diagnostic and pharmaceutical packs and kits comprising one or more containers filled with one or more of the ingredients of the aforementioned compositions of the invention. The ingredient(s) can be present in a useful amount, dosage, formulation or combination. Associated with such container(s) can be a notice in the form prescribed by a governmental agency regulating the manufacture, use or sale of pharmaceuticals or biological products, reflecting approval by the agency of the manufacture, use or sale of the product for human administration.

In connection with the present invention any disease related use as disclosed herein such as, e. g. use of the pharmaceutical composition or vaccine, is particularly a disease or diseased condition which is caused by, linked or associated with Chlamydiaceae bacteria, more preferably, C. pneumoniae. In connection therewith it is to be noted that C. pneumoniae comprises several strains including those disclosed herein. A disease related, caused or associated with the bacterial infection to be prevented and/or treated according to the present invention includes besides others community-acquired pneumoniae, bronchitis, pharyngitis, sinusitis in humans.

In a still further embodiment the present invention is related to a screening method using any of the hyperimmune serum reactive antigens or nucleic acids according to the present invention. Screening methods as such are known to the one skilled in the art and can be designed such that an agonist or an antagonist is screened. Preferably an antagonist is screened which in the present case inhibits or prevents the binding of any hyperimmune serum reactive antigen and fragment thereof according to the present invention to an interaction partner. Such interaction partner can be a naturally occurring interaction partner or a non-naturally occurring interaction partner.

The invention also provides a method of screening compounds to identify those, which enhance (agonist) or block (antagonist) the function of hyperimmune serum reactive antigens and fragments thereof or nucleic acid molecules of the present invention, such as its interaction with a binding molecule. The method of screening may involve high-throughput.

For example, to screen for agonists or antagonists, the interaction partner of the nucleic acid molecule and nucleic acid, respectively, according to the present invention, maybe a synthetic reaction mix, a cellular compartment, such as a membrane, cell envelope or cell wall, or a preparation of any thereof, may be prepared from a cell that expresses a molecule that binds to the hyperimmune serum reactive antigens and fragments thereof of the present invention. The preparation is incubated with labelled hyperimmune serum reactive antigens and fragments thereof in the absence or the presence of a candidate molecule, which may be an agonist or antagonist. The ability of the candidate molecule to bind the binding molecule is reflected in decreased binding of the labelled ligand. Molecules which bind gratuitously, i. e., without inducing the functional effects of the hyperimmune serum reactive antigens and fragments thereof, are most likely to be good antagonists. Molecules that bind well and elicit functional effects that are the same as or closely related to the hyperimmune serum reactive antigens and fragments thereof are good agonists.

The functional effects of potential agonists and antagonists may be measured, for instance, by determining the activity of a reporter system following interaction of the candidate molecule with a cell or appropriate cell preparation, and comparing the effect with that of the hyperimmune serum reactive antigens and fragments thereof of the present invention or molecules that elicit the same effects as the hyperimmune serum reactive antigens and fragments thereof. Reporter systems that may be useful in this regard include but are not limited to colorimetric labelled substrate converted into product, a reporter gene that is responsive to changes in the functional activity of the hyperimmune serum reactive antigens and fragments thereof, and binding assays known in the art.

Another example of an assay for antagonists is a competitive assay that combines the hyperimmune serum reactive antigens and fragments thereof of the present invention and a potential antagonist with membrane-bound binding molecules, recombinant binding molecules, natural substrates or ligands, or substrate or ligand mimetics, under appropriate conditions for a competitive inhibition assay. The hyperimmune serum reactive antigens and fragments thereof can be labelled such as by radioactivity or a colorimetric compound, such that the molecule number of hyperimmune serum reactive antigens and fragments thereof bound to a binding molecule or converted to product can be determined accurately to assess the effectiveness of the potential antagonist.

Potential antagonists include small organic molecules, peptides, polypeptides and antibodies that bind to a hyperimmune serum reactive antigen and fragments thereof of the invention and thereby inhibit or extinguish its activity. Potential antagonists also may be small organic molecules, a peptide, a polypeptide such as a closely related protein or antibody that binds to the same sites on a binding molecule without inducing functional activity of the hyperimmune serum reactive antigens and fragments thereof of the invention.

Potential antagonists include a small molecule, which binds to and occupies the binding site of the hyperimmune serum reactive antigens and fragments thereof thereby preventing binding to cellular binding molecules, such that normal biological activity is prevented. Examples of small molecules include but are not limited to small organic molecules, peptides or peptide-like molecules.

Other potential antagonists include antisense molecules (see {Okano, H. et al., 1991}; OLIGODEOXYNUCLEOTIDES AS ANTISENSE INHIBITORS OF GENE EXPRESSION; CRC Press, Boca Ration, Fla. (1988), for a description of these molecules).

Preferred potential antagonists include derivatives of the hyperimmune serum reactive antigens and fragments thereof of the invention.

As used herein the activity of a hyperimmune serum reactive antigen and fragment thereof according to the present invention is its capability to bind to any of its interaction partner or the extent of such capability to bind to its or any interaction partner.

In a particular aspect, the invention provides the use of the hyperimmune serum reactive antigens and fragments thereof, nucleic acid molecules or inhibitors of the invention to interfere with the initial physical interaction between a pathogen and mammalian host responsible for sequelae of infection. In particular the molecules of the invention may be used: i) in the prevention of adhesion of C. pneumoniae to mammalian extracellular matrix proteins at mucosal surfaces and on in-dwelling devices or to extracellular matrix proteins in wounds; ii) to block bacterial adhesion between mammalian extracellular matrix proteins and bacterial proteins which mediate tissue damage or invasion iii) or lead to evasion of immune defence; iv) to block the normal progression of pathogenesis in infections initiated other than by the implantation of in-dwelling devices or by other surgical techniques, e.g. through inhibiting nutrient acquisition {Brown, J. et al., 2001}.

Each of the DNA coding sequences provided herein may be used in the discovery and development of antibacterial compounds. The encoded protein upon expression can be used as a target for the screening of antibacterial drugs. Additionally, the DNA sequences encoding the amino terminal regions of the encoded protein or Shine-Delgarno or other translation facilitating sequences of the respective mRNA can be used to construct antisense sequences to control the expression of the coding sequence of interest.

The antagonists and agonists may be employed, for instance, to inhibit diseases arising from infection with Chlamydiaceae, especially C. pneumoniae, such as pneumonia.

In a still further aspect the present invention is related to an affinity device such affinity device comprises as least a support material and any of the hyperimmune serum reactive antigens and fragments thereof according to the present invention, which is attached to the support material. Because of the specificity of the hyperimmune serum reactive antigens and fragments thereof according to the present invention for their target cells or target molecules or their interaction partners, the hyperimmune serum reactive antigens and fragments thereof allow a selective removal of their interaction partner(s) from any kind of sample applied to the support material provided that the conditions for binding are met. The sample may be a biological or medical sample, including but not limited to, fermentation broth, cell debris, cell preparation, tissue preparation, organ preparation, blood, urine, lymph liquid, liquor and the like.

The hyperimmune serum reactive antigens and fragments thereof may be attached to the matrix in a covalent or non-covalent manner. Suitable support material is known to the one skilled in the art and can be selected from the group comprising cellulose, silicon, glass, aluminium, paramagnetic beads, starch and dextrane.

The present invention is further illustrated by the following figures, examples and the sequence listing, from which further features, embodiments and advantages may be taken. It is to be understood that the present examples are given by way of illustration only and not by way of limitation of the disclosure.

In connection with the present invention

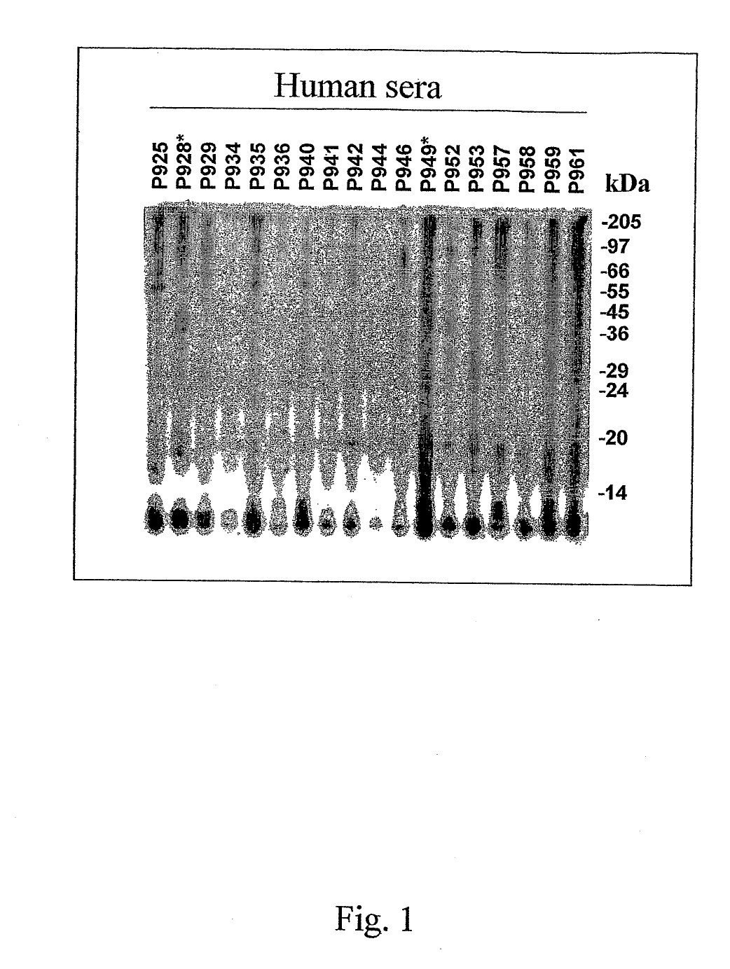

FIG. 1 shows the characterization of C. pneumoniae specific human sera.

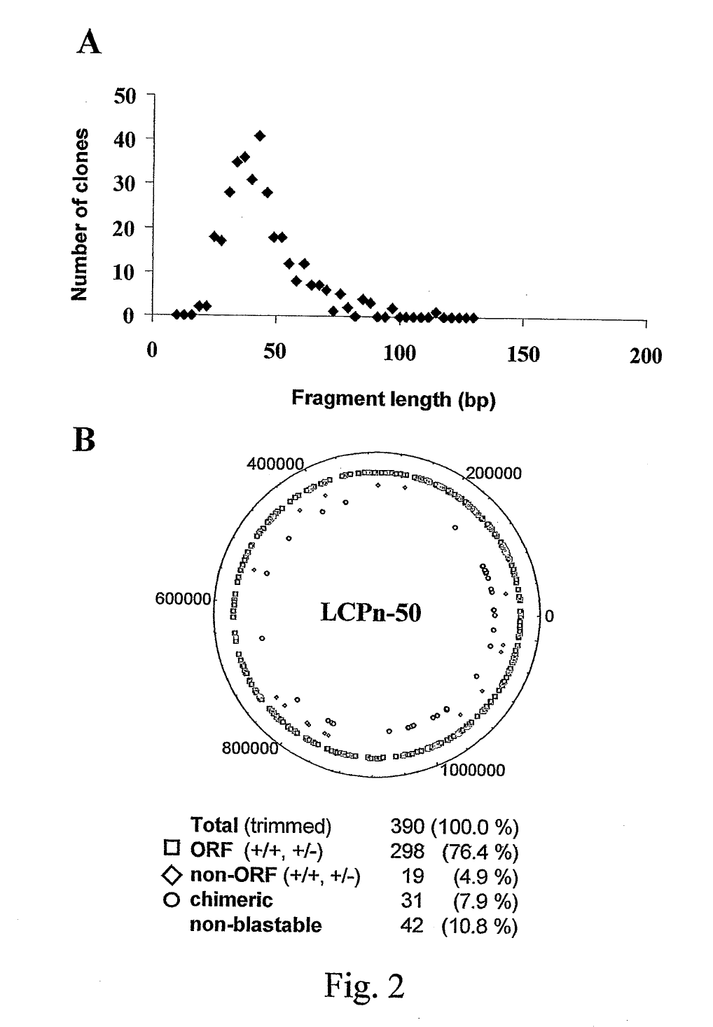

FIG. 2 shows the characterization of the small fragment genomic library, LCPn-50, from Chlamydia pneumoniae AR39.

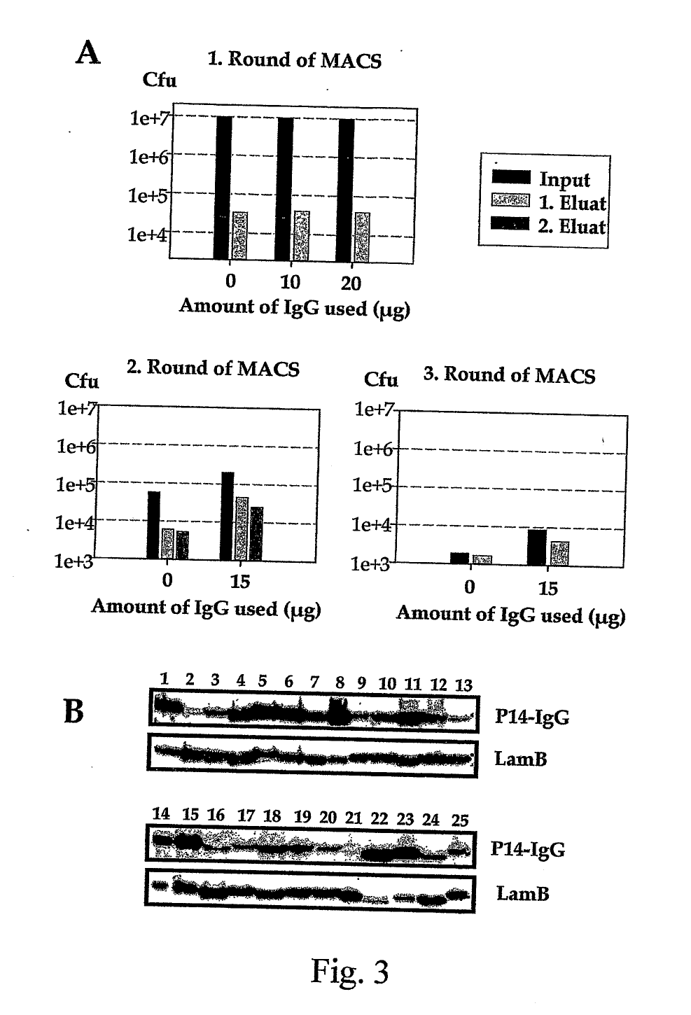

FIG. 3 shows the selection of bacterial cells by MACS using biotinylated human IgGs.

Table 1 shows the summary of the screens performed with genomic C. pneumoniae libraries and human serum.

Table 2 shows the summary of epitope serology analysis with human sera.

The figures to which it might be referred to in the specification are described in the following in more details.

FIG. 1 shows the characterization of human sera for anti-C. pneumoniae antibodies as measured by immunoblotting. Sera were preselected for high anti-C. pneumoniae IgG antibody levels by Chlamydien-IgG-ELISA medac. Proteins of the elementary bodies (EB) isolated from C. pneumoniae AR39 infected HeLa cells were separated on SDS-PAGE gels and transferred to nitrocellulose membrane. Results of a representative experiment using selected patients' sera at 5.000× dilution are shown. Blots were developed with anti-human IgG secondary antibody reagent. The most reactive samples were selected into screening pools (indicated with *). Mw: molecular weight markers.

FIG. 2(A) shows the fragment size distribution of the Chlamydia pneumoniae AR39 small fragment genomic library, LCPn-50. After sequencing 480 randomly selected clones sequences were trimmed to eliminate vector residues and the number of clones with various genomic fragment sizes were plotted. (B) shows the graphic illustration of the distribution of the same set of randomly sequenced clones of LCPn-50 over the C. pneumoniae chromosome (according to the AR39 genome data). Rectangles indicate matching sequences to annotated ORFs and diamonds represent fully matched clones to non-coding chromosomal sequences in +/+ or +/− orientation, respectively. Circles position all clones with chimeric sequences. Numeric distances in base pairs are indicated over the circular genome for orientation. Partitioning of various clone sets within the library is given in numbers and percentage at the bottom of the figure.

FIG. 3(A) shows the MACS selection with biotinylated human IgGs. The LCPn-50 library in pMAL9.1 was screened with 10 to 20 μg biotinylated IgG (P14-IgG, purified from human serum). As negative control, no serum was added to the library cells for screening. Number of cells selected after the 1^{st} and/or 2^{nd} elution are shown for each of the three selection rounds. (B) shows the reactivity of specific clones (1-25) selected by bacterial surface display as analysed by immunoblot analysis with the human serum IgG pool (P14-IgG, 4 μg/μl) used for selection by MACS at a dilution of 1:3,000. As a loading control the same blot was also analysed with antibodies directed against the platform protein LamB at a dilution of 1:5,000 of immune rabbit serum.

Table 1: Immunogenic Proteins Identified by Bacterial Surface Display.

A, 50 bp library (LCPn-50) of C. pneumoniae AR39 in lamB with P14-IgG (number of clones after trimming: 755), B, 300 bp library (LCPn-300) in fhuA with P14-IgG (669); The number of selected clones per ORF is listed as well as the immunogenic region delineated by the selected clones. CP0018, annotated reading frame of C. pneumoniae; ARF0217, predicted novel ORF in alternative reading frame of CP0217; CRF0014, predicted novel ORF on complement reading frame of CP0014. *, prediction of sequences longer than 5 amino acids capable of inducing an antibody response was performed with the program ANTIGENIC {Kolaskar, A. et al., 1990}; **, prediction of sequences capable of inducing a class II-restricted T cell response was performed with the program TEPITOPE {Bian, H. et al., 2003}. Epitopes or regions are shown that are identified in at least four of the eight MHC types analysed with a threshold of 5%. ***, prediction of nonameric sequences capable of inducing a class I-restricted T cell response was performed with the program SYFPEITHI {Rammensee, H. et al., 1999}. Epitopes are shown that are identified individually for four MHC types (A0201, B0702, A03, A2402) with a score above 20.

Table 2: Epitope Serology with Human Sera.

Immune reactivity of individual synthetic peptides representing selected epitopes with human sera strongly reactive against C. pneumoniae is shown. The extent of reactivity is expressed as scores, which were calculated based on the sum of ELISA reactivities with 22 individual sera, based on the following calculations: −=0; +=1; ++=2; +++=3. Positivity was assessed based on OD_{450 nm} readings at two different serum dilutions after correction for background. Locations of synthetic peptides within the antigenic ORFs according to the genome annotation of the C. pneumoniae AR39 strain are given in the 2nd column indicating the first and last amino acid residue, respectively. Peptide names: CP0018.1, present in annotated CP0018; ARF0271.1, present in potential novel ORF in alternative reading-frame of CP0271; CRF1083.1, present in potential novel ORF on complement of CP 1083.

### EXAMPLES

### Example 1

### Characterization and Selection of Human Sera Based Anti-C. pneumoniae Antibodies, Preparation of Antibody Screening Reagents

Experimental Procedures

Enzyme Linked Immune Assay (ELISA).

A commercially available ELISA kit, Chlamydien-IgG-ELISA medac (Medac GmbH, Germany), which employs a highly purified and specific antigen was used to measure anti-C. pneumoniae antibody titers. Three dilutions of sera, 400×, 200×, 100× were tested, and reactivities were expressed as titers >1:400; 1:400; 1:200; 1:100 and <1:100.

Immunoblotting

Elementary bodies (EB), used as bacterial antigen extract were isolated from C. pneumoniae AR39 infected HeLa cell cultures according to {Wang, S. et al., 1991}. The infectivity of EBs was destroyed and proteins were solubilized by adding SDS-PAGE sample buffer containing SDS and 2-mercaptoethanol. Approximately 5 μg total protein was separated by SDS-PAGE using the BioRad Mini-Protean 3 Cell electrophoresis system and proteins were transferred to nitrocellulose membrane (ECL, Amersham Pharmacia). After overnight blocking in 5% milk, human sera were added at 5,000× dilution, and HRPO labeled anti-human IgG was used for detection.

Purification of antibodies for genomic screening. Five sera were selected based on the overall anti-chlamydial titers for a serum pool used in the screening procedure. Antibodies against E. coli proteins were removed by incubating the heat-inactivated sera with whole cell E. coli cells (DH5alpha, transformed with pHIE11, grown under the same condition as used for bacterial surface display). Highly enriched preparations of IgGs from the pooled, depleted sera were generated by protein G affinity chromatography, according to the manufacturer's instructions (UltraLink Immobilized Protein G, Pierce). The efficiency of IgG purification was checked by SDS-PAGE and protein concentration measurements (OD_{280 nm}).

Results

The antibodies produced against C. pneumoniae by the human immune system and present in human sera are indicative of the in vivo expression of the antigenic proteins and their immunogenicity. These molecules are essential for the identification of individual antigens in the approach as described in the present invention, which is based on the interaction of the specific anti-chlamydial antibodies and the corresponding C. pneumoniae peptides or proteins. To gain access to relevant antibody repertoires, human sera were collected from patients with symptoms of C. pneumoniae related infections, such as pneumonia, and bronchitis. C. pneumoniae was indicated to be the causative agent by medical serological tests.

Infections with Chlamydia pneumoniae are detected and diagnosed by serology, since the pathogen is not culturable with routine microbiological methods. Highly specific and sensitive diagnostic kits based on antigen detection have been developed and are available commercially. We have selected patients' sera having a high titer against C. pneumoniae detected by a standard Chlamydia ELISA kit routinely used in the clinic for diagnosis of acute, chronic and persistent infections caused by Chlamydia species. 185 serum samples were tested, all derived from individuals selected for diagnostic testing for the presence of Chlamydia pneumoniae specific antibodies based on clinical symptoms. 83 sera showed antibody titers >1:400; 34 sera showed titers of approximately 1:400; 14 sera of 1:200; 20 sera of 1:100 and 34 sera had titers <1:100. According to epidemiologic studies C. pneumoniae carriage and infection is widespread, with frequent reinfection during lifetime. For that reason, primary selection of sera aimed at the identification of samples with the highest IgG titer (>400) to reduce the risk of nonspecific, false positive diagnosis.

Subsequently, pre-selected sera were analyzed by immunoblotting to ensure antibody reactivities against multiple proteinaceous antigens present in C. pneumoniae. The representative immunoblot shown in FIG. 1 demonstrates that different patterns of reactivities were detected with the individual sera when tested against proteins of elementary bodies, isolated from infected human cells (HeLa) in in vitro cultures. Special attention was made to select sera displaying different pattern of reactivities based on these immunoblot analysis.

Five selected sera were pooled to further enrich for abundant antibodies, but still having a representation of antibody repertoires of different individuals. IgG antibodies were purified from pooled sera by affinity chromatography and depleted of E. coli-reactive antibodies to avoid background in the bacterial surface display screen.

### Example 2

### Generation of Highly Random, Frame-Selected, Small-Fragment, Genomic DNA Libraries of Chlamydia pneumoniae AR39

Experimental Procedures

Preparation of chlamydial genomic DNA. C. pneumoniae AR39 was cultivated as described in {Campbell, L. et al., 1989}. Elementary bodies (EB) were isolated and used for the preparation of genomic DNA. Genomic DNA from C. pneumoniae EBs was prepared as described by {Cox, R. et al., 1988}. The final genomic DNA preparation was dissolved in ddH₂O.

Preparation of small genomic DNA fragments. Genomic DNA from C. pneumoniae AR39 was mechanically sheared into fragments ranging in size between 150 and 300 by using a cup-horn sonicator (Bandelin Sonoplus UV 2200 sonicator equipped with a BB5 cup horn, 10 sec. pulses at 100% power output) or into fragments of size between 50 and 70 by by mild DNase I treatment (Novagen). It was observed that sonication yielded a much tighter fragment size distribution when breaking the DNA into fragments of the 150-300 by size range. However, despite extensive exposure of the DNA to ultrasonic wave-induced hydromechanical shearing force, subsequent decrease in fragment size could not be efficiently and reproducibly achieved. Therefore, fragments of 50 to 70 by in size were obtained by mild DNase I treatment using Novagen's shotgun cleavage kit. A 1:20 dilution of DNase I provided with the kit was prepared and the digestion was performed in the presence of MnCl₂ in a 60 μl volume at 20° C. for 5 min to ensure double-stranded cleavage by the enzyme. Reactions were stopped with 2 μl of 0.5 M EDTA and the fragmentation efficiency was evaluated on a 2% TAE-agarose gel. This treatment resulted in total fragmentation of genomic DNA into near 50-70 by fragments. Fragments were then blunt-ended twice using T4 DNA Polymerase in the presence of 100 μM each of dNTPs to ensure efficient flushing of the ends. Fragments were used immediately in ligation reactions or frozen at −20° C. for subsequent use.

Description of the vectors. The vector pMAL4.31 was constructed on a pASK-IBA backbone {Skerra, A., 1994} with the beta-lactamase (bla) gene exchanged with the Kanamycin resistance gene. In addition the bla gene was cloned into the multiple cloning site. The sequence encoding mature beta-lactamase is preceded by the leader peptide sequence of ompA to allow efficient secretion across the cytoplasmic membrane. Furthermore a sequence encoding the first 12 amino acids (spacer sequence) of mature beta-lactamase follows the ompA leader peptide sequence to avoid fusion of sequences immediately after the leader peptidase cleavage site, since e.g. clusters of positive charged amino acids in this region would decrease or abolish translocation across the cytoplasmic membrane {Kajava, A. et al., 2000}. A SmaI restriction site serves for library insertion. An upstream FseI site and a downstream NotI site, which were used for recovery of the selected fragment, flank the SmaI site. The three restriction sites are inserted after the sequence encoding the 12 amino acid spacer sequence in such a way that the bla gene is transcribed in the −1 reading frame resulting in a stop codon 15 by after the NotI site. A +1 by insertion restores the bla ORF so that beta-lactamase protein is produced with a consequent gain of Ampicillin resistance.

The vector pMAL9.1 was constructed by cloning the lamB gene into the multiple cloning site of pEH1 {Hashemzadeh-Bonehi, L. et al., 1998}. Subsequently, a sequence was inserted in lamB after amino acid 154, containing the restriction sites FseI, SmaI and NotI. The reading frame for this insertion was constructed in such a way that transfer of frame-selected DNA fragments excised by digestion with FseI and NotI from plasmid pMAL4.31 yields a continuous reading frame of lamB and the respective insert.

The vector pHIE11 was constructed by cloning the fhuA gene into the multiple cloning site of pEH1. Thereafter, a sequence was inserted in fhuA after amino acid 405, containing the restriction site FseI, XbaI and NotI. The reading frame for this insertion was chosen in a way that transfer of frame-selected DNA fragments excised by digestion with FseI and NotI from plasmid pMAL4.31 yields a continuous reading frame of fhuA and the respective insert.

Cloning and evaluation of the library for frame selection. Genomic C. pneumoniae AR39 DNA fragments were ligated into the SmaI site of the vector pMAL4.31. Recombinant DNA was electroporated into DH10B electrocompetent E. coli cells (GIBCO BRL) and transformants plated on LB-agar supplemented with Kanamycin (50 μg/ml) and Ampicillin (50 μg/ml). Plates were incubated over night at 37° C. and colonies collected for large scale DNA extraction. A representative plate was stored and saved for collecting colonies for colony PCR analysis and large-scale sequencing. A simple colony PCR assay was used to initially determine the rough fragment size distribution as well as insertion efficiency. From sequencing data the precise fragment size was evaluated, junction intactness at the insertion site as well as the frame selection accuracy (3n+1 rule).

Cloning and evaluation of the library for bacterial surface display. Genomic DNA fragments were excised from the pMAL4.31 vector, containing the C. pneumoniae library with the restriction enzymes FseI and NotI. The entire population of fragments was then transferred into plasmids pMAL9.1 (LamB) or pHIE11 (FhuA), which have been digested with FseI and NotI. Using these two restriction enzymes, which recognise an 8 by GC rich sequence, the reading frame that was selected in the pMAL4.31 vector is maintained in each of the platform vectors. The plasmid library was then transformed into E. coli DH5alpha cells by electroporation. Cells were plated onto large LB-agar plates supplemented with 50 μg/ml Kanamycin and grown over night at 37° C. at a density yielding clearly visible single colonies. Cells were then scraped off the surface of these plates, washed with fresh LB medium and stored in aliquots for library screening at −80° C.

Results

Libraries for frame selection. Two libraries (LCPn-50 and LCPn-300) were generated in the pMAL4.31 vector with sizes of approximately 50 and 300 bp, respectively. For each library, ligation and subsequent transformation of approximately 1 μg of pMAL4.31 plasmid DNA and 50 ng of fragmented genomic C. pneumoniae AR39 DNA yielded 6×10⁴ to 3×10⁵ clones after frame selection. To assess the randomness of the libraries, 480 randomly chosen clones of LCPn-50 were sequenced. After trimming of the vector sequences, 390 could be subjected to bioinformatic analysis, showing that of these clones only very few were present more than once. Furthermore, it was shown that 98% of the clones fell in the size range between 25 and 100 by with an average size of 46 by (FIG. 2). Almost all sequences followed the 3n+1 rule, showing that all clones were properly frame selected.

Bacterial surface display libraries. The display of peptides on the surface of E. coli required the transfer of the inserts from the LCPn libraries from the frame selection vector pMAL4.31 to the display plasmids pMAL9.1 (LamB) or pHIE11 (FhuA). Genomic DNA fragments were excised by FseI and NotI restriction and ligation of 5 ng inserts with 0.1 μg plasmid DNA and subsequent transformation into DH5alpha cells resulted in 2×10⁵ to 2×10⁶ clones. The clones were scraped off the LB plates and frozen without further amplification.

### Example 3

### Identification of Highly Immunogenic Peptide Sequences from C. pneumoniae Using Bacterial surface Displayed Genomic Libraries and Human Serum

Experimental Procedures

MACS screening. Approximately 2.5×10⁸ cells from a given library were grown in 5 ml LB-medium supplemented with 50 μg/ml Kanamycin for 2 h at 37° C. Expression was induced by the addition of 1 mM IPTG for 30 min. Cells were washed twice with fresh LB medium and approximately 2×10⁷ cells re-suspended in 100 μl LB medium and transferred to an Eppendorf tube.

Ten to 20 μg of biotinylated, human IgGs purified from serum was added to the cells and the suspension incubated overnight at 4° C. with gentle shaking. 900 μl of LB medium was added, the suspension mixed and subsequently centrifuged for 10 min at 6,000 rpm at 4° C. (For IgA screens, 10 to 20 μg of purified IgAs were used and these captured with biotinylated anti-human-IgG secondary antibodies). Cells were washed once with 1 ml LB and then re-suspended in 100 μl LB medium. 10 μl of MACS microbeads coupled to streptavidin (Miltenyi Biotech, Germany) were added and the incubation continued for 20 min at 4° C. Thereafter 900 μl of LB medium was added and the MACS microbead cell suspension was loaded onto the equilibrated MS column (Miltenyi Biotech, Germany) which was fixed to the magnet. (The MS columns were equilibrated by washing once with 1 ml 70% EtOH and twice with 2 ml LB medium.)

The column was then washed three times with 3 ml LB medium. After removal of the magnet, cells were eluted by washing with 2 ml LB medium. After washing the column with 3 ml LB medium, the 2 ml eluate was loaded a second time on the same column and the washing and elution process repeated. The loading, washing and elution process was performed a third time, resulting in a final eluate of 2 ml.

A second round of screening was performed as follows. The cells from the final eluate were collected by centrifugation and re-suspended in 1 ml LB medium supplemented with 50 μg/ml Kanamycin. The culture was incubated at 37° C. for 90 min and then induced with 1 mM IPTG for 30 min. Cells were subsequently collected, washed once with 1 ml LB medium and suspended in 10 μl LB medium. 10 μg of human, biotinylated IgGs were added again and the suspension incubated over night at 4° C. with gentle shaking. All further steps were exactly the same as in the first selection round. Cells selected after two rounds of selection were either subjected to a third round of selection or plated onto LB-agar plates supplemented with 50 μg/ml Kanamycin and grown over night at 37° C.

Evaluation of selected clones by sequencing and Western blot analysis. Selected clones were grown overnight at 37° C. in 3 ml LB medium supplemented with 50 μg/ml Kanamycin to prepare plasmid DNA using standard procedures. Sequencing was performed at MWG (Germany).

For Western blot analysis approximately 10 to 20 μg of total cellular protein was separated by 10% SDS-PAGE and blotted onto HybondC membrane (Amersham Pharmacia Biotech, England). The LamB or FhuA fusion proteins were detected using human serum as the primary antibody at a dilution of approximately 1:5,000 and anti-human IgG or IgA antibodies coupled to HRP at a dilution of 1:5,000 as secondary antibodies. Detection was performed using the ECL detection kit (Amersham Pharmacia Biotech, England). Alternatively, rabbit anti-FhuA or rabbit anti-LamB polyclonal immune sera were used as primary antibodies in combination with the respective secondary antibodies coupled to HRP for the detection of the fusion proteins.

Results

Screening of bacterial surface display libraries by magnetic activated cell sorting (MACS) using biotinylated Igs. The libraries LCPn-50 in pMAL9.1 and LCPn-300 in pHIE11 were screened with a pool of biotinylated, human IgGs from patient sera (see Example 1: Preparation of antibodies from human serum). The selection procedure was performed as described under Experimental procedures. FIG. 3A shows the data obtained with the screen of the LCPn-50 library and P14-IgGs. As can be seen from the colony count after the first selection cycle from MACS screening, the total number of cells recovered at the end is drastically reduced from 1×10⁷ cells to approximately 6×10⁴ cells, but the selection without antibodies added showed a similar reduction to a number of about 5×10³ cells (FIG. 3A). After the second round, a similar number of cells was recovered with P14-IgGs, while only 7×10³ cells were recovered when no IgGs from human serum were added, clearly showing that selection was dependent on C. pneumoniae specific antibodies. The third round reduced the number of cells in the sample with P14-IgGs and without IgG added, but it is clearly obvious that selection of cells was specific for the C. pneumoniae antibodies present in the human serum applied for the screen. To evaluate the performance of the screen, 25 selected clones were picked randomly and subjected to immunoblot analysis with the screening IgG pool (P14-IgG) (FIG. 3B). This analysis revealed that more than 90% of the selected clones showed reactivity with antibodies present in the relevant serum, whereas the control strain expressing LamB without a C. pneumoniae specific insert did not react with the same serum (not shown). In general, the rate of reactivity was observed to lie within the range of 35 to 95%. Colony PCR analysis showed that all selected clones contained an insert in the expected size range.

Subsequent sequencing of a larger number of randomly picked clones (600 to 800 per screen) led to the identification of the gene and the corresponding peptide or protein sequence that was specifically recognized by the human serum antibodies used for screening. The frequency with which a specific clone is selected reflects at least in part the abundance and/or affinity of the specific antibodies in the serum used for selection and recognizing the epitope presented by this clone. In that regard it is striking that clones derived from some ORFs (e.g. CP0051, CP0070) were picked very frequently (40 to 200 times), indicating their highly immunogenic property. Table 1 summarizes the data obtained for the two performed screens. All clones that are presented in Table 1 have been verified by immunoblot analysis using whole cellular extracts from single clones to show the indicated reactivity with the pool of human serum used in the respective screen. As can be seen from Table 1, distinct regions of the identified ORF are identified as immunogenic, since variably sized fragments of the proteins are displayed on the surface by the platform proteins.

It is further worth noticing that a large number of the genes identified by the bacterial surface display screen encode proteins of C. pneumoniae, which have no assigned function or may even constitute proteins, which have not been predicted by previous bioinformatic analysis. Thus, many of these candidates constitute novel antigenic proteins of C. pneumoniae.

### Example 4

### Assessment of the Reactivity of Highly Immunogenic Peptide Sequences with Individual Human Sera

Experimental Procedures

Peptide Synthesis

Peptides were synthesized in small scale (4 mg resin; up to 288 in parallel) using standard F-moc chemistry on a Rink amide resin (PepChem, Tubingen, Germany) using a SyroII synthesizer (Multisyntech, Witten, Germany). After the sequence was assembled, peptides were elongated with Fmoc-epsilon-aminohexanoic acid (as a linker) and biotin (Sigma, St. Louis, Mo.; activated like a normal amino acid). Peptides were cleaved off the resin with 93% TFA, 5% triethylsilane, and 2% water for one hour. Peptides were dried under vacuum and freeze dried three times from acetonitrile/water (1:1). The presence of the correct mass was verified by mass spectrometry on a Reflex III MALDI-TOF (Broker, Bremen Germany). The peptides were used without further purification.

Enzyme Linked Immune Assay (ELISA).

Biotin-labeled peptides (at the N-terminus) were coated on Streptavidin ELISA plates at 10 μg/ml concentration. Streptavidin plates were prepared by coating with Streptavidin (Sigma) at 5 μg/ml concentration overnight. Human sera were tested at two serum dilutions, 200× and 1,000×. Highly specific Horse Radish Peroxidase (HRP)-conjugated anti-human IgG secondary antibodies (Southern Biotech) were used according to the manufacturers' recommendations (dilution: 1,000×). Following manual coating, peptide plates were processed and analyzed by the Gemini 160 ELISA robot (TECAN) with a built-in ELISA reader (GENIOS, TECAN).

Results

Following the bioinformatic analysis of selected clones, corresponding peptides were designed and synthesized. In case of epitopes with more than 26 amino acid residues, overlapping peptides were made. All peptides were synthesized with a N-terminal biotin-tag and used as coating reagents on Streptavidin-coated ELISA plates.

The analysis was performed with 20 selected highest titer sera—among those the ones included in screening pools—and with two negative controls, having lower titers according to ELISA. A summary for serum reactivity of 80 peptides representing 58 C. pneumoniae antigens identified in the genomic screens is shown in Table 2. The 80 peptides represent 29 ORFs, 13 ARFs and 16 CRFs. The peptides were compared by the score calculated for each peptide based on the number of positive sera and the extent of reactivity. Extent of reactivity was expressed as scores, which were calculated based on the sum of ELISA reactivities with 22 individual sera, based on the following calculations: −=0; +=1; ++=2; +++=3. Positivity was assessed based on OD_{405 nm} readings at two different serum dilutions after correction for background. Peptides ranged from highly and widely reactive to weakly positive ones. The highest possible score, 122, would belong to a peptide, which displays +++ reactivity with all 22 sera at both 200× and 1000× serum dilutions (3×22×2=122). Among the most reactive ones with scores greater than 20, there are alternative and complementary strand antigens (ARF1062 and CRF0016, CRF1073), as well as epitopes present in annotated ORFs (CP0161, CP0282, CP0316, CP0426, CP0693 and CP0737). The lower scoring peptides were mainly reactive with the sera used for their identification, but did not show wide reactivity with other serum samples.

These data suggest that individual patients infected with C. pneumoniae recognize different patterns of antigens and different antigenic epitopes within the antigens. However, there is a substantial overlap among the antigen specificities of anti-C. pneumoniae antibody repertoires of individual patients against certain epitopes identified by the method of the present invention exemplified by the identification of high scoring peptides.

### Example 5

### Identification of HLA Class I-Restricted and HLA Class II-Restricted T Cell Epitopes or Epitope Regions Within the Selected Antigens

Experimental Procedures

HLA Class I-Restricted Epitope Prediction

The prediction of HLA class I-restricted epitopes within the antigen identified by bacterial display was performed using the program SYFPEITHI as described by {Rammensee, H. et al., 1999}.

(http://syfpeithi.bmi-heidelberg.com/Scripts/MHCServer.dll/EpPredict.htm)

The prediction was performed for the four MHC types HLA*A0201, B0702, A03 and A2402. For all four MHC types, only predicted epitopes of a length of 9 amino acids with a score above 20 are listed.

HLA Class II-Restricted Epitope Prediction

The prediction of HLA class II-restricted epitopes within the antigen identified by bacterial display was performed using the program TEPITOPE as described by {Bian, H. et al., 2003}. The prediction was performed for the eight MHC types DRB1*0101, 0301, 0401, 0701, 0801, 1101, 1501 and DRB*0101. For all predictions, those epitopes or regions are listed, which showed a hit with a threshold of 5% for at least 4 MHC types. The listed epitopes or regions are selected in such a way that a region as small as possible, but in any case smaller than 25 amino acids contains a hit in at least 4 MHC types. Only in cases where epitopes overlap continuously in a larger region, the whole region (potentially larger than 25 amino acids) is depicted.

Results

T cell epitopes are the minimal essential units of information derived from nonself (or self) proteins that stimulate cellular (T cell) immune responses. They are presented in the cleft of MHC class I or class II molecules at the surface of the antigen-presenting cell to the T cell receptor (TCR). The following cascade of cellular events triggered by the interaction of a TCR and the pathogen-derived peptide epitope in the cleft of an MHC molecule serves to inform the cellular immune system that bacteria, viruses or parasites are present. Induction of epitope-specific T cell responses may improve immune responses to pathogens for which no conventional vaccines currently exist and thus provide a means to allow protection from infection or to clear an infection by the respective pathogen. The accuracy of the bioinformatic prediction methods for T cell epitopes are remarkable {Martin, W. et al., 2003} and thus offer a complementary method to the described antigen identification approach by bacterial surface display, which is based on the experimental identification on B cell epitopes. Since the ORFs, corresponding to the antigens identified on the basis of recognition by antibodies in human sera, most likely also contain linear T-cell epitopes it was the aim of this invention to provide also a set of T cell epitopes for the listed antigens.

The molecular definition of the corresponding HLA class II helper-epitopes is useful for the design of synthetic anti-chlamydial vaccines, which can induce immunological memory, because the helper-epitopes derived from the chlamydial antigens provide “cognate help” to the B-cell response against these antigens or fragments thereof Moreover it is possible to use these helper-epitopes to induce memory to T-independent antigens like for instance carbohydrates (conjugate vaccines). MHC class II molecules bind peptides consisting of 11 to 25 amino acids and are predominantly recognized by CD4+ helper T cells. As is evident from Table 1, almost all antigens identified by bacterial surface display contain a number of potential MHC class II-restricted epitopes, which may also overlap with the identified B cell epitopes (e.g. CP0426).

More importantly, intracellular Chlamydia pneumoniae can be eliminated by CD8+ cytotoxic T-cells, which recognize HLA class I-restricted epitopes. MHC class I molecules present in general peptides of 8 to 10 amino acids in length with two conserved anchor residues. The four assessed MHC types as listed in Table 1 comprise approximately 70% of all MHC types in individuals worldwide, so that the occurrence of epitopes within an antigen for these four MHC types provides a broad coverage. While most of the identified antigens belonging to annotated ORFs contain epitopes for all four MHC types (e.g. CP0134, CP0578), only one of the in general much shorter putative novel ORFs (CRF1083), which were not previously annotated, contains epitopes for all four MHC types, but a number of them possesses epitopes for at least 2 or 3 MHC types (e.g. ARF1071, CRF0551). In the context of a protective immune response, epitope-specific T cells can persist as memory cells, thus allowing a more rapid response to the pathogen upon encounter. Therefore and since the two types of cellular immune response are complementary, preventive as well as therapeutic vaccines should be designed to contain both class I-restricted and class II-restricted epitopes.

The identified peptides or fragments thereof (for instance overlapping 15-mers) can be synthesized and tested for their ability to bind to various MHC molecules in vitro. Their immunogenicity can be tested by assessing the peptide (antigen)-driven proliferation (BrdU or 3H-thymidine incorporation) or the secretion of cytokines (ELIspot, intracellular cytokine staining) of T-cells in vitro ({Schmittel, A. et al., 2000}; {Sester, M. et al., 2000}). In this regard it will be interesting to determine quantitative and qualitative differences in the T-cell response to the chlamydial antigens or the selected promiscuous peptides or fragments thereof e.g. in populations of patients with different chlamydial infections, or in colonized versus healthy individuals neither recently infected nor colonized. In addition, the immunogenicity of the predicted peptides can be tested in HLA-transgenic mice {Sonderstrup, G. et al., 1999}.

Furthermore, the antigens/epitopes may be injected into mice and the induced antibodies and T cells responses can then be determined. The protective capacity of the antibodies and T cells induced by the antigens through vaccination can be assessed in animal models. All these approaches are well available to the skilled man in the art.

TABLE 1
Immunogenic proteins identified by bacterial surface display.

| Chlamydia | Putative | | Predicted class |
|---|---|---|---|
| pneumoniae | function | | II-restricted T |
| antigenic | (by | predicted | cell |
| protein | homology) | immunogenic aa* | epitope/regions** |
| CP0018 | conserved | 18-29, 60-78, 89-95, 100-105, | 55-84, 190-207, |
| | hypothetical | 124-143, 166-180, | 323-331, 370-390, |
| | protein | 187-194, 196-208, | 551-570, |
| | | 224-242, 285-294, | 606-614, 633-647 |
| | | 305-311, 313-320, | |
| | | 351-360, 368-373, | |
| | | 390-403, 411-429, | |
| | | 432-470, 483-489, | |
| | | 513-523, 535-543, | |
| | | 548-564, 579-587, | |
| | | 589-598, 604-612, | |
| | | 622-627, 632-648 | |
| CP0051 | major outer | 4-29, 31-38, 46-64, 66-80, | 65-82, 123-165, |
| | membrane | 109-115, 131-139, | 268-290, 299-307, |
| | protein, | 152-160, 170-183, | 320-329, |
| | MOMP | 198-234, 239-255, | 336-347 |
| | | 267-290, 301-313, | |
| | | 318-324, 336-345, | |
| | | 350-365, 380-386 | |
| CP0069 | hypothetical | 20-33, 35-43, 47-60, 77-92, | 66-75 |
| | protein | 113-124, 137-145, | |
| | | 185-196 | |
| CP0070 | hypothetical | 47-64, 137-155, 157-167, | 58-72, 183-196, |
| | protein | 182-198, 212-233, | 249-261, 315-323, |
| | | 247-259, 291-303, | 334-342, |
| | | 315-337, 345-350, | 347-356, 358-366 |
| | | 355-368, 373-379 | |
| CP0134 | Protease IV, | 4-36, 43-49, 60-75, 96-107, | 5-38, 67-77, 113-127, |
| | putative | 113-123, 132-172, | 134-145, |
| | | 186-193, 217-229, 231-250, | 147-156, 220-236, |
| | | 260-282, 284-290, | 271-283, |
| | | 298-312, 315-330 | 285-293, 296-304, |
| | | | 309-321 |
| CP0161 | conserved | 5-26, 29-50, 52-61, 65-74, | 1-9, 30-38, 53-63, |
| | hypothetical | 89-96, 140-147, 153-162, | 70-78, 92-107, |
| | protein | 183-188, 191-197, | 141-149, |
| | | 203-210, 213-225 | 158-166, 174-191, |
| | | | 205-224 |
| CP0177 | hypothetical | 31-36, 46-54, 65-80, 86-102, | 182-193, 202-211, |
| | protein | 168-175, 179-186, | 279-294, |
| | | 188-194, 200-208, | 311-319, 369-377, |
| | | 210-216, 225-231, | 468-476, |
| | | 243-257, 289-296, | 547-558, 579-587, |
| | | 362-387, 460-474, | 681-700, |
| | | 476-486, 504-511, | 731-740 |
| | | 518-525, 569-579, | |
| | | 581-600, 665-684, | |
| | | 688-694, 700-705, | |
| | | 717-735 | |
| CP0254 | prolyl-tRNA | 4-9, 17-24, 27-52, 66-77, | 31-69, 115-127, |
| | synthetase | 91-98, 104-124, 127-139, | 132-143, 145-165, |
| | | 178-199, 211-219, | 176-187, |
| | | 221-228, 234-244, | 190-204, 212-220, |
| | | 246-255, 263-286, | 266-286, |
| | | 303-312, 316-321, | 304-316, 403-423, |
| | | 337-346, 356-362, | 440-456, |
| | | 367-372, 377-390, | 523-544 |
| | | 402-416, 449-459, | |
| | | 465-479, 491-501, | |
| | | 503-508, 523-541, | |
| | | 551-558, 560-565 | |
| CP0282 | hypothetical | 34-42, 52-63, 71-87, 112-120, | 95-103, 114-123, |
| | protein | 142-147, 154-159, | 180-195, 205-220, |
| | | 166-177, 180-197, | 240-248, |
| | | 204-224, 237-256, | 370-400, 481-495, |
| | | 260-268, 280-286, | 588-596, |
| | | 312-324, 338-343, | 707-715, 750-765 |
| | | 372-412, 456-463, | |
| | | 479-490, 494-504, | |
| | | 506-512, 518-524, | |
| | | 538-548, 562-573, | |
| | | 585-591, 597-606, | |
| | | 674-690, 703-712, | |
| | | 714-740, 749-766 | |
| CP0286 | polymorphic | 4-44, 50-55, 59-67, 73-83, | 6-23, 28-36, 64-75, |
| | membrane | 91-98, 101-109, 131-145, | 134-150, |
| | protein, E/F | 230-236, 267-273, | 182-192, 227-236, |
| | family | 293-300, 303-310, | 306-316, |
| | | 349-354, 375-397, | 340-350, 376-387, |
| | | 404-416, 434-441, | 421-435, |
| | | 445-452, 456-468, | 449-460, 527-535, |
| | | 479-485, 487-512, | 553-569, |
| | | 544-568, 571-579, | 587-595, 641-657, |
| | | 593-599, 604-610, | 668-676, |
| | | 614-621, 642-656, | 683-694, 743-755, |
| | | 665-678, 706-716, | 800-819, |
| | | 729-736, 748-756, | 843-865, 861-886, |
| | | 780-795, 797-814, | 894-915, |
| | | 827-844, 850-861, | 929-938 |
| | | 864-882, 889-900, | |
| | | 906-933 | |
| CP0306 | polymorphic | 4-32, 73-82, 90-101, 116-132, | 7-16, 90-107, |
| | membrane | 144-160, 171-182, | 110-137, 170-187, |
| | protein, G | 195-200, 227-234, | 197-213, |
| | family | 255-271, 293-300, | 233-251, 277-287, |
| | | 313-336, 344-350, | 291-314, |
| | | 369-375, 381-398, | 361-390, 412-425, |
| | | 413-421, 436-465, | 451-465, |
| | | 487-496, 503-508, | 489-498, 513-521, |
| | | 510-527, 538-546, | 570-580, |
| | | 552-562, 608-614, | 619-637, 662-679, |
| | | 617-636, 663-674, | 713-721, |
| | | 679-691, 705-730, | 725-733, 745-754, |
| | | 734-748, 769-807, | 766-781, |
| | | 825-834, 848-861, | 790-805, 817-834, |
| | | 864-871, 891-902 | 868-883, |
| | | | 888-903 |
| CP0316 | ATP- | 10-18, 30-52, 63-70, 72-79, | 34-56, 73-89, |
| | dependent | 96-133, 146-158, 168-175, | 103-130, 146-154, |
| | Clp protease, | 184-193, 203-210, | 184-205, |
| | ATP-binding | 213-222, 227-234, | 213-227, 245-257, |
| | subunit | 237-257, 263-273, | 258-278, |
| | | 285-291, 297-312, | 292-316, 331-341, |
| | | 320-338, 359-378, | 358-369, |
| | | 385-393, 395-410, | 372-383, 388-397, |
| | | 412-421, 490-510, | 410-418, |
| | | 521-527, 540-548, | 503-514, 524-530, |
| | | 563-571, 573-585, | 548-556, |
| | | 592-598, 615-620, | 565-573, 584-595, |
| | | 632-641, 652-661, | 637-646, |
| | | 672-679, 704-711, | 656-663, 673-686, |
| | | 717-723, 729-736, | 734-742, |
| | | 742-751, 766-778, | 745-754, 757-768, |
| | | 788-808, 817-824, | 770-781, |
| | | 836-842 | 816-828 |
| CP0339 | conserved | 4-14, 20-33, 36-63, 71-93, | 35-66, 70-85, |
| | hypothetical | 96-104, 106-117, 120-128, | 107-118, 124-132, |
| | protein | 131-147, 161-172, | 165-179, |
| | | 174-186, 195-210, | 186-196, 197-205, |
| | | 212-247, 269-286, | 276-289, |
| | | 288-301, 306-322, | 292-300, 348-368, |
| | | 324-332, 348-354, | 369-381, |
| | | 356-363, 384-391 | 385-394 |
| CP0353 | A/G-specific | 12-20, 37-48, 51-58, 69-75, | 31-39, 40-55, 62-74, |
| | adenine | 86-98, 113-136, 141-161, | 121-137, |
| | glycosylase | 171-216, 222-254, | 148-164, 170-178, |
| | | 264-273, 291-301, | 223-253, |
| | | 311-345, 351-361 | 309-329, 354-369 |
| CP0426 | conserved | 30-36, 50-56, 96-102, | 53-62, 92-107, |
| | hypothetical | 110-116, 125-131, | 192-203, 315-323, |
| | protein | 162-174, 179-187, | 436-452, |
| | | 189-201, 223-230, | 464-483, 502-524 |
| | | 232-239, 266-278, | |
| | | 320-328, 330-337, | |
| | | 339-350, 388-400, | |
| | | 408-413, 417-423, | |
| | | 435-447, 456-480, | |
| | | 499-524, 526-534 | |
| CP0578 | conserved | 7-32, 36-56, 77-82, 88-100, | 6-31, 37-48, 58-69, |
| | hypothetical | 117-144, 153-166, | 90-105, 110-118, |
| | protein | 173-180, 188-226, | 134-142, |
| | | 256-297, 300-316, | 146-157, 210-220, |
| | | 323-337, 339-348, | 267-276, |
| | | 361-384, 390-427, | 291-300, 319-330, |
| | | 438-455, 476-488, | 362-372, |
| | | 516-523, 535-566, | 393-401, 405-421, |
| | | 580-586, 597-607, | 447-456, |
| | | 615-621, 626-634, | 463-471, 517-525, |
| | | 639-649, 654-660, | 574-582, |
| | | 668-673, 677-688, | 597-612, 618-626, |
| | | 707-714, 716-728, | 642-650, |
| | | 730-742, 746-756, | 656-668, 668-678, |
| | | 763-772, 801-808, | 683-695, |
| | | 820-829, 840-875, | 725-733, 778-791, |
| | | 882-888, 895-911, | 840-849, |
| | | 914-920, 928-948, | 894-917, 927-939, |
| | | 953-961, 987-995, | 954-963, |
| | | 999-1005, 1007-1026, | 966-974, 978-998, |
| | | 1053-1060, 1071-1079, | 1010-1021, |
| | | 1082-1117, 1123-1129 | 1056-1067, |
| | | | 1070-1083, |
| | | | 1090-1104 |
| CP0581 | hypothetical | 11-19, 34-53, 55-91, 113-119, | 33-54, 69-95, |
| | protein | 122-129, 131-140, | 210-221, 244-254, |
| | | 157-170, 173-179, | 257-269 |
| | | 188-195, 200-206, | |
| | | 208-220, 222-232, | |
| | | 236-244, 250-265, | |
| | | 267-274, 282-290, | |
| | | 293-301, 317-323, | |
| | | 336-343, 355-361, | |
| | | 372-384 | |
| CP0618 | leucyl-tRNA | 31-55, 58-64, 69-75, 81-90, | 1-9, 31-46, 52-61, |
| | synthetase | 129-150, 154-167, | 60-78, 132-148, |
| | | 179-184, 189-208, | 182-199, |
| | | 227-237, 248-271, | 214-229, 249-264, |
| | | 277-284, 313-340, | 280-293, |
| | | 350-358, 361-368, | 320-341, 347-355, |
| | | 371-378, 384-390, | 386-411, |
| | | 418-425, 438-444, | 486-502, 553-575, |
| | | 455-468, 487-506, | 624-634, |
| | | 514-523, 525-550, | 673-689, 690-700, |
| | | 558-569, 572-578, | 702-714, |
| | | 588-598, 607-618, | 721-735, 736-746, |
| | | 645-651, 653-665, | 757-777, |
| | | 672-684, 708-715, | 788-798, 810-818 |
| | | 717-742, 754-771, | |
| | | 776-782, 786-802, | |
| | | 806-817 | |
| CP0693 | DNA- | 13-19, 22-28, 61-67, 74-81, | 25-43, 81-92, |
| | directed RNA | 86-103, 110-122, 141-155, | 111-141, 150-159, |
| | polymerase, | 162-169, 171-177, | 213-220, |
| | beta′ subunit | 181-186, 192-199, | 222-242, 243-254, |
| | | 201-207, 225-238, | 256-267, |
| | | 246-263, 273-279, | 276-288, 289-307, |
| | | 287-300, 307-313, | 381-397, |
| | | 331-336, 351-367, | 398-409, 422-438, |
| | | 370-376, 380-392, | 441-464, |
| | | 395-402, 415-422, | 485-500, 515-528, |
| | | 424-451, 454-465, | 542-553, |
| | | 473-492, 496-509, | 569-585, 591-601, |
| | | 515-523, 541-547, | 639-649, |
| | | 569-582, 589-601, | 656-664, 709-719, |
| | | 613-636, 638-647, | 725-734, |
| | | 653-679, 702-714, | 739-753, 841-850, |
| | | 721-729, 739-748, | 883-893, |
| | | 768-779, 799-813, | 902-911, 912-926, |
| | | 821-828, 832-840, | 935-948, |
| | | 847-853, 857-873, | 960-969, 976-984, |
| | | 886-892, 894-905, | 994-1008, |
| | | 917-926, 958-971, | 1037-1047, |
| | | 974-981, 983-989, | 1073-1085, |
| | | 997-1004, 1006-1032, | 1100-1108, |
| | | 1034-1049, 1054-1061, | 1124-1134, |
| | | 1063-1069, 1073-1081, | 1167-1179, |
| | | 1083-1095, 1097-1115, | 1194-1203, |
| | | 1122-1132, 1143-1153, | 1220-1254, |
| | | 1164-1171, 1178-1185, | 1258-1277, |
| | | 1193-1213, 1216-1251, | 1308-1319, |
| | | 1258-1272, 1277-1283, | 1348-1366 |
| | | 1305-1317, 1324-1330, | |
| | | 1333-1355, 1383-1390 | |
| CP0737 | phosphoenol | 16-23, 25-47, 49-59, 64-72, | 12-21, 28-39, 52-67, |
| | pyruvate- | 79-91, 95-105, 113-122, | 115-124, |
| | protein | 133-145, 148-162, | 189-204, 224-232, |
| | phosphotransferase | 169-176, 179-188, | 234-242, |
| | | 190-200, 202-218, | 263-284, 302-322, |
| | | 232-239, 250-283, | 363-385, |
| | | 299-333, 337-344, | 389-397, 446-463, |
| | | 349-355, 364-406, | 479-488, |
| | | 430-437, 439-449, | 513-522, 528-552 |
| | | 452-460, 464-490, | |
| | | 492-503, 505-530, | |
| | | 533-562 | |
| CP0840 | fumarate | 8-16, 36-54, 59-76, 85-92, | 18-27, 36-56, |
| | hydratase | 104-124, 137-180, | 101-120, 145-158, |
| | | 199-248, 255-298, | 165-173, |
| | | 300-307, 324-339, | 179-189, 239-255, |
| | | 356-373, 381-393, | 255-270, |
| | | 402-442, 448-455 | 330-346, 355-375, |
| | | | 383-394, |
| | | | 403-421 |
| CP0888 | conserved | 29-69, 71-88, 95-104, | 24-40, 46-64, 65-79, |
| | hypothetical | 106-130, 143-189, | 83-105, 121-129, |
| | protein | 205-232 | 144-199, |
| | | | 206-236 |
| CP0897 | polymorphic | 4-46, 51-66, 77-88, 102-110, | 1-28, 109-124, |
| | membrane | 115-126, 142-148, | 208-220, 261-280, |
| | protein, D | 171-181, 183-192, | 286-296, |
| | family | 202-212, 227-234, | 310-324, 398-405, |
| | | 251-261, 263-278, | 425-433, |
| | | 283-316, 319-325, | 439-454, 504-517, |
| | | 336-352, 362-371, | 535-555, |
| | | 386-393, 399-406, | 570-591, 599-614, |
| | | 410-425, 427-437, | 620-630, |
| | | 441-450, 457-464, | 691-699, 711-719, |
| | | 471-476, 490-496, | 729-739, |
| | | 514-521, 549-557, | 751-760, 783-791, |
| | | 571-578, 601-611, | 843-855, |
| | | 618-623, 627-646, | 878-886, 890-900, |
| | | 657-670, 672-689, | 940-955, |
| | | 696-704, 726-740, | 984-1003, 1007-1026, |
| | | 742-756, 765-776, | 1065-1073, |
| | | 778-784, 792-801, | 1106-1122, |
| | | 822-836, 862-868, | 1136-1149, |
| | | 875-881, 887-898, | 1188-1198, |
| | | 914-919, 941-948, | 1203-1211, |
| | | 963-969, 971-978, | 1227-1235, |
| | | 996-1004, 1007-1016, | 1249-1256, |
| | | 1036-1051, 1068-1080, | 1298-1308, |
| | | 1082-1090, 1092-1098, | 1374-1392, |
| | | 1104-1127, 1135-1144, | 1398-1409, |
| | | 1156-1177, 1181-1195, | 1414-1429, |
| | | 1197-1206, 1214-1231, | 1436-1444, |
| | | 1243-1263, 1278-1284, | 1456-1490, |
| | | 1295-1303, 1305-1323, | 1504-1521, |
| | | 1337-1346, 1355-1374, | 1530-1547, |
| | | 1376-1383, 1406-1423, | 1592-1609 |
| | | 1455-1463, 1465-1489, | |
| | | 1506-1518, 1527-1552, | |
| | | 1555-1570, 1581-1589 | |
| CP0945 | conserved | 15-25, 41-102, 111-117, | 10-30, 36-44, 46-59, |
| | hypothetical | 127-134, 145-170, | 57-98, 122-138, |
| | protein | 194-201, 207-225 | 144-160, |
| | | | 162-173, 194-217 |
| CP0973 | transketolase | 7-54, 65-94, 97-103, 154-163, | 8-31, 43-59, 61-75, |
| | | 170-180, 182-199, | 93-104, 126-144, |
| | | 216-222, 227-234, | 179-201, |
| | | 243-256, 267-273, | 244-254, 289-302, |
| | | 286-298, 314-322, | 330-338, |
| | | 324-353, 363-380, | 364-382, 413-421, |
| | | 393-401, 424-431, | 428-466, |
| | | 434-441, 447-470, | 476-525, 582-599, |
| | | 475-495, 506-532, | 602-619, |
| | | 540-548, 554-592, | 621-632 |
| | | 594-607, 609-617, | |
| | | 619-626, 628-634, | |
| | | 656-662 | |
| CP0981 | RNA | 5-21, 32-56, 88-99, 117-124, | 50-65, 67-87, 96-104, |
| | methyltransferase, | 128-138, 143-150, | 144-153, |
| | TrmA | 168-180, 183-189, | 156-164, 169-177, |
| | family | 196-213, 220-240, | 199-220, |
| | | 254-263, 266-289, | 259-289, 324-333, |
| | | 300-313, 321-330, | 339-360, |
| | | 335-358, 361-371, | 372-385 |
| | | 380-398 | |
| CP1063 | conserved | 12-23, 44-50, 54-60, 91-97, | 133-159, 208-222, |
| | hypothetical | 103-109, 119-125, | 354-368 |
| | protein | 131-137, 141-151, | |
| | | 172-183, 201-226, | |
| | | 230-238, 252-265, | |
| | | 315-321, 331-345, | |
| | | 360-370, 376-386, | |
| | | 392-406, 410-416, | |
| | | 422-431 | |
| CP1075 | hypothetical | 4-16, 29-36, 39-64, 69-75, | 3-12, 23-38, 27-38, |
| | protein | 79-87, 90-122, 126-134, | 43-56, 93-107, |
| | | 139-173, 184-190, | 123-137, |
| | | 195-203, 206-213, | 144-154, 175-199, |
| | | 216-228, 234-246, | 229-244, |
| | | 250-257, 260-266, | 288-303, 308-316, |
| | | 274-282, 291-312, | 323-337, |
| | | 318-325, 340-345, | 410-423, 455-473, |
| | | 348-361, 364-388, | 488-496, |
| | | 399-437, 439-448, | 531-551, 560-577, |
| | | 451-464, 467-473, | 577-591, |
| | | 480-510, 514-520, | 619-637, 646-660, |
| | | 534-553, 561-574, | 664-672 |
| | | 579-589, 593-599, | |
| | | 616-655, 658-671 | |
| CP1121 | conserved | 4-31, 50-80, 83-93, 97-103, | 1-17, 20-30, 66-80, |
| | hypothetical | 111-116, 123-132, | 100-119, |
| | protein | 134-163, 170-199, | 139-150, 171-182, |
| | | 205-210, 215-220, | 186-198, |
| | | 230-247, 249-278, | 207-221, 228-242, |
| | | 280-308, 311-329, | 258-274, |
| | | 337-347, 349-358, | 286-308, 314-330, |
| | | 365-371, 376-401, | 337-352, |
| | | 417-430, 434-446, | 355-376, 383-391, |
| | | 459-505, 511-518, | 417-432, |
| | | 527-535, 537-545, | 437-446, 462-473, |
| | | 547-565, 573-581, | 479-488, |
| | | 592-601 | 496-507, 514-522, |
| | | | 541-554, |
| | | | 557-565, 576-585, |
| | | | 589-605 |
| CP1126 | conserved | 9-60, 67-73, 79-93, 109-122, | 16-27, 49-60, 99-122, |
| | hypothetical | 134-142, 144-153, | 136-145, |
| | protein | 165-192, 197-225, | 148-162, 186-194, |
| | | 235-244, 259-279, | 213-221, |
| | | 289-299, 308-317, | 225-246, 261-275, |
| | | 321-332, 338-347, | 281-292, |
| | | 350-361, 373-387, | 353-361, 390-401, |
| | | 402-409, 411-421, | 451-470, |
| | | 439-445, 450-456, | 486-494, 497-516 |
| | | 462-468, 470-479, | |
| | | 490-501, 503-516 | |
| ARF0271 | 31aa (M at 2) | 4-10, 16-28 | 3-14, 16-30 |
| ARF0276 | 33aa (none) | 8-18, 20-30 | none |
| ARF0280.1 | 30aa (none) | 4-16, 18-27 | 2-13, 20-30 |
| ARF0280.2 | 101aa (none) | 36-57, 62-92 | 46-66 |
| ARF0294 | 21aa (V at 4) | 4-18 | 1-16 |
| ARF0311 | 63aa (none) | 13-27, 38-52 | 1-13, 11-25, 27-37 |
| ARF0524 | 69aa (M at 16) | 4-17, 27-40, 55-62 | 9-25, 34-46, 50-64 |
| ARF0636 | 12aa (none) | 4-9 | none |
| ARF0857 | 25aa (none) | none | 3-14 |
| ARF1016 | 32aa (none) | 7-12, 24-29 | 22-30 |
| ARF1046 | 33aa (none) | 14-30 | 15-30 |
| ARF1062 | 20aa (none) | none | none |
| ARF1071 | 113aa (M at 8) | 4-27, 31-59, 75-86, 93-103, | 15-44, 51-61, 79-95 |
| | | 105-110 | |
| ARF1081 | 20aa (none) | 4-13 | none |
| CRF0014 | 55a (M at 27) | 4-15, 17-23, 39-52 | 4-13, 16-29, 40-50 |
| CRF0016 | 26aa (none) | none | none |
| CRF0177 | 128aa (M at | 8-19, 40-47, 67-86, 88-125 | 15-25, 48-59, 64-80, |
| | 31) no | | 108-118 |
| | homology | | |
| CRF0434 | 49aa (V at 8) | 4-27, 41-46, | none |
| CRF0435 | 48aa (V at 10) | 21-28, 34-43 | 8-16 |
| CRF0485 | 116aa (M at 5) | 8-20, 24-37, 39-50, 61-67, | 4-16, 31-42, 84-93 |
| | No | 69-91 | |
| | Homology | | |
| CRF0507 | 148aa (M at | 4-25, 31-39, 59-97, 100-118, | 26-40, 49-57, 66-95, |
| | 8), No | 120-129 | 97-128, 131-139 |
| | homology | | |
| CRF0551 | 60aa (V at 10) | 7-24, 32-43, 45-57 | 32-48 |
| CRF0586 | 63aa (M at 1) | 4-18, 20-26, 31-37 | 3-17, 33-43 |
| CRF0686 | 85aa (M at 3) | 15-23, 25-39, 43-50, 62-70 | 16-32, 61-73 |
| CRF0754 | 45aa (none) | 4-13, 28-42 | 3-14, 28-39 |
| CRF0944 | 29aa (none) | 4-10, 19-26 | 21-29 |
| CRF0961 | 86aa (none) | 4-22, 40-46, 51-57, 64-76 | 2-10, 45-53, 58-72, |
| | | | 73-82 |
| CRF1037 | 45aa (none) | 12-24, 27-42 | 13-30, 34-44 |
| CRF1073 | 69aa (none) | 4-55 | 5-15, 17-33 |
| CRF1083 | 95aa (M at 8) | 31-42, 45-52, 86-92 | 8-16, 35-52, 83-91 |

| | | No. of | Location of | |
|---|---|---|---|---|
| Chlamydia | | selected | identified | Seq. |
| pneumoniae | Predicted class I- | clones per | immunogenic | ID |
| antigenic | restricted T cell | ORF and | region | (DNA, |
| protein | epitope/regions*** | screen | (aa) | Prot.) |
| CP0018 | A0201: 60, 63, 67, | A: 4, B: 18 | 39-129, 224-296, | 1, 61 |
| | 70, 126, 129, 133, | | 464-609 | |
| | 136, 169, 186, 200, | | | |
| | 308, 371, 414, 421, | | | |
| | 434, 444, 459, 503, | | | |
| | 512, 532, 540, 547, | | | |
| | 601, 625, 632, 634, | | | |
| | 637 | | | |
| | B0702: 99, 529 | | | |
| | A03: 25, 38, 59, 155, | | | |
| | 278, 285, 412, 420, | | | |
| | 441, 451, 457, 481, | | | |
| | 506, 510, 524, 536, | | | |
| | 539, 554, 578, 596, | | | |
| | 638 | | | |
| | A2402: 179, 604 | | | |
| CP0051 | A0201: 4, 13, 69, 93, | A: 40, B: 3 | 76-103, 226-239, | 2, 62 |
| | 149, 174, 273, 277, | | 267-333 | |
| | 298, 305, 312, 319, | | | |
| | 375 | | | |
| | B0702: 28, 303 | | | |
| | A03: 3, 58, 73, 100, | | | |
| | 153, 191, 223, 227, | | | |
| | 232, 251, 269, 286, | | | |
| | 343, 374 | | | |
| | A2402: 238 | | | |
| CP0069 | A0201: 32, 48, 49, | A: 14, B: 19 | 92-214 | 3, 63 |
| | 113 | | | |
| | B0702: 77, 118, 139, | | | |
| | 185 | | | |
| | A03: 2, 24, 120 | | | |
| | A2402: none | | | |
| CP0070 | A0201: 135, 160, | A: 14, B: 187 | 6-188 | 4, 64 |
| | 183, 184, 204, 249, | | | |
| | 256, 293, 296, 318, | | | |
| | 319, 356, 372 | | | |
| | B0702: 94 | | | |
| | A03: 13, 60, 159, | | | |
| | 163, 189, 204, 220, | | | |
| | 233, 300, 333, 335, | | | |
| | 356, 362 | | | |
| | A2402: 198, 289 | | | |
| CP0134 | A0201: 3, 10, 14, 17, | B: 13 | 159-217 | 5, 65 |
| | 24, 46, 59, 133, 155, | | | |
| | 220, 270, 312 | | | |
| | B0702: 233 | | | |
| | A03: 2, 22, 31, 36, | | | |
| | 62, 65, 122, 140, 155, | | | |
| | 162, 170, 189, 235, | | | |
| | 248, 260, 286, 298 | | | |
| | A2402: 156, 183, 325 | | | |
| CP0161 | A0201: 31, 33, 39, | A: 4 | 97-113 | 6, 66 |
| | 56, 63, 78, 119, 136, | | | |
| | 196 | | | |
| | B0702: none | | | |
| | A03: 14, 35, 38, 55, | | | |
| | 97, 98, 146, 156, 158, | | | |
| | 215 | | | |
| | A2402: 88, 214 | | | |
| CP0177 | A0201: 28, 78, 285, | A: 2, B: 6 | 92-177, 591-604, | 7, 67 |
| | 309, 321, 376, 379, | | | |
| | 388, 468, 475, 479, | | | |
| | 500, 571, 624, 668, | | | |
| | 716 | | | |
| | B0702: 360, 455, 669 | | | |
| | A03: 185, 190, 204, | | | |
| | 264, 281, 292, 478, | | | |
| | 502, 588, 675, 680, | | | |
| | 716, 730 | | | |
| | A2402: none | | | |
| CP0254 | A0201: 17, 24, 31, | A: 7 | 9-22 | 8, 68 |
| | 45, 53, 56, 63, 69, | | | |
| | 107, 129, 150, 171, | | | |
| | 178, 189, 191, 217, | | | |
| | 255, 273, 277, 305, | | | |
| | 312, 451, 458, 470, | | | |
| | 478, 506, 522 | | | |
| | B0702: 71, 379 | | | |
| | A03: 20, 29, 34, 44, | | | |
| | 119, 133, 276, 284, | | | |
| | 300, 328, 404, 465, | | | |
| | 470, 529, 543 | | | |
| | A2402: 182, 551 | | | |
| CP0282 | A0201: 179, 206, | A: 4, B: 4 | 160-253, | 9, 69 |
| | 209, 213, 216, 255, | | 630-717 | |
| | 286, 300, 304, 324, | | | |
| | 365, 369, 373, 376, | | | |
| | 377, 380, 381, 384, | | | |
| | 562, 694, 720, 721, | | | |
| | 729, 749, 752, 755 | | | |
| | B0702: 197, 330, | | | |
| | 559, 592, 600, 714, | | | |
| | 751 | | | |
| | A03: 91, 111, 140, | | | |
| | 167, 191, 315, 388, | | | |
| | 393, 402, 458, 463, | | | |
| | 587, 720, 762 | | | |
| | A2402: 748 | | | |
| CP0286 | A0201: 7, 8, 15, 73, | B: 3 | 603-669 | 10, 70 |
| | 80, 133, 134, 138, | | | |
| | 182, 194, 271, 272, | | | |
| | 298, 432, 438, 457, | | | |
| | 458, 487, 490, 527, | | | |
| | 548, 568, 616, 644, | | | |
| | 647, 667, 741, 782, | | | |
| | 801, 829, 866 | | | |
| | B0702: 126, 259, 792 | | | |
| | A03: 15, 20, 133, | | | |
| | 155, 160, 232, 299, | | | |
| | 458, 464, 552, 558, | | | |
| | 560, 605, 607, 654, | | | |
| | 670, 672, 768, 810, | | | |
| | 840, 852, 877, 900 | | | |
| | A2402: 167, 380, | | | |
| | 425, 593, 907 | | | |
| CP0306 | A0201: 8, 23, 53, 57, | A: 7 | 529-542 | 11, 71 |
| | 128, 169, 178, 239, | | | |
| | 263, 290, 297, 310, | | | |
| | 324, 331, 339, 365, | | | |
| | 398, 436, 443, 450, | | | |
| | 470, 485, 488, 513, | | | |
| | 514, 520, 614, 669, | | | |
| | 711, 723, 771, 824, | | | |
| | 849, 895 | | | |
| | B0702: 316, 861 | | | |
| | A03: 118, 135, 196, | | | |
| | 225, 284, 290, 370, | | | |
| | 454, 489, 492, 521, | | | |
| | 557, 624, 632, 745, | | | |
| | 778, 783, 850, 868, | | | |
| | 910 | | | |
| | A2402: 226, 383 | | | |
| CP0316 | A0201: 27, 32, 36, | B: 3 | 14-101 | 12, 72 |
| | 65, 109, 112, 120, | | | |
| | 127, 186, 249, 250, | | | |
| | 262, 267, 297, 301, | | | |
| | 353, 360, 367, 410, | | | |
| | 418, 436, 465, 472, | | | |
| | 505, 518, 522, 565, | | | |
| | 576, 585, 638, 645, | | | |
| | 650, 676, 687, 724, | | | |
| | 745, 756, 763, 795 | | | |
| | B0702: 164, 411, | | | |
| | 510, 560, 569, 647, | | | |
| | 766, 780 | | | |
| | A03: 14, 39, 48, 65, | | | |
| | 74, 129, 175, 215, | | | |
| | 217, 229, 230, 240, | | | |
| | 253, 257, 262, 269, | | | |
| | 308, 317, 322, 327, | | | |
| | 352, 371, 372, 373, | | | |
| | 374, 417, 443, 454, | | | |
| | 472, 514, 525, 567, | | | |
| | 629, 637, 657, 662, | | | |
| | 683, 698, 731, 744, | | | |
| | 752, 763, 769, 787, | | | |
| | 790, 802, 815, 819 | | | |
| | A2402: 26, 102, 381, | | | |
| | 704 | | | |
| CP0339 | A0201: 34, 41, 50, | A: 2 | 139-151 | 13, 73 |
| | 53, 109, 127, 134, | | | |
| | 153, 165, 271, 286, | | | |
| | 297, 340, 384 | | | |
| | B0702: 80, 321, 334, | | | |
| | 354 | | | |
| | A03: 33, 57, 110, | | | |
| | 153, 178, 276, 284, | | | |
| | 383 | | | |
| | A2402: 79, 99, 123 | | | |
| CP0353 | A0201: 46, 95, 103, | A: 7 | 246-275 | 14, 74 |
| | 110, 143, 156, 178, | | | |
| | 186, 190, 236, 242, | | | |
| | 244, 291, 294, 315, | | | |
| | 333, 353 | | | |
| | B0702: 125, 183, | | | |
| | 256, 326 | | | |
| | A03: 3, 68, 82, 102, | | | |
| | 131, 177, 185, 190, | | | |
| | 193, 223, 224, 244, | | | |
| | 250, 295, 340, 349, | | | |
| | 354 | | | |
| | A2402: 88, 89 | | | |
| CP0426 | A0201: 126, 174, | B: 2 | 61-138 | 15, 75 |
| | 225, 267, 309, 316, | | | |
| | 320, 337, 436, 466, | | | |
| | 467, 473, 474 | | | |
| | B0702: 14, 128, 143, | | | |
| | 228, 347, 494 | | | |
| | A03: 2, 52, 112, 201, | | | |
| | 209, 217, 230, 235, | | | |
| | 236, 337, 381, 395, | | | |
| | 413, 419, 454, 466, | | | |
| | 510, 515, 556 | | | |
| | A2402: none | | | |
| CP0578 | A0201: 11, 18, 22, | B: 5 | 325-389 | 16, 76 |
| | 41, 48, 86, 104, 156, | | | |
| | 190, 197, 221, 286, | | | |
| | 290, 334, 343, 345, | | | |
| | 407, 442, 509, 538, | | | |
| | 575, 596, 597, 598, | | | |
| | 636, 678, 685, 723, | | | |
| | 754, 757, 779, 818, | | | |
| | 850, 857, 864, 893, | | | |
| | 900, 901, 907, 918, | | | |
| | 927, 934, 972, 988, | | | |
| | 1018, 1025, 1034, | | | |
| | 1048, 1065, 1072, | | | |
| | 1089, 1094, 1101, | | | |
| | 1108 | | | |
| | B0702: 127, 336, | | | |
| | 411, 806, 852 | | | |
| | A03: 28, 68, 90, 91, | | | |
| | 93, 158, 293, 310, | | | |
| | 350, 368, 380, 394, | | | |
| | 425, 441, 461, 554, | | | |
| | 569, 597, 628, 667, | | | |
| | 684, 724, 737, 752, | | | |
| | 761, 767, 804, 851, | | | |
| | 897, 907, 933, 979, | | | |
| | 1030, 1032, 1051, | | | |
| | 1075, 1090, 1125 | | | |
| | A2402: 133, 308, | | | |
| | 502, 797, 939, 960 | | | |
| CP0581 | A0201: 32, 37, 43, | A: 2 | 324-351 | 17, 77 |
| | 47, 50, 53, 57, 64, 68, | | | |
| | 71, 73, 74, 78, 80, 82, | | | |
| | 113, 120, 155, 162, | | | |
| | 194, 205, 209, 231, | | | |
| | 235, 238, 252, 259, | | | |
| | 266, 273, 280, 287, | | | |
| | 294, 301, 308, 315, | | | |
| | 333 | | | |
| | B0702: 8, 16, 18, 66, | | | |
| | 377 | | | |
| | A03: 36, 44, 81, 99, | | | |
| | 124, 193, 261, 319 | | | |
| | A2402: none | | | |
| CP0618 | A0201: 51, 82, 139, | A: 7 | 90-100 | 18, 78 |
| | 186, 193, 197, 200, | | | |
| | 239, 248, 249, 250, | | | |
| | 257, 311, 325, 326, | | | |
| | 520, 555, 556, 589, | | | |
| | 606, 651, 716, 723, | | | |
| | 730, 737, 758, 761, | | | |
| | 772, 788 | | | |
| | B0702: 39, 41, 569, | | | |
| | 695, 709, 783 | | | |
| | A03: 51, 60, 89, 110, | | | |
| | 141, 207, 216, 295, | | | |
| | 301, 395, 404, 518, | | | |
| | 527, 555, 568, 593, | | | |
| | 596, 673, 691, 722, | | | |
| | 757, 772, 790, 799 | | | |
| | A2402: 130, 131, 179, | | | |
| | 402, 414, 701 | | | |
| CP0693 | A0201: 107, 110, | A: 3 | 273-290 | 19, 79 |
| | 112, 133, 152, 200, | | | |
| | 204, 223, 244, 251, | | | |
| | 271, 289, 291, 305, | | | |
| | 323, 360, 380, 407, | | | |
| | 422, 428, 440, 491, | | | |
| | 507, 512, 536, 616, | | | |
| | 625, 628, 648, 650, | | | |
| | 665, 668, 748, 768, | | | |
| | 784, 797, 801, 826, | | | |
| | 858, 859, 903, 910, | | | |
| | 913, 925, 932, 959, | | | |
| | 960, 968, 993, 1008, | | | |
| | 1020, 1068, 1072, | | | |
| | 1138, 1141, 1142, | | | |
| | 1193, 1201, 1218, | | | |
| | 1226, 1237, 1261, | | | |
| | 1271, 1311, 1348, | | | |
| | 1349, 1377 | | | |
| | B0702: 126, 375, | | | |
| | 433, 477, 608, 658, | | | |
| | 852, 1106, 1121, | | | |
| | 1303, 1362 | | | |
| | A03: 24, 102, 151, | | | |
| | 164, 169, 211, 229, | | | |
| | 245, 274, 279, 285, | | | |
| | 333, 348, 361, 382, | | | |
| | 391, 397, 428, 447, | | | |
| | 453, 480, 496, 590, | | | |
| | 591, 595, 615, 623, | | | |
| | 629, 638, 664, 669, | | | |
| | 672, 738, 744, 775, | | | |
| | 789, 840, 910, 917, | | | |
| | 939, 966, 977, 1057, | | | |
| | 1084, 1096, 1119, | | | |
| | 1127, 1128, 1145, | | | |
| | 1163, 1167, 1202, | | | |
| | 1214, 1238, 1244, | | | |
| | 1260, 1279, 1335 | | | |
| | A2402: 145, 355, | | | |
| | 961, 1053, 1103, | | | |
| | 1245 | | | |
| CP0737 | A0201: 23, 30, 58, | A: 9 | 401-419 | 20, 80 |
| | 78, 84, 97, 98, 120, | | | |
| | 123, 133, 162, 169, | | | |
| | 189, 215, 218, 236, | | | |
| | 309, 312, 316, 365, | | | |
| | 372, 384, 388, 391, | | | |
| | 426, 446, 453, 465, | | | |
| | 466, 478, 508, 513, | | | |
| | 515, 523, 530, 536, | | | |
| | 543, 554 | | | |
| | B0702: 333, 467 | | | |
| | A03: 13, 19, 115, | | | |
| | 130, 181, 195, 225, | | | |
| | 262, 270, 275, 311, | | | |
| | 313, 325, 342, 390, | | | |
| | 391, 398, 461, 530 | | | |
| | A2402: 116, 188, 229 | | | |
| CP0840 | A0201: 5, 102, 149, | B: 3 | 83-232 | 21, 81 |
| | 156, 160, 164, 185, | | | |
| | 186, 204, 208, 211, | | | |
| | 221, 232, 264, 270, | | | |
| | 273, 277, 280, 284, | | | |
| | 287, 317, 329, 362, | | | |
| | 387, 398, 402, 404, | | | |
| | 422, 429, 431, 449 | | | |
| | B0702: 37, 298, 359 | | | |
| | A03: 9, 17, 35, 40, | | | |
| | 41, 105, 111, 146, | | | |
| | 166, 234, 279, 343, | | | |
| | 384, 412 | | | |
| | A2402: 365 | | | |
| CP0888 | A0201: 30, 37, 66, | A: 3 | 182-199 | 22, 82 |
| | 77, 81, 84, 112, 118, | | | |
| | 141, 144, 145, 146, | | | |
| | 149, 150, 153, 167, | | | |
| | 169, 170, 178, 196, | | | |
| | 213, 215, 220 | | | |
| | B0702: none | | | |
| | A03: 13, 21, 39, 44, | | | |
| | 62, 75, 78, 97, 119, | | | |
| | 124, 145, 148, 154, | | | |
| | 177, 190, 207 | | | |
| | A2402: 22, 216 | | | |
| CP0897 | A0201: 26, 33, 79, | A: 5 | 911-935 | 23, 83 |
| | 170, 200, 265, 290, | | | |
| | 297, 302, 304, 333, | | | |
| | 334, 377, 412, 414, | | | |
| | 415, 431, 436, 458, | | | |
| | 465, 481, 494, 536, | | | |
| | 546, 568, 605, 678, | | | |
| | 690, 697, 703, 724, | | | |
| | 729, 730, 735, 737, | | | |
| | 767, 776, 797, 840, | | | |
| | 861, 938, 968, 999, | | | |
| | 1072, 1079, 1085, | | | |
| | 1094, 1113, 1160, | | | |
| | 1163, 1180, 1188, | | | |
| | 1195, 1217, 1245, | | | |
| | 1250, 1273, 1302, | | | |
| | 1358, 1362, 1363, | | | |
| | 1401, 1408, 1465, | | | |
| | 1469, 1481, 1507 | | | |
| | B0702: 178, 960, | | | |
| | 1034 | | | |
| | A03: 6, 21, 38, 159, | | | |
| | 204, 248, 260, 306, | | | |
| | 337, 349, 384, 425, | | | |
| | 438, 458, 481, 502, | | | |
| | 521, 546, 605, 690, | | | |
| | 730, 731, 819, 860, | | | |
| | 915, 946, 967, 1007, | | | |
| | 1018, 1065, 1113, | | | |
| | 1187, 1188, 1205, | | | |
| | 1223, 1409, 1414, | | | |
| | 1495, 1526, 1531, | | | |
| | 1537 | | | |
| | A2402: 101, 255, | | | |
| | 1421, 1457, 1538, | | | |
| | 1580, 1589 | | | |
| CP0945 | A0201: 12, 16, 37, | A: 7 | 118-131 | 24, 84 |
| | 46, 61, 82, 121, 128, | | | |
| | 149, 157, 162, 197, | | | |
| | 204, 212 | | | |
| | B0702: 39 | | | |
| | A03: 2, 23, 53, 68, | | | |
| | 97, 107, 121, 127, | | | |
| | 156, 169, 196 | | | |
| | A2402: 9, 13, 114 | | | |
| CP0973 | A0201: 9, 10, 13, 35, | A: 2 | 115-128 | 25, 85 |
| | 46, 76, 77, 83, 151, | | | |
| | 165, 179, 187, 195, | | | |
| | 283, 326, 338, 342, | | | |
| | 360, 365, 368, 375, | | | |
| | 415, 450, 485, 508, | | | |
| | 556, 565, 569, 576, | | | |
| | 602 | | | |
| | B0702: none | | | |
| | A03: 5, 20, 130, 181, | | | |
| | 251, 271, 288, 294, | | | |
| | 333, 355, 356, 364, | | | |
| | 446, 451, 467, 483, | | | |
| | 486, 523, 544, 611 | | | |
| | A2402: 214, 219, | | | |
| | 323, 399, 424, 458 | | | |
| CP0981 | A0201: 26, 33, 49, | A: 2 | 74-93 | 26, 86 |
| | 88, 96, 129, 169, 170, | | | |
| | 198, 257, 268, 281, | | | |
| | 337, 342, 366, 391, | | | |
| | 393 | | | |
| | B0702: 39, 122, 248 | | | |
| | A03: 76, 106, 117, | | | |
| | 185, 190, 198, 238, | | | |
| | 257, 266, 280, 341, | | | |
| | 344, 350, 367 | | | |
| | A2402: 304, 384 | | | |
| CP1063 | A0201: 47, 134, 140, | B: 4 | 1-88 | 27, 87 |
| | 143, 203, 204, 210, | | | |
| | 254, 355, 358, 359, | | | |
| | 362, 369, 417 | | | |
| | B0702: 119 | | | |
| | A03: 17, 128, 129, | | | |
| | 141, 143, 153, 208, | | | |
| | 232, 245, 278, 301, | | | |
| | 313, 327, 328, 384, | | | |
| | 395 | | | |
| | A2402: none | | | |
| CP1075 | A0201: 36, 101, 123, | A: 1 | 553-570 | 28, 88 |
| | 129, 136, 146, 156, | | | |
| | 160, 194, 205, 219, | | | |
| | 236, 245, 283, 289, | | | |
| | 350, 402, 413, 437, | | | |
| | 475, 505, 517, 542, | | | |
| | 585, 605, 620, 627, | | | |
| | 657 | | | |
| | B0702: 34, 52, 88, | | | |
| | 358, 540, 656 | | | |
| | A03: 3, 8, 13, 32, 82, | | | |
| | 105, 111, 117, 137, | | | |
| | 167, 173, 180, 182, | | | |
| | 262, 300, 306, 350, | | | |
| | 409, 412, 423, 499, | | | |
| | 500, 563, 568, 581, | | | |
| | 585, 627, 628 | | | |
| | A2402: 554, 638 | | | |
| CP1121 | A0201: 4, 65, 66, | A: 6 | 49-60, 582-607, | 29, 89 |
| | 120, 121, 144, 170, | | | |
| | 174, 208, 226, 233, | | | |
| | 276, 278, 285, 286, | | | |
| | 298, 336, 348, 355, | | | |
| | 363, 382, 384, 395, | | | |
| | 457, 458, 494, 501, | | | |
| | 578 | | | |
| | B0702: 133, 278, | | | |
| | 294, 551 | | | |
| | A03: 53, 89, 110, | | | |
| | 159, 186, 232, 290, | | | |
| | 324, 406, 431, 458, | | | |
| | 463, 480, 490, 513, | | | |
| | 541, 549, 558, 585 | | | |
| | A2402: 22, 137, 152, | | | |
| | 189, 227, 255, 261, | | | |
| | 291, 419, 569 | | | |
| CP1126 | A0201: 15, 22, 28, | A: 4 | 478-490 | 30, 90 |
| | 29, 48, 49, 106, 107, | | | |
| | 114, 147, 170, 177, | | | |
| | 188, 208, 209, 212, | | | |
| | 256, 280, 287, 316, | | | |
| | 451, 468, 489 | | | |
| | B0702: 33, 217 | | | |
| | A03: 36, 98, 124, | | | |
| | 136, 142, 153, 177, | | | |
| | 188, 251, 262, 291, | | | |
| | 320, 323, 383, 417, | | | |
| | 464, 487, 491, 492, | | | |
| | 505 | | | |
| | A2402: 44, 86, 146, | | | |
| | 411, 437, 499 | | | |
| ARF0271 | A0201: none | A: 4, B: 7 | 2-16 | 31, 91 |
| | B0702: none | | | |
| | A03: 1, 15 | | | |
| | A2402: none | | | |
| ARF0276 | A0201: none | A: 2 | 7-15 | 32, 92 |
| | B0702: none | | | |
| | A03: none | | | |
| | A2402: none | | | |
| ARF0280.1 | A0201: 22 | A: 1 | 10-29 | 33, 93 |
| | B0702: none | | | |
| | A03: 1 | | | |
| | A2402: none | | | |
| ARF0280.2 | A0201: 84 | A: 3 | 27-35 | 34, 94 |
| | B0702: none | | | |
| | A03: none | | | |
| | A2402: none | | | |
| ARF0294 | A0201: 1, 9 | A: 8 | 5-12 | 35, 95 |
| | B0702: 2 | | | |
| | A03: none | | | |
| | A2402: none | | | |
| ARF0311 | A0201: 16, 37 | A: 3 | 17-36 | 36, 96 |
| | B0702: none | | | |
| | A03: 20 | | | |
| | A2402: none | | | |
| ARF0524 | A0201: 7, 10 | A: 3 | 47-62 | 37, 97 |
| | B0702: none | | | |
| | A03: 11, 14, 58 | | | |
| | A2402: none | | | |
| ARF0636 | A0201: none | A: 4 | 1-10 | 38, 98 |
| | B0702: none | | | |
| | A03: none | | | |
| | A2402: none | | | |
| ARF0857 | A0201: 2 | A: 5 | 7-20 | 39, 99 |
| | B0702: none | | | |
| | A03: 1 | | | |
| | A2402: none | | | |
| ARF1016 | A0201: none | A: 3 | 7-21 | 40, 100 |
| | B0702: none | | | |
| | A03: 4, 9 | | | |
| | A2402: none | | | |
| ARF1046 | A0201: none | A: 2 | 3-18 | 41, 101 |
| | B0702: none | | | |
| | A03: 1, 20 | | | |
| | A2402: none | | | |
| ARF1062 | A0201: none | A: 8, B: 10 | 3-17 | 42, 102 |
| | B0702: none | | | |
| | A03: 1 | | | |
| | A2402: none | | | |
| ARF1071 | A0201: 11, 15, 24, | A: 7 | 41-50 | 43, 103 |
| | 28, 31, 35, 36, 42, 48, | | | |
| | 49, 53, 78, 79, 97 | | | |
| | B0702: none | | | |
| | A03: 20, 28, 35, 37, | | | |
| | 43, 49, 60, 65, 77, 85, | | | |
| | 86 | | | |
| | A2402: 21, 103 | | | |
| ARF1081 | A0201: none | A: 6 | 2-14 | 44, 104 |
| | B0702: none | | | |
| | A03: 7, 10 | | | |
| | A2402: none | | | |
| CRF0014 | A0201: 3, 38 | A: 4 | 33-41 | 45, 105 |
| | B0702: none | | | |
| | A03: 14, 41 | | | |
| | A2402: none | | | |
| CRF0016 | A0201: none | A: 18 | 4-25 | 46, 106 |
| | B0702: none | | | |
| | A03: none | | | |
| | A2402: none | | | |
| CRF0177 | A0201: 7, 110 | A: 5 | 60-70 | 47, 107 |
| | B0702: none | | | |
| | A03: 16, 34, 109 | | | |
| | A2402: none | | | |
| CRF0434 | A0201: 19 | A: 3 | 30-47 | 48, 108 |
| | B0702: none | | | |
| | A03: 1, 23 | | | |
| | A2402: none | | | |
| CRF0435 | A0201: 34 | A: 8 | 23-42 | 49, 109 |
| | B0702: none | | | |
| | A03: 19, 28, 39 | | | |
| | A2402: none | | | |
| CRF0485 | A0201: 4, 24, 79, 83 | A: 7 | 42-59 | 50, 110 |
| | B0702: none | | | |
| | A03: 7, 25, 71, 79, 91 | | | |
| | A2402: none | | | |
| CRF0507 | A0201: 8, 24, 61, 67, | A: 8 | 38-47 | 51, 111 |
| | 72, 103, 112 | | | |
| | B0702: none | | | |
| | A03: 3, 39, 74, 110, | | | |
| | 119 | | | |
| | A2402: none | | | |
| CRF0551 | A0201: 14, 18 | A: 5 | 27-43 | 52, 112 |
| | B0702: none | | | |
| | A03: 38, 47 | | | |
| | A2402: 14 | | | |
| CRF0586 | A0201: 3, 7, 10 | A: 7 | 34-53 | 53, 113 |
| | B0702: none | | | |
| | A03: 9 | | | |
| | A2402: none | | | |
| CRF0686 | A0201: none | A: 4 | 67-84 | 54, 114 |
| | B0702: 8 | | | |
| | A03: 64 | | | |
| | A2402: none | | | |
| CRF0754 | A0201: 31 | A: 12 | 1-20 | 55, 115 |
| | B0702: none | | | |
| | A03: 7 | | | |
| | A2402: 5 | | | |
| CRF0944 | A0201: none | A: 12 | 5-13 | 56, 116 |
| | B0702: none | | | |
| | A03: none | | | |
| | A2402: none | | | |
| CRF0961 | A0201: 35, 76 | A: 9, B: 2 | 33-45 | 57, 117 |
| | B0702: 3 | | | |
| | A03: 1, 66 | | | |
| | A2402: none | | | |
| CRF1037 | A0201: 36 | A: 5 | 1-9 | 58, 118 |
| | B0702: none | | | |
| | A03: 15, 18 | | | |
| | A2402: none | | | |
| CRF1073 | A0201: 14 | A: 8 | 26-45 | 59, 119 |
| | B0702: none | | | |
| | A03: 53 | | | |
| | A2402: none | | | |
| CRF1083 | A0201: 86 | B: 26 | 27-93 | 60, 120 |
| | B0702: 56 | | | |
| | A03: 21 | | | |
| | A2402: 4 | | | |

A 50 bp library of C. pneumoniae AR39 in lamB with P14-IgG (755),
B 300 bp library in fhuA with P14-IgG (669);
*prediction of antigenic sequences longer than 5 amino acids was performed with the program ANTIGENIC (Kolaskar and Tongaonkar, 1990).

TABLE 2
Immunogenicity of peptide epitopes with human sera

| | location in | | |
|---|---|---|---|
| | protein | | |
| Peptide | (aa) | Score | Seq. ID |
| CP0018.1 | 237-256 | 6 | 61 |
| CP0018.2 | 508-530 | 1 | 61 |
| CP0051.3 | 227-239 | 5 | 62 |
| CP0069.1 | 141-160 | 1 | 63 |
| CP0069.2 | 168-187 | 1 | 63 |
| CP0069.3 | 155-173 | 3 | 63 |
| CP0070.1 | 101-124 | 2 | 64 |
| CP0070.2 | 161-187 | 1 | 64 |
| CP0070.4 | 59-85 | 1 | 64 |
| CP0070.5 | 80-106 | 1 | 64 |
| CP0161.1 | 97-112 | 38 | 66 |
| CP0177.3 | 139-165 | 1 | 67 |
| CP0254.1 | 10-21 | 6 | 68 |
| CP0282.1 | 667-688 | 15 | 69 |
| CP0282.2 | 677-696 | 15 | 69 |
| CP0282.3 | 161-187 | 24 | 69 |
| CP0282.4 | 183-209 | 9 | 69 |
| CP0282.5 | 205-231 | 6 | 69 |
| CP0282.6 | 226-252 | 5 | 69 |
| CP0286.1 | 603-629 | 7 | 70 |
| CP0286.2 | 622-648 | 8 | 70 |
| CP0286.3 | 643-669 | 4 | 70 |
| CP0306.1 | 529-541 | 11 | 71 |
| CP0316.1 | 12-34 | 12 | 72 |
| CP0316.2 | 29-51 | 35 | 72 |
| CP0316.3 | 46-67 | 5 | 72 |
| CP0316.4 | 62-83 | 5 | 72 |
| CP0339.1 | 139-151 | 4 | 73 |
| CP0353.1 | 246-262 | 11 | 74 |
| CP0353.2 | 251-275 | 16 | 74 |
| CP0426.1 | 61-84 | 12 | 75 |
| CP0426.2 | 79-102 | 23 | 75 |
| CP0426.3 | 97-120 | 7 | 75 |
| CP0426.4 | 115-138 | 5 | 75 |
| CP0578.1 | 325-350 | 5 | 76 |
| CP0578.2 | 345-370 | 6 | 76 |
| CP0578.3 | 365-389 | 1 | 76 |
| CP0581.1 | 324-349 | 11 | 77 |
| CP0581.2 | 336-351 | 8 | 77 |
| CP0618.1 | 90-100 | 2 | 78 |
| CP0693.1 | 274-290 | 26 | 79 |
| CP0737.1 | 401-419 | 25 | 80 |
| CP0840.1 | 84-107 | 3 | 81 |
| CP0840.2 | 101-123 | 3 | 81 |
| CP0840.3 | 117-139 | 11 | 81 |
| CP0888.1 | 182-199 | 9 | 82 |
| CP0897.1 | 911-935 | 14 | 83 |
| CP0945.1 | 118-131 | 11 | 84 |
| CP0973.1 | 115-128 | 1 | 85 |
| CP0981.1 | 74-93 | 5 | 86 |
| CP1063.2 | 21-43 | 5 | 87 |
| CP1063.4 | 54-76 | 2 | 87 |
| CP1075.1 | 554-570 | 8 | 88 |
| CP1126.2 | 478-490 | 4 | 90 |
| ARF0271.1 | 2-14 | 4 | 91 |
| ARF0276.1 | 7-15 | 3 | 92 |
| ARF0280.1 | 10-28 | 4 | 93 |
| ARF0280.2 | 27-34 | 1 | 94 |
| ARF0311.1 | 17-35 | 6 | 96 |
| ARF0524.1 | 47-61 | 6 | 97 |
| ARF0636.1 | 1-10 | 1 | 98 |
| ARF0857.1 | 7-20 | 9 | 99 |
| ARF1016.1 | 7-20 | 2 | 100 |
| ARF1046.1 | 3-17 | 7 | 101 |
| ARF1062.1 | 3-17 | 59 | 102 |
| ARF1071.1 | 41-50 | 1 | 103 |
| ARF1081.1 | 2-14 | 1 | 104 |
| CRF0014.1 | 33-41 | 1 | 105 |
| CRF0016.1 | 4-25 | 77 | 106 |
| CRF0177.1 | 60-69 | 2 | 107 |
| CRF0435.1 | 23-41 | 13 | 109 |
| CRF0485.1 | 42-59 | 4 | 110 |
| CRF0507.1 | 38-46 | 1 | 111 |
| CRF0551.1 | 27-43 | 13 | 112 |
| CRF0586.1 | 34-53 | 6 | 113 |
| CRF0686.1 | 67-84 | 2 | 114 |
| CRF0754.1 | 1-20 | 4 | 115 |
| CRF0961.1 | 33-45 | 6 | 117 |
| CRF1073.1 | 26-45 | 25 | 119 |
| CRF1083.1 | 27-53 | 8 | 120 |

### REFERENCES

Aldous, M., et al. (1992). J Infect Dis 166: 646-9.

Altschul, S., et al. (1990). Journal of Molecular Biology 215: 403-10.

Bennett, D., et al. (1995). J Mol Recognit 8: 52-8.

Bian, H., et al. (2003). Methods 29: 299-309.

Braun, J., et al. (1994). Ann Rheum Dis 53: 100-5.

Brown, J., et al. (2001). Infect Immun 69: 6702-6.

Campbell, L., et al. (1989). Infect Immun 57: 71-5.

Clackson, T., et al. (1991). Nature 352: 624-8.

Cox, R., et al. (1988). Int J Syst Bacteriol 38: 265-8.

Devereux, J., et al. (1984). Nucleic acids research 12: 387-95.

Doherty, E., et al. (2001). Annu Rev Biophys Biomol Struct 30: 457-475.

Dowell, S., et al. (2001). Clin Infect Dis 33: 492-503.

Eisenbraun, M., et al. (1993). DNA Cell Biol 12: 791-7.

Etz, H., et al. (2001). J Bacteriol 183: 6924-35.

Everett, K., et al. (1999). Int J Syst Bacteriol 49: 415-40.

Falsey, A., et al. (1993). J Infect Dis 168: 493-6.

Ganz, T. (1999). Science 286: 420-421.

Gaydos, C., et al. (1994). J Clin Microbiol 32: 903-5.

Georgiou, G. (1997). Nature Biotechnology 15: 29-34.

Grayston, J. (1992). Clin Infect Dis 15: 757-61.

Grayston, J. (1996). Rev Med Interne 17: 45S-47S.

Grayston, J., et al. (1986). N Engl J Med 315: 161-8.

Hahn, D., et al. (1991). JAMA 266: 225-30.

Haidl, S., et al. (1992). N Engl J Med 326: 576-7.

Hashemzadeh-Bonehi, L., et al. (1998). Mol Microbiol 30: 676-678.

Heinje von G. (1987). Sequence Analysis in Molecular Biology, Academic Press

Hemmer, B., et al. (1999). Nat Med 5: 1375-82.

Hoe, N., et al. (2001). J Infect Dis 183: 633-9.

Hornef, M., et al. (2002). Nat Immunol 3: 1033-40.

Johanson, K., et al. (1995). J Biol Chem 270: 9459-71.

Jones, P., et al. (1986). Nature 321: 522-5.

Kajava, A., et al. (2000). J Bacteriol 182: 2163-9.

Kalman, S., et al. (1999). Nat Genet 21: 385-9.

Kleemola, M., et al. (1988). J Infect Dis 157: 230-6.

Kohler, G., et al. (1975). Nature 256: 495-7.

Kolaskar, A., et al. (1990). FEBS Lett 276: 172-4.

Kuo, C., et al. (1995). Clin Microbiol Rev 8: 451-61.

Lewin, A., et al. (2001). Trends Mol Med 7: 221-8.

Marks, J., et al. (1992). Biotechnology (NY) 10: 779-83.

Martin, W., et al. (2003). Methods 29: 289-98.

McCafferty, J., et al. (1990). Nature 348: 552-4.

Montigiani, S., et al. (2002). Infect Immun 70: 368-79.

Murdin, A., et al. (2000). J Infect Dis 181: S544-51.

Ogawa, H., et al. (1992). J Laryngol Otol 106: 490-2.

Okano, H., et al. (1991). J Neurochem 56: 560-7.

Oligodeoxynucleotides as Antisense Inhibitors of Gene Expression: CRC Cress. Boca Tation, Fla. (1988)

Rammensee, H., et al. (1999). Immunogenetics 50: 213-9.

Read, T., et al. (2000). Nucleic acids research 28: 1397-406.

Read, T., et al. (2003). Nucleic Acids Res 31: 2134-47.

Schmittel, A., et al. (2000). J Immunother 23: 289-95.

Seeger, C., et al. (1984). Proc Natl Acad Sci USA 81: 5849-52.

Sester, M., et al. (2000). AIDS 14: 2653-60.

Shirai, M., et al. (2000). Nucleic Acids Res 28: 2311-4.

Shor, A., et al. (1992). S Afr Med J 82: 158-61.

Skerra, A. (1994). Gene 151: 131-5.

Sonderstrup, G., et al. (1999). Immunol Rev 172: 335-43.

Sundelof, B., et al. (1993). Scand J Infect Dis 25: 259-61.

Tang, D., et al. (1992). Nature 356: 152-4.

Tempest, P., et al. (1991). Biotechnology (NY) 9: 266-71.

Tong, C., et al. (1993). J Clin Pathol. 1993 April; 46(4):313-7. 46: 313-7.

Tourdot, S., et al. (2000). Eur J Immunol 30: 3411-21.

Wang, S., et al. (1991). J Clin Microbiol 29: 1539-41.

Wiley John & Sons, Inc. (1987), Current Protocols in Molecular Biology

Wizel, B., et al. (2002). J Immunol 169: 2524-35.

1. An isolated antigen comprising the amino acid sequence from any of SEQ ID NOs: 61-90 or 92-120, or an antigenic fragment of at least 6 contiguous amino acids of SEQ ID NOs: 61-90 or 92-120.

2. The isolated antigen of claim 1, wherein the isolated antigen comprises the amino acid sequence of amino acids: 18-29, 60-78, 89-95, 100-105, 124-143, 166-180, 187-194, 196-208, 224-242, 285-294, 305-311, 313-320, 351-360, 368-373, 390-403, 411-429, 432-470, 483-489, 513-523, 535-543, 548-564, 579-587, 589-598, 604-612, 622-627, 632-648, 55-84, 190-207, 323-331, 370-390, 551-570, 606-614, 633-647, 39-129, 224-296 and 464-609 of SEQ ID NO:61; and fragments in 9 amino acid length starting from the position of: 60, 63, 67, 70, 126, 129, 133, 136, 169, 186, 200, 308, 371, 414, 421, 434, 444, 459, 503, 512, 532, 540, 547, 601, 625, 632, 634, 637, 99, 529, 25, 38, 59, 155, 278, 285, 412, 420, 441, 451, 457, 481, 506, 510, 524, 536, 539, 554, 578, 596, 638, 179 and 604 of SEQ ID NO:61; 4-29, 31-38, 46-64, 66-80, 109-115, 131-139, 152-160, 170-183, 198-234, 239-255, 267-290, 301-313, 318-324, 336-345, 350-365, 380-386, 65-82, 123-165, 268-290, 299-307, 320-329, 336-347, 76-103, 226-239 and 267-333 of SEQ ID NO:62; and fragments in 9 amino acid length starting from the position of: 4, 13, 69, 93, 149, 174, 273, 277, 298, 305, 312, 319, 375, 28, 303, 3, 58, 73, 100, 153, 191, 223, 227, 232, 251, 269, 286, 343, 374 and 238 of SEQ ID NO:62; 20-33, 35-43, 47-60, 77-92, 113-124, 137-145, 185-196, 66-75 and 92-214 of SEQ ID NO:63; and fragments in 9 amino acid length starting from the position of: 32, 48, 49, 113, 77, 118, 139, 185, 2, 24 and 120 of SEQ ID NO:63; 47-64, 137-155, 157-167, 182-198, 212-233, 247-259, 291-303, 315-337, 345-350, 355-368, 373-379, 58-72, 183-196, 249-261, 315-323, 334-342, 347-356, 358-366 and 6-188 of SEQ ID NO:64; and fragments in 9 amino acid length starting from the position of: 135, 160, 183, 184, 204, 249, 256, 293, 296, 318, 319, 356, 372, 94, 13, 60, 159, 163, 189, 204, 220, 233, 300, 333, 335, 356, 362, 198 and 289 of SEQ ID NO:64; 4-36, 43-49, 60-75, 96-107, 113-123, 132-172, 186-193, 217-229, 231-250, 260-282, 284-290, 298-312, 315-330, 5-38, 67-77, 113-127, 134-145, 147-156, 220-236, 271-283, 285-293, 296-304, 309-321 and 159-217 of SEQ ID NO:65; and fragments in 9 amino acid length starting from the position of: 3, 10, 14, 17, 24, 46, 59, 133, 155, 220, 270, 312, 233, 2, 22, 31, 36, 62, 65, 122, 140, 155, 162, 170, 189, 235, 248, 260, 286, 298, 156, 183 and 325 of SEQ ID NO:65; 5-26, 29-50, 52-61, 65-74, 89-96, 140-147, 153-162, 183-188, 191-197, 203-210, 213-225, 1-9, 30-38, 53-63, 70-78, 92-107, 141-149, 158-166, 174-191, 205-224 and 97-113 of SEQ ID NO:66; and fragments in 9 amino acid length starting from the position of: 31, 33, 39, 56, 63, 78, 119, 136, 196, 14, 35, 38, 55, 97, 98, 146, 156, 158, 215, 88 and 214 of SEQ ID NO:66; 31-36, 46-54, 65-80, 86-102, 168-175, 179-186, 188-194, 200-208, 210-216, 225-231, 243-257, 289-296, 362-387, 460-474, 476-486, 504-511, 518-525, 569-579, 581-600, 665-684, 688-694, 700-705, 717-735, 182-193, 202-211, 279-294, 311-319, 369-377, 468-476, 547-558, 579-587, 681-700, 731-740, 92-177 and 591-604 of SEQ ID NO:67; and fragments in 9 amino acid length starting from the position of: 28, 78, 285, 309, 321, 376, 379, 388, 468, 475, 479, 500, 571, 624, 668, 716, 360, 455, 669, 185, 190, 204, 264, 281, 292, 478, 502, 588, 675, 680, 716 and 730 of SEQ ID NO:67; 4-9, 17-24, 27-52, 66-77, 91-98, 104-124, 127-139, 178-199, 211-219, 221-228, 234-244, 246-255, 263-286, 303-312, 316-321, 337-346, 356-362, 367-372, 377-390, 402-416, 449-459, 465-479, 491-501, 503-508, 523-541, 551-558, 560-565, 31-69, 115-127, 132-143, 145-165, 176-187, 190-204, 212-220, 266-286, 304-316, 403-423, 440-456, 523-544 and 9-22 of SEQ ID NO:68; and fragments in 9 amino acid length starting from the position of: 17, 24, 31, 45, 53, 56, 63, 69, 107, 129, 150, 171, 178, 189, 191, 217, 255, 273, 277, 305, 312, 451, 458, 470, 478, 506, 522, 71, 379, 20, 29, 34, 44, 119, 133, 276, 284, 300, 328, 404, 465, 470, 529, 543, 182 and 551 of SEQ ID NO:68; 34-42, 52-63, 71-87, 112-120, 142-147, 154-159, 166-177, 180-197, 204-224, 237-256, 260-268, 280-286, 312-324, 338-343, 372-412, 456-463, 479-490, 494-504, 506-512, 518-524, 538-548, 562-573, 585-591, 597-606, 674-690, 703-712, 714-740, 749-766, 95-103, 114-123, 180-195, 205-220, 240-248, 370-400, 481-495, 588-596, 707-715, 750-765, 160-253 and 630-717 of SEQ ID NO:69; and fragments in 9 amino acid length starting from the position of: 179, 206, 209, 213, 216, 255, 286, 300, 304, 324, 365, 369, 373, 376, 377, 380, 381, 384, 562, 694, 720, 721, 729, 749, 752, 755, 197, 330, 559, 592, 600, 714, 751, 91, 111, 140, 167, 191, 315, 388, 393, 402, 458, 463, 587, 720, 762 and 748 of SEQ ID NO:69; 4-44, 50-55, 59-67, 73-83, 91-98, 101-109, 131-145, 230-236, 267-273, 293-300, 303-310, 349-354, 375-397, 404-416, 434-441, 445-452, 456-468, 479-485, 487-512, 544-568, 571-579, 593-599, 604-610, 614-621, 642-656, 665-678, 706-716, 729-736, 748-756, 780-795, 797-814, 827-844, 850-861, 864-882, 889-900, 906-933, 6-23, 28-36, 64-75, 134-150, 182-192, 227-236, 306-316, 340-350, 376-387, 421-435, 449-460, 527-535, 553-569, 587-595, 641-657, 668-676, 683-694, 743-755, 800-819, 843-865, 861-886, 894-915, 929-938 and 603-669 of SEQ ID NO:70; and fragments in 9 amino acid length starting from the position of: 7, 8, 15, 73, 80, 133, 134, 138, 182, 194, 271, 272, 298, 432, 438, 457, 458, 487, 490, 527, 548, 568, 616, 644, 647, 667, 741, 782, 801, 829, 866, 126, 259, 792, 15, 20, 133, 155, 160, 232, 299, 458, 464, 552, 558, 560, 605, 607, 654, 670, 672, 768, 810, 840, 852, 877, 900, 167, 380, 425, 593 and 907 of SEQ ID NO:70; 4-32, 73-82, 90-101, 116-132, 144-160, 171-182, 195-200, 227-234, 255-271, 293-300, 313-336, 344-350, 369-375, 381-398, 413-421, 436-465, 487-496, 503-508, 510-527, 538-546, 552-562, 608-614, 617-636, 663-674, 679-691, 705-730, 734-748, 769-807, 825-834, 848-861, 864-871, 891-902, 7-16, 90-107, 110-137, 170-187, 197-213, 233-251, 277-287, 291-314, 361-390, 412-425, 451-465, 489-498, 513-521, 570-580, 619-637, 662-679, 713-721, 725-733, 745-754, 766-781, 790-805, 817-834, 868-883, 888-903 and 529-542 of SEQ ID NO:71; and fragments in 9 amino acid length starting from the position of: 8, 23, 53, 57, 128, 169, 178, 239, 263, 290, 297, 310, 324, 331, 339, 365, 398, 436, 443, 450, 470, 485, 488, 513, 514, 520, 614, 669, 711, 723, 771, 824, 849, 895, 316, 861, 118, 135, 196, 225, 284, 290, 370, 454, 489, 492, 521, 557, 624, 632, 745, 778, 783, 850, 868, 910, 226 and 383 of SEQ ID NO:71; 10-18, 30-52, 63-70, 72-79, 96-133, 146-158, 168-175, 184-193, 203-210, 213-222, 227-234, 237-257, 263-273, 285-291, 297-312, 320-338, 359-378, 385-393, 395-410, 412-421, 490-510, 521-527, 540-548, 563-571, 573-585, 592-598, 615-620, 632-641, 652-661, 672-679, 704-711, 717-723, 729-736, 742-751, 766-778, 788-808, 817-824, 836-842, 34-56, 73-89, 103-130, 146-154, 184-205, 213-227, 245-257, 258-278, 292-316, 331-341, 358-369, 372-383, 388-397, 410-418, 503-514, 524-530, 548-556, 565-573, 584-595, 637-646, 656-663, 673-686, 734-742, 745-754, 757-768, 770-781, 816-828 and 14-101 of SEQ ID NO:72; and fragments in 9 amino acid length starting from the position of: 27, 32, 36, 65, 109, 112, 120, 127, 186, 249, 250, 262, 267, 297, 301, 353, 360, 367, 410, 418, 436, 465, 472, 505, 518, 522, 565, 576, 585, 638, 645, 650, 676, 687, 724, 745, 756, 763, 795, 164, 411, 510, 560, 569, 647, 766, 780, 14, 39, 48, 65, 74, 129, 175, 215, 217, 229, 230, 240, 253, 257, 262, 269, 308, 317, 322, 327, 352, 371, 372, 373, 374, 417, 443, 454, 472, 514, 525, 567, 629, 637, 657, 662, 683, 698, 731, 744, 752, 763, 769, 787, 790, 802, 815, 819, 26, 102, 381 and 704 of SEQ ID NO:72; 4-14, 20-33, 36-63, 71-93, 96-104, 106-117, 120-128, 131-147, 161-172, 174-186, 195-210, 212-247, 269-286, 288-301, 306-322, 324-332, 348-354, 356-363, 384-391, 35-66, 70-85, 107-118, 124-132, 165-179, 186-196, 197-205, 276-289, 292-300, 348-368, 369-381, 385-394 and 139-151 of SEQ ID NO:73; and fragments in 9 amino acid length starting from the position of: 34, 41, 50, 53, 109, 127, 134, 153, 165, 271, 286, 297, 340, 384, 80, 321, 334, 354, 33, 57, 110, 153, 178, 276, 284, 383, 79, 99 and 123 of SEQ ID NO:73; 12-20, 37-48, 51-58, 69-75, 86-98, 113-136, 141-161, 171-216, 222-254, 264-273, 291-301, 311-345, 351-361, 31-39, 40-55, 62-74, 121-137, 148-164, 170-178, 223-253, 309-329, 354-369 and 246-275 of SEQ ID NO:74; and fragments in 9 amino acid length starting from the position of: 46, 95, 103, 110, 143, 156, 178, 186, 190, 236, 242, 244, 291, 294, 315, 333, 353, 125, 183, 256, 326, 3, 68, 82, 102, 131, 177, 185, 190, 193, 223, 224, 244, 250, 295, 340, 349, 354, 88 and 89 of SEQ ID NO:74; 30-36, 50-56, 96-102, 110-116, 125-131, 162-174, 179-187, 189-201, 223-230, 232-239, 266-278, 320-328, 330-337, 339-388-400, 408-413, 417-423, 435-447, 456-480, 499-524, 526-534, 53-62, 92-107, 192-203, 315-323, 436-452, 464-483, 502-524 and 61-138 of SEQ ID NO:75; and fragments in 9 amino acid length starting from the position of: 126, 174, 225, 267, 309, 316, 320, 337, 436, 466, 467, 473, 474, 14, 128, 143, 228, 347, 494, 2, 52, 112, 201, 209, 217, 230, 235, 236, 337, 381, 395, 413, 419, 454, 466, 510, 515 and 556 of SEQ ID NO:75; 7-32, 36-56, 77-82, 88-100, 117-144, 153-166, 173-180, 188-226, 256-297, 300-316, 323-337, 339-348, 361-384, 390-427, 438-455, 476-488, 516-523, 535-566, 580-586, 597-607, 615-621, 626-634, 639-649, 654-660, 668-673, 677-688, 707-714, 716-728, 730-742, 746-756, 763-772, 801-808, 820-829, 840-875, 882-888, 895-911, 914-920, 928-948, 953-961, 987-995, 999-1005, 1007-1026, 1053-1060, 1071-1079, 1082-1117, 1123-1129, 6-31, 37-48, 58-69, 90-105, 110-118, 134-142, 146-157, 210-220, 267-276, 291-300, 319-330, 362-372, 393-401, 405-421, 447-456, 463-471, 517-525, 574-582, 597-612, 618-626, 642-650, 656-668, 668-678, 683-695, 725-733, 778-791, 840-849, 894-917, 927-939, 954-963, 966-974, 978-998, 1010-1021, 1056-1067, 1070-1083, 1090-1104 and 325-389 of SEQ ID NO:76; and fragments in 9 amino acid length starting from the position of: 11, 18, 22, 41, 48, 86, 104, 156, 190, 197, 221, 286, 290, 334, 343, 345, 407, 442, 509, 538, 575, 596, 597, 598, 636, 678, 685, 723, 754, 757, 779, 818, 850, 857, 864, 893, 900, 901, 907, 918, 927, 934, 972, 988, 1018, 1025, 1034, 1048, 1065, 1072, 1089, 1094, 1101, 1108, 127, 336, 411, 806, 852, 28, 68, 90, 91, 93, 158, 293, 310, 350, 368, 380, 394, 425, 441, 461, 554, 569, 597, 628, 667, 684, 724, 737, 752, 761, 767, 804, 851, 897, 907, 933, 979, 1030, 1032, 1051, 1075, 1090, 1125, 133, 308, 502, 797, 939 and 960 of SEQ ID NO:76; 11-19, 34-53, 55-91, 113-119, 122-129, 131-140, 157-170, 173-179, 188-195, 200-206, 208-220, 222-232, 236-244, 250-265, 267-274, 282-290, 293-301, 317-323, 336-343, 355-361, 372-384, 33-54, 69-95, 210-221, 244-254, 257-269 and 324-351 of SEQ ID NO:77; and fragments in 9 amino acid length starting from the position of: 32, 37, 43, 47, 50, 53, 57, 64, 68, 71, 73, 74, 78, 80, 82, 113, 120, 155, 162, 194, 205, 209, 231, 235, 238, 252, 259, 266, 273, 280, 287, 294, 301, 308, 315, 333, 8, 16, 18, 66, 377, 36, 44, 81, 99, 124, 193, 261 and 319 of SEQ ID NO:77; 31-55, 58-64, 69-75, 81-90, 129-150, 154-167, 179-184, 189-208, 227-237, 248-271, 277-284, 313-340, 350-358, 361-368, 371-378, 384-390, 418-425, 438-444, 455-468, 487-506, 514-523, 525-550, 558-569, 572-578, 588-598, 607-618, 645-651, 653-665, 672-684, 708-715, 717-742, 754-771, 776-782, 786-802, 806-817, 1-9, 31-46, 52-61, 60-78, 132-148, 182-199, 214-229, 249-264, 280-293, 320-341, 347-355, 386-411, 486-502, 553-575, 624-634, 673-689, 690-700, 702-714, 721-735, 736-746, 757-777, 788-798, 810-818 and 90-100 of SEQ ID NO:78; and fragments in 9 amino acid length starting from the position of: 51, 82, 139, 186, 193, 197, 200, 239, 248, 249, 250, 257, 311, 325, 326, 520, 555, 556, 589, 606, 651, 716, 723, 730, 737, 758, 761, 772, 788, 39, 41, 569, 695, 709, 783, 51, 60, 89, 110, 141, 207, 216, 295, 301, 395, 404, 518, 527, 555, 568, 593, 596, 673, 691, 722, 757, 772, 790, 799, 130, 131, 179, 402, 414 and 701 of SEQ ID NO:78;13-19, 22-28, 61-67, 74-81, 86-103, 110-122, 141-155, 162-169, 171-177, 181-186, 192-199, 201-207, 225-238, 246-263, 273-279, 287-300, 307-313, 331-336, 351-367, 370-376, 380-392, 395-402, 415-422, 424-451, 454-465, 473-492, 496-509, 515-523, 541-547, 569-582, 589-601, 613-636, 638-647, 653-679, 702-714, 721-729, 739-748, 768-779, 799-813, 821-828, 832-840, 847-853, 857-873, 886-892, 894-905, 917-926, 958-971, 974-981, 983-989, 997-1004, 1006-1032, 1034-1049, 1054-1061, 1063-1069, 1073-1081, 1083-1095, 1097-1115, 1122-1132, 1143-1153, 1164-1171, 1178-1185, 1193-1213, 1216-1251, 1258-1272, 1277-1283, 1305-1317, 1324-1330, 1333-1355, 1383-1390, 25-43, 81-92, 111-141, 150-159, 213-220, 222-242, 243-254, 256-267, 276-288, 289-307, 381-397, 398-409, 422-438, 441-464, 485-500, 515-528, 542-553, 569-585, 591-601, 639-649, 656-664, 709-719, 725-734, 739-753, 841-850, 883-893, 902-911, 912-926, 935-948, 960-969, 976-984, 994-1008, 1037-1047, 1073-1085, 1100-1108, 1124-1134, 1167-1179, 1194-1203, 1220-1254, 1258-1277, 1308-1319, 1348-1366 and 273-290 of SEQ ID NO:79; and fragments in 9 amino acid length starting from the position of: 107, 110, 112, 133, 152, 200, 204, 223, 244, 251, 271, 289, 291, 305, 323, 360, 380, 407, 422, 428, 440, 491, 507, 512, 536, 616, 625, 628, 648, 650, 665, 668, 748, 768, 784, 797, 801, 826, 858, 859, 903, 910, 913, 925, 932, 959, 960, 968, 993, 1008, 1020, 1068, 1072, 1138, 1141, 1142, 1193, 1201, 1218, 1226, 1237, 1261, 1271, 1311, 1348, 1349, 1377, 126, 375, 433, 477, 608, 658, 852, 1106, 1121, 1303, 1362, 24, 102, 151, 164, 169, 211, 229, 245, 274, 279, 285, 333, 348, 361, 382, 391, 397, 428, 447, 453, 480, 496, 590, 591, 595, 615, 623, 629, 638, 664, 669, 672, 738, 744, 775, 789, 840, 910, 917, 939, 966, 977, 1057, 1084, 1096, 1119, 1127, 1128, 1145, 1163, 1167, 1202, 1214, 1238, 1244, 1260, 1279, 1335, 145, 355, 961, 1053, 1103 and 1245 of SEQ ID NO:79; 16-23, 25-47, 49-59, 64-72, 79-91, 95-105, 113-122, 133-145, 148-162, 169-176, 179-188, 190-200, 202-218, 232-239, 250-283, 299-333, 337-344, 349-355, 364-406, 430-437, 439-449, 452-460, 464-490, 492-503, 505-530, 533-562, 12-21, 28-39, 52-67, 115-124, 189-204, 224-232, 234-242, 263-284, 302-322, 363-385, 389-397, 446-463, 479-488, 513-522, 528-552 and 401-419 of SEQ ID NO:80; and fragments in 9 amino acid length starting from the position of: 23, 30, 58, 78, 84, 97, 98, 120, 123, 133, 162, 169, 189, 215, 218, 236, 309, 312, 316, 365, 372, 384, 388, 391, 426, 446, 453, 465, 466, 478, 508, 513, 515, 523, 530, 536, 543, 554, 333, 467, 13, 19, 115, 130, 181, 195, 225, 262, 270, 275, 311, 313, 325, 342, 390, 391, 398, 461, 530, 116, 188 and 229 of SEQ ID NO:80;8-16, 36-54, 59-76, 85-92, 104-124, 137-180, 199-248, 255-298, 300-307, 324-339, 356-373, 381-393, 402-442, 448-455, 18-27, 36-56, 101-120, 145-158, 165-173, 179-189, 239-255, 255-270, 330-346, 355-375, 383-394, 403-421 and 83-232 of SEQ ID NO:81; and fragments in 9 amino acid length starting from the position of: 5, 102, 149, 156, 160, 164, 185, 186, 204, 208, 211, 221, 232, 264, 270, 273, 277, 280, 284, 287, 317, 329, 362, 387, 398, 402, 404, 422, 429, 431, 449, 37, 298, 359, 9, 17, 35, 40, 41, 105, 111, 146, 166, 234, 279, 343, 384, 412 and 365 of SEQ ID NO:81; 29-69, 71-88, 95-104, 106-130, 143-189, 205-232, 24-40, 46-64, 65-79, 83-105, 121-129, 144-199, 206-236 and 182-199 of SEQ ID NO:82; and fragments in 9 amino acid length starting from the position of: 30, 37, 66, 77, 81, 84, 112, 118, 141, 144, 145, 146, 149, 150, 153, 167, 169, 170, 178, 196, 213, 215, 220, 13, 21, 39, 44, 62, 75, 78, 97, 119, 124, 145, 148, 154, 177, 190, 207, 22 and 216 of SEQ ID NO:82; 4-46, 51-66, 77-88, 102-110, 115-126, 142-148, 171-181, 183-192, 202-212, 227-234, 251-261, 263-278, 283-316, 319-325, 336-352, 362-371, 386-393, 399-406, 410-425, 427-437, 441-450, 457-464, 471-476, 490-496, 514-521, 549-557, 571-578, 601-611, 618-623, 627-646, 657-670, 672-689, 696-704, 726-740, 742-756, 765-776, 778-784, 792-801, 822-836, 862-868, 875-881, 887-898, 914-919, 941-948, 963-969, 971-978, 996-1004, 1007-1016, 1036-1051, 1068-1080, 1082-1090, 1092-1098, 1104-1127, 1135-1144, 1156-1177, 1181-1195, 1197-1206, 1214-1231, 1243-1263, 1278-1284, 1295-1303, 1305-1323, 1337-1346, 1355-1374, 1376-1383, 1406-1423, 1455-1463, 1465-1489, 1506-1518, 1527-1552, 1555-1570, 1581-1589, 1-28, 109-124, 208-220, 261-280, 286-296, 310-324, 398-405, 425-433, 439-454, 504-517, 535-555, 570-591, 599-614, 620-630, 691-699, 711-719, 729-739, 751-760, 783-791, 843-855, 878-886, 890-900, 940-955, 984-1003, 1007-1026, 1065-1073, 1106-1122, 1136-1149, 1188-1198, 1203-1211, 1227-1235, 1249-1256, 1298-1308, 1374-1392, 1398-1409, 1414-1429, 1436-1444, 1456-1490, 1504-1521, 1530-1547, 1592-1609 and 911-935 of SEQ ID NO:83; and fragments in 9 amino acid length starting from the position of: 26, 33, 79, 170, 200, 265, 290, 297, 302, 304, 333, 334, 377, 412, 414, 415, 431, 436, 458, 465, 481, 494, 536, 546, 568, 605, 678, 690, 697, 703, 724, 729, 730, 735, 737, 767, 776, 797, 840, 861, 938, 968, 999, 1072, 1079, 1085, 1094, 1113, 1160, 1163, 1180, 1188, 1195, 1217, 1245, 1250, 1273, 1302, 1358, 1362, 1363, 1401, 1408, 1465, 1469, 1481, 1507, 178, 960, 1034, 6, 21, 38, 159, 204, 248, 260, 306, 337, 349, 384, 425, 438, 458, 481, 502, 521, 546, 605, 690, 730, 731, 819, 860, 915, 946, 967, 1007, 1018, 1065, 1113, 1187, 1188, 1205, 1223, 1409, 1414, 1495, 1526, 1531, 1537, 101, 255, 1421, 1457, 1538, 1580 and 1589, of SEQ ID NO:83;15-25, 41-102, 111-117, 127-134, 145-170, 194-201, 207-225, 10-30, 36-44, 46-59, 57-98, 122-138, 144-160, 162-173, 194-217 and 118-131 of SEQ ID NO:84; and fragments in 9 amino acid length starting from the position of: 12, 16, 37, 46, 61, 82, 121, 128, 149, 157, 162, 197, 204, 212, 39, 2, 23, 53, 68, 97, 107, 121, 127, 156, 169, 196, 9, 13 and 114 of SEQ ID NO:84; 7-54, 65-94, 97-103, 154-163, 170-180, 182-199, 216-222, 227-234, 243-256, 267-273, 286-298, 314-322, 324-353, 363-380, 393-401, 424-431, 434-441, 447-470, 475-495, 506-532, 540-548, 554-592, 594-607, 609-617, 619-626, 628-634, 656-662, 8-31, 43-59, 61-75, 93-104, 126-144, 179-201, 244-254, 289-302, 330-338, 364-382, 413-421, 428-466, 476-525, 582-599, 602-619 621-632 and 115-128 of SEQ ID NO:85; and fragments in 9 amino acid length starting from the position of: 9, 10, 13, 35, 46, 76, 77, 83, 151, 165, 179, 187, 195, 283, 326, 338, 342, 360, 365, 368, 375, 415, 450, 485, 508, 556, 565, 569, 576, 602, 5, 20, 130, 181, 251, 271, 288, 294, 333, 355, 356, 364, 446, 451, 467, 483, 486, 523, 544, 611, 214, 219, 323, 399, 424 and 458, of SEQ ID NO:85; 5-21, 32-56, 88-99, 117-124, 128-138, 143-150, 168-180, 183-189, 196-213, 220-240, 254-263, 266-289, 300-313, 321-330, 335-358, 361-371, 380-398, 50-65, 67-87, 96-104, 144-153, 156-164, 169-177, 199-220, 259-289, 324-333, 339-360, 372-385 and 74-93 of SEQ ID NO:86; and fragments in 9 amino acid length starting from the position of: 26, 33, 49, 88, 96, 129, 169, 170, 198, 257, 268, 281, 337, 342, 366, 391, 393, 39, 122, 248, 76, 106, 117, 185, 190, 198, 238, 257, 266, 280, 341, 344, 350, 367, 304 and 384 of SEQ ID NO:86; 12-23, 44-50, 54-60, 91-97, 103-109, 119-125, 131-137, 141-151, 172-183, 201-226, 230-238, 252-265, 315-321, 331-345, 360-370, 376-386, 392-406, 410-416, 422-431, 133-159, 208-222, 354-368 and 1-88 of SEQ ID NO:87; and fragments in 9 amino acid length starting from the position of: 47, 134, 140, 143, 203, 204, 210, 254, 355, 358, 359, 362, 369, 417, 119, 17, 128, 129, 141, 143, 153, 208, 232, 245, 278, 301, 313, 327, 328, 384 and 395 of SEQ ID NO:87; 4-16, 29-36, 39-64, 69-75, 79-87, 90-122, 126-134, 139-173, 184-190, 195-203, 206-213, 216-228, 234-246, 250-257, 260-266, 274-282, 291-312, 318-325, 340-345, 348-361, 364-388, 399-437, 439-448, 451-464, 467-473, 480-510, 514-520, 534-553, 561-574, 579-589, 593-599, 616-655, 658-671, 3-12, 23-38, 27-38, 43-56, 93-107, 123-137, 144-154, 175-199, 229-244, 288-303, 308-316, 323-337, 410-423, 455-473, 488-496, 531-551, 560-577, 577-591, 619-637, 646-660, 664-672 and 553-570 of SEQ ID NO:88; and fragments in 9 amino acid length starting from the position of: 36, 101, 123, 129, 136, 146, 156, 160, 194, 205, 219, 236, 245, 283, 289, 350, 402, 413, 437, 475, 505, 517, 542, 585, 605, 620, 627, 657, 34, 52, 88, 358, 540, 656, 3, 8, 13, 32, 82, 105, 111, 117, 137, 167, 173, 180, 182, 262, 300, 306, 350, 409, 412, 423, 499, 500, 563, 568, 581, 585, 627, 628, 554 and 638 of SEQ ID NO:88; 4-31, 50-80, 83-93, 97-103, 111-116, 123-132, 134-163, 170-199, 205-210, 215-220, 230-247, 249-278, 280-308, 311-329, 337-347, 349-358, 365-371, 376-401, 417-430, 434-446, 459-505, 511-518, 527-535, 537-545, 547-565, 573-581, 592-601, 1-17, 20-30, 66-80, 100-119, 139-150, 171-182, 186-198, 207-221, 228-242, 258-274, 286-308, 314-330, 337-352, 355-376, 383-391, 417-432, 437-446, 462-473, 479-488, 496-507, 514-522, 541-554, 557-565, 576-585, 589-605, 49-60 and 582-607 of SEQ ID NO:89; and fragments in 9 amino acid length starting from the position of: 4, 65, 66, 120, 121, 144, 170, 174, 208, 226, 233, 276, 278, 285, 286, 298, 336, 348, 355, 363, 382, 384, 395, 457, 458, 494, 501, 578, 133, 278, 294, 551, 53, 89, 110, 159, 186, 232, 290, 324, 406, 431, 458, 463, 480, 490, 513, 541, 549, 558, 585, 22, 137, 152, 189, 227, 255, 261, 291, 419 and 569 of SEQ ID NO:89; 9-60, 67-73, 79-93, 109-122, 134-142, 144-153, 165-192, 197-225, 235-244, 259-279, 289-299, 308-317, 321-332, 338-347, 350-361, 373-387, 402-409, 411-421, 439-445, 450-456, 462-468, 470-479, 490-501, 503-516, 16-27, 49-60, 99-122, 136-145, 148-162, 186-194, 213-221, 225-246, 261-275, 281-292, 353-361, 390-401, 451-470, 486-494, 497-516 and 478-490 of SEQ ID NO:90; and fragments in 9 amino acid length starting from the position of: 15, 22, 28, 29, 48, 49, 106, 107, 114, 147, 170, 177, 188, 208, 209, 212, 256, 280, 287, 316, 451, 468, 489, 33, 217, A03: 36, 98, 124, 136, 142, 153, 177, 188, 251, 262, 291, 320, 323, 383, 417, 464, 487, 491, 492, 505, 44, 86, 146, 411, 437 and 499 of SEQ ID NO:90; 8-18, 20-30 and 7-15 of SEQ ID NO:92; 4-16, 18-27, 2-13, 20-30 and 10-29 of SEQ ID NO:93; and fragments in 9 amino acid length starting from the position of: 22 and 1 of SEQ ID NO:93; 36-57, 62-92, 46-66 and 27-35 of SEQ ID NO:94; and fragments in 9 amino acid length starting from the position of: 84 of SEQ ID NO:94; 4-18, 1-16 and 5-12 of SEQ ID NO:95; and fragments in 9 amino acid length starting from the position of: 1, 9 and 2 of SEQ ID NO:95; 13-27, 38-52, 1-13, 11-25, 27-37 and 17-36 of SEQ ID NO:96; and fragments in 9 amino acid length starting from the position of: 16, 37 and 20 of SEQ ID NO:96; 4-17, 27-40, 55-62, 9-25, 34-46, 50-64 and 47-62 of SEQ ID NO:97; and fragments in 9 amino acid length starting from the position of: 7, 10, 11, 14 and 58 of SEQ ID NO:97; 4-9, 1-10 of SEQ ID NO:98; 3-14 and 7-20 of SEQ ID NO:99; and fragments in 9 amino acid length starting from the position of: 2 and 1 of SEQ ID NO:99; 7-12, 24-29, 22-30 and 7-21 of SEQ ID NO:100; and fragments in 9 amino acid length starting from the position of: 4 and 9 of SEQ ID NO:100; 14-30, 15-30 and 3-18 of SEQ ID NO:101; and fragments in 9 amino acid length starting from the position of: 1 and 20 of SEQ ID NO:101; 3-17 of SEQ ID NO:102; and fragments in 9 amino acid length starting from the position of: 1 of SEQ ID NO:102; 4-27, 31-59, 75-86, 93-103, 105-110, 15-44, 51-61, 79-95 and 41-50 of SEQ ID NO:103; and fragments in 9 amino acid length starting from the position of: 11, 15, 24, 28, 31, 35, 36, 42, 48, 49, 53, 78, 79, 97, 20, 28, 35, 37, 43, 49, 60, 65, 77, 85, 86, 21 and 103 of SEQ ID NO:103; 4-13 and 2-14 of SEQ ID NO:104; and fragments in 9 amino acid length starting from the position of: 7 and 10 of SEQ ID NO:104; 4-15, 17-23, 39-52, 4-13, 16-29, 40-50 and 33-41 of SEQ ID NO:105; and fragments in 9 amino acid length starting from the position of: 3, 38, 14 and 41 of SEQ ID NO:105; 4-25 of SEQ ID NO:106; 8-19, 40-47, 67-86, 88-125, 15-25, 48-59, 64-80, 108-118 and 60-70 of SEQ ID NO:107; and fragments in 9 amino acid length starting from the position of: 7, 110, 16, 34 and 109 of SEQ ID NO:107; 4-27, 41-46, and 30-47 of SEQ ID NO:108; and fragments in 9 amino acid length starting from the position of: 19, 1 and 23 of SEQ ID NO:108; 21-28, 34-43, 8-16 and 23-42 of SEQ ID NO:109; and fragments in 9 amino acid length starting from the position of: 34, 19, 28 and 39 of SEQ ID NO:109; 8-20, 24-37, 39-50, 61-67, 69-91, 4-16, 31-42, 84-93 and 42-59 of SEQ ID NO:110; and fragments in 9 amino acid length starting from the position of: 4, 24, 79, 83, 7, 25, 71, 79 and 91 of SEQ ID NO:110; 4-25, 31-39, 59-97, 100-118, 120-129, 26-40, 49-57, 66-95, 97-128, 131-139, 38-47 of SEQ ID NO:111; and fragments in 9 amino acid length starting from the position of: 8, 24, 61, 67, 72, 103, 112, 3, 39, 74, 110 and 119 of SEQ ID NO:111; 7-24, 32-43, 45-57, 32-48 and 27-43 of SEQ ID NO:112; and fragments in 9 amino acid length starting from the position of: 14, 18, 38, 47 and 14 of SEQ ID NO:112; 4-18, 20-26, 31-37, 3-17, 33-43 and 34-53 of SEQ ID NO:113; and fragments in 9 amino acid length starting from the position of: 3, 7, 10 and 9 of SEQ ID NO:113; 15-23, 25-39, 43-50, 62-70, 16-32, 61-73 and 67-84 of SEQ ID NO:114; and fragments in 9 amino acid length starting from the position of: 8 and 64 of SEQ ID NO:114; 4-13, 28-42, 3-14, 28-39 and 1-20 of SEQ ID NO:115; and fragments in 9 amino acid length starting from the position of: 31, 7 and 5 of SEQ ID NO:115; 4-10, 19-26, 21-29 and 5-13 of SEQ ID NO:116; 4-22, 40-46, 51-57, 64-76, 2-10, 45-53, 58-72, 73-82 and 33-45 of SEQ ID NO:117; and fragments in 9 amino acid length starting from the position of: 35, 76, 3, 1 and 66 of SEQ ID NO:117; 12-24, 27-42, 13-30, 34-44 and 1-9 of SEQ ID NO:118; and fragments in 9 amino acid length starting from the position of: 36, 15 and 18 of SEQ ID NO:118; 4-55, 5-15, 17-33 and 26-45 of SEQ ID NO:119; and fragments in 9 amino acid length starting from the position of: 14 and 53 of SEQ ID NO:119; 31-42, 45-52, 86-92, 8-16, 35-52, 83-91 and 27-93 of SEQ ID NO:120; and fragments in 9 amino acid length starting from the position of: 86, 56, 21 and 4 of SEQ ID NO:120; 237-256, 508-530 of SEQ ID NO:61; 227-239 of SEQ ID NO:62; 141-160, 168-187, 155-173 of SEQ ID NO:63; 101-124, 161-187, 59-85, 80-106 of SEQ ID NO:64; 97-112 of SEQ ID NO:66; 139-165 of SEQ ID NO:67; 10-21 of SEQ ID NO:68; 667-688, 677-696, 161-187, 183-209, 205-231, 226-252 of SEQ ID NO:69; 603-629, 622-648, 643-669 of SEQ ID NO:70; 529-541 of SEQ ID NO:71; 12-34, 29-51, 46-67, 62-83 of SEQ ID NO:72; 139-151 of SEQ ID NO:73; 246-262, 251-275 of SEQ ID NO:74; 61-84, 79-102, 97-120, 115-138 of SEQ ID NO:75; 325-350, 345-370, 365-389 of SEQ ID NO:76; 324-349, 336-351 of SEQ ID NO:77; 90-100 of SEQ ID NO:78; 274-290 of SEQ ID NO:79; 401-419 of SEQ ID NO:80; 84-107, 101-123, 117-139 of SEQ ID NO:81; 182-199 of SEQ ID NO:82; 911-935 of SEQ ID NO:83; 118-131 of SEQ ID NO:84; 115-128 of SEQ ID NO:85; 74-93 of SEQ ID NO:86; 21-43, 54-76 of SEQ ID NO:87; 554-570 of SEQ ID NO:88; 478-490 of SEQ ID NO:90; 2-14 of SEQ ID NO:91; 7-15 of SEQ ID NO:92; 10-28 of SEQ ID NO:93; 27-34 of SEQ ID NO:94; 17-35 of SEQ ID NO:96; 47-61 of SEQ ID NO:97; 1-10 of SEQ ID NO:98; 7-20 of SEQ ID NO:99; 7-20 of SEQ ID NO:100; 3-17 of SEQ ID NO:101; 3-17 of SEQ ID NO:102; 41-50 of SEQ ID NO:103; 2-14 of SEQ ID NO:104; 33-41 of SEQ ID NO:105; 4-25 of SEQ ID NO:106; 60-69 of SEQ ID NO:107; 23-41 of SEQ ID NO:109; 42-59 of SEQ ID NO:110; 38-46 of SEQ ID NO:111; 27-43 of SEQ ID NO:112; 34-53 of SEQ ID NO:113; 67-84 of SEQ ID NO:114; 1-20 of SEQ ID NO:115; 33-45 of SEQ ID NO:117; 26-45 of SEQ ID NO:119; 27-53 of SEQ ID NO:120.

3. The isolated antigen of claim 1, comprising at least 8 contiguous amino acids of any of SEQ ID NOs: 61-90 or 92-120.

4. The isolated antigen of claim 1, comprising at least 10 contiguous amino acids of any of SEQ ID NOs: 61-90 or 92-120.

5. An immunogenic composition comprising the isolated antigen of claim 1.

6. The immunogenic composition of claim 5, comprising at least two different isolated antigens of claim 1.

7. The immunogenic composition of claim 5, further comprising an immunostimulatory substance.

8. The immunogenic composition of claim 7, wherein the immunostimulatory substance is a polycationic polymer, an immunostimulatory deoxynucleotide (ODN), a peptide containing at least two LysLeuLys motifs, a neuroactive compound, alum, or a Freund's complete or incomplete adjuvant.

9. The immunogenic composition of claim 8, wherein the polycationic polymer is a polycationic peptide.

10. The immunogenic composition of claim 8, wherein the neuroactive compound is human growth hormone.

11. A method of eliciting an immune response in a subject comprising:
obtaining a immunogenic composition of claim 5; and
administering the immunogenic composition to a subject;
wherein an immune response is elicited in the subject.

12. The method of claim 11, wherein the subject is a human.


SEQUENCE LISTING (122 sequences)

SEQ ID NO: 1 - DNA, 1956 bp, Artificial Sequence
  Description of Artificial Sequence  Synthetic Primer
atggttaatcctattggtccaggtcctatagacgaaacagaacgcacacctcccgcagatctttctgctcaaggattggaggcgagtgcagcaaataagagtgcggaagctcaaagaatagcaggtgcggaagctaagcctaaagaatctaagaccgattctgtagagcgatggagcatcttgcgttctgcagtgaatgctctcatgagtctggcagataagctgggtattgcttctagtaacagctcgtcttctactagcagatctgcagacgtggactcaacgacagcgaccgcacctacgcctcctccacccacgtttgatgattataagactcaagcgcaaacagcttacgatactatctttacctcaacatcactagctgacatacaggctgctttggtgagcctccaggatgctgtcactaatataaaggatacagcggctactgatgaggaaaccgcaatcgctgcggagtgggaaactaagaatgccgatgcagttaaagttggcgcgcaaattacagaattagcgaaatatgcttcggataaccaagcgattcttgactctttaggtaaactgacttccttcgacctcttacaggctgctcttctccaatctgtagcaaacaataacaaagcagctgagcttcttaaagagatgcaagataacccagtagtcccagggaaaacgcctgcaattgctcaatctttagttgatcagacagatgctacagcgacacagatagagaaagatggaaatgcgattagggatgcatattttgcaggacagaacgctagtggagctgtagaaaatgctaaatctaataacagtataagcaacatagattcagctaaagcagcaatcgctactgctaagacacaaatagctgaagctcagaaaaagttccccgactctccaattcttcaagaagcggaacaaatggtaatacaggctgagaaagatcttaaaaatatcaaacctgcagatggttctgatgttccaaatccaggaactacagttggaggctccaagcaacaaggaagtagtattggtagtattcgtgtttccatgctgttagatgatgctgaaaatgagaccgcttccattttgatgtctgggtttcgtcagatgattcacatgttcaatacggaaaatcctgattctcaagctgcccaacaggagctcgcagcacaagctagagcagcgaaagccgctggagatgacagtgctgctgcagcgctggcagatgctcagaaagctttagaagcggctctaggtaaagctgggcaacaacagggcatactcaatgctttaggacagatcgcttctgctgctgttgtgagcgcaggagttcctcccgctgcagcaagttctatagggtcatctgtaaaacagctttacaagacctcaaaatctacaggttctgattataaaacacagatatcagcaggttatgatgcttacaaatccatcaatgatgcctatggtagggcacgaaatgatgcgactcgtgatgtgataaacaatgtaagtacccccgctctcacacgatccgttcctagagcacgaacagaagctcgaggaccagaaaaaacagatcaagccctcgctagggtgatttctggcaatagcagaactcttggagatgtctatagtcaagtttcggcactacaatctgtaatgcagatcatccagtcgaatcctcaagcgaataatgaggagatcagacaaaagcttacatcggcagtgacaaagcctccacagtttggctatccttatgtgcaactttctaatgactctacacagaagttcatagctaaattagaaagtttgtttgctgaaggatctaggacagcagctgaaataaaagcactttcctttgaaacgaactccttgtttattcagcaggtgctggtcaatatcggctctctatattctggttatctccaataa

SEQ ID NO: 2 - DNA, 1170 bp, Artificial Sequence
  Description of Artificial Sequence  Synthetic Primer
atgaaaaaactcttaaagtcggcgttattatccgccgcatttgctggttctgtcggctccttacaagccttgcctgtagggaacccttctgatccaagcttattaattgatggtacaatatgggaaggtgctgcaggagatccttgcgatccttgcgctacttggtgcgacgctattagcttacgtgctggattttacggagactatgttttcgaccgtatcttaaaagtagatgcacctaaaacattttctatgggagccaagcctactggatccgctgctgcaaactatactactgccgtagatagacctaacccggcctacaataagcatttacacgatgcagagtggttcactaatgcaggcttcattgccttaaacatttgggatcgctttgatgttttctgtactttaggagcttctaatggttacattagaggaaactctacagcgttcaatctcgttggtttattcggagttaaaggtactactgtaaatgcaaatgaactaccaaacgtttctttaagtaacggagttgttgaactttacacagacacctctttctcttggagcgtaggcgctcgtggagccttatgggaatgcggttgtgcaactttgggagctgaattccaatatgcacagtccaaacctaaagttgaagaacttaatgtgatctgtaacgtatcgcaattctctgtaaacaaacccaagggctataaaggcgttgctttccccttgccaacagacgctggcgtagcaacagctactggaacaaagtctgcgaccatcaattatcatgaatggcaagtaggagcctctctatcttacagactaaactctttagtgccatacattggagtacaatggtctcgagcaacttttgatgctgataacatccgcattgctcagccaaaactacctacagctgttttaaacttaactgcatggaacccttctttactaggaaatgccacagcattgtctactactgattcgttctcagacttcatgcaaattgtttcctgtcagatcaacaagtttaaatctagaaaagcttgtggagttactgtaggagctactttagttgatgctgataaatggtcacttactgcagaagctcgtttaattaacgagagagctgctcacgtatctggtcagttcagattctaa

SEQ ID NO: 3 - DNA, 642 bp, Artificial Sequence
  Description of Artificial Sequence  Synthetic Primer
atgtctgttaatccatcaggaaattccaagaacgatctctggattacgggagctcatgatcagcatcccgatgttaaagaatccggggttacaagtgctaacctaggaagtcatagagtgactgcctcaggaggacgccaagggttattagcacgaatcaaagaagcagtaaccgggttttttagtcggatgagcttcttcagatcgggagctccaagaggtagccaacaaccctctgctccatctgcagatactgtacgtagcccgttgccgggaggggatgctcgcgctaccgagggagctggtaggaacttaattaaaaaagggtaccaaccagggatgaaagtcactatcccacaggttcctggaggaggggcccaacgttcatcaggtagcacgacactaaagcctacgcgtccggcacccccacctcctaaaacgggtggaactaatgcaaaacgtccggcaacgcacgggaagggtccagcaccccagcctcctaaaacaggtgggaccaatgctaagcgcgcagcaacgcatgggaaaggtccagcacctcaacctcctaagggcattttgaaacagcctgggcagtctgggacttcaggaaagaagcgtgtcagctggtctgacgaagattaa

SEQ ID NO: 4 - DNA, 1149 bp, Artificial Sequence
  Description of Artificial Sequence  Synthetic Primer
atgggaatcaatccttcgggtaatagatcaccagatgatgtatgggttagaggagctcaaggcgatagctccagtacccaaggtacaggagctacaaactcaaatcttggtgctcacaacgtaactacatcaacctcacagccgcaagttgcttctaaagcaaagcagttatggcagacggtaagggagttctttttagggaagaaatcacccgattcttctcagggtgcttcgggacctgcaatgcaaagtccttcaggacctacaatacggcctacgcgtccggcacctccacctcctacaacgggtggggctaatgcgaaacgtcccgcaacgcatgggaaaggtcgagcacctcaacctcctacggcggggtcttcttcaggatcagagcaacctactgccatgagttctgaagtcgctaaacttgtgagtgaattaaaagatgcagtccatagtcatgcggagtctcaaaaagtacttaaaaaggtatctcaagagctacaaacaaagtggacggattgggaaaataataggggtccagactatcttttgcatggttatcgtgtcattgctcgagctttgcagcaaacatacacagaacaatctatgcttatcgaagggacttcatctacaggaccagttccgcaagcagtgactgtagctaaggatgctgtaactcagacagttagaggcgcaattaagaatttagaaaatcctaagccaggtaatgatcctgatggtgtactcatgcaagtggttataagcttaggtatcgaaggacctacattagacccaggagaatctatccaaaactttttagaaactagggtttcggatttcggtggagatgatagcgacatagattatacaagtgatatagctcgattagggtcagctttagatcgggtacgcgaaaatcatcctaatgagatgcctagaatatggatagcattagcacgagaactcggtgcggctgtacactctcatgctacttccgtccgaatcgcaaatgcaggaaagaatcacactcgtgacgttgtgcgaatggccaatgagtcgagtagactacttcaaggtatgaaagtgttatcggtcggagcttgggcgaatacaatgacagttttaatcggggatctttttgaataa

SEQ ID NO: 5 - DNA, 1002 bp, Artificial Sequence
  Description of Artificial Sequence  Synthetic Primer
atgaaaacgttgtggcacttcgtatccaaagcctttttatcgatagtaggactgtgttgcggagttgttcttgcttttgtcgttatattcgcactcatagcttcctctttaggaaatggggatgctaccttcgttagcttgcctgacgcccaaggagaagtaaaagatctagggaaaacagcccccattattgctgttatcgaaatgaaagatgtaattgcttcttcaaaaaatacggccaaaacgattcagaatattttagaaggatttgagaaagctcctcttaaagatcgtgtcaaaggtattgtcattgatatggattgcccaggaggcgaggtctttgaaatagatagaatttactctatgcttcgcttttggaaagaacgtaagggattccctatttatatttatgtgaatggtctttgtgcttcgggaggctattatgtatcctgcgctgcaactaaaatttatgccacctcctcctctcttatcggttctatcggagtgcgttctggaccattcttcaatgtaaaagaaggtttaaatcgctacggagttgaaagtgatctgctgacagctggaaaagataaggctccaatgaatccttatacaccgtggacttctcatgatagagaagaacggcaagcgactcttgattttctctacggacaatttgttgatatagttacacaaaaccgtcctctgcttactaaagagaagttagttcacactctcggagcacgtattttttctccagagaaggccaaacaagaaggctatattgatgttgtaggcgcaactaaagaacaagtccttcaagacatagttgctgtttgtaagattgaagataactatagagtgattggctctggtggtgatggttggtggaagcgggtggcttcagctgcagcttcaagtccattagttactggcatgattaaacacgatattctgcctttatcccatgacgctgcatacatacctccctacttggcactgtag

SEQ ID NO: 6 - DNA, 687 bp, Artificial Sequence
  Description of Artificial Sequence  Synthetic Primer
atgagacctcatcgtaaacacgtatcatctaaaagcttagctttaaagcaatctgcatcaactcatgtagagatcacaacaaaagcctttcgtctctctatgcctctaaaacagctgatcctagagaaaagcgaccacctcccccctatggaaacaatccgtgtggtgctaacctctcataaagataagctaggcaccgaggtgcatgttgtagcttctcatggcaaagaaatccttcaaactaaggttcataacgcaaacccatacactgcagtgatcaatgcttttaagaaaatccgcaccatggcaaataagcactccaataaacgtaaagacaggacaaaacatgatctaggtcttgcagcaaaagaagaacgtatcgcaatacaggaagaacaagaagatcgccttagcaacgagtggcttcctgtcgaaggcctcgatgcctgggattctctaaaaactcttgggtatgttcccgcatcagcgaaaaagaagatctccaagaaaaagatgagcattcgtatgctatctcaagacgaggctatccgccagctagagtctgccgcagaaaacttcctgatcttcttgaacgagcaagagcataaaatccaatgcatttataaaaaacatgacggcaactatgtccttattgaaccttccctcaagccaggattctgcatctga

SEQ ID NO: 7 - DNA, 2268 bp, Artificial Sequence
  Description of Artificial Sequence  Synthetic Primer
atggcagctcctatcaaccaaccatcgacaacgactcagataactcaaactgggcagactacaacgacaacaacggtaggatcattaggagagcattctgttacaacaacaggatctggggcagcagcacaaacatctcagacagtaactctaattgcagatcacgaaatgcaagaaattgcaagtcaagatggatccgcggtaagcttttctgctgagcactctttttctaccctccctccagagactggaagtgttggagctacagcacaatccgctcaatctgcggggctattttcattatcaggtcgtacacaaagaagagattcggagatttcttcctcttctgacggcagttcgatatctagaactagctcaaacgcatcttctggagaaacaagcagagctgaaagtagtcctgatctaggcgacttggatagcttatcaggaagcgagcgcgctgaaggagccgaaggacctgaaggacctggaggcttacctgaaagtacgattccacattatgatcctaccgataaagcgtctattttgaacttcttgaaaaatcctgcagttcagcagaaaatgcagaccaaaggaggccactttgtttatgtagatgaagccagaagtagtttcatttttgtccgcaatggtgactggtcaactgctgagtctataaaagtttctaatgcaaaaaccaaagaaaatattactaagcctgcggacttagaaatgtgcatcgctaaattctgtgtgggatatgaaaccatccactcggattggacgggacgcgtaaaacctacaatggaagagcgctcgggagccacaggaaattacaatcatctgatgctcagcatgaaatttaaaactgctgtagtctacggtccttggaatgctaaagaatctagtagtggatatacaccctctgcatggcgtcgtggagcaaaagtagaaacaggtccgatttgggatgatgttgggggcttgaaaggcattaactggaaaacgaccccagctccagacttctcctttataaatgaaactccaggtggaggggctcactcgacgtctcatacaggtcctggcactccagtaggagctactgtggttcctaatgtgaatgtcaacttgggaggcattaaggttgatctgggtggcatcaatttaggtggaattacaacgaatgtcactacagaagaaggtggtggaaccaacataacatctacgaaatccacatctactgatgataaagtctcaataacatctacaggatctcaaagtacgatcgaagaagacactatacaatttgacgatcctggtcagggagaggatgataacgcaattcccggcacaaacacacctcctcctccaggtcctccgccaaatctaagcagttctcgcttgctgactatttcgaatgcgtccttgaaccaagtcttacagaatgtccgacaacatctgaatacggcttatgattcgaatggtaattcagtctcagatctcaatcaggatttaggccaggtagtaaaaaacagtgaaaacggagtgaacttccctactgtgattcttcctaaaactactggcgatacagatccatccggtcaagcaaccggaggagtcactgaaggcggcggtcatatccgtaatattatccaaaggaatacacaatctacggggcaaagtgaaggagcaacacctacacctcaacctactatagcaaagatagtgacttccctgagaaaagcaaatgtaagttccagctctgtgctaccacaaccacaagtagctacgacgatcacccctcaagcgagaacggccagtacatctacaacgagcataggaaccgggacagaaagcacatctacaacaagtacgggaacgggaacaggaagtgtctccacacaaagtactggcgtagggacaccaactacgacgactcgatctacaggaacttcggcgacaaccacaacatcatcagcttcgacacaaacaccccaagcgcctcttccctctgggaccaggcatgttgctacaatctccttagtgcgtaatgctgcaggaaggtctattgtattacaacaagggggtcgatctcaaagcttcccgatccctccctcagggactggaacacagaatatgggggcacaattgtgggctgcagcaagtcaagttgcttccactttaggccaggtcgtgaatcaagcagctacagcaggttctcaaccctcctctcgtagatcttccccaacaagtccacgaagaaaatag

SEQ ID NO: 8 - DNA, 1707 bp, Artificial Sequence
  Description of Artificial Sequence  Synthetic Primer
atgaaaacgtctcaactcttttataagacttcaaaaaatgcaaataaaagcgctgctgtgctctcaaacgagctcctagaaaaggcaggatacctatttaaagtaagtaaaggagtctatacctatacacccctgttatggcgcgtggtctccaagatgatgaacatcattagagaggaacttaatgcgattggaggtcaagaacttctactcccacttctccacaatgctgaactttggcaacatacagggagatgggaggcatttacttcggaaggactgctctacactctcaaagaccgcgaaggaaaatctcattgcctagctcctacacatgaagaggtcatctgctcttttgttgcacaatggctctcctcaaaaagacaacttcctctccacctttaccaaattgctacaaaattccgagacgagattcgccctcgattcggtctcattcgctctcgagagctccttatggaagacagctataccttctcagactctcccgaacaaatgaacgagcaatatgaaaaactccgctctgcgtatagtaagatctttgatcgtctcggtcttgcctatgtcatcgttacagctgatggagggaaaatcggcaaaggaaagtctgaggaatttcaggtcctttgctctctaggcgaggacacgatctgcgtcagcggttcctatggagctaatattgaggctgctgtctccattcctccacagcatgcctacgatcgcgagtttcttcccgtcgaagaagtggccacccctgggattacaacaatagaagctctagcaaacttcttctctatccccttacataaaattttaaaaacccttgtcgtaaaactctcctactcaaatgaagaaaaattcattgccattggaatgagaggagatcggcaagtcaacctagtgaaggtcgcttccaaactgaatgccgatgatattgctctagcttctgatgaagaaatcgaacgcgttctaggcacagaaaaaggattcatcggtcccctaaactgtcccatagactttttcgcagacgaaacaacgtccccaatgacgaactttgtttgtgcgggcaatgctaaagataagcactacgtaaatgtaaactgggatcgcgacctcctccccccccaatacggtgactttctactcgctgaagagggagacacatgtcctgaaaatcctggccatccttaccgcatttatcaaggcatagaagttgctcatattttcaatctcgggacacgctataccgatagttttgaggtaaacttccaagatgaacacgggcaaacccagcagtgctggatggggacctacggcattggagtcggaagaacattagccgcttgtgtagaacagcttgccgacgaccgtggtattgtttggccaaaagcactcgctcccttctctatcactatcgcctttaacggaggagacactgtatctcaagagcttgcggaaactatttatcatgagctacaaagtcaaggctatgagccccttcttgatgatcgagatgaaagactcggatttaaacttaaagacagtgaccttatcggcattccttataagcttattttaggaaagtcctaccaatcttcgggaatattcgaaattgaatcccgatctggagaaaagtatacagtctccccggaggccttccctacttggtgtcagaatcacttagcctag

SEQ ID NO: 9 - DNA, 2328 bp, Artificial Sequence
  Description of Artificial Sequence  Synthetic Primer
atggcatcaggaatcggaggatctagtggattaggaaagattccacctaaagataatggggatagaagtcgatcgccctctcctaagggagaacttggcagccacgagatttccctgcctcctcaagaacatggagaggaaggagcttcaggatcttcgcatatacatagcagttcctcttttctaccagaagatcaggagtctcagagctcttcttcggcagcttctagcccgggatttttttctcgcgtacgttctggggtagacagggccttaaaatcatttggcaactttttttccgcagagtctacgagtcaagcgcgtgaaacgcgacaagcttttgttagattatcaaaaaccatcaccgcggatgagagacgggatgtcgattcatcaagtgctgctgctacagaagcccgagtggcagaggacgcgagtgtttcaggcgaaaatccttctcagggggttccagaaacctcttctggaccagaacctcagcgtttattttctcttccttcagtaaaaaaacagagcggtttgggtcggttggtacagacagttcgcgatcgcatagtacttcctagtggggctccacctacagacagcgagcctttaagtctctacgagctaaacctccgtttgagtagtttacgtcaggagctctctgacatacaaagtaatgatcagttgactccagaggaaaaagcagaagccacagttaccatacaacagctgatccaaattacagaattccaatgcggctatatggaggcaacacaatcttcggtatctctagcagaagctcgttttaagggggtagaaactagtgatgagatcaattccctctgttcagaactgacagatcctgagcttcaagaactcatgagtgatggagactctcttcaaaacctattagatgagactgccgacgatttagaagctgctttgtcccatgctcgattgagtttttctttagacgataatccaactccgatagacaataatccaactctgatttctcaagaagagcctatttatgaggaaatcggaggagctgcagatcctcaaagaactcgggaaaactggtctacaagattatggaatcagattcgcgaggctctggtttctcttttaggaatgattttaagcattctagggtccatcttgcacaggttgcgtattgctcgtcatgcagctgctgaagcagtgggtcgttgttgcacgtgccgaggagaagagtgtacttcttctgaagaggactcgatgtcggtggggtctccttcagaaattgatgaaactgaaagaacgggctctccgcatgacgttccacgcagaaatggaagtccacgtgaagattctccattgatgaatgccttagtaggatgggcacataagcacggtgctaaaaccaaggagagttcagaatcaagtaccccggaaatttcgatttctgctcccatagtgagaggttggagtcaagacagttccgtcagttttattgttatggaagatgatcatattttctatgatgttcctcgtagaaaagatggaatctatgacgttcctagttcccctagatggagtcctgcgcgagagttggaagaggatgtttttggagattatgaagttcctataacctctgctgaaccatctaaagacaagaacatctacatgacacctagattagcaactcctgctatctatgatcttccttcacgtccaggatcgtctggaagctcacgttctccgtcttcagatcgcgtacgaagcagctcaccaaatagacggggtgtgcctcttcctccagttccttcacctgctatgagtgaggaggggagcatttatgaggatatgagcggtgcttcaggtgcaggtgaaagtgattatgaagatatgagccgttccccctctcctagaggcgacttggatgaacccatatatgctaatactcctgaagataatccatttactcagagaaatatagatagaattttacaggagaggtcaggcggtgcttccgcttctcctgtagagcctatttatgatgagatcccatggattcatggcaggccccctgctacacttccaagacccgagaatacattgactaatgtttcgcttagagtgagcccagggtttggaccagaagtaagagccgctttgcttagcgagagcgtgagtgctgttatggtcgaagcagagagtattgttcctccaacagagccgggggacggagaatcagaatatctagagcccttagggggacttgtagctacaacgaaaatcttactacaaaaaggatggcctcgtggagagtcgaatgcttag

SEQ ID NO: 10 - DNA, 2817 bp, Artificial Sequence
  Description of Artificial Sequence  Synthetic Primer
atgcgctttttttgcttcggaatgttgcttccttttacttttgtattggctaatgaaggtctccaacttcctttggagacctatattacattaagtcctgaatatcaagcagcccctcaagtagggtttactcataaccaaaatcaagatctcgcaattgtcgggaatcacaatgatttcatcttggactataagtactatcggtcgaatggaggtgctcttacctgtaagaatcttctgatctctgaaaatatagggaatgtcttctttgagaagaatgtctgtcccaattctggcggggcaatttatgctgctcaaaattgcacgatctccaagaatcagaactatgcatttactacaaacttggtctctgacaatcctacagccactgcgggatcactattgggtggagctctctttgccataaattgctctattactaataacctaggacagggaactttcgttgacaatctcgctttaaataaggggggtgccctctatactgagacgaacttatctattaaagacaataaaggcccgatcataatcaagcagaatcgggcactaaattcggacagtttaggaggagggatttatagtgggaactctctaaatatagagggaaattctggagctatacagatcacaagcaactcttcaggatctgggggaggcatattttctacccaaacactcacgatctcctcgaataaaaaactcatagaaatcagtgaaaattccgcgttcgcaaataactatggatcgaacttcaatccaggaggaggaggtcttactaccaccttttgcacgatattgaacaaccgagaaggggtactctttaacaataaccaaagccagagcaacggtggagccattcatgcgaaatctatcattatcaaagaaaatggtcctgtatactttttaaataacactgcaactcggggaggggctctcctcaacttatcagcaggttctggaaacggaagcttcatcttatctgcagataatggagatattatctttaacaataatacggcctccaagcatgccctcaatcctccatacagaaacgccattcactcgactcctaatatgaatctgcaaataggagcccgtcccggctatcgagtgctgttctatgatcccatagaacatgagctcccttcctccttccccatactctttaatttcgaaaccggtcatacaggtacagttttattttcaggggaacatgtacaccagaactttaccgatgaaatgaatttcttttcctatttaaggaacacttcggaactacgtcaaggagtccttgctgttgaagatggtgcggggctggcctgctataagttcttccaacgaggaggcactctacttctaggtcaaggtgcggtgatcacgacagcaggaacgattcccacaccatcctcaacaccaacgacagtaggaagtactataactttaaatcacattgccattgaccttccttctattctttcttttcaagctcaggctccaaaaatttggatttaccccacaaaaacaggatctacctatactgaagattccaacccgacaatcacaatctcaggaactctcaccttacgcaacagcaacaacgaagatccctacgatagtctggatctctcgcactctcttgagaaagttccccttctttatattgtcgatgtcgctgcacaaaaaattaactcttcgcaactggatctatccacattaaattctggcgaacactatgggtatcaaggcatctggtcgacctattgggtagaaactacaacaatcacgaaccctacatctctactaggcgcgaatacaaaacacaagctgctctatgcaaactggtctcctctaggctaccgtcctcatcccgaacgtcgaggagaattcattacgaatgccttgtggcaatcggcatatacggctcttgcaggactccactccctctcctcctgggatgaagagaagggtcatgcagcttccctacaaggcattggtcttctggttcatcaaaaagacaaaaacggttttaagggatttcgtagtcatatgacaggttatagtgctaccaccgaagcaacctcttctcaaagtccgaatttctctttaggatttgctcagttcttctccaaagctaaagaacatgaatctcaaaatagcacgtcctctcaccactatttctctggaatgtgcatagaaaatactctcttcaaagagtggatacgtctatctgtgtctcttgcttatatgtttacctcggaacatacccatacaatgtatcagggtctcctggaagggaactctcagggatctttccacaaccataccttagcaggggctctctcctgtgttttcttacctcaacctcacggcgagtccctgcagatctatccctttattactgccttagccatccgaggaaatcttgctgcgtttcaagaatctggagaccatgctcgggaattttccctacaccgccccctaacggacgtctccctccctgtaggaatccgcgcttcttggaagaaccaccaccgagttcccctagtctggctcacagaaatttcctatcgctctactctctataggcaagatcctgaactccactcgaaattactgattagccaaggtacgtggacgacgcaggccactcctgtgacctacaatgctttagggatcaaagtgaaaaataccatgcaggtgtttcctaaagtcactctctccttagattactctgcggatatttcttcctccacgctgagtcactacttaaacgtggcgagtagaatgagattttaa

SEQ ID NO: 11 - DNA, 2787 bp, Artificial Sequence
  Description of Artificial Sequence  Synthetic Primer
atgaaatcctctcttcattggtttttaatctcgtcatctttagcacttcccttgtcactaaatttctctgcgtttgctgctgttgttgaaatcaatctaggacctaccaatagcttctctggaccaggaacctacactcctccagcccaaacaacaaatgcagatggaactatctataatctaacaggggatgtctcaatcaccaatgcaggatctccgacagctctaaccgcttcctgctttaaagaaactactgggaatctttctttccaaggccacggctaccaatttctcctacaaaatatcgatgcgggagcgaactgtacctttaccaatacagctgcaaataagcttctctccttttcaggattctcctatttgtcactaatacaaaccacgaatgctaccacaggaacaggagccatcaagtccacaggagcttgttctattcagtcgaactatagttgctactttggccaaaacttttctaatgacaatggaggcgccctccaaggcagctctatcagtctatcgctaaaccccaacctaacgtttgccaaaaacaaagcaacgcaaaaagggggtgccctctattccacgggagggattacaattaacaatacgttaaactcagcatcattttctgaaaataccgcggcgaacaatggcggagccatttacacggaagctagcagttttattagcagcaacaaagcaattagctttataaacaatagtgtgaccgcaacctcagctacagggggagccatttactgtagtagtacatcagcccccaaaccagtcttaactctatcagacaacggggaactgaactttataggaaatacagcaattactagtggtggggcgatttatactgacaatctagttctttcttctggaggacctacgctttttaaaaacaactctgctatagatactgcagctcccttaggaggagcaattgcgattgctgactctggatctttgagtctttcggctcttggtggagacatcacttttgaaggaaacacagtagtcaaaggagcttcttcgagtcagaccactaccagaaattctattaacatcggaaacaccaatgctaagattgtacagctgcgagcctctcaaggcaatactatctacttctatgatcctataacaactagcatcactgcagctctctcagatgctctaaacttaaatggtcctgaccttgcagggaatcctgcatatcaaggaaccatcgtattttctggagagaagctctcggaagcagaagctgcagaagctgataatctcaaatctacaattcagcaacctctaactcttgcgggagggcaactctctcttaaatcaggagtcactctagttgccaagtccttttcgcaatctccgggctctaccctcctcatggatgcagggaccacattagaaaccgctgatgggatcactatcaataatcttgttctcaatgtagattccttaaaagagaccaagaaggctacgctaaaagcaacacaagcaagtcagacagtcactttatctggatcgctctctcttgtagatccttctggaaatgtctacgaagatgtctcttggaataaccctcaagtcttttcttgtctcactcttactgctgacgaccccgcgaatattcacatcacagacttagctgctgatcccctagaaaaaaatcctatccattggggataccaagggaattgggcattatcttggcaagaggatactgcgactaaatccaaagcagcgactcttacctggacaaaaacaggatacaatccgaatcctgagcgtcgtggaaccttagttgctaacacactatggggatcctttgttgatgtgcgctccatacaacagcttgtagccactaaagtacgccaatctcaagaaactcgcggcatctggtgtgaagggatctcgaacttcttccataaagatagcacgaagataaataaaggttttcgccacataagtgcaggttatgttgtaggagcgactacaacattagcttctgataatcttatcactgcagccttctgccaattattcgggaaagatagagatcactttataaataaaaatagagcttctgcctatgcagcttctctccatctccagcatctagcgaccttgtcttctccaagcttgttacgctaccttcctggatctgaaagtgagcagcctgtcctctttgatgctcagatcagctatatctatagtaaaaatactatgaaaacctattacacccaagcaccaaagggagagagctcgtggtataatgacggttgcgctctggaacttgcgagctccctaccacacactgctttaagccatgagggtctcttccacgcgtattttcctttcatcaaagtagaagcttcgtacatacaccaagatagcttcaaagaacgtaatactaccttggtacgatctttcgatagcggtgatttaattaacgtctctgtgcctattggaattaccttcgagagattctcgagaaacgagcgtgcgtcttacgaagctactgtcatctacgttgccgatgtctatcgtaagaatcctgactgcacgacagctctcctaatcaacaatacctcgtggaaaactacaggaacgaatctctcaagacaagctggtatcggaagagcagggatcttttatgccttctctccaaatcttgaggtcacaagtaacctatctatggaaattcgtggatcttcacgcagctacaatgcagatcttggaggtaagttccagttctaa

SEQ ID NO: 12 - DNA, 2538 bp, Artificial Sequence
  Description of Artificial Sequence  Synthetic Primer
atgtttgagaagttcactaatagagcaaaacaagtcattaaactggcgaaaaaggaggctcagcgtttaaatcataactacctgggtactgagcacatcctgcttggtcttctcaaacttggtcaaggggtagctgttaatgtattacgcaacctcggtatagattttgatacggcacggcaagaggtggaacgcctgattggttatggtccagaaattcaagtctacggagaccctgcccttacaggaagagtaaaaaaatcttttgaatcagcaaatgaagaggccagccttttagagcacaattatgtcgggacggagcatttactcttagggatcctacatcaatcagatagtgtcgctcttcaggtattagaaaacttacatatcgatccaagagaggttcgtaaggaaattcttaaagaattagagaccttcaatctacaacttcctccttcgtcgtcgtcttcttcctcatcctctcgaagcaacccttcatcttcaaaatctcctttaggtcatagcttaggttctgacaaaaacgaaaagctttctgctctgaaagcatatggttatgatttaacggagatggtccgagagtctaagctcgatcctgtcattggtcgttcttcagaagtcgaacggttgattttgattctttgccgaagaagaaaaaacaatcctgtacttattggagaagctggagttggtaagactgcaattgttgagggtctggctcaaaaaatcattctgaatgaggttcctgatgccttacggaaaaagcgactgattactctagatctagcattaatgattgctggaacaaaatatcgagggcaatttgaggaacggatcaaagctgtcatggatgaagttcgcaagcatggaaacatcttgctcttcattgacgagctccacacgattgtaggagcaggagcagctgaaggtgctatcgatgcttcaaacattttaaaacctgcgttagcgcgaggtgaaattcagtgtattggagcaactacgatagatgagtatcgcaagcacatagaaaaagacgcagctttagaacgtcgtttccaaaaaatcgtggttcaccctcctagtgtagatgagactattgagattttacgtggcctcaagaaaaagtatgaagaacatcacaatgtcttcattactgaagaagctttaaaagcagctgcgactctttctgatcaatatgttcatggacgtttcctccctgataaagcaatagatcttttagatgaagctggggctcgtgtccgtgtgaatacaatgggtcagcctacagatttaatgaagctagaggctgaaatcgaaaatacaaaattggccaaagagcaggccattggaactcaagaatacgaaaaagctgcaggtttacgtgatgaagagaaaaaacttcgcgaacgtctgcaaagtatgaaacaggaatgggaaaatcataaagaagagcaccaagttcctgtagatgaagaagcagtcgctcaggtagtttctctacaaacaggaattccctcagcaaggctcacagaagctgaaagtgagaagcttctgaagttagaagacacgttaagaagaaaagtcattggtcaaaatgatgccgttaccagcatttgccgtgccatccgacgttctcgaacagggatcaaagatcctaaccgacctacgggctccttcctattccttgggcctaccggtgtagggaaaagcctgctcgcccaacaaattgctatagagatgttcggtggtgaagacgctctgattcaggtagacatgtcagagtacatggagaaatttgctgctaccaagatgatgggatcacctccaggatatgtaggtcatgaagaagggggccaccttacggaacaggtacgtcgccgtccttactgcgttgttctctttgatgagatagaaaaggcacacccagacattatggacctgatgttgcaaattttagagcaaggacgtcttactgattcttttggtcgcaaagtggatttccgtcatgccattattatcatgacctccaatttgggagctgatctcattcgtaaaagcggagaaattggttttggcttgaagtcccatatggactataaggtcatccaagagaaaatcgaacatgctatgaagaaacacttaaagcctgagttcattaaccgtttggatgaaagtgtgattttccgtcccctcgagaaagaatctctatcggagatcatccatttagagatcaacaaactggactcgagactgaaaaactaccaaatggctttgaacatcccagactctgtgatttccttcctagtaacgaaggggcattctccagaaatgggagcacgtcctctacgccgtgtcattgagcagtaccttgaagatcctctagcggagctcttgcttaaagagtcctgccgtcaagaagctcgcaagctacgagcaaccttggttgaaaatcgcgttgcctttgaaagggaagaagaggagcaggaagctgctctccctagccctcacttggaatcatag

SEQ ID NO: 13 - DNA, 1215 bp, Artificial Sequence
  Description of Artificial Sequence  Synthetic Primer
atggggctgcaatccaggttacaacattgtatagaagtgtcccagaattcgaactttgattcacaagtaaaacagtttatctatgcgtgccaagataagacattaaggcagtctgtactcaagattttccgctaccatcctttactaaaaattcatgatattgctcgggccgtctatcttttgatggccttagaagaaggcgaggatttaggcttaagctttttaaatgtacagcagtacccttcaggtgctgtagaactgttttcttgtgggggatttccttggaaaggattaccttatcctgcagaacatgcggaatttggcctactcctgttacagatcgcagagttttatgaagagagtcaggcatacgtctctaaaatgagtcattttcaacaggcactctttgatcaccaagggagcgtctttccctctctctggagccaggagaactctcgactcctaaaagaaaagacaactcttagccaatcgtttctcttccaattaggaatgcaaattcacccagaatacagtcttgaggatcctgcactagggttctggatgcaaagaacgcgttcttcatccgcttttgtagccgcttcaggatgtcaaagtagcttgggagcgtattcctcaggggatgtcggtgttatcgcttatggaccttgctctggagacattagtgattgttattattttggatgttgtggaatcgctaaagagttcgtgtgccaaaaatctcaccaaactacagagatttcttttctcacctctacaggaaagcctcatcccagaaatacgggattttcctaccttcgagattcctatgtacatctgccgatccgctgtaagatcactatttccgacaagcaatatcgcgtgcacgctgcgttggctgaggccacctctgccatgacgttttctattttctgtaaggggaagaattgtcaggttgttgacggccctcgcttgcgctcctgttccctagattcttataaaggtcccggaaacgacattatgattcttggggaaaatgacgcaatcaacattgtttctgcaagtccctatatggaaatttttgctttgcaaggcaaagaaaaattttggaatgcagactttttgattaatattccttacaaagaagagggcgtcatgttaatttttgaaaaaaaagtgacctctgagaaaggaagattctttacgaagatgaattaa

SEQ ID NO: 14 - DNA, 1110 bp, Artificial Sequence
  Description of Artificial Sequence  Synthetic Primer
atgacaaagatagctttttctgaaaaggcaaagaattttcctgtagaggcattaaaaaaatggtttgaaaaaaataaacgatctcttccttggagagataacccgactccctatagtgtgtgggtttccgaagttatgctacagcaaacgcgagctgaagttgttatagattattttaatcagtggatggagagatttcctaccatagagtctttagctgcagcaaaagaagaagatgtcattaagttatgggagggattgggttattattctcgagcgcgccatcttttagagggagctcgcatggttatggaggagtttcatggaaagatccctgatgatgccatttccttagctcaaattcgtggagttggtccttatacggttcatgctattctagcctttgcttttaagaggcgtgctgctgctgtggatggcaatgtcttgcgtgttcttagccggatatttttgatagaaacttctatagacttagaatcaactcgtacttgggtttctaggattgctcaagcgcttcttcctcataagagtcccgaggttatagctgaggctctgatagagttgggagcttgtatctgtaaaaaagttcctcaatgtcatcgttgtcctgtccgtcaagcatgtggagcttggagggagaacaaacagttcgtattgccggtacgtcatgccagaaaaaaggtcatctttttgcatcgtttggtagcgattgtattgtacgatggctctttggttgtcgagaagagacgtcctaaagaaatgatggcaggcttatatgaatttccttatattgaagttgaaccagaggaaggtcttcaagatatagaaggatttactaagaagatggagctttctttagaaagccctttggaattcttaggtaaccttaaagaacagcggcatgcgtttactaatcataaggttcatttgtgtcctataatttttaaagccacttctctgcctcagttcggggaattgcatcttttgagtgatatagatcacttagctttttcttcaggacacaaaaagattaaagatgctttgctaatctacctcggggatgtcaggtctagagaatcaataggagtatag

SEQ ID NO: 15 - DNA, 1740 bp, Artificial Sequence
  Description of Artificial Sequence  Synthetic Primer
atggcagtttcaggtggcggaggggttcagccttcttcggatccaggaaagtggaatcctgctctgcaaggagagcaggcagaaggcccgtctccgctaaaagaatctatattttctgaaaccaagcaggcctcctctgctgcgaagcaggaaagcttagtgcgttcaggatctacaggaatgtatgcaacagaatctcagataaataaggctaagtatcgtaaagctcaagatcgatcatcaacctctccaaaatccaaattgaaaggtacattttctaaaatgcgcgctagtgtgcaaggattcatgtcaggattcggatctcgggcttcgagagtgtcagcaaagcgtgcttccgatagtggtgagggaacatccttattgccgacagagatggatgttgctctaaagaagggaaaccgtatttcacctgaaatgcagggatttttcttagatgcttcgggtatgggagggagttcctctgatatttctcagctttctttagaggctttgaaatcttcagcattttcaggtgccaggagtttaagtttaagctcttcagaatctagttccgtggcttcgtttggatctttccaaaaggccatagagcctatgagtgaggagaaggtaaatgcttggacagtggctcgtttaggaggggagatggtcagctctcttctcgatcccaatgttgagacctcatcattagtgcgcagggcaatggcaacaggcaacgaaggcatgatagatctttctgatttaggacaggaagaggtcagtacagccatgacatctcccagagcagtagaaggaaaagtaaaggtatcttcttctgattctccagaagcgaatccaacaggaattccaaattctaatactttagaaagggcggaaaaggaagcagagaaacaagaaagtcgagagcagttgagtgaggatcagatgatgcttgcacgtgctatggctgggcttcttacaggggcagcgcctcaagaggtattgagtaattctgtttggtctggtccttctacagtatttcctcctcccaagttttcaggaactttacccacccagagatcgggagataaatcaaagcataaatctccaggaatagagaagagtacgaaccatacgaacttttctcctcttcgggaaggtactgtgaagagtgctgaggttaaaagtttgcctcatccagaaagtatgtatcgttttcctaaagatagcatcgtttccagggaggaacctgaagccgttgttaaagaatctacggcattcaaaaatccagagaatagcagtcaaaactttctccctattgctgtggagagtgttttccctaaggaaagtggtacgggaggggctttaggaagtgatgctgtgagttcctcatatcatttccttgcgcaacgtggagtgtctttactcgctcctctacctcgtgctactgatgactataaagagaagctcgaagctcataaaggtcctggaggtcctccagatcctttgatttatcagtatcgaaatgttgctgttgagccgccaattgttctccgttctccccagccgttttcaggatcttcacgtctatcggttcaaggaaagcctgaagctgcttcagttcatgacgatggtggggggggaaatagtggtggttttagcggagatcaaagaagaggatcttcgggccagaaagcttcccgtcaggaaaagaagggaaaaaaattatctacggatatttag

SEQ ID NO: 16 - DNA, 3429 bp, Artificial Sequence
  Description of Artificial Sequence  Synthetic Primer
atgaaacgaagatcatggcttaaaattttgggaatctgtttaggcagcagcattgtcttgggattccttatattcttgccccaactactttcaacagaatcagggaaataccttgtgttttccctgattcataaagaatccggactctcgtgttctgctgaagaacttaagatttcatggtttggacggcaaacagctagaaaaataaaactcactggagaagctaaagatgaggtcttttctgctgagaaattcgaactcgacggatctctattacgtcttctgatttataaaaagcctaaagggattactctatcaggatggtctttaaaaattaatgagcctgcctctatagaccatccttctgtgagtcacttagatccaggatctttacttacctacctaaatgactgcaagattatttctgagcacggatttatcactatgaagacagtatcaggatcttcattatctgtatcagggttttatctagagaaatcctcagaaaagttcatgacgaaatgcgtggtctctgaagatcagcaatccgggaacatctttatagagagtgtactttctcctgatgtcagtatttccgctcagttttcttcagttcccgttgcattttttaaaatttttatagcttcccctttctgggaccatcttctctcttatgaagacataatcaatctatcagcagaggcaacacataccaatgatggtaagatttctatgacagcctctggcgagggaaatcaaattcaaatgaagcttcaaggccatattcataaatccacattttatattgtagaagggagttcttcgttcatagaacttaaacctgagctcgcctcagctctttgcaaccagatcattccgctgtccacacccattactagtaagcaaatccatgctacggtctcttatgctaaaattcccttggatattacgaaatggaaacatattgaaattacctctcaagcacagctccctgaagtcgcaatacatccgaaagaccctaatcttgcattacagctgcgcgacacaaagctaggaattaaaaagacggagaaattctcagacatccgttactcctcatctacagtcttaggaggagcttctccctctcaccttaatggtttaatcagtatagataacaaaaaacatcttactaaatttcgtctacaacaagcacaactcccccacacctatctaagagccattttccctcaacctttcgtgatcaatgttcccctggatgttgcttattattcattaaatatcgaagggacgtacaaaaatgctcacttagaggcagatgctatcctagataacccgctattgaaattgtcatgctccatgtctggagcatggaaaaattttctttttaaagggcaaggaacgtaccactttaataaaaaatggcaggagattctctctccccacttctcttacgctgaagctagattctcaggaaaagcacaaattaccgatacgaatctctttttccctaaattttctggaaaaattactgcaagagaaaatgagctgctcatccatgcaaaatttggttcccctaatgaacctataaaacctgaaactacctctatactcatccacggacaattttgttctctgccactcagcctagtttctaatcacctagcccccttccatttgaagaaattgacattttccttccatacggatggaggtaagtttgtaaccaaaggaaacctccaagctcttattgagaatccagactatcccgacctaaataatacgcgtatcctaatccctgatcttcttctttctcttgatgaatcctcaacttcaccttcttcaaaagacttgaaaatccagggttctggagagatattttctttgcctctggattctattactaagacctatgggaaacaagtgcgtctctctccttattttggttcctctggagacttgaactttgtagtaaactacaatcctaaagatcagaataagctcacactactatctaactttaagtcagaagctctcctaggagaactgaagttagtcatggacttttctatgaagctatcttcaggaactcagggaactctccagtgggaagtgagcccagaacgttatgcaagtttctttaaaaacgcatcatgctctcccacctgtttgcttcatagaactgcaaatgtacgcttagacatctcaaaactctcttgtccagaggaaaccaaaggtttatcttgtctcacgcttcttgccgcaggaggacttgaaggttcattagaagcaacaccgttgatcttctatgataatgtgtctaaagagacttttattattaatgactttaaaggttctttgcgagccaacaatttagacgctaaaatagaatatgatcttaaaggctcgtgtctagctcctaggcaagactctaaaactcttgcagaattttcattagaaggacaggtagatcatctgttctctccagagtctcgagaatttaaacaaactgcaaattggattcacataccctcttcgttcattgctggaatcattcccatgtctccaggattgaaagctcagatatcctcgcttgcaggccctagaatcaacgtatcaattaaaaatgcgttccgatttggagaaggccctgtcgacattatggtcgactctgaaaaccttcaagctcagattccactgatcttaaacgaaaagtccatcttactgagagagaatctaacagcgcaccttagtataaatgaagatgtaaataaggctttcctacaagagtttaatcccctcttagcagggggagcctactcacaatacccagtaaccttagagatcgataaacaaaacttctatctccctatacgcccgtattcttttgaagaattccgcatccaatccgcaacattggatatggggaaaatctcaatagcaaatacaggaactatgtatgctcttttccaattccttgatattacggatcaaaagcaatttgtagaatcttggttcactccaattttcttttctgtacaaaaaggctctatcatttgtaagcgcytcgacgcccttatcgatcgtagaatccgccttgctctatgggggaaaactgatatcgctcatgatcgtctgtttatgaccttgggtatcgatcctgaagttattaagaaatactttcataacacctctttaaaaactaaaaacttcttccttataaaaatccgaggatccatctcgtctcctgaagtggactggtcttcagcttacgctagaatcgctctattaaaaagctacagtcttgggaacccgtttagtagtcttgccgataagctattctcttctcttggcgactctacccccccaccaacagtacaccccttcccttgggaaaaatctaattttgattctatagaaaataaatag

SEQ ID NO: 17 - DNA, 1173 bp, Artificial Sequence
  Description of Artificial Sequence  Synthetic Primer
atgtcatctcctgtaaataacacaccctcagcaccaaacattccaataccagcgcccacgactccaggtattcctacaacaaaacctcgttctagtttcattgaaaaggttatcattgtagctaagtacatactatttgcaattgcagccacatcaggagcactcggaacaattctaggtctatctggagcgctaaccccaggaataggtattgcccttcttgttatcttctttgtttctatggtgcttttaggtttaatccttaaagattctataagtggaggagaagaacgcaggctcagagaagaggtctctcgatttacaagtgagaatcaacggttgacagtcataaccacaacacttgagactgaagtaaaggatttaaaagcagctaaagatcaacttacacttgaaatcgaagcatttagaaatgaaaacggtaatttaaaaacaactgctgaggacttagaagagcaggtttctaaacttagcgaacaattagaagcactagagcgaattaatcaacttatccaagcaaacgctggagatgctcaagaaatttcgtctgaactaaagaaattaataagcggttgggattccaaagttgttgaacagataaatacttctattcaagcattgaaagtgttattgggtcaagagtgggtgcaagaggctcaaacacacgttaaagcaatgcaagagcaaattcaagcattgcaagctgaaattctaggaatgcacaatcaatctacagcattgcaaaagtcagttgagaatctattagtacaagatcaagctctaacaagagtagtaggtgagttgttagagtctgagaacaagctaagccaagcttgttctgcgctacgtcaagaaatagaaaagttggcccaacatgaaacatctttgcaacaacgtattgatgcgatgctagcccaagagcaaaatttggcagagcaggtcacagcccttgaaaaaatgaaacaagaagctcagaaggctgagtccgagttcattgcttgtgtacgtgatcgaactttcggacgtcgtgaaacacctccaccaacaacacctgtagttgaaggtgatgaaagtcaagaagaagacgaaggaggtactcccccagtatcacaaccatcttcacccgtagatagagcaacaggagatggtcagtaa

SEQ ID NO: 18 - DNA, 2463 bp, Artificial Sequence
  Description of Artificial Sequence  Synthetic Primer
atgcgatatgaccccaacttaatagaaaaaaaatggcaacaattttggaaagaacatcgaagctttcaagcaaatgaagacgaggataaagtaaaatattatgttttagacatgttcccttatccttcaggagcaggtctacatgtaggccaccttattggctatacagcgacagatattgttgcgagatataaaagagcacggggattctcagttcttcatcctatgggctgggatagctttggtttgcccgcagaacaatatgcgattcggacaggaacccatcctaaagtcacgacccagaagaatatcgctaattttaaaaaacagctctccgctatgggattttcgtatgatgaaggacgagaatttgctacgagtgatcccgactattatcattggactcagaaacttttcctttttctttatgatcaaggactcgcctatatggccgacatggcagtgaactactgtccagaacttggtaccgtattatcgaatgaagaagttgaaaatggattctcaatagaagggggatatcctgtagagcggaaaatgcttcgtcagtggattctcaaaatcacagcatatgccgataagttattagaaggtctcgatgccctagattggcccgaaaatgtaaagcagttacagaaaaattggatagggaaatctgaaggggctctcgtaacatttcatttgacgcaagagggcagtctagaagccttcactacccgcctagacactttattaggggtgagtttcttagtgattgctcctgagcacccagatttagattctatagtgagtgaagagcaaagagacgaagtcacagcctatgtacaagagagtctcaggaaaagtgaacgagatcgcattagctctgttaagacaaaaacaggggtctttacaggaaactatgccaagcaccccattacagggaaccttttacctgtttggatttcagattatgtcgtcttaggctatggcacaggcgtagttatgggagtcccagcgcatgacgagagagatcgagagtttgctgaaatgttttctcttccgattcatgaggtgattgatgataacggggtttgtattcatagcaattacaacgacttttgtcttaatggcttgtctgggcaagaagctaaagattatgtaatcaactacctggagatgcgttctctcggaagagctaagactatgtacaggctgcgagactggctcttctctagacagagatattggggagagcctatccccatcattcattttgaagatggaacgcaccgtcctttagaagatgatgagctgcctcttctccctccgaatattgatgactatcgtcccgaaggattcggtcagggtcctttagcgaaggctcaagattgggtgcatatctacgacgagaagacaggtagaccaggatgtagagagacttatactatgccacagtgggcaggctcttgctggtattatcttcgtttctgtgatgcacacaactctcagttgccttggagtaaagaaaaagaaagctattggatgcctgtagatctttacattggaggtgcagaacacgctgttcttcatcttctttactcgagattttggcatcgagtcttctatgacgcgggtcttgtctcaacaccagaaccttttaagaaactgatcaaccagggacttgtgttagcctcttcataccgaattcctggtaagggatacgtaagcatagaagacgttagggaagaaaatggaacgtggatctcaacttgtggagagattgtggaagttagacaagagaaaatgtctaaatcgaaactcaatggtgtggatcctcaggttttgattgaagagtatggtgcagatgccttacgtatgtacgctatgttttcgggacccttggataaaaataaaacctggtccaatgaaggtgttggggggtgccgtcgtttcctaaatcgtttttatgatttggtgacttcgtcagaggttcaagatatagaagaccgtgacgggctggttctcgctcacaaattggtgtttaggattacagaacatattgaaaaaatgtctttgaataccataccgtcttcatttatggaatttctgaacgatttttcaaagcttccagtctattctaaacgtgccttgtctatggctgttcgtgtattggagcctatagctccgcatatcagcgaagagttatgggttatattgggaaacccaccagggattgatcaagcagcatggcctcaaatagacgagagttacctagttgctcaaactgtgacttttgttgttcaggttaatgggaagttacgaggacgtctcgaggtagccaaagaagctcctaaagaagaagttttatctttgtctcgaagtgtagttgcaaagtatctagagaacgctcaaatacgaaaagaaatttatgttcctaataaactagtgaattttgtcctatga

SEQ ID NO: 19 - DNA, 4194 bp, Artificial Sequence
  Description of Artificial Sequence  Synthetic Primer
ttggagaaaataatgttcggagaaaattctcgagacattggagttctttctaaagaaggactatttgataaattagagataggcatagcttcagatattacaattcgtgataaatggtcttgtggagaaatcaaaaagccagaaactataaattaccgtacgtttaaacctgaaaagggcggtctattttgtgaaaaaatctttggtcctactaaagattgggaatgttgctgcggaaaatataaaaaaataaaacataaaggaattgtctgcgatcgatgcggagttgaagttactctttcaaaagtccgtcgtgaacgtatggctcatatcgagttagcagttcctattgtccatatttggtttttcaaaacaactccatcacgcattggtaatgttcttggaatgacagcttcggatctggaacgtgtcatttattatgaagaatatgtagttattgacccaggtaagacagacctaactaaaaaacaacttcttaatgatgcgcaatatcgtgaagttgttgagaagtggggtaaggacgctttcgttgctaaaatgggtggcgaagctatctatgatttgcttaaatccgaagatctccaaagcttgcttaaagatcttaaagagcgtttacgcaaaacaaaatctcagcaagcgagaatgaagttagccaaacgtcttaaaatcattgagggatttgtttcttcatccaaccacccggagtggatggtattaaaaaatatcccagtagttccacctgatctccgtcctcttgttcctttagatggcggtcgttttgcgacttctgatttaaacgatctctaccgccgtgtaattaatcgtaacaatcgtcttaaagcgatcttacgtttaaaaacaccagaggttattgttcgtaatgaaaagcgtatgcttcaagaagctgttgatgctctttttgataacggtcgacatggtcatccggtcatgggagctggaaaccgaccattgaaatccttgtcagaaatgttaaagggaaaaaatggacgcttccgtcaaaatcttttaggaaaacgtgttgactactctggacgttctgtaattattgttggtcctgaattgaagtttaatcaatgcggattgcctaaggaaatggctttagagctattcgaaccctttattattaaaagactaaaagatcaaggcagcgtttataccattcgttctgctaagaaaatgattcaacgaggagccccagaagtttgggacgttctcgaagagatcattaagggacatccagtacttcttaaccgagcacctacattgcaccgtttaggaattcaagctttcgaacctgtattgatagaaggtaaagcgattcgtatacaccccctagtttgcgcagcgtttaacgctgacttcgacggagaccaaatggccgtgcacgttcctctatctgtagaggcacaactggaagctaaagttttaatgatggctccagacaacatcttccttccttcctcaggaaagcctgtggctattccttcgaaagatatgactttaggattatattatctgatggcagatcctacctattttcctgaagaacatggaggaaaaactaagatatttaaagatgaaatcgaagtattgcgtgctttaaataacggtggattcattgatgatgttttcggagatcgtcgtgatgaaacaggacgcggtatccatattcatgaaaagattaaagtgcgtattgatggacaaattattgagacaaccccaggaagggtattgttcaacagaattgttcctaaagaactcggcttccaaaattacagcatgccaagtaagcgtataagtgagcttattttacagtgctataagaaagtcggtttagaagctactgtacgtttcttagatgaccttaaagatcttggatttattcaagctacaaaagccgcaatctctatgggattgaaggatgttcgtattcctgatatcaagagtcatatcctcaaagatgcctacgataaggttgctatcgtcaaaaaacaatatgatgatgggatcattactgaaggggagcgtcattccaaaactattagtatttggactgaagtttccgaacagctttcagatgccctctatgttgaaattagcaaacaaacacgtagcaagcataaccccttgttcctgatgattgattctggagcccgaggtaataaatcccagttgaaacagttgggagcgttacgaggattaatggcgaagccaaacggagcaattattgaatctccaattacttcgaactttagagaaggattgacagttttagagtactccatctcctcacacggtgcgagaaaaggtttagccgatacagctctaaaaactgccgactccggatacttaacacgtagacttgtagacgtagcccaagacgtgatcattaccgaaaaagattgcggtacgttaaatcacattgagatttctgcaataggtcaaggttctgaagaactcttgcctcttaaagatcgtatctatggacgtactgtagctgaagatgtctatcaaccaggtgataaaagtcgactacttgctcaatcgggtgatgtactcaactccgtacaagcagaagcaattgatgatgccggtattgagacaattaagattcgttctacattaacgtgcgaaagtcctcgcggagtttgtgcaaagtgttacggcctcaatttagctaatggtagactcattggcatgggtgaagctgttggtattattgctgctcagtcgattggggaacctggaactcagttaacaatgagaacgttccacctagggggtattgctgctacgtcttcaactcctgagattattacgaatagtgatggtatcttagtctacatggatctccgtgttgttctggggcaagaaggtcacaatcttgtcttgaataagaagggagctttacatgttgtaggtgatgaaggtcgtactctcaatgagtataaaaagctgctttcaaccaagtctatagaaagcctagaggtatttcctgtagaactaggagtgaaaattcttgttgctgacggaactcctgtttctcaaggacaaagaatcgcagaagttgaactacacaatattcctatcatttgcgataagcctggctttattaaatatgaagatttggttgagggaatctctacagagaaagttgtgaacaagaacacaggacttgttgaacttattgtgaaacagcaccgaggggagttacatcctcagattgctatctatgatgatgctgacttgtcagaacttgtcggaacctatgcgattccttcaggagcgattatctctgtagaagaaggacaacgggttgatccaggtatgttgttagctagacttcctcgcggagctatcaaaacaaaagatattactggcggtttgcctcgtgttgctgaattagtagaagctcgtaaacctgaagatgctgctgacatcgccaaaattgatggtgttgttgacttcaaaggaattcaaaagaacaaacgtattcttgttgtctgtgatgaaatgacaggtatggaagaagaacatctgattccattaaccaaacatttgattgtacaacgtggagatagtgtgattaaagggcagcagcttaccgatggtttagttgttcctcatgaaatcctagaaatttgcggagttcgtgaacttcagaagtacctggtaaatgaggtgcaggaagtttaccgtctgcagggcgttgacattaacgataagcatattgaaattattgttcgtcagatgttacaaaaagtacgaattactgacccaggtgatacgactctgctctttggcgaagacgtgaataagaaagagttttatgaagaaaatcgtcgtaccgaagaagacggtggtaagccagctcaagctgttcccgtcttattgggaattacgaaagcttctttgggtacggaatcgtttatatcagcagcttctttccaagacacaactcgagtcttaacagatgcagcttgttgtagcaaaaccgactaccttcttggatttaaggaaaatgtgatcatgggtcatatgattcctggtggtacaggctttgaaacgcataagcgtattaagcagtatctagaaaaagaacaagaagatctcgtttttgattttgttagtgaaacagagtgtgtttgttaa

SEQ ID NO: 20 - DNA, 1716 bp, Artificial Sequence
  Description of Artificial Sequence  Synthetic Primer
atggatacacagtcctctataggtaacgaagaatggcgtattgcaggaacctctgtagtttctgggatggccttaggtaaagtattttttttgggaacatcccccttgcatgttcgtgagctgactctacctcaagaagaagtcgaacatgaaatacatcgttattataaagctttgaatcgctcgaagtctgatatcgtagctttagaacaggaagttacgggacagcaaggccttcaagaggtttcctctatcctacaagcacacttggagattatgaaagaccctctccttacggaggaggtggtcaatactatccgtaaggatcgtaaaaatgcagaatatgtcttttcttcagtcatgggtaaaatagaagagtcgttaacagcagtccgcgggatgccttctgttgtagatcgtgttcaagatatccatgatatctccaatagagttatcggccatctgtgttgccaacataagagttctttaggagaatctgatcagaatttgatcatattctctgaggaattgaccccctcagaagtcgccagtgctaactctgcctatatccgagggtttgtctcattagtgggagcagccacatcacatacagctatcgtctcgcgagcaaagagcattccctatcttgctaatatctccgaggagctttggaacatcgcaaagcgatataatggcaagttagtcttaatcgacggttatcgtggagagctaatctttaatcctaaaccagcgactctacaaagctgctataaaaaagagctttccgtggttgcccatacctctcagagattagtaagaaagtccctacacccgattgtttcttcgcatgcaggcagtgataaggacgtagaagatctattagagaacttccctcaaacctccataggcctctttcgttctgagtttttagctgtaattttaggacgcctacctacactaagagagcaagtagatctttacgagaagctcgcacgttttcctggagattcgccctcagtactgcgcctctttgattttggtgaagacaaaccttgtcctggaataaaaaataagaaagaacgttctatacgatggttgctagactatagtgtgattcttgaggatcagctccaagcaattgctaaagcctctttgcaaggctccataaaggttctcattccaggagtgtctgacgtttctgagattatagaagtcaaaaagaaatgggagaccatccagacgaggttccctaaaggccataaggtttcttgggggactatgatagaatttccttctgcagtttggatgattgaagagatccttcctgaatgtgattttctctctatagggacgaatgaccttgtccaatatactttgggaatttccagggaatccgctcttcctaaacatctaaatgtaactttgcccccagcagtgatccgcatgattcaccatgtacttcaagctgcgaagcaaaatcaggttcctgttagcatttgtggagaggccgcagggcagctcagtctgactcctttatttataggcctaggagttcaagagctctcagtagctatgcctgtaatcaatagacttcgcaatcatatcgccctgctagagttgaactcctgccttgaaattacagaagcccttttacaagctaaaacatgctctgaagttgaagaacttttaaatagaaacaacaaaatcacatcataa

SEQ ID NO: 21 - DNA, 1383 bp, Artificial Sequence
  Description of Artificial Sequence  Synthetic Primer
atgcgacaagaaaaggatagtttaggaatcgtagaagttcctgaggataagttatatggagctcaaactatgcgttctaggaattttttttcttggggacctgagttgatgccttatgaggtaatacgagctctcgtatggattaaaaaatgtgctgctcaggcgaatcaagatttaggatttttggattccaagcattgcgatatgattgttgctgctgccgatgagattttagagggaggttttgaagagcatttccctttaaaagtttggcagacagggagcggcacacaatctaatatgaatgtgaatgaggtgattgcgaatcttgccattcgtcatcacggaggggtgttaggcagtaaggatcctatccatcctaatgatcatgtgaataagtcccaatcgtccaatgatgttttccctacagcaatgcatatcgctgctgtgattagtttaaaaaataagttaattccagctttagatcatatgattcgggtgttagatgctaaagtggaagaatttcgtcatgatgtaaagataggacggacccatcttatggatgcggtgcctatgacgttgggtcaggaattttctggttatagcagtcaattgcgtcactgcttagagagtatagcattttctttagctcatttatatgaacttgcgattggagctactgctgtagggactgggttgaatgttcctgaagggttcgtggaaaagatcatccattatttaaggaaggaaacagatgaaccgtttattccagcttccaattatttttcagcactgtcttgtcacgatgctttagtagatgcccatgggtctttagcaactttagcatgtgctttaactaagatagctacggatttgagctttttaggttcaggacccaggtgtgggttgggtgagttatttttccctgaaaatgaaccaggatcttctatcatgcctggtaaagtcaatcctacgcagtgtgaagctctccaaatggtttgtgctcaagttcttgggaataatcaaacagtgattattggaggaagtcgaggaaattttgagcttaatgtgatgaagcctgtgatcatctataatttcctgcagtctgtggatctcctttctgaggggatgagggctttctctgaattctttgtgaaaggattaaaagtaaataaagctcgtttacaagataatatcaataattctttgatgttggttacagctttagctcctgtattaggttacgacaagtgttcgaaagcagcactgaaagcatttcatgaatctatatctttgaaggaggcgtgtctagctttgggatatctttctgagaaggaatttgatcgtttagtggttcctgagaatatggtgggaaaccattag

SEQ ID NO: 22 - DNA, 717 bp, Artificial Sequence
  Description of Artificial Sequence  Synthetic Primer
atgggactatatgatcgtgactatatacaagattctcgagtgcagggaacttttgcttcaagagtctatgggtggatgacagcagggctaatcgtaacttcatgtgttgccctgggtctttatttttctggattatacagaagtttattttctttttggtgggtgtggtgtttcgctacgctaggcgtgtctttctttatcaactctaaaatccagacactatcggtttctgctgtagggggccttttccttctctactcaacattagaaggaatgttttttggaaccttacttcctgtctacgctgctcaatatggcggaggggtgatctgggccgcttttggatcagcagccttggtatttggcttagcagcagtatacggagcgtttacaaaaagcgatcttactaaaattagtaagattatgacttttgctttgataggacttctgctagtgactctagtctttgctgtggtttcgatgtttgtatctatgcctttaatctacttattgatttgctatctagggctcgtcatctttgttggattgacagctgctgatgcgcaagcaattcgtcggatttcttctactataggggataacaataccttgagttacaaactctctttgatgtttgctcttaagatgtattgcaatgtcatcatggtattttggtatctgctgcagattttctcatcttcaggaaaccgagactaa

SEQ ID NO: 23 - DNA, 4830 bp, Artificial Sequence
  Description of Artificial Sequence  Synthetic Primer
atggtagcgaaaaaaacagtacgatcttataggtcttcattttctcattccgtaatagtagcaatattgtcagcaggcattgcttttgaagcacattccttacacagctcagaactagatttaggtgtattcaataaacagtttgaggaacattctgctcatgttgaagaggctcaaacatctgttttaaagggatcagatcctgtaaatccctctcagaaagaatccgagaaggttttgtacactcaagtgcctcttacccaaggaagctctggagagagtttggatctcgccgatgctaatttcttagagcattttcagcatctttttgaagagactacagtatttggtatcgatcaaaagctggtttggtcagatttagatactaggaatttttcccaacccactcaagaacctgatacaagtaatgctgtaagtgagaaaatctcctcagataccaaagagaatagaaaagacctagagactgaagatccttcaaaaaaaagtggccttaaagaagtttcatcagatctccctaaaagtcctgaaactgcagtagcagctatttctgaagatcttgaaatctcagaaaacatttcagcaagagatcctcttcagggtttagcatttttttataaaaatacatcttctcagtctatctctgaaaaggattcttcatttcaaggaattatcttttctggttcaggagctaattcagggctaggttttgaaaatcttaaggcgccgaaatctggggctgcagtttattctgatcgagatattgtttttgaaaatcttgttaaaggattgagttttatatcttgtgaatctttagaagatggctctgccgcaggtgtaaacattgttgtgacccattgtggtgatgtaactctcactgattgtgccactggtttagaccttgaagctttacgtctggttaaagatttttctcgtggaggagctgttttcactgctcgcaaccatgaagtgcaaaataaccttgcaggtggaattctatccgttgtaggcaataaaggagctattgttgtagagaaaaatagtgctgagaagtccaatggaggagcttttgcttgcggaagttttgtttacagtaacaacgaaaacaccgccttgtggaaagaaaatcaagcattatcaggaggagccatatcctcagcaagtgatattgatattcaagggaactgtagcgctattgaattttcaggaaaccagtctctaattgctcttggagagcatatagggcttacagattttgtaggtggaggagctttagctgctcaagggacgcttaccttaagaaataatgcagtagtgcaatgtgttaaaaacacttctaaaacacatggtggagctattttagcaggtactgttgatctcaacgaaacaattagcgaagttgcctttaagcagaatacagcagctctaactggaggtgctttaagtgcaaatgataaggttataattgcaaataactttggagaaattctttttgagcaaaacgaagtgaggaatcacggaggagccatttattgtggatgtcgatctaatcctaagttagaacaaaaggattctggagagaacatcaatattattggaaactccggagctatcacttttttaaaaaataaggcttctgttttagaagtgatgacacaagctgaagattatgctggtggaggcgctttatgggggcataatgttcttctagattccaatagtgggaatattcaatttataggaaatataggtggaagtaccttctggataggagaatatgtcggtggtggtgcgattctctctactgatagagtgacaatttctaataactctggagatgttgtttttaaaggaaacaaaggccaatgtcttgctcaaaaatatgtagctcctcaagaaacagctcccgtggaatcagatgcttcatctacaaataaagacgagaagagccttaatgcttgtagtcatggagatcattatcctcctaaaactgtagaagaggaagtgccaccttcattgttagaagaacatcctgttgtttcttcgacagatattcgtggtggtggggccattctagctcaacatatctttattacagataatacaggaaatctgagattctctgggaaccttggtggtggtgaagagtcttctactgtcggtgatttagctatcgtaggaggaggtgctttgctttctactaatgaagttaatgtttgcagtaaccaaaatgttgttttttctgataacgtgacttcaaatggttgtgattcagggggagctattttagctaaaaaagtagatatctccgcgaaccactcggttgaatttgtctctaatggttcagggaaattcggtggtgccgtttgcgctttaaacgaatcagtaaacattacggacaatggctcggcagtatcattctctaaaaatagaacacgtcttggcggtgctggagttgcagctcctcaaggctctgtaacgatttgtggaaatcagggaaacatagcatttaaagagaactttgtttttggctctgaaaatcaaagatcaggtggaggagctatcattgctaactcttctgtaaatattcaggataacgcaggagatatcctatttgtaagtaactctacgggatcttatggaggtgctatttttgtaggatctttggttgcttctgaaggcagcaacccacgaacgcttacaattacaggcaacagtggggatatcctatttgctaaaaatagcacgcaaacagccgcttctttatcagaaaaagattcctttggtggaggggccatctatacacaaaacctcaaaattgtaaagaatgcagggaacgtttctttctatggcaacagagctcctagtggtgctggtgtccaaattgcagacggaggaactgtttgtttagaggcttttggaggagatatcttatttgaagggaatatcaattttgatgggagtttcaatgcgattcacttatgcgggaatgactcaaaaatcgtagagctttctgctgttcaagataaaaatattattttccaagatgcaattacttatgaagagaacacaattcgtggcttgccagataaagatgtcagtcctttaagtgccccttcattaatttttaactccaagccacaagatgacagcgctcaacatcatgaagggacgatacggttttctcgaggggtatctaaaattcctcagattgctgctatacaagagggaaccttagctttatcacaaaacgcagagctttggttggcaggacttaaacaggaaacaggaagttctatcgtattgtctgcgggatctattctccgtatttttgattcccaggttgatagcagtgcgcctcttcctacagaaaataaagaggagactcttgtttctgccggagttcaaattaacatgagctctcctacacccaataaagataaagctgtagatactccagtacttgcagatatcataagtattactgtagatttgtcttcatttgttcctgagcaagacggaactcttcctcttcctcctgaaattatcattcctaagggaacaaaattacattctaatgccatagatcttaagattatagatcctaccaatgtgggatatgaaaatcatgctcttctaagttctcataaagatattccattaatttctcttaagacagcggaaggaatgacagggacgcctacagcagatgcttctctatctaatataaaaatagatgtatctttaccttcgatcacaccagcaacgtatggtcacacaggagtttggtctgaaagtaaaatggaagatggaagacttgtagtcggttggcaacctacgggatataagttaaatcctgagaagcaaggggctctagttttgaataatctctggagtcattatacagatcttagagctcttaagcaggagatctttgctcatcatacgatagctcaaagaatggagttagatttctcgacaaatgtctggggatcaggattaggtgttgttgaagattgtcagaacatcggagagtttgatgggttcaaacatcatctcacagggtatgccctaggcttggatacacaactagttgaagacttcttaattggaggatgtttctcacagttctttggtaaaactgaaagccaatcctacaaagctaagaacgatgtgaagagttatatgggagctgcttatgcggggattttagcaggtccttggttaataaaaggagcttttgtttacggtaatataaacaacgatttgactacagattacggtactttaggtatttcaacaggttcatggataggaaaagggtttatcgcaggcacaagcattgattaccgctatattgtaaatcctcgacggtttatatcggcaatcgtatccacagtggttccttttgtagaagccgagtatgtccgtatagatcttccagaaattagcgaacagggtaaagaggttagaacgttccaaaaaactcgttttgagaatgtcgccattccttttggatttgctttagaacatgcttattcgcgtggctcacgtgctgaagtgaacagtgtacagcttgcttacgtctttgatgtatatcgtaagggacctgtctctttgattacactcaaggatgctgcttattcttggaagagttatggggtagatattccttgtaaagcttggaaggctcgcttgagcaataatacggaatggaattcatatttaagtacgtatttagcgtttaattatgaatggagagaagatctgatagcttatgacttcaatggtggtatccgtattattttctag

SEQ ID NO: 24 - DNA, 762 bp, Artificial Sequence
  Description of Artificial Sequence  Synthetic Primer
atgctaatcaagctatggcgagctacttatgaagggatgtatacatttcttgtgggtgcgttgctaaagttgcgctaccgtatgcaagttgaagggtgggacaccttaaacataaatcctaagcaggggtgtttgtttcttgccaatcacgttgcagaagtggaccctattatcttagaatatctattttggagtcgattccatgttcgtcccatggcggtcgagtatttgtttcatagtcgcgttgttcaatggtttttaaattctgtaagatccattcctatccctcaacttgttcccggtaaagagagtaagcgctctttagaacgtatgaacgtatgttatgaagaagcctcacgagctttaaatagaggggaaagcctccttctttatccttcaggaaggttatcgagaacagggaaagaggaaatcgttaatcagtattctgcttatgtgttattacatagagttatggaatgcaacgtggttttagtcagagtttcaggcttatgggggagtgcgttttcgcgctataagcagaactctacacctaagttaggccctgcgtttaaagaagcttttcgagctttactgcgtcgtgggattttttttatgcctaaaaggtttgtaaaaattactctatgtcaagtagatcatctttttttaaagcaatttccaacaaaacaagacctaaatacttttttggcttcttggtttaatcaaggagatgacaatttgcccatagaagttccttacgcataa

SEQ ID NO: 25 - DNA, 1998 bp, Artificial Sequence
  Description of Artificial Sequence  Synthetic Primer
atgatcaataaagaattagatattggtattttaggaaaaattgctggggctattaaacaaattagtattgaatccatccaaaaggcctcttctggtcatccaggacttcccctaggatgcgcagaacttgctgcctatttatatggttatgtcctgcggcaaaatccacgtgatccccattggattaatagggatcggtttgtcttgtcagcaggtcatggatctgytcttctctattcttgtttgcatcttgccggatttgatgtttccttagaggatcttcaggaatttcgccagcttcactcccgaactcccgggcatcccgagtacggcgaaactgtgggagtcgaagcaacgacaggtcctctgggacagggattaggcaatgctgtcggcatggcgctctctatgaagatgttggaatcccgattcaatcgcccaggacatgagatttttaacggaaaaatctattgtttggcaggggatggctgttttatggagggagtcagccacgaagtttgtagctttgcaggctctttaaatttaaataatcttgtggtcatctatgactacaataatgttgttctcgatggatatcttaatgaaattagtgttgaggatacaaaaaaacgttttgaagcctatggctgggatgtatatgaaattgatgggtatgattttacccatattcatgagacattctcgagcatcaaacgggggcaggaacgtcctgtattagtgattgcacatacaattattggtcatggttcgcctaaggaagggacaaataaggctcatggttctcctttaggagtcgaagggactcatgaaacaaaacagttttggcatctccctgaagaaaagttttttgtccctcctgcagtaaagaacttctttgctcataaaatacaagaagatcgaaaagcacaggagcaatggctggatgaagttcgtgtttggtcaaaacagttcccagaattacacgaagaattcgttgcgttgacctctcataagttacctaaaaacttagaatccttggtgcagagtgtagaaatgccagactctatagctggccgggctgcttcaaataaactgatccaagtattagtacagcacattccttatttgattggagggtccgcagatctttcaagctcagatggaacttggattgcgaatgagaaagtcatccatacgtatgacttctctggaaggaacattaaatacggcgttcgtgagtttggtatggccacaatcatgaatggtttagcttatagccaggtatttcgtccttttggtggaacatttttagttttttctgactatatgcgtaatgcaattcgcttggccgcattatctaaattaccagtcatctatcaatttacccatgattctatatttgttggagaagatggacctacgcatcagcctgtggaacaattgatgtctttgcgcgcgatccctggattgtatgtaatacgtcctgccgatgctaatgaagttagaggagcgtggattgcaggattaaagcacacaggtcctacagtcattgtcttgtcgcgacaagcattgcccacactgcctgctgcgcatcggccttttaaagatggtgtaggtcgtggtgcttatattgtcttaaaagagtcaggagaaaaaccagactatactctcttcgctacagggtcagaagtttctttagctctttctgtagctaaagaactcgaacacttggataagcaagtgcgtgtagtttctttcccttgttgggagctttttgaagctcaagatgtggactacaaacagagtattgtaggcggagatcttggaattcgtgtctctatagaagcaggatctgctttggggtggtataagtatatcggatctgaaggtttagctatcgctatggatagattcggatactcaggagcttctgatgatgtatcagaagaatgtggctttactacagagcaaatccttcagaggattctctctcaatag

SEQ ID NO: 26 - DNA, 1206 bp, Artificial Sequence
  Description of Artificial Sequence  Synthetic Primer
atgtctaccatgcaaaattgtccacattttggtgtatgtggaggatgctcgtttcctcagtccaattattctgactccttaaagaaaaaggaagaactccttcatcagttattcgctcctttagttccctcggatatgattgctcctatcatcccatgttctccctctttaagaggaagaaataaaatggaattctccttttttcaaacttatgaaggagaaaaaagtttaggattcatcagctctacaaaaccaaagaaaggaattccagtgactacatgtctgcttatccatgagcagactatggatattttaaaactcactcgcgaatggtgggataagcacccagagcttatggcgtacttcccccctaaaaacaaaggctcgttatgcacactcaccgtccgtacggggagcccgcagcaaaactttatggtgatcctaacaacatcaggaactccagaatatagggtaaacgaagcctgtatagatgaatggaaagagattctcctatcgtcttctctaaacatagcatcgatctattgggaagaaaaggtagctgcacgtgggatttctacatattatgaaactaaactgctatacggagccccctcgatacagcaaaaactgtccttacctagtgacggtaactctgcctcttttagtttgcgtcccagaagtttcttccaacctcagattactcaggcagcgaaaattatagaaactgcgaaagagtttataaaccccgagggttcggaaacgcttcttgatctctattgtggagcaggaactatagggattatgctctctccctatgtcaaaaatgtgattggcgttgagattattcctgatgctgtagcttcggctcaggagaacatcaaagcgaataacaaagaagattgcgtagaagtctatttagaagatgcgaaagcgttctgcaaaaggaatgaaaattgtaaagctcctgatgtcattattattgatcccccacgttgtggtatgcaaagtaaagtacttaaatatattttacgtataggatctccaaaaattgtctatatctcttgcaaccctaaaacacagtttcaagagtgcgcggacctaatctctgggggatatcgcataaaaaagatgcagcctattgaccaatttccctattcgacgcacctagaaaatattattttactagaaagagagatcgatctctag

SEQ ID NO: 27 - DNA, 1335 bp, Artificial Sequence
  Description of Artificial Sequence  Synthetic Primer
atgacatcaggagttagtggaagttcaagtcaggatcccacattggctgcgcaattggcacaatcctctcaaaaagcaggaaatgctcagagcggtcacgatactaagaatgtaacaaagcaaggggctcaagcagaagtcgctgctggaggctttgaggatctcattcaagacgcttcagcgcaaagtacaggaaaaaaagaagctacatcttcaactactaaaagtagtaagggagaaaaaagcgaaaaatctgggaaatctaaatcctcaacttcggtagcaagcgcaagtgaaactgcaacggcacaagccgtacaaggtcctaagggattaagacaaaataattatgactcacccagccttcctactcctgaggcacagaccatcaatggtattgtacttaaaaaaggcatgggtacgttagcactcctcggtttagttatgaccttgatggccaatgctgccggagagtcttggaaagcttcattccaatcacaaaaccaagcgatccggtcacaagtcgaatccgctcctgctattggagaagctattaaaaggcaagcgaaccatcaagcatcagctacagaagcacaggcaaaacagtccttgattagtggtattgtaaacattgtaggatttacagtttctgtaggagcgggtattttctctgctgcaaaaggagctacctcggctttaaaatctgcttcctttgccaaagaaacaggtgcctcagctgctggaggtgctgcttccaaagctttaacttctgcaagttcttctgttcaacagactatggcttcgactgccaaggcggcaacaacagcagcgagttctgcgggatcagcagccaccaaagctgcagcgaacctaaccgatgatatggccgcagcagcttcaaaaatggcctctgatggagcttcgaaagcttcaggaggactctttggcgaagtcttaaataaacctaactggtctgaaaaagtctccagaggtatgaacgtagtgaaaactcagggagcgcgtgttgcatcatttgcaggaaatgctctttcttcctctatgcaaatgagtcaattaatgcatggactcacagcggctgttgaaggtctttctgcggggcagacaggaatagaagtcgcccatcatcagcggttagcaggacaagcagaggctcaagcagaagtcttgaaacaaatgtcttcggtttatggtcagcaagcaggacaagcaggacaattacaagagcaagctatgcaatcatttaatactgccttacaaacactacaaaatattgcggactctcaaacacaaacaacatctgctatctttaattaa

SEQ ID NO: 28 - DNA, 2025 bp, Artificial Sequence
  Description of Artificial Sequence  Synthetic Primer
atgagtatagtcagaaattctgcattgccacttccgtgtttaagcagatccgaaacctttaaaaaagttaggtcgcatatgaaatttatgaaagtccttactccatggatttatcgaaaagatctttgggtaacagcattcttactgacagcaattccaggatcttttgcacatactcttgttgatatagcaggagaacctcggcatgctgctcaagcaacaggagtttctggagatggtaaaattgttataggaatgaaagttccggatgatccttttgctataactgtaggatttcaatatattgatgggcatttgcaacccttagaggcagtacgtcctcaatgctctgtataccctaatggtataaccccggacggaacggttattgtgggtacaaactatgccatcgggatgggtagtgttgctgtgaaatgggtaaatggcaaggtttctgaacttcccatgctccctgacaccctcgattctgtagcatcggcagtttctgcagatggaagagtgattggagggaatagaaatataaatcttggcgcttctgttgctgtgaaatgggaggacgacgtgattacacaacttccttctcttcctgatgctatgaatgcttgtgttaacggaatttcttcagatggttctataattgtaggaaccatggtagacgtgtcatggagaaataccgcagtacaatggatcggggatcagctctctgttattgggactttaggaggaactacttctgttgctagtgcaatctcaacagatggcactgtgattgtaggaggttctgaaaatgcagattctcagactcatgcctatgcttataaaaacggtgttatgagcgatatagggaccctcggaggtttttattctttagcacatgcagtatcttcagatggttctgtgattgtaggagtatccacgaactctgagcatagatatcatgcattccaatatgctgatggacagatggtagatttaggaactttaggagggcctgaatcttatgctcaaggtgtgtctggagatggaaaggtaattgtgggtagagcacaagtaccatctggagattggcatgcgttcctatgtcctttccaagctccgagccctgctcctgtccatgggggaagcactgtcgtaactagccagaatccacgtggaatggtagatatcaatgctacgtactcctctttgaaaaatagccaacaacaactacaaagattgcttatccagcatagtgcaaaagttgaaagtgtatcctcaggagcaccatcttttacaagtgtgaaaggtgcgatctcaaaacagagccctgcagtgcaaaatgatgtacagaaagggacgtttttaagttaccgttcccaagttcatggaaacgtgcagaatcagcaattgctcacaggagcttttatggactggaaactcgcttcagctcctaaatgcggctttaaagtagctctccactatggctctcaagatgctctcgtagaacgtgcagctcttccttacacagaacaaggcttaggaagcagtgtcttgtcaggttttggaggacaagttcaaggacgctatgactttaatttaggagaaactgttgttctgcaaccctttatgggcattcaagttctccacctaagtagagaagggtattctgagaagaatgttcgatttcctgtaagctatgattctgtagcctactcagcagctactagctttatgggtgcgcatgtatttgcctccctaagccctaaaatgagtacagcagcaactttaggtgtggagagagatctgaattcacatatagatgaatttaagggatccgtctctgctatgggaaactttgtcttggaaaattctacagtgagtgttttaagaccttttgcttctcttgctatgtactatgacgtaagacaacagcaactcgtgacgttgtcagtagttatgaatcaacaacccttaacaggcacactaagcttagtaagccaaagtagctataatcttagcttctaa

SEQ ID NO: 29 - DNA, 1830 bp, Artificial Sequence
  Description of Artificial Sequence  Synthetic Primer
atgtttcgttgcatattgtttggtattttcctactcacgtgtttttcttctggcggggtgttatattacttattctgttcccatgatttttctatagggcctaaggaaaaatcacgatccgtgtggattgaggaagaaaaagagttcacggattccgtattacatcatctgccatcgcaacatcagcatttgcatattctttgtttccaagggtttttactacagaagcaacaaaagttttctcaagcagaaaagattttctctaaagtttacgacgaggctcaggacggtccttttctttttaaggaggaaattttaggatcccgactgatcaacagtttttttttagaaaaaacagacgtcatggagaccattctttgtcttctgaatcagcgctgtcccaactccccttactaccacttatttaaggctctagtatgctataagcaaaagctataccgtgaggtcatagagcaactagcctactggcaagaagagaaaactcgagcgcttgctcctttattgaatataagtattgaacagctgctaacagattttctgttagattatatttctgcgcattctctgatagaacagaaaatgttccccgaaggcagagtaattcttaatcgcaatatcaataggttattaaaacacgaatgtgagtggaatgcgaagacatacgatcgtattgcgattcttcttagccggagttattttctagagttggtagaatctaagtctgcagatatttattttgattattatgagatggtgcttttctatctcaaaaagatctatattttagagcagtgtccttatgcagaacttctccccgaggaagagcttgtttccttgattatggaacacgtgtttatccttcctaaagataaattatatcctttaattcagctcctagagatgtggcagaagcattatgttcacccaaatagttctttagtagttcagatattggtagaccgcttttctacacatatggaaggggctattcggttttgtgaggctttagtttctttctctggattggaagaattacatcagcaaattattaccacttttgaagagctgctttcaaataaagtacagcagataaaaactgaagaggctaaacaatgtgttgccctacttcatattttggatccttctatttccattagtgaaaaattagctctttcttcggatacattacaaaatatagtttctggggacgacgagcagcatacaaaactccgcaattacctagatctttgggaagccatacagtcttatgatattgatcgccaacagctcgttcatcacttagtttatggtgcaaaagatctttggaaaaaaggaggaaatgatgaaaaggcattgaaccttcttcagctggtcttgaggtttacaagctacgatatagaatgcgaaagtgttgtgtttctttttataaaacaggcgtataagcaagcactgtcttcccatgccattgctcgtcttttaaagttagaaaaatttatatcggaagcgaatattccctctatagtgattagtgaggctgagaaggccaatttcttagcagatgctgaatatctttttgctcatgaagactatgacaaatgctatttgtatagcatgtggttgactaaggtggccccctcccctcaatcctatcgcttagcagggttatgcctgatggaaaataagcgttacgacgaagctttagaatttctctgtatgctctcacccaataatagtatcaacgactataagacgcagaaggcattagcattttgccaaaaacatcaatctaaggaccgagctgcctcttag

SEQ ID NO: 30 - DNA, 1596 bp, Artificial Sequence
  Description of Artificial Sequence  Synthetic Primer
atgttgggcaaagaagaagagtttacgtgtaaacaaaagcagtgtttgtcacattttgttaccaatctgacgtccgatgtatttgctttaaaaaatcttccagaagtcgttaagggagctttattttctaaatactcccgttcagttttaggtttgcgagcacttttgttaaaagaatttctatctaatgaagaggatggagatgtttgtgacgaagcctatgacttcgaaaccgatgtacagaaagctgcggacttttaccaaagggttcttgataattttggggatgattctgtaggagagcttggcggagcccacctggctatggaaaatgtctctattttggctgctaaagttttagaggatgctcgaattggcggatccccgctagaaaagtccacaagatacgtctatttcgatcaaaaggtacggggggagtatttatattaccgagaccctattttgatgacttcggcctttaaagacatgtttttgggtacttgtgattttttattcgatacctattctgctttaatccctcaagttcgtgcctattttgaaaaactgtatcctaaagattctaaaacacccgcatctgcctatgccacatcattacgagctaaagttttagattgtatacggggacttcttcctgcggcaactttgacaaatctaggatttttcggtaacggtaggttttggcaaaatctgattcacaagttacaaggtcataaccttgcagagttgcgacgtttaggagatgaatccctaacagagcttatgaaagttattccttcatttgtaagtagagccgagcctcatcatcaccatcatcaagctatgatgcaatatcgaagagctttaaaagagcagctcaagggacttgctgaacaagcaacatttagtgaggagatgtcttcttcaccgagtgttcagttggtatacggagaccctgatggcatttataaagtagctgctggatttctttttccttattcaaatcgttctcttacagatctcatagactattgtaaaaaaatgcctcatgaagatcttgtacagattttagagagcagtgtttctgcaagagaaaaccgccggcataagtctcctcgtggtttagaatgcgtagaatttggctttgatatacttgctgatttcggtgcataccgcgatttgcaacgacatcggacgctgactcaagaacgacagttactctctacacatcatggatacaattttcctgtggagcttctagatactcctatggaaaaatcttatcgagaagctatggagagggcgaatgaaacctataatgagattgttcaggagttccctgaggaagctcagtatatggttcccatggcttacaatatacgttggtttttccatgtaaatgctcgggctttgcaatggatttgtgagttacgctcacagcctcaaggtcatcaaaattaccgcactatagctacaggtttagtgcgagaggttgtcaagttcaatcctatgtacgaattatttttcaaatttgtagattattctgacatagatttaggacggttaaatcaggaaatgcgaaaagaaccaacgacctaa

SEQ ID NO: 31 - DNA, 93 bp, Artificial Sequence
  Description of Artificial Sequence  Synthetic Primer
cgcgtgatgaaggcagtggtctctcacaagagcaggacctcctctatacacaggcagtacagctcttattctttattttacagcatcctcaag

SEQ ID NO: 32 - DNA, 99 bp, Artificial Sequence
  Description of Artificial Sequence  Synthetic Primer
gacggagttaattttggaaatcttttccagccgtgcccttactgcagaggcaaatatccaagtccaacttgcacaagcacgttatctccttcctcgtct

SEQ ID NO: 33 - DNA, 90 bp, Artificial Sequence
  Description of Artificial Sequence  Synthetic Primer
ggcttacgacggttttgcaaacgatactccatagtagtatcggaaagtggagagcctttctgcttactcaagaaaaaaaagatctttttg

SEQ ID NO: 34 - DNA, 303 bp, Artificial Sequence
  Description of Artificial Sequence  Synthetic Primer
aacttcccaatctgtgatagaagttccagattcagaggggattgcagagacgaggatctctgcggaagaaatcgatacgaggctttccctgacgacaagacagaaggtcatctttgctcttgcgacactcttgctcttagcaagtattgctgccttcatagtcacgggatttggtggattgacagtcatgcaagttctccttgttgcttctgtaggatcggcggttgcttctgtaacactccctatggtttcctcaggattttcctacgtcgcctaccaactgaaagcaagattgaatatcag

SEQ ID NO: 35 - DNA, 63 bp, Artificial Sequence
  Description of Artificial Sequence  Synthetic Primer
tttcttcctgtgttgccgggattattactaggtcctcctttacctcagatgtcgttcagattg

SEQ ID NO: 36 - DNA, 189 bp, Artificial Sequence
  Description of Artificial Sequence  Synthetic Primer
tttttcataaaatattctctttcaaacggatatgggattcaaaaatatctgcaaacaaggctctcagctatacctgaatggcatttttccggaacgaatactagctcgaaaattaaaaaactgtgcgaagagctatcccagaactgctcttaccatagaagtactggtatcctcggtcttaggagctct

SEQ ID NO: 37 - DNA, 207 bp, Artificial Sequence
  Description of Artificial Sequence  Synthetic Primer
tgcagctactctgtttactcactatggagcctcctacaacttttgatgctgatgaaatcagaaaagaaaaaatcaaaattcttcaacgtatctcaccattttcagaaggttcttcgattgtccgaggacaatatggtccaggaacggttcaaggagtctcggtccttggctatcgtgaagaagagaatgttgacaaagattcccgag

SEQ ID NO: 38 - DNA, 36 bp, Artificial Sequence
  Description of Artificial Sequence  Synthetic Primer
gcacaagcgttcgggagcctactcctcaggatgcta

SEQ ID NO: 39 - DNA, 75 bp, Artificial Sequence
  Description of Artificial Sequence  Synthetic Primer
gagctactcatttcctaccagagaaaaacaagctcagctattggaaaaaagaactttacgaccagctcgcagtgt

SEQ ID NO: 40 - DNA, 96 bp, Artificial Sequence
  Description of Artificial Sequence  Synthetic Primer
gaaggaagagaagatcttccatcagcacttaggaaaggcagccccacaagcggcaacagcaacttcaggagtgcagcctactgcggatcctgttgc

SEQ ID NO: 41 - DNA, 99 bp, Artificial Sequence
  Description of Artificial Sequence  Synthetic Primer
cgccttaggagtacaacaaacacctcccagtcaagtcctcaatgggattgcactcatcctatctatttatgtgatgttccccacgggagtggctatgta

SEQ ID NO: 42 - DNA, 60 bp, Artificial Sequence
  Description of Artificial Sequence  Synthetic Primer
gctccccaagcccggggtgacaccaagatcagaggttatcgaaatcggactcgcgcttgc

SEQ ID NO: 43 - DNA, 339 bp, Artificial Sequence
  Description of Artificial Sequence  Synthetic Primer
ggacgcttactgaaacagtgtatgctatcaagcttgcgcaaatggctggctatactacaattatttctcatcgctcaggagaaactacggacactacgattgcagatcttgctgttgccttcaacgctggtcaaatcaaaacaggctctttatcacgttctgagcgtgttgcaaaatacaatagactcatggaaattgaagaagagcttggatccgaagcaattttcacagattctaatgtattttcttacgaggattctgaggaatagagggattttctctatttctatcttatctccaaatcaagaatacatcgcagatctatgggccttatctttc

SEQ ID NO: 44 - DNA, 60 bp, Artificial Sequence
  Description of Artificial Sequence  Synthetic Primer
agcacaaatccaacagttgctctcgcatcaatatttgatgcaaaaactacaaaatgtcca

SEQ ID NO: 45 - DNA, 165 bp, Artificial Sequence
  Description of Artificial Sequence  Synthetic Primer
accgacatactcgtcaaattcattaagaacatcttcccacccctgtggagaggtaatgtcgtaccccgcagcaaaaatatgacaagtatctacacacacgccaatgggaatttgatttttcaaattctgaacgaggtaacccaattcttcaaagttactcccaat

SEQ ID NO: 46 - DNA, 78 bp, Artificial Sequence
  Description of Artificial Sequence  Synthetic Primer
tcttctcgctgtacacaacgagaaattgctgggaggagaacggtaaacacaccaaagccaaagagatgtatggggagc

SEQ ID NO: 47 - DNA, 384 bp, Artificial Sequence
  Description of Artificial Sequence  Synthetic Primer
attgatgccacccagatcaaccttaatgcctcccaagttgacattcacattaggaaccacagtagctcctactggagtgccaggacctgtatgagacgtcgagtgagcccctccacctggagtttcatttataaaggagaagtctggagctggggtcgttttccagttaatgcctttcaagcccccaacatcatcccaaatcggacctgtttctacttttgctccacgacgccatgcagagggtgtatatccactactagattctttagcattccaaggaccgtagactacagcagttttaaatttcatgctgagcatcagatgattgtaatttcctgtggctcccgagcgctcttccattgtaggttttacgcgtcccgtcca

SEQ ID NO: 48 - DNA, 147 bp, Artificial Sequence
  Description of Artificial Sequence  Synthetic Primer
gcccttttatcacgagcttgtgtggcatcaatcataccagctcgtaagtccgcatcaatcgccatctgtttacctggcatcgcatccaatcggaatcgggcagcaacttcggcaacacgctcggcacccttagttactacgataaac

SEQ ID NO: 49 - DNA, 144 bp, Artificial Sequence
  Description of Artificial Sequence  Synthetic Primer
aataaggtaattacaggatcatggcgagtgggagcttgtgagagcatggaaaccagaaagccacctaaatgcttgaaaaaaaaggtcgatagggagaaagccgtaaacatagagacgataaaggtaaccgcagaaggaaaatcc

SEQ ID NO: 50 - DNA, 348 bp, Artificial Sequence
  Description of Artificial Sequence  Synthetic Primer
ggcatctctgtgatgatgatgctctcacgatgtttatcttcattctcttctacatgcagacgagctcgaactttaatctttcctcgtcctgtagtatacgtggagcgaattccttcagaaccgcagataattcctcctgtagggaaatcagggcctggcatgacttgtaaaatctcatccacagaagcctgtggatttgcaagcagaagaagtgtagcttctataagttcccctaaattgtgtggggggatattcgttgccatcccaacagcaattcctgaagaaccattgcaaagaagattcggaaatttagaaggaaaaactacaggctcgtgttttgtttcatca

SEQ ID NO: 51 - DNA, 444 bp, Artificial Sequence
  Description of Artificial Sequence  Synthetic Primer
agtggcagaattatctccgtcatgttgtccgctccaccgtgtgagctgcatagtgacttgatagatcccgacttgtttgagtttaaccacaggctcaatatctgcatatctgctgaagtcaggggtcgcgttacgactcacacttttagaggtgatagctgcaacatgagtttcaattgtagtgttaggggcaacgtaataaccattcccaggataatacgtatagagattcgcagcctgacatccagtttttctataataactaaatgtaagcgcatctcgagcagactgaggatcactaatattattgcatactggagtttgcgatacgtctgtcttagaatagatattaaaaccgtcagagagtgtagtagcattaagttgagaaccttccgcagacacattacccttcacaataaattcacatggagaagtcgtggtatc

SEQ ID NO: 52 - DNA, 180 bp, Artificial Sequence
  Description of Artificial Sequence  Synthetic Primer
agtgatcgtaactctttttcaatctctgtgagtttttcaaaatacgcagattttatagcacctctgaactggagcttacgcaccagaagggcgttcaacccagtagaagccgccctcattcgattttcacattcttcaagagttaggccagaagttactgttccagaaataaggaactca

SEQ ID NO: 53 - DNA, 189 bp, Artificial Sequence
  Description of Artificial Sequence  Synthetic Primer
atgccttggattttctataaacttttcaatataaacattggggttattaaaaccggcttcggcttctgcacgtgcggcagaaaacgctctatagaattcgtccttttctttaacaatacgaattcctcttcccccacctccagcaacggctttaataacaatagggaaacctattttttcagctatttt

SEQ ID NO: 54 - DNA, 255 bp, Artificial Sequence
  Description of Artificial Sequence  Synthetic Primer
atttccatgtcttccatagaaaccccttcttcaccgacaacttccatacgcttgccgatttctgaacctttctctagaaactgtgctgcttttttcacagcaccaccccagcctgaaaccttctcttctaaaatttgtcctacctggttcaaatattttgaccaagaaatcaaagggtctattgaaggatacctacgtgcgtcagctcgtgcttttgaaagaccacagaacgctccgactacagctaatgtagat

SEQ ID NO: 55 - DNA, 135 bp, Artificial Sequence
  Description of Artificial Sequence  Synthetic Primer
ggagaccttgaacattataaacacatccatggacctttttcaaaatcaagccaccatggcaaacgtcataagcgccacaattatttaatgttcctacagtgtcgatataagactctatgcggtgatcaggaatca

SEQ ID NO: 56 - DNA, 87 bp, Artificial Sequence
  Description of Artificial Sequence  Synthetic Primer
agggtatctttcaatgcatccccgccgatcacaacaagtcgtagggactccaaacaggaattttgtttttttgctgttttcagaata

SEQ ID NO: 57 - DNA, 258 bp, Artificial Sequence
  Description of Artificial Sequence  Synthetic Primer
tcaataaacccactagcaccacagcgttcattaggccctgccatacaataccagctagaagaatggccgatacaggacacttcgatttcacgagaccctaagcgactttcctccacaaacacatccgtgtcatatagaaatgcttctgagatcttttcttgtaattcctctttatcacggactaaaaatatcccaatactagatcccaaatgtgcagtttttacaatcatagggaaagaaaatgtctctataagattc

SEQ ID NO: 58 - DNA, 135 bp, Artificial Sequence
  Description of Artificial Sequence  Synthetic Primer
ccactaggtcccgcaccaataacaacacaatcaaattcttgggtcatattctcactcactgtaatcaagatttcaaaaagaaccccccttcataaatgcatgcatctcattaagaagaggaccctgtgcttattt

SEQ ID NO: 59 - DNA, 207 bp, Artificial Sequence
  Description of Artificial Sequence  Synthetic Primer
ggagaacaaccattcctgtttcatcccacttcacagcaaagtttgtctctgtatcatcactatataggcaccctacaatcgtctttccatcactggaaacaccctctgcaaaagattgcattccctcagaaaatattccaagatcaacaagtgcaccgtttacccacttcacagcgcggcaagacggatcttgatccgagattccta

SEQ ID NO: 60 - DNA, 285 bp, Artificial Sequence
  Description of Artificial Sequence  Synthetic Primer
gtgacatctaagtacgcttccatgcgtccatcaattcctaatccagcttcacatacacgtaaaatagcacgcgctcctacatctcggctgacaagattcccatacgcaggatacatatcttctaagaaataccaaggagctcctgtctctccacaaggacgttccgaaccatctggaaatactatgcgctttgaagaatccccaggcacccacacacgaccgccctcaccacgcacagactctgaaattaatcgtagcttatcccttccaggaattgctgtaggg

SEQ ID NO: 61 - protein, 651 aa, Artificial Sequence
  Description of Artificial Sequence  Synthetic Peptide
MVNPIGPGPIDETERTPPADLSAQGLEASAANKSAEAQRIAGAEAKPKESKTDSVERWSILRSAVNALMSLADKLGIASSNSSSSTSRSADVDSTTATAPTPPPPTFDDYKTQAQTAYDTIFTSTSLADIQAALVSLQDAVTNIKDTAATDEETAIAAEWETKNADAVKVGAQITELAKYASDNQAILDSLGKLTSFDLLQAALLQSVANNNKAAELLKEMQDNPVVPGKTPAIAQSLVDQTDATATQIEKDGNAIRDAYFAGQNASGAVENAKSNNSISNIDSAKAAIATAKTQIAEAQKKFPDSPILQEAEQMVIQAEKDLKNIKPADGSDVPNPGTTVGGSKQQGSSIGSIRVSMLLDDAENETASILMSGFRQMIHMFNTENPDSQAAQQELAAQARAAKAAGDDSAAAALADAQKALEAALGKAGQQQGILNALGQIASAAVVSAGVPPAAASSIGSSVKQLYKTSKSTGSDYKTQISAGYDAYKSINDAYGRARNDATRDVINNVSTPALTRSVPRARTEARGPEKTDQALARVISGNSRTLGDVYSQVSALQSVMQIIQSNPQANNEEIRQKLTSAVTKPPQFGYPYVQLSNDSTQKFIAKLESLFAEGSRTAAEIKALSFETNSLFIQQVLVNIGSLYSGYLQ

SEQ ID NO: 62 - protein, 389 aa, Artificial Sequence
  Description of Artificial Sequence  Synthetic Peptide
MKKLLKSALLSAAFAGSVGSLQALPVGNPSDPSLLIDGTIWEGAAGDPCDPCATWCDAISLRAGFYGDYVFDRILKVDAPKTFSMGAKPTGSAAANYTTAVDRPNPAYNKHLHDAEWFTNAGFIALNIWDRFDVFCTLGASNGYIRGNSTAFNLVGLFGVKGTTVNANELPNVSLSNGVVELYTDTSFSWSVGARGALWECGCATLGAEFQYAQSKPKVEELNVICNVSQFSVNKPKGYKGVAFPLPTDAGVATATGTKSATINYHEWQVGASLSYRLNSLVPYIGVQWSRATFDADNIRIAQPKLPTAVLNLTAWNPSLLGNATALSTTDSFSDFMQIVSCQINKFKSRKACGVTVGATLVDADKWSLTAEARLINERAAHVSGQFRF

SEQ ID NO: 63 - protein, 213 aa, Artificial Sequence
  Description of Artificial Sequence  Synthetic Peptide
MSVNPSGNSKNDLWITGAHDQHPDVKESGVTSANLGSHRVTASGGRQGLLARIKEAVTGFFSRMSFFRSGAPRGSQQPSAPSADTVRSPLPGGDARATEGAGRNLIKKGYQPGMKVTIPQVPGGGAQRSSGSTTLKPTRPAPPPPKTGGTNAKRPATHGKGPAPQPPKTGGTNAKRAATHGKGPAPQPPKGILKQPGQSGTSGKKRVSWSDED

SEQ ID NO: 64 - protein, 382 aa, Artificial Sequence
  Description of Artificial Sequence  Synthetic Peptide
MGINPSGNRSPDDVWVRGAQGDSSSTQGTGATNSNLGAHNVTTSTSQPQVASKAKQLWQTVREFFLGKKSPDSSQGASGPAMQSPSGPTIRPTRPAPPPPTTGGANAKRPATHGKGRAPQPPTAGSSSGSEQPTAMSSEVAKLVSELKDAVHSHAESQKVLKKVSQELQTKWTDWENNRGPDYLLHGYRVIARALQQTYTEQSMLIEGTSSTGPVPQAVTVAKDAVTQTVRGAIKNLENPKPGNDPDGVLMQVVISLGIEGPTLDPGESIQNFLETRVSDFGGDDSDIDYTSDIARLGSALDRVRENHPNEMPRIWIALARELGAAVHSHATSVRIANAGKNHTRDVVRMANESSRLLQGMKVLSVGAWANTMTVLIGDLFE

SEQ ID NO: 65 - protein, 333 aa, Artificial Sequence
  Description of Artificial Sequence  Synthetic Peptide
MKTLWHFVSKAFLSIVGLCCGVVLAFVVIFALIASSLGNGDATFVSLPDAQGEVKDLGKTAPIIAVIEMKDVIASSKNTAKTIQNILEGFEKAPLKDRVKGIVIDMDCPGGEVFEIDRIYSMLRFWKERKGFPIYIYVNGLCASGGYYVSCAATKIYATSSSLIGSIGVRSGPFFNVKEGLNRYGVESDLLTAGKDKAPMNPYTPWTSHDREERQATLDFLYGQFVDIVTQNRPLLTKEKLVHTLGARIFSPEKAKQEGYIDVVGATKEQVLQDIVAVCKIEDNYRVIGSGGDGWWKRVASAAASSPLVTGMIKHDILPLSHDAAYIPPYLAL

SEQ ID NO: 66 - protein, 228 aa, Artificial Sequence
  Description of Artificial Sequence  Synthetic Peptide
MRPHRKHVSSKSLALKQSASTHVEITTKAFRLSMPLKQLILEKSDHLPPMETIRVVLTSHKDKLGTEVHVVASHGKEILQTKVHNANPYTAVINAFKKIRTMANKHSNKRKDRTKHDLGLAAKEERIAIQEEQEDRLSNEWLPVEGLDAWDSLKTLGYVPASAKKKISKKKMSIRMLSQDEAIRQLESAAENFLIFLNEQEHKIQCIYKKHDGNYVLIEPSLKPGFCI

SEQ ID NO: 67 - protein, 755 aa, Artificial Sequence
  Description of Artificial Sequence  Synthetic Peptide
MAAPINQPSTTTQITQTGQTTTTTTVGSLGEHSVTTTGSGAAAQTSQTVTLIADHEMQEIASQDGSAVSFSAEHSFSTLPPETGSVGATAQSAQSAGLFSLSGRTQRRDSEISSSSDGSSISRTSSNASSGETSRAESSPDLGDLDSLSGSERAEGAEGPEGPGGLPESTIPHYDPTDKASILNFLKNPAVQQKMQTKGGHFVYVDEARSSFIFVRNGDWSTAESIKVSNAKTKENITKPADLEMCIAKFCVGYETIHSDWTGRVKPTMEERSGATGNYNHLMLSMKFKTAVVYGPWNAKESSSGYTPSAWRRGAKVETGPIWDDVGGLKGINWKTTPAPDFSFINETPGGGAHSTSHTGPGTPVGATVVPNVNVNLGGIKVDLGGINLGGITTNVTTEEGGGTNITSTKSTSTDDKVSITSTGSQSTIEEDTIQFDDPGQGEDDNAIPGTNTPPPPGPPPNLSSSRLLTISNASLNQVLQNVRQHLNTAYDSNGNSVSDLNQDLGQVVKNSENGVNFPTVILPKTTGDTDPSGQATGGVTEGGGHIRNIIQRNTQSTGQSEGATPTPQPTIAKIVTSLRKANVSSSSVLPQPQVATTITPQARTASTSTTSIGTGTESTSTTSTGTGTGSVSTQSTGVGTPTTTTRSTGTSATTTTSSASTQTPQAPLPSGTRHVATISLVRNAAGRSIVLQQGGRSQSFPIPPSGTGTQNMGAQLWAAASQVASTLGQVVNQAATAGSQPSSRRSSPTSPRRK

SEQ ID NO: 68 - protein, 568 aa, Artificial Sequence
  Description of Artificial Sequence  Synthetic Peptide
MKTSQLFYKTSKNANKSAAVLSNELLEKAGYLFKVSKGVYTYTPLLWRVVSKMMNIIREELNAIGGQELLLPLLHNAELWQHTGRWEAFTSEGLLYTLKDREGKSHCLAPTHEEVICSFVAQWLSSKRQLPLHLYQIATKFRDEIRPRFGLIRSRELLMEDSYTFSDSPEQMNEQYEKLRSAYSKIFDRLGLAYVIVTADGGKIGKGKSEEFQVLCSLGEDTICVSGSYGANIEAAVSIPPQHAYDREFLPVEEVATPGITTIEALANFFSIPLHKILKTLVVKLSYSNEEKFIAIGMRGDRQVNLVKVASKLNADDIALASDEEIERVLGTEKGFIGPLNCPIDFFADETTSPMTNFVCAGNAKDKHYVNVNWDRDLLPPQYGDFLLAEEGDTCPENPGHPYRIYQGIEVAHIFNLGTRYTDSFEVNFQDEHGQTQQCWMGTYGIGVGRTLAACVEQLADDRGIVWPKALAPFSITIAFNGGDTVSQELAETIYHELQSQGYEPLLDDRDERLGFKLKDSDLIGIPYKLILGKSYQSSGIFEIESRSGEKYTVSPEAFPTWCQNHLA

SEQ ID NO: 69 - protein, 775 aa, Artificial Sequence
  Description of Artificial Sequence  Synthetic Peptide
MASGIGGSSGLGKIPPKDNGDRSRSPSPKGELGSHEISLPPQEHGEEGASGSSHIHSSSSFLPEDQESQSSSSAASSPGFFSRVRSGVDRALKSFGNFFSAESTSQARETRQAFVRLSKTITADERRDVDSSSAAATEARVAEDASVSGENPSQGVPETSSGPEPQRLFSLPSVKKQSGLGRLVQTVRDRIVLPSGAPPTDSEPLSLYELNLRLSSLRQELSDIQSNDQLTPEEKAEATVTIQQLIQITEFQCGYMEATQSSVSLAEARFKGVETSDEINSLCSELTDPELQELMSDGDSLQNLLDETADDLEAALSHARLSFSLDDNPTPIDNNPTLISQEEPIYEEIGGAADPQRTRENWSTRLWNQIREALVSLLGMILSILGSILHRLRIARHAAAEAVGRCCTCRGEECTSSEEDSMSVGSPSEIDETERTGSPHDVPRRNGSPREDSPLMNALVGWAHKHGAKTKESSESSTPEISISAPIVRGWSQDSSVSFIVMEDDHIFYDVPRRKDGIYDVPSSPRWSPARELEEDVFGDYEVPITSAEPSKDKNIYMTPRLATPAIYDLPSRPGSSGSSRSPSSDRVRSSSPNRRGVPLPPVPSPAMSEEGSIYEDMSGASGAGESDYEDMSRSPSPRGDLDEPIYANTPEDNPFTQRNIDRILQERSGGASASPVEPIYDEIPWIHGRPPATLPRPENTLTNVSLRVSPGFGPEVRAALLSESVSAVMVEAESIVPPTEPGDGESEYLEPLGGLVATTKILLQKGWPRGESNA

SEQ ID NO: 70 - protein, 938 aa, Artificial Sequence
  Description of Artificial Sequence  Synthetic Peptide
MRFFCFGMLLPFTFVLANEGLQLPLETYITLSPEYQAAPQVGFTHNQNQDLAIVGNHNDFILDYKYYRSNGGALTCKNLLISENIGNVFFEKNVCPNSGGAIYAAQNCTISKNQNYAFTTNLVSDNPTATAGSLLGGALFAINCSITNNLGQGTFVDNLALNKGGALYTETNLSIKDNKGPIIIKQNRALNSDSLGGGIYSGNSLNIEGNSGAIQITSNSSGSGGGIFSTQTLTISSNKKLIEISENSAFANNYGSNFNPGGGGLTTTFCTILNNREGVLFNNNQSQSNGGAIHAKSIIIKENGPVYFLNNTATRGGALLNLSAGSGNGSFILSADNGDIIFNNNTASKHALNPPYRNAIHSTPNMNLQIGARPGYRVLFYDPIEHELPSSFPILFNFETGHTGTVLFSGEHVHQNFTDEMNFFSYLRNTSELRQGVLAVEDGAGLACYKFFQRGGTLLLGQGAVITTAGTIPTPSSTPTTVGSTITLNHIAIDLPSILSFQAQAPKIWIYPTKTGSTYTEDSNPTITISGTLTLRNSNNEDPYDSLDLSHSLEKVPLLYIVDVAAQKINSSQLDLSTLNSGEHYGYQGIWSTYWVETTTITNPTSLLGANTKHKLLYANWSPLGYRPHPERRGEFITNALWQSAYTALAGLHSLSSWDEEKGHAASLQGIGLLVHQKDKNGFKGFRSHMTGYSATTEATSSQSPNFSLGFAQFFSKAKEHESQNSTSSHHYFSGMCIENTLFKEWIRLSVSLAYMFTSEHTHTMYQGLLEGNSQGSFHNHTLAGALSCVFLPQPHGESLQIYPFITALAIRGNLAAFQESGDHAREFSLHRPLTDVSLPVGIRASWKNHHRVPLVWLTEISYRSTLYRQDPELHSKLLISQGTWTTQATPVTYNALGIKVKNTMQVFPKVTLSLDYSADISSSTLSHYLNVASRMRF

SEQ ID NO: 71 - protein, 928 aa, Artificial Sequence
  Description of Artificial Sequence  Synthetic Peptide
MKSSLHWFLISSSLALPLSLNFSAFAAVVEINLGPTNSFSGPGTYTPPAQTTNADGTIYNLTGDVSITNAGSPTALTASCFKETTGNLSFQGHGYQFLLQNIDAGANCTFTNTAANKLLSFSGFSYLSLIQTTNATTGTGAIKSTGACSIQSNYSCYFGQNFSNDNGGALQGSSISLSLNPNLTFAKNKATQKGGALYSTGGITINNTLNSASFSENTAANNGGAIYTEASSFISSNKAISFINNSVTATSATGGAIYCSSTSAPKPVLTLSDNGELNFIGNTAITSGGAIYTDNLVLSSGGPTLFKNNSAIDTAAPLGGAIAIADSGSLSLSALGGDITFEGNTVVKGASSSQTTTRNSINIGNTNAKIVQLRASQGNTIYFYDPITTSITAALSDALNLNGPDLAGNPAYQGTIVFSGEKLSEAEAAEADNLKSTIQQPLTLAGGQLSLKSGVTLVAKSFSQSPGSTLLMDAGTTLETADGITINNLVLNVDSLKETKKATLKATQASQTVTLSGSLSLVDPSGNVYEDVSWNNPQVFSCLTLTADDPANIHITDLAADPLEKNPIHWGYQGNWALSWQEDTATKSKAATLTWTKTGYNPNPERRGTLVANTLWGSFVDVRSIQQLVATKVRQSQETRGIWCEGISNFFHKDSTKINKGFRHISAGYVVGATTTLASDNLITAAFCQLFGKDRDHFINKNRASAYAASLHLQHLATLSSPSLLRYLPGSESEQPVLFDAQISYIYSKNTMKTYYTQAPKGESSWYNDGCALELASSLPHTALSHEGLFHAYFPFIKVEASYIHQDSFKERNTTLVRSFDSGDLINVSVPIGITFERFSRNERASYEATVIYVADVYRKNPDCTTALLINNTSWKTTGTNLSRQAGIGRAGIFYAFSPNLEVTSNLSMEIRGSSRSYNADLGGKFQF

SEQ ID NO: 72 - protein, 845 aa, Artificial Sequence
  Description of Artificial Sequence  Synthetic Peptide
MFEKFTNRAKQVIKLAKKEAQRLNHNYLGTEHILLGLLKLGQGVAVNVLRNLGIDFDTARQEVERLIGYGPEIQVYGDPALTGRVKKSFESANEEASLLEHNYVGTEHLLLGILHQSDSVALQVLENLHIDPREVRKEILKELETFNLQLPPSSSSSSSSSRSNPSSSKSPLGHSLGSDKNEKLSALKAYGYDLTEMVRESKLDPVIGRSSEVERLILILCRRRKNNPVLIGEAGVGKTAIVEGLAQKIILNEVPDALRKKRLITLDLALMIAGTKYRGQFEERIKAVMDEVRKHGNILLFIDELHTIVGAGAAEGAIDASNILKPALARGEIQCIGATTIDEYRKHIEKDAALERRFQKIVVHPPSVDETIEILRGLKKKYEEHHNVFITEEALKAAATLSDQYVHGRFLPDKAIDLLDEAGARVRVNTMGQPTDLMKLEAEIENTKLAKEQAIGTQEYEKAAGLRDEEKKLRERLQSMKQEWENHKEEHQVPVDEEAVAQVVSLQTGIPSARLTEAESEKLLKLEDTLRRKVIGQNDAVTSICRAIRRSRTGIKDPNRPTGSFLFLGPTGVGKSLLAQQIAIEMFGGEDALIQVDMSEYMEKFAATKMMGSPPGYVGHEEGGHLTEQVRRRPYCVVLFDEIEKAHPDIMDLMLQILEQGRLTDSFGRKVDFRHAIIIMTSNLGADLIRKSGEIGFGLKSHMDYKVIQEKIEHAMKKHLKPEFINRLDESVIFRPLEKESLSEIIHLEINKLDSRLKNYQMALNIPDSVISFLVTKGHSPEMGARPLRRVIEQYLEDPLAELLLKESCRQEARKLRATLVENRVAFEREEEEQEAALPSPHLES

SEQ ID NO: 73 - protein, 404 aa, Artificial Sequence
  Description of Artificial Sequence  Synthetic Peptide
MGLQSRLQHCIEVSQNSNFDSQVKQFIYACQDKTLRQSVLKIFRYHPLLKIHDIARAVYLLMALEEGEDLGLSFLNVQQYPSGAVELFSCGGFPWKGLPYPAEHAEFGLLLLQIAEFYEESQAYVSKMSHFQQALFDHQGSVFPSLWSQENSRLLKEKTTLSQSFLFQLGMQIHPEYSLEDPALGFWMQRTRSSSAFVAASGCQSSLGAYSSGDVGVIAYGPCSGDISDCYYFGCCGIAKEFVCQKSHQTTEISFLTSTGKPHPRNTGFSYLRDSYVHLPIRCKITISDKQYRVHAALAEATSAMTFSIFCKGKNCQVVDGPRLRSCSLDSYKGPGNDIMILGENDAINIVSASPYMEIFALQGKEKFWNADFLINIPYKEEGVMLIFEKKVTSEKGRFFTKMN

SEQ ID NO: 74 - protein, 369 aa, Artificial Sequence
  Description of Artificial Sequence  Synthetic Peptide
MTKIAFSEKAKNFPVEALKKWFEKNKRSLPWRDNPTPYSVWVSEVMLQQTRAEVVIDYFNQWMERFPTIESLAAAKEEDVIKLWEGLGYYSRARHLLEGARMVMEEFHGKIPDDAISLAQIRGVGPYTVHAILAFAFKRRAAAVDGNVLRVLSRIFLIETSIDLESTRTWVSRIAQALLPHKSPEVIAEALIELGACICKKVPQCHRCPVRQACGAWRENKQFVLPVRHARKKVIFLHRLVAIVLYDGSLVVEKRRPKEMMAGLYEFPYIEVEPEEGLQDIEGFTKKMELSLESPLEFLGNLKEQRHAFTNHKVHLCPIIFKATSLPQFGELHLLSDIDHLAFSSGHKKIKDALLIYLGDVRSRESIGV

SEQ ID NO: 75 - protein, 579 aa, Artificial Sequence
  Description of Artificial Sequence  Synthetic Peptide
MAVSGGGGVQPSSDPGKWNPALQGEQAEGPSPLKESIFSETKQASSAAKQESLVRSGSTGMYATESQINKAKYRKAQDRSSTSPKSKLKGTFSKMRASVQGFMSGFGSRASRVSAKRASDSGEGTSLLPTEMDVALKKGNRISPEMQGFFLDASGMGGSSSDISQLSLEALKSSAFSGARSLSLSSSESSSVASFGSFQKAIEPMSEEKVNAWTVARLGGEMVSSLLDPNVETSSLVRRAMATGNEGMIDLSDLGQEEVSTAMTSPRAVEGKVKVSSSDSPEANPTGIPNSNTLERAEKEAEKQESREQLSEDQMMLARAMAGLLTGAAPQEVLSNSVWSGPSTVFPPPKFSGTLPTQRSGDKSKHKSPGIEKSTNHTNFSPLREGTVKSAEVKSLPHPESMYRFPKDSIVSREEPEAVVKESTAFKNPENSSQNFLPIAVESVFPKESGTGGALGSDAVSSSYHFLAQRGVSLLAPLPRATDDYKEKLEAHKGPGGPPDPLIYQYRNVAVEPPIVLRSPQPFSGSSRLSVQGKPEAASVHDDGGGGNSGGFSGDQRRGSSGQKASRQEKKGKKLSTDI

SEQ ID NO: 76 - protein, 1142 aa, Artificial Sequence
  Description of Artificial Sequence  Synthetic Peptide
MKRRSWLKILGICLGSSIVLGFLIFLPQLLSTESGKYLVFSLIHKESGLSCSAEELKISWFGRQTARKIKLTGEAKDEVFSAEKFELDGSLLRLLIYKKPKGITLSGWSLKINEPASIDHPSVSHLDPGSLLTYLNDCKIISEHGFITMKTVSGSSLSVSGFYLEKSSEKFMTKCVVSEDQQSGNIFIESVLSPDVSISAQFSSVPVAFFKIFIASPFWDHLLSYEDIINLSAEATHTNDGKISMTASGEGNQIQMKLQGHIHKSTFYIVEGSSSFIELKPELASALCNQIIPLSTPITSKQIHATVSYAKIPLDITKWKHIEITSQAQLPEVAIHPKDPNLALQLRDTKLGIKKTEKFSDIRYSSSTVLGGASPSHLNGLISIDNKKHLTKFRLQQAQLPHTYLRAIFPQPFVINVPLDVAYYSLNIEGTYKNAHLEADAILDNPLLKLSCSMSGAWKNFLFKGQGTYHFNKKWQEILSPHFSYAEARFSGKAQITDTNLFFPKFSGKITARENELLIHAKFGSPNEPIKPETTSILIHGQFCSLPLSLVSNHLAPFHLKKLTFSFHTDGGKFVTKGNLQALIENPDYPDLNNTRILIPDLLLSLDESSTSPSSKDLKIQGSGEIFSLPLDSITKTYGKQVRLSPYFGSSGDLNFVVNYNPKDQNKLTLLSNFKSEALLGELKLVMDFSMKLSSGTQGTLQWEVSPERYASFFKNASCSPTCLLHRTANVRLDISKLSCPEETKGLSCLTLLAAGGLEGSLEATPLIFYDNVSKETFIINDFKGSLRANNLDAKIEYDLKGSCLAPRQDSKTLAEFSLEGQVDHLFSPESREFKQTANWIHIPSSFIAGIIPMSPGLKAQISSLAGPRINVSIKNAFRFGEGPVDIMVDSENLQAQIPLILNEKSILLRENLTAHLSINEDVNKAFLQEFNPLLAGGAYSQYPVTLEIDKQNFYLPIRPYSFEEFRIQSATLDMGKISIANTGTMYALFQFLDITDQKQFVESWFTPIFFSVQKGSIICKRXDALIDRRIRLALWGKTDIAHDRLFMTLGIDPEVIKKYFHNTSLKTKNFFLIKIRGSISSPEVDWSSAYARIALLKSYSLGNPFSSLADKLFSSLGDSTPPPTVHPFPWEKSNFDSIENK

SEQ ID NO: 77 - protein, 390 aa, Artificial Sequence
  Description of Artificial Sequence  Synthetic Peptide
MSSPVNNTPSAPNIPIPAPTTPGIPTTKPRSSFIEKVIIVAKYILFAIAATSGALGTILGLSGALTPGIGIALLVIFFVSMVLLGLILKDSISGGEERRLREEVSRFTSENQRLTVITTTLETEVKDLKAAKDQLTLEIEAFRNENGNLKTTAEDLEEQVSKLSEQLEALERINQLIQANAGDAQEISSELKKLISGWDSKVVEQINTSIQALKVLLGQEWVQEAQTHVKAMQEQIQALQAEILGMHNQSTALQKSVENLLVQDQALTRVVGELLESENKLSQACSALRQEIEKLAQHETSLQQRIDAMLAQEQNLAEQVTALEKMKQEAQKAESEFIACVRDRTFGRRETPPPTTPVVEGDESQEEDEGGTPPVSQPSSPVDRATGDGQ

SEQ ID NO: 78 - protein, 820 aa, Artificial Sequence
  Description of Artificial Sequence  Synthetic Peptide
MRYDPNLIEKKWQQFWKEHRSFQANEDEDKVKYYVLDMFPYPSGAGLHVGHLIGYTATDIVARYKRARGFSVLHPMGWDSFGLPAEQYAIRTGTHPKVTTQKNIANFKKQLSAMGFSYDEGREFATSDPDYYHWTQKLFLFLYDQGLAYMADMAVNYCPELGTVLSNEEVENGFSIEGGYPVERKMLRQWILKITAYADKLLEGLDALDWPENVKQLQKNWIGKSEGALVTFHLTQEGSLEAFTTRLDTLLGVSFLVIAPEHPDLDSIVSEEQRDEVTAYVQESLRKSERDRISSVKTKTGVFTGNYAKHPITGNLLPVWISDYVVLGYGTGVVMGVPAHDERDREFAEMFSLPIHEVIDDNGVCIHSNYNDFCLNGLSGQEAKDYVINYLEMRSLGRAKTMYRLRDWLFSRQRYWGEPIPIIHFEDGTHRPLEDDELPLLPPNIDDYRPEGFGQGPLAKAQDWVHIYDEKTGRPGCRETYTMPQWAGSCWYYLRFCDAHNSQLPWSKEKESYWMPVDLYIGGAEHAVLHLLYSRFWHRVFYDAGLVSTPEPFKKLINQGLVLASSYRIPGKGYVSIEDVREENGTWISTCGEIVEVRQEKMSKSKLNGVDPQVLIEEYGADALRMYAMFSGPLDKNKTWSNEGVGGCRRFLNRFYDLVTSSEVQDIEDRDGLVLAHKLVFRITEHIEKMSLNTIPSSFMEFLNDFSKLPVYSKRALSMAVRVLEPIAPHISEELWVILGNPPGIDQAAWPQIDESYLVAQTVTFVVQVNGKLRGRLEVAKEAPKEEVLSLSRSVVAKYLENAQIRKEIYVPNKLVNFVL

SEQ ID NO: 79 - protein, 1397 aa, Artificial Sequence
  Description of Artificial Sequence  Synthetic Peptide
LEKIMFGENSRDIGVLSKEGLFDKLEIGIASDITIRDKWSCGEIKKPETINYRTFKPEKGGLFCEKIFGPTKDWECCCGKYKKIKHKGIVCDRCGVEVTLSKVRRERMAHIELAVPIVHIWFFKTTPSRIGNVLGMTASDLERVIYYEEYVVIDPGKTDLTKKQLLNDAQYREVVEKWGKDAFVAKMGGEAIYDLLKSEDLQSLLKDLKERLRKTKSQQARMKLAKRLKIIEGFVSSSNHPEWMVLKNIPVVPPDLRPLVPLDGGRFATSDLNDLYRRVINRNNRLKAILRLKTPEVIVRNEKRMLQEAVDALFDNGRHGHPVMGAGNRPLKSLSEMLKGKNGRFRQNLLGKRVDYSGRSVIIVGPELKFNQCGLPKEMALELFEPFIIKRLKDQGSVYTIRSAKKMIQRGAPEVWDVLEEIIKGHPVLLNRAPTLHRLGIQAFEPVLIEGKAIRIHPLVCAAFNADFDGDQMAVHVPLSVEAQLEAKVLMMAPDNIFLPSSGKPVAIPSKDMTLGLYYLMADPTYFPEEHGGKTKIFKDEIEVLRALNNGGFIDDVFGDRRDETGRGIHIHEKIKVRIDGQIIETTPGRVLFNRIVPKELGFQNYSMPSKRISELILQCYKKVGLEATVRFLDDLKDLGFIQATKAAISMGLKDVRIPDIKSHILKDAYDKVAIVKKQYDDGIITEGERHSKTISIWTEVSEQLSDALYVEISKQTRSKHNPLFLMIDSGARGNKSQLKQLGALRGLMAKPNGAIIESPITSNFREGLTVLEYSISSHGARKGLADTALKTADSGYLTRRLVDVAQDVIITEKDCGTLNHIEISAIGQGSEELLPLKDRIYGRTVAEDVYQPGDKSRLLAQSGDVLNSVQAEAIDDAGIETIKIRSTLTCESPRGVCAKCYGLNLANGRLIGMGEAVGIIAAQSIGEPGTQLTMRTFHLGGIAATSSTPEIITNSDGILVYMDLRVVLGQEGHNLVLNKKGALHVVGDEGRTLNEYKKLLSTKSIESLEVFPVELGVKILVADGTPVSQGQRIAEVELHNIPIICDKPGFIKYEDLVEGISTEKVVNKNTGLVELIVKQHRGELHPQIAIYDDADLSELVGTYAIPSGAIISVEEGQRVDPGMLLARLPRGAIKTKDITGGLPRVAELVEARKPEDAADIAKIDGVVDFKGIQKNKRILVVCDEMTGMEEEHLIPLTKHLIVQRGDSVIKGQQLTDGLVVPHEILEICGVRELQKYLVNEVQEVYRLQGVDINDKHIEIIVRQMLQKVRITDPGDTTLLFGEDVNKKEFYEENRRTEEDGGKPAQAVPVLLGITKASLGTESFISAASFQDTTRVLTDAACCSKTDYLLGFKENVIMGHMIPGGTGFETHKRIKQYLEKEQEDLVFDFVSETECVC

SEQ ID NO: 80 - protein, 571 aa, Artificial Sequence
  Description of Artificial Sequence  Synthetic Peptide
MDTQSSIGNEEWRIAGTSVVSGMALGKVFFLGTSPLHVRELTLPQEEVEHEIHRYYKALNRSKSDIVALEQEVTGQQGLQEVSSILQAHLEIMKDPLLTEEVVNTIRKDRKNAEYVFSSVMGKIEESLTAVRGMPSVVDRVQDIHDISNRVIGHLCCQHKSSLGESDQNLIIFSEELTPSEVASANSAYIRGFVSLVGAATSHTAIVSRAKSIPYLANISEELWNIAKRYNGKLVLIDGYRGELIFNPKPATLQSCYKKELSVVAHTSQRLVRKSLHPIVSSHAGSDKDVEDLLENFPQTSIGLFRSEFLAVILGRLPTLREQVDLYEKLARFPGDSPSVLRLFDFGEDKPCPGIKNKKERSIRWLLDYSVILEDQLQAIAKASLQGSIKVLIPGVSDVSEIIEVKKKWETIQTRFPKGHKVSWGTMIEFPSAVWMIEEILPECDFLSIGTNDLVQYTLGISRESALPKHLNVTLPPAVIRMIHHVLQAAKQNQVPVSICGEAAGQLSLTPLFIGLGVQELSVAMPVINRLRNHIALLELNSCLEITEALLQAKTCSEVEELLNRNNKITS

SEQ ID NO: 81 - protein, 460 aa, Artificial Sequence
  Description of Artificial Sequence  Synthetic Peptide
MRQEKDSLGIVEVPEDKLYGAQTMRSRNFFSWGPELMPYEVIRALVWIKKCAAQANQDLGFLDSKHCDMIVAAADEILEGGFEEHFPLKVWQTGSGTQSNMNVNEVIANLAIRHHGGVLGSKDPIHPNDHVNKSQSSNDVFPTAMHIAAVISLKNKLIPALDHMIRVLDAKVEEFRHDVKIGRTHLMDAVPMTLGQEFSGYSSQLRHCLESIAFSLAHLYELAIGATAVGTGLNVPEGFVEKIIHYLRKETDEPFIPASNYFSALSCHDALVDAHGSLATLACALTKIATDLSFLGSGPRCGLGELFFPENEPGSSIMPGKVNPTQCEALQMVCAQVLGNNQTVIIGGSRGNFELNVMKPVIIYNFLQSVDLLSEGMRAFSEFFVKGLKVNKARLQDNINNSLMLVTALAPVLGYDKCSKAALKAFHESISLKEACLALGYLSEKEFDRLVVPENMVGNH

SEQ ID NO: 82 - protein, 238 aa, Artificial Sequence
  Description of Artificial Sequence  Synthetic Peptide
MGLYDRDYIQDSRVQGTFASRVYGWMTAGLIVTSCVALGLYFSGLYRSLFSFWWVWCFATLGVSFFINSKIQTLSVSAVGGLFLLYSTLEGMFFGTLLPVYAAQYGGGVIWAAFGSAALVFGLAAVYGAFTKSDLTKISKIMTFALIGLLLVTLVFAVVSMFVSMPLIYLLICYLGLVIFVGLTAADAQAIRRISSTIGDNNTLSYKLSLMFALKMYCNVIMVFWYLLQIFSSSGNRD

SEQ ID NO: 83 - protein, 1609 aa, Artificial Sequence
  Description of Artificial Sequence  Synthetic Peptide
MVAKKTVRSYRSSFSHSVIVAILSAGIAFEAHSLHSSELDLGVFNKQFEEHSAHVEEAQTSVLKGSDPVNPSQKESEKVLYTQVPLTQGSSGESLDLADANFLEHFQHLFEETTVFGIDQKLVWSDLDTRNFSQPTQEPDTSNAVSEKISSDTKENRKDLETEDPSKKSGLKEVSSDLPKSPETAVAAISEDLEISENISARDPLQGLAFFYKNTSSQSISEKDSSFQGIIFSGSGANSGLGFENLKAPKSGAAVYSDRDIVFENLVKGLSFISCESLEDGSAAGVNIVVTHCGDVTLTDCATGLDLEALRLVKDFSRGGAVFTARNHEVQNNLAGGILSVVGNKGAIVVEKNSAEKSNGGAFACGSFVYSNNENTALWKENQALSGGAISSASDIDIQGNCSAIEFSGNQSLIALGEHIGLTDFVGGGALAAQGTLTLRNNAVVQCVKNTSKTHGGAILAGTVDLNETISEVAFKQNTAALTGGALSANDKVIIANNFGEILFEQNEVRNHGGAIYCGCRSNPKLEQKDSGENINIIGNSGAITFLKNKASVLEVMTQAEDYAGGGALWGHNVLLDSNSGNIQFIGNIGGSTFWIGEYVGGGAILSTDRVTISNNSGDVVFKGNKGQCLAQKYVAPQETAPVESDASSTNKDEKSLNACSHGDHYPPKTVEEEVPPSLLEEHPVVSSTDIRGGGAILAQHIFITDNTGNLRFSGNLGGGEESSTVGDLAIVGGGALLSTNEVNVCSNQNVVFSDNVTSNGCDSGGAILAKKVDISANHSVEFVSNGSGKFGGAVCALNESVNITDNGSAVSFSKNRTRLGGAGVAAPQGSVTICGNQGNIAFKENFVFGSENQRSGGGAIIANSSVNIQDNAGDILFVSNSTGSYGGAIFVGSLVASEGSNPRTLTITGNSGDILFAKNSTQTAASLSEKDSFGGGAIYTQNLKIVKNAGNVSFYGNRAPSGAGVQIADGGTVCLEAFGGDILFEGNINFDGSFNAIHLCGNDSKIVELSAVQDKNIIFQDAITYEENTIRGLPDKDVSPLSAPSLIFNSKPQDDSAQHHEGTIRFSRGVSKIPQIAAIQEGTLALSQNAELWLAGLKQETGSSIVLSAGSILRIFDSQVDSSAPLPTENKEETLVSAGVQINMSSPTPNKDKAVDTPVLADIISITVDLSSFVPEQDGTLPLPPEIIIPKGTKLHSNAIDLKIIDPTNVGYENHALLSSHKDIPLISLKTAEGMTGTPTADASLSNIKIDVSLPSITPATYGHTGVWSESKMEDGRLVVGWQPTGYKLNPEKQGALVLNNLWSHYTDLRALKQEIFAHHTIAQRMELDFSTNVWGSGLGVVEDCQNIGEFDGFKHHLTGYALGLDTQLVEDFLIGGCFSQFFGKTESQSYKAKNDVKSYMGAAYAGILAGPWLIKGAFVYGNINNDLTTDYGTLGISTGSWIGKGFIAGTSIDYRYIVNPRRFISAIVSTVVPFVEAEYVRIDLPEISEQGKEVRTFQKTRFENVAIPFGFALEHAYSRGSRAEVNSVQLAYVFDVYRKGPVSLITLKDAAYSWKSYGVDIPCKAWKARLSNNTEWNSYLSTYLAFNYEWREDLIAYDFNGGIRIIF

SEQ ID NO: 84 - protein, 253 aa, Artificial Sequence
  Description of Artificial Sequence  Synthetic Peptide
MLIKLWRATYEGMYTFLVGALLKLRYRMQVEGWDTLNINPKQGCLFLANHVAEVDPIILEYLFWSRFHVRPMAVEYLFHSRVVQWFLNSVRSIPIPQLVPGKESKRSLERMNVCYEEASRALNRGESLLLYPSGRLSRTGKEEIVNQYSAYVLLHRVMECNVVLVRVSGLWGSAFSRYKQNSTPKLGPAFKEAFRALLRRGIFFMPKRFVKITLCQVDHLFLKQFPTKQDLNTFLASWFNQGDDNLPIEVPYA

SEQ ID NO: 85 - protein, 665 aa, Artificial Sequence
  Description of Artificial Sequence  Synthetic Peptide
MINKELDIGILGKIAGAIKQISIESIQKASSGHPGLPLGCAELAAYLYGYVLRQNPRDPHWINRDRFVLSAGHGSXLLYSCLHLAGFDVSLEDLQEFRQLHSRTPGHPEYGETVGVEATTGPLGQGLGNAVGMALSMKMLESRFNRPGHEIFNGKIYCLAGDGCFMEGVSHEVCSFAGSLNLNNLVVIYDYNNVVLDGYLNEISVEDTKKRFEAYGWDVYEIDGYDFTHIHETFSSIKRGQERPVLVIAHTIIGHGSPKEGTNKAHGSPLGVEGTHETKQFWHLPEEKFFVPPAVKNFFAHKIQEDRKAQEQWLDEVRVWSKQFPELHEEFVALTSHKLPKNLESLVQSVEMPDSIAGRAASNKLIQVLVQHIPYLIGGSADLSSSDGTWIANEKVIHTYDFSGRNIKYGVREFGMATIMNGLAYSQVFRPFGGTFLVFSDYMRNAIRLAALSKLPVIYQFTHDSIFVGEDGPTHQPVEQLMSLRAIPGLYVIRPADANEVRGAWIAGLKHTGPTVIVLSRQALPTLPAAHRPFKDGVGRGAYIVLKESGEKPDYTLFATGSEVSLALSVAKELEHLDKQVRVVSFPCWELFEAQDVDYKQSIVGGDLGIRVSIEAGSALGWYKYIGSEGLAIAMDRFGYSGASDDVSEECGFTTEQILQRILSQ

SEQ ID NO: 86 - protein, 401 aa, Artificial Sequence
  Description of Artificial Sequence  Synthetic Peptide
MSTMQNCPHFGVCGGCSFPQSNYSDSLKKKEELLHQLFAPLVPSDMIAPIIPCSPSLRGRNKMEFSFFQTYEGEKSLGFISSTKPKKGIPVTTCLLIHEQTMDILKLTREWWDKHPELMAYFPPKNKGSLCTLTVRTGSPQQNFMVILTTSGTPEYRVNEACIDEWKEILLSSSLNIASIYWEEKVAARGISTYYETKLLYGAPSIQQKLSLPSDGNSASFSLRPRSFFQPQITQAAKIIETAKEFINPEGSETLLDLYCGAGTIGIMLSPYVKNVIGVEIIPDAVASAQENIKANNKEDCVEVYLEDAKAFCKRNENCKAPDVIIIDPPRCGMQSKVLKYILRIGSPKIVYISCNPKTQFQECADLISGGYRIKKMQPIDQFPYSTHLENIILLEREIDL

SEQ ID NO: 87 - protein, 444 aa, Artificial Sequence
  Description of Artificial Sequence  Synthetic Peptide
MTSGVSGSSSQDPTLAAQLAQSSQKAGNAQSGHDTKNVTKQGAQAEVAAGGFEDLIQDASAQSTGKKEATSSTTKSSKGEKSEKSGKSKSSTSVASASETATAQAVQGPKGLRQNNYDSPSLPTPEAQTINGIVLKKGMGTLALLGLVMTLMANAAGESWKASFQSQNQAIRSQVESAPAIGEAIKRQANHQASATEAQAKQSLISGIVNIVGFTVSVGAGIFSAAKGATSALKSASFAKETGASAAGGAASKALTSASSSVQQTMASTAKAATTAASSAGSAATKAAANLTDDMAAAASKMASDGASKASGGLFGEVLNKPNWSEKVSRGMNVVKTQGARVASFAGNALSSSMQMSQLMHGLTAAVEGLSAGQTGIEVAHHQRLAGQAEAQAEVLKQMSSVYGQQAGQAGQLQEQAMQSFNTALQTLQNIADSQTQTTSAIFN

SEQ ID NO: 88 - protein, 674 aa, Artificial Sequence
  Description of Artificial Sequence  Synthetic Peptide
MSIVRNSALPLPCLSRSETFKKVRSHMKFMKVLTPWIYRKDLWVTAFLLTAIPGSFAHTLVDIAGEPRHAAQATGVSGDGKIVIGMKVPDDPFAITVGFQYIDGHLQPLEAVRPQCSVYPNGITPDGTVIVGTNYAIGMGSVAVKWVNGKVSELPMLPDTLDSVASAVSADGRVIGGNRNINLGASVAVKWEDDVITQLPSLPDAMNACVNGISSDGSIIVGTMVDVSWRNTAVQWIGDQLSVIGTLGGTTSVASAISTDGTVIVGGSENADSQTHAYAYKNGVMSDIGTLGGFYSLAHAVSSDGSVIVGVSTNSEHRYHAFQYADGQMVDLGTLGGPESYAQGVSGDGKVIVGRAQVPSGDWHAFLCPFQAPSPAPVHGGSTVVTSQNPRGMVDINATYSSLKNSQQQLQRLLIQHSAKVESVSSGAPSFTSVKGAISKQSPAVQNDVQKGTFLSYRSQVHGNVQNQQLLTGAFMDWKLASAPKCGFKVALHYGSQDALVERAALPYTEQGLGSSVLSGFGGQVQGRYDFNLGETVVLQPFMGIQVLHLSREGYSEKNVRFPVSYDSVAYSAATSFMGAHVFASLSPKMSTAATLGVERDLNSHIDEFKGSVSAMGNFVLENSTVSVLRPFASLAMYYDVRQQQLVTLSVVMNQQPLTGTLSLVSQSSYNLSF

SEQ ID NO: 89 - protein, 609 aa, Artificial Sequence
  Description of Artificial Sequence  Synthetic Peptide
MFRCILFGIFLLTCFSSGGVLYYLFCSHDFSIGPKEKSRSVWIEEEKEFTDSVLHHLPSQHQHLHILCFQGFLLQKQQKFSQAEKIFSKVYDEAQDGPFLFKEEILGSRLINSFFLEKTDVMETILCLLNQRCPNSPYYHLFKALVCYKQKLYREVIEQLAYWQEEKTRALAPLLNISIEQLLTDFLLDYISAHSLIEQKMFPEGRVILNRNINRLLKHECEWNAKTYDRIAILLSRSYFLELVESKSADIYFDYYEMVLFYLKKIYILEQCPYAELLPEEELVSLIMEHVFILPKDKLYPLIQLLEMWQKHYVHPNSSLVVQILVDRFSTHMEGAIRFCEALVSFSGLEELHQQIITTFEELLSNKVQQIKTEEAKQCVALLHILDPSISISEKLALSSDTLQNIVSGDDEQHTKLRNYLDLWEAIQSYDIDRQQLVHHLVYGAKDLWKKGGNDEKALNLLQLVLRFTSYDIECESVVFLFIKQAYKQALSSHAIARLLKLEKFISEANIPSIVISEAEKANFLADAEYLFAHEDYDKCYLYSMWLTKVAPSPQSYRLAGLCLMENKRYDEALEFLCMLSPNNSINDYKTQKALAFCQKHQSKDRAAS

SEQ ID NO: 90 - protein, 531 aa, Artificial Sequence
  Description of Artificial Sequence  Synthetic Peptide
MLGKEEEFTCKQKQCLSHFVTNLTSDVFALKNLPEVVKGALFSKYSRSVLGLRALLLKEFLSNEEDGDVCDEAYDFETDVQKAADFYQRVLDNFGDDSVGELGGAHLAMENVSILAAKVLEDARIGGSPLEKSTRYVYFDQKVRGEYLYYRDPILMTSAFKDMFLGTCDFLFDTYSALIPQVRAYFEKLYPKDSKTPASAYATSLRAKVLDCIRGLLPAATLTNLGFFGNGRFWQNLIHKLQGHNLAELRRLGDESLTELMKVIPSFVSRAEPHHHHHQAMMQYRRALKEQLKGLAEQATFSEEMSSSPSVQLVYGDPDGIYKVAAGFLFPYSNRSLTDLIDYCKKMPHEDLVQILESSVSARENRRHKSPRGLECVEFGFDILADFGAYRDLQRHRTLTQERQLLSTHHGYNFPVELLDTPMEKSYREAMERANETYNEIVQEFPEEAQYMVPMAYNIRWFFHVNARALQWICELRSQPQGHQNYRTIATGLVREVVKFNPMYELFFKFVDYSDIDLGRLNQEMRKEPTT

SEQ ID NO: 91 - protein, 31 aa, Artificial Sequence
  Description of Artificial Sequence  Synthetic Peptide
RVMKAVVSHKSRTSSIHRQYSSYSLFYSILK

SEQ ID NO: 92 - protein, 33 aa, Artificial Sequence
  Description of Artificial Sequence  Synthetic Peptide
DGVNFGNLFQPCPYCRGKYPSPTCTSTLSPSSS

SEQ ID NO: 93 - protein, 30 aa, Artificial Sequence
  Description of Artificial Sequence  Synthetic Peptide
GLRRFCKRYSIVVSESGEPFCLLKKKKIFL

SEQ ID NO: 94 - protein, 101 aa, Artificial Sequence
  Description of Artificial Sequence  Synthetic Peptide
NFPICDRSSRFRGDCRDEDLCGRNRYEAFPDDKTEGHLCSCDTLALSKYCCLHSHGIWWIDSHASSPCCFCRIGGCFCNTPYGFLRIFLRRLPTESKIEYQ

SEQ ID NO: 95 - protein, 21 aa, Artificial Sequence
  Description of Artificial Sequence  Synthetic Peptide
FLPVLPGLLLGPPLPQMSFRL

SEQ ID NO: 96 - protein, 63 aa, Artificial Sequence
  Description of Artificial Sequence  Synthetic Peptide
FFIKYSLSNGYGIQKYLQTRLSAIPEWHFSGTNTSSKIKKLCEELSQNCSYHRSTGILGLRSS

SEQ ID NO: 97 - protein, 69 aa, Artificial Sequence
  Description of Artificial Sequence  Synthetic Peptide
CSYSVYSLWSLLQLLMLMKSEKKKSKFFNVSHHFQKVLRLSEDNMVQERFKESRSLAIVKKRMLTKIPE

SEQ ID NO: 98 - protein, 12 aa, Artificial Sequence
  Description of Artificial Sequence  Synthetic Peptide
AQAFGSLLLRML

SEQ ID NO: 99 - protein, 25 aa, Artificial Sequence
  Description of Artificial Sequence  Synthetic Peptide
ELLISYQRKTSSAIGKKNFTTSSQC

SEQ ID NO: 100 - protein, 32 aa, Artificial Sequence
  Description of Artificial Sequence  Synthetic Peptide
EGREDLPSALRKGSPTSGNSNFRSAAYCGSCC

SEQ ID NO: 101 - protein, 33 aa, Artificial Sequence
  Description of Artificial Sequence  Synthetic Peptide
RLRSTTNTSQSSPQWDCTHPIYLCDVPHGSGYV

SEQ ID NO: 102 - protein, 20 aa, Artificial Sequence
  Description of Artificial Sequence  Synthetic Peptide
APQARGDTKIRGYRNRTRAC

SEQ ID NO: 103 - protein, 113 aa, Artificial Sequence
  Description of Artificial Sequence  Synthetic Peptide
GRLLKQCMLSSLRKWLAILQLFLIAQEKLRTLRLQILLLPSTLVKSKQALYHVLSVLQNTIDSWKLKKSLDPKQFSQILMYFLTRILRNRGIFSISILSPNQEYIADLWALSF

SEQ ID NO: 104 - protein, 20 aa, Artificial Sequence
  Description of Artificial Sequence  Synthetic Peptide
STNPTVALASIFDAKTTKCP

SEQ ID NO: 105 - protein, 55 aa, Artificial Sequence
  Description of Artificial Sequence  Synthetic Peptide
TDILVKFIKNIFPPLWRGNVVPRSKNMTSIYTHANGNLIFQILNEVTQFFKVTPN

SEQ ID NO: 106 - protein, 26 aa, Artificial Sequence
  Description of Artificial Sequence  Synthetic Peptide
SSRCTQREIAGRRTVNTPKPKRCMGS

SEQ ID NO: 107 - protein, 128 aa, Artificial Sequence
  Description of Artificial Sequence  Synthetic Peptide
IDATQINLNASQVDIHIRNHSSSYWSARTCMRRRVSPSTWSFIYKGEVWSWGRFPVNAFQAPNIIPNRTCFYFCSTTPCRGCISTTRFFSIPRTVDYSSFKFHAEHQMIVISCGSRALFHCRFYASRP

SEQ ID NO: 108 - protein, 49 aa, Artificial Sequence
  Description of Artificial Sequence  Synthetic Peptide
ALLSRACVASIIPARKSASIAICLPGIASNRNRAATSATRSAPLVTTIN

SEQ ID NO: 109 - protein, 48 aa, Artificial Sequence
  Description of Artificial Sequence  Synthetic Peptide
NKVITGSWRVGACESMETRKPPKCLKKKVDREKAVNIETIKVTAEGKS

SEQ ID NO: 110 - protein, 116 aa, Artificial Sequence
  Description of Artificial Sequence  Synthetic Peptide
GISVMMMLSRCLSSFSSTCRRARTLIFPRPVVYVERIPSEPQIIPPVGKSGPGMTCKISSTEACGFASRRSVASISSPKLCGGIFVAIPTAIPEEPLQRRFGNLEGKTTGSCFVSS

SEQ ID NO: 111 - protein, 148 aa, Artificial Sequence
  Description of Artificial Sequence  Synthetic Peptide
SGRIISVMLSAPPCELHSDLIDPDLFEFNHRLNICISAEVRGRVTTHTFRGDSCNMSFNCSVRGNVITIPRIIRIEIRSLTSSFSIITKCKRISSRLRITNIIAYWSLRYVCLRIDIKTVRECSSIKLRTFRRHITLHNKFTWRSRGI

SEQ ID NO: 112 - protein, 60 aa, Artificial Sequence
  Description of Artificial Sequence  Synthetic Peptide
SDRNSFSISVSFSKYADFIAPLNWSLRTRRAFNPVEAALIRFSHSSRVRPEVTVPEIRNS

SEQ ID NO: 113 - protein, 63 aa, Artificial Sequence
  Description of Artificial Sequence  Synthetic Peptide
MPWIFYKLFNINIGVIKTGFGFCTCGRKRSIEFVLFFNNTNSSSPTSSNGFNNNRETYFFSYF

SEQ ID NO: 114 - protein, 85 aa, Artificial Sequence
  Description of Artificial Sequence  Synthetic Peptide
ISMSSIETPSSPTTSIRLPISEPFSRNCAAFFTAPPQPETFSSKICPTWFKYFDQEIKGSIEGYLRASARAFERPQNAPTTANVD

SEQ ID NO: 115 - protein, 45 aa, Artificial Sequence
  Description of Artificial Sequence  Synthetic Peptide
GDLEHYKHIHGPFSKSSHHGKRHKRHNYLMFLQCRYKTLCGDQES

SEQ ID NO: 116 - protein, 29 aa, Artificial Sequence
  Description of Artificial Sequence  Synthetic Peptide
RVSFNASPPITTSRRDSKQEFCFFAVFRI

SEQ ID NO: 117 - protein, 86 aa, Artificial Sequence
  Description of Artificial Sequence  Synthetic Peptide
SINPLAPQRSLGPAIQYQLEEWPIQDTSISRDPKRLSSTNTSVSYRNASEIFSCNSSLSRTKNIPILDPKCAVFTIIGKENVSIRF

SEQ ID NO: 118 - protein, 45 aa, Artificial Sequence
  Description of Artificial Sequence  Synthetic Peptide
PLGPAPITTQSNSWVIFSLTVIKISKRTPLHKCMHLIKKRTLCLF

SEQ ID NO: 119 - protein, 69 aa, Artificial Sequence
  Description of Artificial Sequence  Synthetic Peptide
GEQPFLFHPTSQQSLSLYHHYIGTLQSSFHHWKHPLQKIAFPQKIFQDQQVHRLPTSQRGKTDLDPRFL

SEQ ID NO: 120 - protein, 95 aa, Artificial Sequence
  Description of Artificial Sequence  Synthetic Peptide
VTSKYASMRPSIPNPASHTRKIARAPTSRLTRFPYAGYISSKKYQGAPVSPQGRSEPSGNTMRFEESPGTHTRPPSPRTDSEINRSLSLPGIAVG

SEQ ID NO: 121 - protein, 11 aa, Artificial Sequence
  Description of Artificial Sequence  Synthetic Peptide
KLKLLLLLKLK

SEQ ID NO: 122 - protein, 36 aa, Artificial Sequence
  Description of Artificial Sequence  Synthetic Peptide
RLAGLLRKGGEKIGEKLKKIGQKIKNFFQKLVPQPE